(19) United States
(12) Patent Application Publication
(10) Pub. No.: US 2024/0018110 A1
(43) Pub. Date: Jan. 18, 2024

(54) RADIOLABELED COMPOUNDS TARGETING THE PROSTATE-SPECIFIC MEMBRANE ANTIGEN

(71) Applicants: THE UNIVERSITY OF BRITISH COLUMBIA, Vancouver (CA); PROVINCIAL HEALTH SERVICES AUTHORITY, Vancouver (CA); ALPHA-9 THERANOSTICS INC., Vancouver (CA)

(72) Inventors: François BÉNARD, Vancouver (CA); Kuo-Shyan LIN, Richmond (CA); Chengcheng ZHANG, Richmond (CA); David PERRIN, Vancouver (CA); Aron ROXIN, Vancouver (CA); Zhengxing ZHANG, Langley (CA); Antoine DOUCHEZ, Vancouver (CA); Pargol DANESHMANDKASHANI, Vancouver (CA); Samson LAI, Vancouver (CA)

(21) Appl. No.: 18/267,987

(22) PCT Filed: Dec. 16, 2021
(86) PCT No.: PCT/CA2021/051826

§ 371 Date: Jun. 16, 2023

Related U.S. Application Data
(60) Provisional application No. 63/126,448, filed on Dec. 16, 2020.

(51) Int. Cl. C07D 257/02; C07C 235/78; A61K 51/04

(52) U.S. Cl. CPC C07D 257/02; C07C 235/78; A61K 51/0482; A61K 2123/00; C07B 2200/07; A61K 2121/00; C07B 2200/05

(57) ABSTRACT

The present invention relates to radiolabelled compounds for in vivo imaging or treatment of diseases or conditions characterized by expression of prostate-specific membrane antigen.

### CROSS-REFERENCE TO RELATED APPLICATIONS

This application claims priority to U.S. Provisional Application No. 63/126,448, filed Dec. 16, 2020, the disclosures of which are hereby incorporated by reference in their entireties for all purposes.

### FIELD OF INVENTION

The present invention relates to radiolabelled compounds for in vivo imaging or treatment of diseases or conditions characterized by expression of prostate-specific membrane antigen.

### BACKGROUND OF THE INVENTION

Prostate-specific membrane antigen (PSMA) is a transmembrane protein that catalyzes the hydrolysis of N-acetyl-aspartylglutamate to glutamate and N-acetylaspartate. PSMA is selectively overexpressed in certain diseases and conditions compared to most normal tissues. For example, PSMA is overexpressed up to 1,000-fold in prostate tumors and metastases. Due to its pathological expression pattern, various radiolabeled PSMA-targeting constructs have been designed and evaluated for imaging of PSMA-expressing tissues and/or for therapy of diseases or conditions characterized by PSMA expression.

A number of radiolabeled PSMA-targeting derivatives of lysine-urea-glutamate (Lys-ureido-Glu) have been developed, including ¹⁸F-DCFBC, ¹⁸F-DCFPyL, ⁶⁸Ga-PSMA-HBED-CC, ⁶⁸Ga-PSMA-617, ⁶⁸Ga-PSMA I & T (see FIG. 1) as well as versions of the foregoing labelled with alpha emitters (such as ²²⁵Ac) or beta emitters (such as ¹⁷⁷Lu or ⁹⁰Y)

In clinical trials, PSMA-617 radiolabeled with therapeutic radionuclides, such as ¹⁷⁷L and ²²⁵Ac, has shown promise as an effective systemic treatment for metastatic castration resistant prostate cancer (mCRPC). However, dry mouth (xerostomia), altered taste and adverse renal events are common side effects of this treatment, due to high salivary gland and kidney accumulation of the radiotracer (Hofman et al., 2018 The Lancet 16(6):825-833; Rathke et al. 2019 Eur J Nucl Med Mol Imaging 46(1):139-147; Sathekge et al. 2019 Eur J Nucl Med Mol Imaging 46(1):129-138). Radiotracer accumulation in the kidneys and salivary gland is therefore a limiting factor that reduces the maximal cumulative administered activity that can be safely given to patients, which limits the potential therapeutic effectiveness of Lys-urea-Glu based radiopharmaceuticals (Violet et al. 2019 J Nucl Med. 60(4):517-523). There is therefore a need for new radiolabeled PSMA-targeting compounds, particularly compounds that have low accumulation in the salivary glands and/or kidneys, or other advantages.

No admission is necessarily intended, nor should it be construed, that any of the preceding information constitutes prior art against the present invention.

### SUMMARY

Various embodiments disclosed herein relate to compounds of Formulas A′, A, B′, B I-a, I-b, II, III-a, III-b, IV-a, and IV-b, and their use, when radiolabeled, in imaging and/or treating conditions or diseases characterized by expression of PSMA in a subject.

The present disclosure relates to compounds useful as imaging agents and/or therapeutic agents. In some embodiments, the compound of the present disclosure relates to a compound of Formula F:

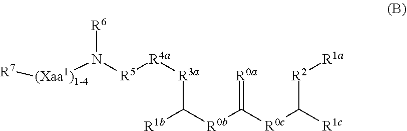

or a salt, a solvate, or a stereoisomer thereof, wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

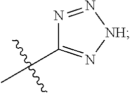 [SMILES: *c1nn[nH]n1]

R^{1b} is —CO₂H, —SO₂H, —SO₃H, —PO₂H, —PO₃H₂, —B(OH)₂, or

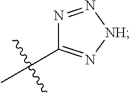 [SMILES: *c1nn[nH]n1]

R^{1c} is —CO₂H, —SO₂H, —SO₃H, —PO₂H, —PO₃H₂, —B(OH)₂, or

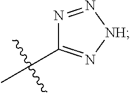 [SMILES: *c1nn[nH]n1]

R² is —CH₂—, —(CH₂)₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂—O—CH(CO₂H)—CH₂—, —CH₂—Se—CH(CO₂H)—CH₂—, —CH₂—CH(CO₂H)—(CH₂)₂—, —(CH₂)₂—CH(CO₂H)—, —CH₂—CH(CO₂H)—CH₂—, —(CH₂)₁₋₂—R^{3h}—(CH₂)₀₋₂—, —(CH₂)₀₋₂—R^{3h}—(CH₂)₁₋₂— or —(CH₂)₁₋₃—NH—C(O)—C(R^{3b})₂—;
R^{3h} is

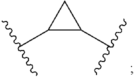 [SMILES: *C1CC1*]

each R^{3b} is, independently, hydrogen, methyl, or ethyl, or together—C(R^{3b})₂— forms cyclopropylenyl;
R⁴° is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, N₃, CN, SMe, CF₃, CHF₂, halogen, C₁-C₆ alkyl, or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
R⁷ is R^{X}-(Xaa²)₀₋₄-,

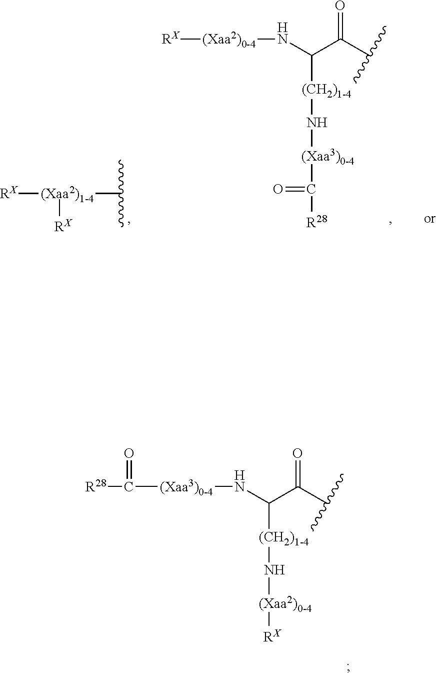

R²⁸ is an albumin binder;
Xaa² and Xaa³, when present, are each independently —N(R¹³)R¹⁴C(O)—, wherein each R¹³ is independently hydrogen or methyl, and wherein each R¹⁴ is, independently, a linear or branched, cyclic or acyclic, and/or aromatic or non-aromatic C₁-C₂₀ alkylenyl, alkenylenyl or alkynylenyl, or a linear or branched, cyclic or acyclic, and/or aromatic or non-aromatic C₂-C₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
wherein any one or any combination of amide linkages within R⁷-Xaa¹)₁₋₄-N(R⁶)—R⁵—R^{4a}—R^{3a} is optionally independently replaced by linkages selected from the group consisting of, —NHC(S)—, —C(S)NH—, —NHC(O)—,

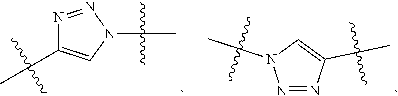 [SMILES: *c1cn(*)nn1 ; *c1cn(*)nn1]

—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

In some embodiments, the compound of the present disclosure relates to a compound of Formula A:

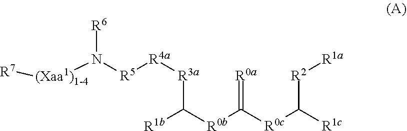

or a salt, a solvate, or a stereoisomer thereof, wherein:
   R^{0a} is O or S;
   R^{0b} is —O—, —S—, —NH—, or

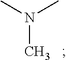

R^{0c} is —O—, —S—, —NH—, or

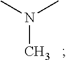

wherein at least one of R^{0b} and R^{0c} is not —NH—;
   R^{1a} is —CO₂H, —SO₂H, —SO₃H, —PO₂H, —PO₃H₂, —OPO₃H₂, —OSO₃H, —B(OH)₂, or

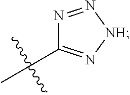 [SMILES: *c1nn[nH]n1]

R^{1b} is —CO₂H, —SO₂H, —SO₃H, —PO₂H, —PO₃H₂, —B(OH)₂, or

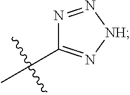 [SMILES: *c1nn[nH]n1]

R^{1c} is —CO₂H, —SO₂H, —SO₃H, —PO₂H, —PO₃H₂, —B(OH)₂, or

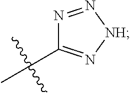 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein each R^{3a} is optionally substituted;
R^{4a} is —O—, —S—, —Se—, —S(O)—, —S(O)₂—,

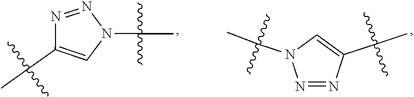 [SMILES: *c1cn(*)nn1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, halogen, C₁-C₆ alkyl, or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms;
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
R⁷ is R^{X}—(Xaa²)₀₋₄—,

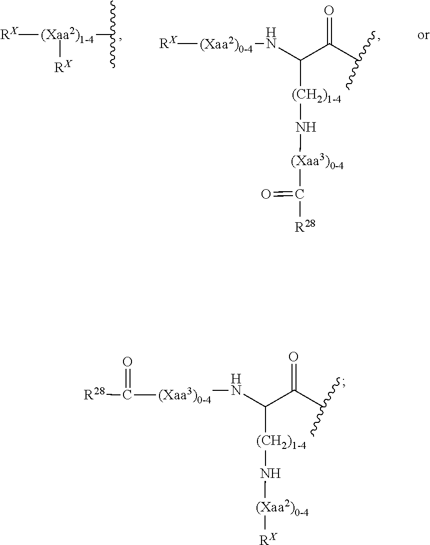

R²⁸ is an albumin binder;
Xaa² and Xaa³, when present, are each independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
wherein any one or any combination of amide linkages within R⁷-Xaa¹)₁₋₄-N(R⁶)—R⁵—R^{4a}—R^{3a} is optionally independently replaced by linkages selected from the group consisting of, —NHC(S)—, —C(S)NH—, —NHC(O)—,

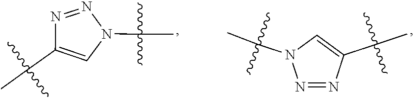 [SMILES: *c1cn(*)nn1 ; *c1cn(*)nn1]

—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

The present disclosure further relates to a method of treating a PSMA-expressing condition or disease, comprising administering to a patient in need thereof a therapeutically effective amount of a compound of the invention.

The present disclosure further relates to a method of imaging PSMA-expressing tissues comprising administering an effective amount of a compound of the invention to a patient in need of such imaging; and imaging the tissues of the subject.

### BRIEF DESCRIPTION OF THE DRAWINGS

The features of the invention will become apparent from the following description in which reference is made to the appended drawings wherein:

FIG. 1 shows examples of prior art PSMA-targeting compounds for prostate cancer imaging.

FIG. 2 shows PET image of ⁶⁸Ga—CCZ02011 in mice bearing LNCaP xenograft at 1 h p.i.

FIG. 3 shows PET image obtained at 1 h following the intravenous injection of ⁶⁸Ga—CCZ02018.

FIG. 4 shows PET image obtained at 1 h following the intravenous injection of ⁶⁸Ga—CCZ01194.

FIG. 5 shows PET image obtained at 1 h following the intravenous injection of ⁶⁸Ga-AR-113-1.

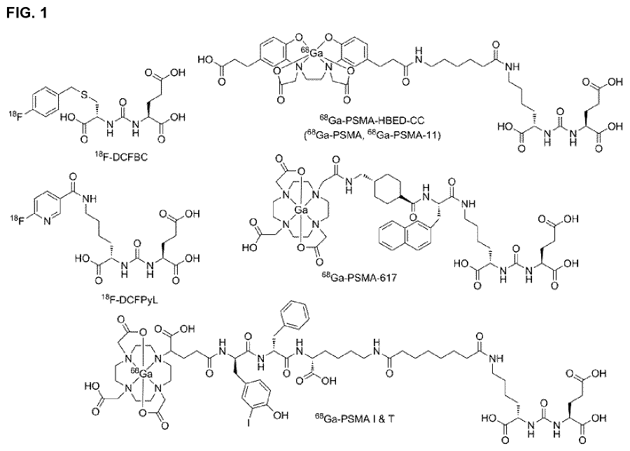

### DETAILED DESCRIPTION

As used herein, the terms “comprising,” “having”, “including” and “containing,” and grammatical variations thereof, are inclusive or open-ended and do not exclude additional, unrecited elements and/or method steps, even if a feature/component defined as a part thereof consists or consists essentially of specified feature(s)/component(s). The term “consisting essentially of” if used herein in connection with a compound, composition, use or method, denotes that additional elements and/or method steps may be present, but that these additions do not materially affect the manner in which the recited compound, composition, method or use functions. The term “consisting of” if used herein in connection with a feature of a composition, use or method, excludes the presence of additional elements and/or method steps in that feature. A compound, composition, use or method described herein as comprising certain elements and/or steps may also, in certain embodiments consist essentially of those elements and/or steps, and in other embodiments consist of those elements and/or steps, whether or not these embodiments are specifically referred to. A use or method described herein as comprising certain elements and/or steps may also, in certain embodiments consist essentially of those elements and/or steps, and in other embodiments consist of those elements and/or steps, whether or not these embodiments are specifically referred to.

A reference to an element by the indefinite article “a” does not exclude the possibility that more than one of the elements is present, unless the context clearly requires that there be one and only one of the elements. The singular forms “a”, “an”, and “the” include plural referents unless the context clearly dictates otherwise. The use of the word “a” or “an” when used herein in conjunction with the term “comprising” may mean “one,” but it is also consistent with the meaning of “one or more,” “at least one” and “one or more than one.”

In this disclosure, the recitation of numerical ranges by endpoints includes all numbers subsumed within that range including all whole numbers, all integers and, where suitable, all fractional intermediates (e.g., 1 to 5 may include 1, 1.5, 2, 2.75, 3, 3.80, 4, and 5 etc.).

Unless otherwise specified, “certain embodiments”, “various embodiments”, “an embodiment” and similar terms includes the particular feature(s) described for that embodiment either alone or in combination with any other embodiment or embodiments described herein, whether or not the other embodiments are directly or indirectly referenced and regardless of whether the feature or embodiment is described in the context of a method, product, use, composition, compound, etcetera.

As used herein, the terms “treat”, “treatment”, “therapeutic” and the like includes ameliorating symptoms, reducing disease progression, improving prognosis and reducing recurrence.

As used herein, the term “diagnostic agent” includes an “imaging agent”. As such, a “diagnostic radiometal” includes radiometals that are suitable for use as imaging agents.

The term “subject” refers to an animal (e.g. a mammal or a non-mammal animal). The subject may be a human or a non-human primate. The subject may be a laboratory mammal (e.g., mouse, rat, rabbit, hamster and the like). The subject may be an agricultural animal (e.g., equine, ovine, bovine, porcine, camelid and the like) or a domestic animal (e.g., canine, feline and the like). In some embodiments, the subject is a human.

The compounds disclosed herein may also include base-free forms, salts or pharmaceutically acceptable salts thereof. Unless otherwise specified, the compounds claimed and described herein are meant to include all racemic mixtures and all individual enantiomers or combinations thereof, whether or not they are explicitly represented herein.

The compounds disclosed herein may be shown as having one or more charged groups, may be shown with ionizable groups in an uncharged (e.g. protonated) state or may be shown without specifying formal charges. As will be appreciated by the person of skill in the art, the ionization state of certain groups within a compound (e.g. without limitation, CO₂H, PO₃H₂, SO₂H, SO₃H, SO₄H, OPO₃H₂ and the like) is dependent, inter alia, on the pKa of that group and the pH at that location. For example, but without limitation, a carboxylic acid group (i.e. COOH) would be understood to usually be deprotonated (and negatively charged) at neutral pH and at most physiological pH values, unless the protonated state is stabilized. Likewise, OSO₃H (i.e. SO₄H) groups, SO₂H groups, SO₃H groups, OPO₃H₂ (i.e. PO₄H₂) groups and PO₃H groups would generally be deprotonated (and negatively charged) at neutral and physiological pH values.

As used herein, the terms “salt” and “solvate” have their usual meaning in chemistry. As such, when the compound is a salt or solvate, it is associated with a suitable counter-ion. It is well known in the art how to prepare salts or to exchange counter-ions. Generally, such salts can be prepared by reacting free acid forms of these compounds with a stoichiometric amount of a suitable base (e.g. without limitation, Na, Ca, Mg, or K hydroxide, carbonate, bicarbonate, or the like), or by reacting free base forms of these compounds with a stoichiometric amount of a suitable acid. Such reactions are generally carried out in water or in an organic solvent, or in a mixture of the two. Counter-ions may be changed, for example, by ion-exchange techniques such as ion-exchange chromatography. All zwitterions, salts, solvates and counter-ions are intended, unless a particular form is specifically indicated.

In certain embodiments, the salt or counter-ion may be pharmaceutically acceptable, for administration to a subject. More generally, with respect to any pharmaceutical composition disclosed herein, non-limiting examples of suitable excipients include any suitable buffers, stabilizing agents, salts, antioxidants, complexing agents, tonicity agents, cryoprotectants, lyoprotectants, suspending agents, emulsifying agents, antimicrobial agents, preservatives, chelating agents, binding agents, surfactants, wetting agents, non-aqueous vehicles such as fixed oils, or polymers for sustained or controlled release. See, for example, Berge et al. 1977. (J. Pharm Sci. 66:1-19), or Remington-The Science and Practice of Pharmacy, 21st edition (Gennaro et al editors. Lippincott Williams & Wilkins Philadelphia), each of which is incorporated by reference in its entirety.

As used herein, a “stereoisomer” refers to a compound made up of the same atoms bonded by the same bonds but having different three-dimensional structures, which are not interchangeable. The present disclosure contemplates various stereoisomers and mixtures thereof and includes enantiomers and diastereomer.

As used herein, the expression “Xy-Xz”, where y and z are integers (e.g. X₁—X₁₅, X₁—X₃₀, X₁—X₁₀₀, and the like), refers to the number of carbons (for alkyls, whether saturated or unsaturated, or aryls) in a compound, R-group or substituent, or refers to the number of carbons plus heteroatoms (for heteroalkyls, whether saturated or unsaturated, or heteroaryls) in a compound, R-group or substituent. Heteroatoms may include any, some or all possible heteroatoms. For example, in some embodiments, the heteroatoms are selected from N, O, S, P and Se. In some embodiments, the heteroatoms are selected from N, O, S and P. Unless otherwise specified, such embodiments are non-limiting. When replacing a carbon with a heteroatom, it will be understood that the replacements only include those that would be reasonably made by the person of skill in the art. For example, —O—O— linkages are explicitly excluded. Such expressions are also intended to include replacement of one carbon, and replacement of multiple carbons, either with the same heteroatom (e.g. one of N, S, or O) or with a combination of different heteroatoms (e.g. combinations of N, S, and/or O in suitable configurations). Alkyls and aryls may alternatively be referred to using the expression “Cy-Cz”, where y and z are integers (e.g. C₃-C₁₅ and the like). Further, when the expression “Cy-Cz” is used in association with heteroalkyls, it is understood that one or more carbon atoms of Cy-Cz alkyl is replaced with a heteroatom, such as N, O, S, P and Se. For example, C₄ heteroalkyl can include CH₃CH₂SCH₃.

Unless explicitly stated otherwise, the terms “alkyl” and “heteroalkyl” each includes any reasonable combination of the following: (1) saturated alkyls as well as unsaturated (including partially unsaturated) alkyls (e.g. alkenyls and alkynyls); (2) linear or branched; (3) acyclic or cyclic (aromatic or nonaromatic), the latter of which may include multi-cyclic (fused rings, multiple non-fused rings or a combination thereof); and (4) unsubstituted or substituted. For example, an alkyl or heteroalkyl (i.e. “alkyl/heteroalkyl”) may be saturated, branched and cyclic, or unsaturated, branched and cyclic, or linear and unsaturated, or any other reasonable combination according to the skill of the person of skill in the art. If unspecified, the size of the alkyl/heteroalkyl is what would be considered reasonable to the person of skill in the art. For example, but without limitation, if unspecified, the size of an alkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons in length, subject to the common general knowledge of the person of skill in the art. Further, but without limitation, if unspecified, the size of a heteroalkyl may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 89, 90, 91, 92, 93, 94, 95, 96, 97, 98, 99, 100 or more than 100 carbons and heteroatoms in length, subject to the common general knowledge of the person of skill in the art. In the context of the expression “alkyl, alkenyl or alkynyl” and similar expressions, the “alkyl” would be understood to be a saturated alkyl. Likewise, in the context of the expression “heteroalkyl, heteroalkenyl or heteroalkynyl” and similar expressions, the “heteroalkyl” would be understood to be a saturated heteroalkyl.

As used herein, in the context of an alkyl/heteroalkyl group of a compound, the term “linear” may be used as it is normally understood to a person of skill in the art and generally refers to a chemical entity that comprises a skeleton or main chain that does not split off into more than one contiguous chain. Non-limiting examples of linear alkyls include methyl, ethyl, n-propyl, and n-butyl.

As used herein, the term “branched” may be used as it is normally understood to a person of skill in the art and generally refers to a chemical entity that comprises a skeleton or main chain that splits off into more than one contiguous chain. The portions of the skeleton or main chain that split off in more than one direction may be linear, cyclic or any combination thereof. Non-limiting examples of a branched alkyl group include tert-butyl and isopropyl.

As used herein the term cyclic alkyl/heteroalkyl refers to saturated, unsaturated, or partially unsaturated cycloalkyl and cycloheteroalkyl groups as well as combinations with linear or branched alkyl/heteroalkyl—for example: -(alkylene)₀₋₁-(cycloalkylene)-(alkylene)₀₋₁-, -(alkylene)₀₋₁-(cycloheteroalkylene)-(alkylene)₀₋₁-, -(alkylene)₀₋₁-(arylene)-(alkylene)₀₋₁-, and -(alkylene)₀₋₁-(heteroarylene)-(alkylene)₀₋₁-are included in said term. In an illustrative example, a divalent aromatic heteroalkyl group can be

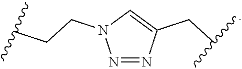 [SMILES: *CCn1cc(C*)nn1]

The term “alkylenyl” refers to a divalent analog of an alkyl group. In the context of the expression “alkylenyl, alkenylenyl or alkynylenyl”, “alkylenyl or alkenylenyl” and similar expressions, the “alkylenyl” would be understood to be a saturated alkylenyl. The term “heteroalkylenyl” refers to a divalent analog of a heteroalkyl group. In the context of the expression “heteroalkylenyl, heteroalkenylenyl or heteroalkynylenyl”, “heteroalkylenyl or heteroalkenylenyl” and similar expressions, the “heteroalkylenyl” would be understood to be a saturated heteroalkylenyl. The term “cyclopropyl-enyl” refers to a divalent analog of a cylcopropyl group, and may also be referred to using the notation —CH[CH₂]CH— to indicate that it is bonded at 2 separate carbons.

As used herein, the term “saturated” when referring to a chemical entity may be used as it is normally understood to a person of skill in the art and generally refers to a chemical entity that comprises only single bonds, and may include linear, branched, and/or cyclic groups. Non-limiting examples of a saturated C₁-C₂₀ alkyl group may include methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, I-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl, 3-methylpentyl, sec-hexyl, t-hexyl, n-heptyl, i-heptyl, sec-heptyl, t-heptyl, n-octyl, i-octyl, sec-octyl, t-octyl, n-nonyl, i-nonyl, sec-nonyl, t-nonyl, n-decyl, i-decyl, sec-decyl, t-decyl, cyclopropanyl, cyclobutanyl, cyclopentanyl, cyclohexanyl, cycloheptanyl, cyclooctanyl, cyclononanyl, cyclodecanyl, and the like. Unless otherwise specified, a C₁-C₂₀ alkylenyl therefore encompasses, without limitation, all divalent analogs of the above-listed saturated alkyl groups.

As used herein, the term “unsaturated” when referring to a chemical entity may be used as it is normally understood to a person of skill in the art and generally refers to a chemical entity that comprises at least one double or triple bond, and may include linear, branched, and/or cyclic groups. Non-limiting examples of a C₂-C₂₀ alkenyl group may include vinyl, allyl, isopropenyl, I-propene-2-yl, 1-butene-1-yl, I-butene-2-yl, I-butene-3-yl, 2-butene-1-yl, 2-butene-2-yl, octenyl, decenyl, cyclopropenyl, cyclobutenyl, cyclopentenyl, cyclohexenyl, cycloheptenyl, cyclooctenyl, cyclononanenyl, cyclodecanenyl, and the like. Unless otherwise specified, a C₁-C₂₀ alkenylenyl therefore encompasses, without limitation, all divalent analogs of the above-listed alkenyl groups. Non-limiting examples of a C₂-C₂₀ alkynyl group may include ethynyl, propynyl, butynyl, pentynyl, hexynyl, heptynyl, octynyl, nonynyl, decynyl, and the like. Unless otherwise specified, a C₁-C₂₀ alkynylenyl therefore encompasses, without limitation, all divalent analogs of the above-listed alkynyl groups. Without limitation, the above-defined saturated C₁-C₂₀ alkyl groups, C₂-C₂₀ alkenyl groups and C₂-C₂₀ alkynyl groups are all encompassed within the term “C₁-C₂₀ alkyl”, unless otherwise indicated. Without limitation, the above-defined saturated C₁-C₂₀ alkylenyl groups, C₂-C₂₀ alkenylenyl groups and C₂-C₂₀ alkynylenyl groups are all encompassed within the term “C₁-C₂₀ alkylenyl”, unless otherwise indicated.

Without limitation, the term “X₁-X₂₀ heteroalkyl” would encompass each of the above-defined saturated C₁-C₂₀ alkyl groups, C₂-C₂₀ alkenyl groups and C₂-C₂₀ alkynyl groups, where one or more of the carbon atoms is independently replaced with a heteroatom. Likewise, without limitation, the term “X₁-X₂₀ heteroalkylenyl” would encompass each of the above-defined saturated C₁-C₂₀ alkylenyl groups, C₂-C₂₀ alkenylenyl groups and C₂-C₂₀ alkynylenyl groups, where one or more of the carbon atoms is independently replaced with a heteroatom. The person of skill in the art would understand that various combinations of different heteroatoms may be used. Non-limiting examples of non-aromatic heterocyclic (can also be referred to as “non-aromatic, cyclic heteroalkyl” in this specification) groups include aziridinyl, azetidinyl, diazetidinyl, pyrrolidinyl, pyrrolinyl, piperidinyl, piperazinyl, imidazolinyl, pyrazolidinyl, imidazolydinyl, phthalimidyl, succinimidyl, oxiranyl, tetrahydropyranyl, oxetanyl, dioxanyl, thietanyl, thiepinyl, morpholinyl, oxathiolanyl, and the like.

Unless further specified, an “aryl” group includes both single aromatic rings as well as fused rings containing at least one aromatic ring. Non-limiting examples of C₃-C₂₀ aryl groups include phenyl (Ph), pentalenyl, indenyl, naphthyl and azulenyl. Non-limiting examples of X₃-X₂₀ aromatic heterocyclic groups (can also be referred to as “heteroaryls” or “aromatic cyclic heteroalkyl” in this specification) include pyrrolyl, imidazolyl, pyrazolyl, pyridinyl, pyridazinyl, pyrimidinyl, pirazinyl, quinolinyl, isoquinolinyl, acridinyl, indolyl, isoindolyl, indolizinyl, purinyl, carbazolyl, indazolyl, phthalazinyl, naphthyridinyl, quinoxalinyl, quinazolinyl, cinnolinyl, pteridinyl, phenanthridinyl, phenazinyl, phenanthrolinyl, perimidinyl, furyl, dibenzofuryl, xanthenyl, benzofuryl, thiophenyl, thianthrenyl, benzothiophenyl, phosphorinyl, phosphinolinyl, phosphindolyl, thiazolyl, oxazolyl, isoxazolyl, and the like. The expression “a linear, branched, and/or cyclic . . . alkylenyl, alkenylenyl or alkynylenyl” and similar expression include, inter alia, divalent analogs of the above-defined linear, branched, and/or cyclic alkyl, alkenyl or alkynyl groups, including all aryl groups encompassed therein.

As used herein, the term “substituted” is used as it would normally be understood to a person of skill in the art and generally refers to a compound or chemical entity that has one chemical group replaced with a different chemical group. Unless otherwise specified, a substituted alkyl is an alkyl in which one or more hydrogen atom(s) are independently each replaced with an atom that is not hydrogen. For example, chloromethyl is a non-limiting example of a substituted alkyl, more particularly an example of a substituted methyl. Aminoethyl is another non-limiting example of a substituted alkyl, more particularly an example of a substituted ethyl. Unless otherwise specified, a substituted compound or group (e.g. alkyl, heteroalkyl, aryl, heteroaryl and the like) may be substituted with any chemical group reasonable to the person of skill in the art. For example, but without limitation, a hydrogen bonded to a carbon or heteroatom (e.g. N) may be substituted with halide (e.g. F, I, Br, Cl), amine, amide, oxo, hydroxyl, thiol (sulfhydryl), phosphate (or phosphoric acid), phosphonate, sulfate, SO₂H (sulfinic acid), SO₃H (sulfonic acid), alkyls, heteroalkyls, aryl, heteroaryl, ketones, carboxaldehyde, carboxylates, carboxamides, nitriles, monohalomethyl, dihalomethyl or trihalomethyl.

As used herein, the term “unsubstituted” is used as it would normally be understood to a person of skill in the art. Non-limiting examples of unsubstituted alkyls include methyl, ethyl, tert-butyl, pentyl and the like. The expression “optionally substituted” is used interchangeably with the expression “unsubstituted or substituted”.

In the structures provided herein, hydrogen may or may not be shown. In some embodiments, hydrogens (whether shown or implicit) may be protium (i.e. ¹H), deuterium (i.e. ²H) or combinations of ¹H and ²H. Methods for exchanging ¹H with ²H are well known in the art. For solvent-exchangeable hydrogens, the exchange of ¹H with ²H occurs readily in the presence of a suitable deuterium source, without any catalyst. The use of acid, base or metal catalysts, coupled with conditions of increased temperature and pressure, can facilitate the exchange of non-exchangeable hydrogen atoms, generally resulting in the exchange of all ¹H to ²H in a molecule.

The term “Xaa” refers to an amino acid residue in a peptide chain or an amino acid that is otherwise part of a compound. Amino acids have both an amino group and a carboxylic acid group, either or both of which can be used for covalent attachment. In attaching to the remainder of the compound, the amino group and/or the carboxylic acid group may be converted to an amide or other structure; e.g. a carboxylic acid group of a first amino acid is converted to an amide (i.e. a peptide bond) when bonded to the amino group of a second amino acid. As such, Xaa may have the formula —N(R^{a})R^{b}C(O)—, where R^{a} and R^{b} are R-groups. R^{a} will typically be hydrogen or methyl (but may be other groups as defined herein). The amino acid residues of a peptide may comprise typical peptide (amide) bonds and may further comprise bonds between side chain functional groups and the side chain or main chain functional group of another amino acid. For example, the side chain carboxylate of one amino acid residue in the peptide (e.g. Asp, Glu, etc.) may be bonded to and the amine of another amino acid residue in the peptide (e.g. Dap, Dab, Orn, Lys). Further details are provided below. Unless otherwise indicated, “Xaa” may be any amino acid, including proteinogenic and nonproteinogenic amino acids. Non-limiting examples of nonproteinogenic amino acids are shown in Table 1 and include: D-amino acids (including without limitation any D-form of the following amino acids), ornithine (Orn), 3-(1-naphtyl)alanine (NaI), 3-(2-naphtyl)alanine (2-NaI), α-aminobutyric acid, norvaline, norleucine (NIe), homonorleucine, beta-(1,2,3-triazol-4-yl)-L-alanine, 1,2,4-triazole-3-alanine, Phe(4-F), Phe(4-Cl), Phe(4-Br), Phe(4-1), Phe(4-NH₂), Phe(4-NO₂), homoarginine (hArg), 2-amino-4-guanidinobutyric acid (Agb), 2-amino-3-guanidinopropionic acid (Agp), p-alanine, 4-aminobutyric acid, 5-aminovaleric acid, 6-aminohexanoic acid, 7-aminoheptanoic acid, 8-aminooctanoic acid, 9-aminononanoic acid, 10-aminodecanoic acid, 2-aminooctanoic acid, 2-amino-3-(anthracen-2-yl)propanoic acid, 2-amino-3-(anthracen-9-yl)propanoic acid, 2-amino-3-(pyren-1-yl) propanoic acid, Trp(5-Br), Trp(5-OCH₃), Trp(6-F), Trp(5-OH) or Trp(CHO), 2-aminoadipic acid (2-Aad), 3-aminoadipic acid (3-Aad), propargylglycine (Pra), homopropargylglycine (Hpg), beta-homopropargylglycine (Bpg), 2,3-diaminopropionic acid (Dap), 2,4-diaminobutyric acid (Dab), azidolysine (Lys(N₃)), azido-ornithine (Orn(N₃)), 2-amino-4-azidobutanoic acid Dab(N₃), Dap(N₃), 2-(5′-azidopentyl)alanine, 2-(6′-azidohexyl)alanine, 4-amino-1-carboxymethyl-piperidine (Pip), 4-(2-aminoethyl)-1-carboxymethyl-piperazine (Acp), and tranexamic acid. If not specified as an L- or D-amino acid, an amino acid shall be understood to encompass both L and D-amino acids.

TABLE 1
List of non-limiting examples of non-proteinogenic amino acids.

| | |
|---|---|
| Any D-amino acid of a proteinogenic amino acid | 10-aminodecanoic acid |
| ornithine (Orn) | 2-aminooctanoic acid |
| 3-(1-naphtyl)alanine (Nal) | 2-amino-3-(anthracen-2-yl)propanoic acid |
| 3-(2-naphtyl)alanine (2-Nal) | 2-amino-3-(anthracen-9-yl)propanoic acid |
| α-aminobutyric acid | 2-amino-3-(pyren-1-yl)propanoic acid |
| norvaline | Trp(5-Br) |
| norleucine (Nle) | Trp(5-OCH₃) |
| homonorleucine | Trp(6-F) |
| beta-(1,2,3-triazol-4-yl)-L-alanine | Trp(5-OH) |
| 1,2,4-triazole-3-alanine | Trp(CHO) |
| Phe(4-F) or (4-F)Phe | 2-aminoadipic acid (2-Aad) |
| Phe(4-Cl) or (4-Cl)Phe | 3-aminoadipic acid (3-Aad) |
| Phe(4-Br) or (4-Br)Phe | propargylglycine (Pra) |
| Phe(4-I) or (4-I)Phe | homopropargylglycine (Hpg) |
| Phe(4-NH₂) or (4-NH₂)Phe | beta-homopropargylglycine (Bpg) |
| Phe(4-NO₂) or (4-NO₂)Phe | 2,3-diaminopropionic acid (Dap) |
| (3-I)Tyr | 2,4-diaminobutyric acid (Dab) |
| homoarginine (hArg) | Cysteic acid (CysAcid) |
| homotyrosine (hTyr) | Nε-isopropyl-lysine (Lys(iPr)) |
| 3-(2-phenanthryl)-L-alanine (2-(Ant)Ala) | Arg(Me) |
| 3-(9-phenanthryl)-L-alanine (9-(Ant)Ala) | Arg(Me)₂ (symmetrical or asymmetrical) |
| 4-(2-aminoethyl)-1-carboxymethyl-piperazine (Acp) | azidolysine (Lys(N₃)) |
| 2-(5′-azidopentyl)alanine | azido-ornithine (Orn(N₃)) |
| 2-(6′-azidohexyl)alanine | amino-4-azidobutanoic acid Dab(N₃) |
| 2-amino-4-guanidinobutyric acid (Agb) | tranexamic acid |
| 2-amino-3-guanidinopropionic acid (Agp) | 4-amino-1-carboxymethyl-piperidine (Pip) |
| β-alanine | NH₂(CH₂)₂O(CH₂)₂C(O)OH |
| 4-aminobutyric acid | NH₂(CH₂)₂[O(CH₂)₂]₂C(O)OH |
| 5-aminovaleric acid | NH₂(CH₂)₂[O(CH₂)₂]₃C(O)OH |
| 6-aminohexanoic acid | NH₂(CH₂)₂[O(CH₂)₂]₄C(O)OH |
| 7-aminoheptanoic acid | NH₂(CH₂)₂[O(CH₂)₂]₅C(O)OH |
| 8-aminooctanoic acid | NH₂(CH₂)₂[O(CH₂)₂]₆C(O)OH |
| 9-aminononanoic acid | Nε-acetyl-lysine (Lys(Ac)) |

The wavy line “ [symbol] ” symbol shown through or at the end of a bond in a chemical formula (e.g. in the definitions R^{4a}, R⁶, R⁷, R⁹ and R¹¹ of Formula I-a, etc.) is intended to define the R group on one side of the wavy line, without modifying the definition of the structure on the opposite side of the wavy line. Where an R group is bonded on two or more sides, any atoms shown outside the wavy lines are intended to clarify orientation of the R group. As such, only the atoms between the two wavy lines constitute the definition of the R group. Unless specified, chemical groups with more than one point of attachment (such as divalent groups) are understood to be placed in any direction a skilled artisan would understand as chemically possible—e.g., —C(O)NH— and —NHC(O)— are interchangeable unless otherwise noted.

In various aspects, there is disclosed a compound of Formula A′ (as defined below), Formula A (as defined below), Formula I-a (as defined below), Formula B′ (as defined below), Formula B (as defined below), Formula I-b (as defined below), Formula III-a (as defined below), Formula III-b (as defined below), Formula IV-a (as defined below), or Formula IV-b (as defined below), or a compound that comprises a PSMA-targeting moiety of Formula II (as defined below), including salts, solvates, stereoisomers, or mixtures of stereoisomers (each compound being a “compound of the invention”) of the foregoing.

The present disclosure relates to a compound of Formula A′:

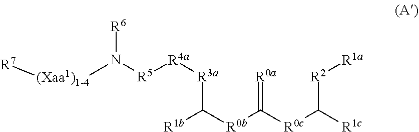

or a salt, a solvate, or a stereoisomer thereof, wherein:
R^{0a} is O or S;
R^{0b} is —O—, —S—, —NH—, or

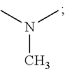

R^{0c} is —O—, —S—, —NH—, or

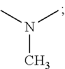

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

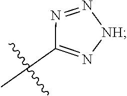 [SMILES: *c1nn[nH]n1]

R^{1b} is —CO₂H, —SO₂H, —SO₃H, —PO₂H, —PO₃H₂, —B(OH)₂, or

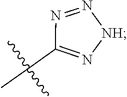 [SMILES: *c1nn[nH]n1]

R^{1c} is —CO₂H, —SO₂H, —SO₃H, —PO₂H, —PO₃H₂, —B(OH)₂, or

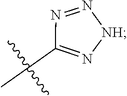 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein each R^{3a} is optionally substituted;
R^{4a} is —O—, —S—, —Se—, —S(O)—, —S(O)₂—,

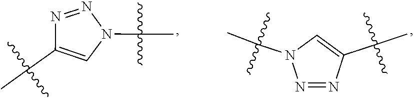 [SMILES: *c1cn(*)nn1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
each Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}—(Xaa²)₀₋₄-,

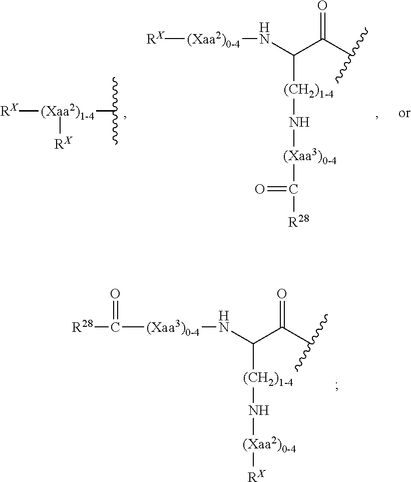

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl, or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(S)—, —C(S)NH—, —NHC(O)—, —C(O)NH—,

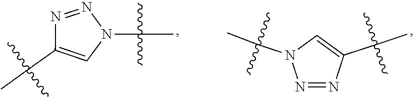 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

The present disclosure also relates to a compound of Formula A:

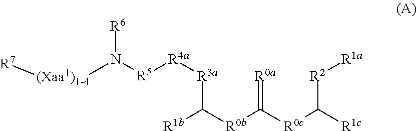

or a salt, a solvate, or a stereoisomer thereof, wherein:
R^{0a} is O or S;
R^{0b} is —O—, —S—, —NH—, or

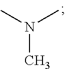

R^{0c} is —O—, —S—, —NH—, or

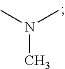

wherein at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

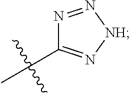 [SMILES: *c1nn[nH]n1]

R^{1b} is —CO₂H, —SO₂H, —SO₃H, —PO₂H, —PO₃H₂, —B(OH)₂, or

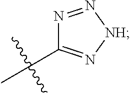 [SMILES: *c1nn[nH]n1]

R^{1c} is —CO₂H, —SO₂H, —SO₃H, —PO₂H, —PO₃H₂, —B(OH)₂, or

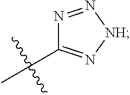 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein each R^{3a} is optionally substituted;
R^{4a} is —O—, —S—, —Se—, —S(O)—, —S(O)₂—,

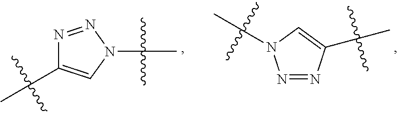 [SMILES: *c1cn(*)nn1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, halogen, C₁-C₆ alkyl, or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms;
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH,
   —NH₂, —NO₂, halogen, —SMe, —CN, C₁-C₆ alkyl, and/or C₁-C₆ alkoxyl groups; or —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
R⁷ is R^{X}-(Xaa²)₀₋₄

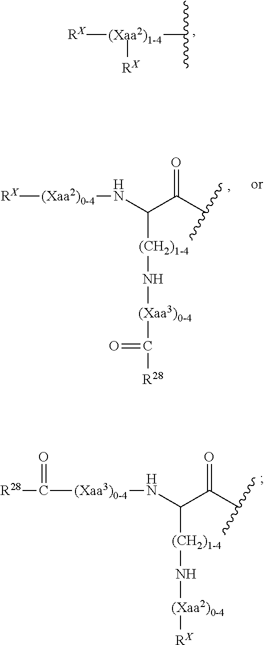

R²⁸ is an albumin binder;
Xaa² and Xaa³, when present, are each independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
   wherein any one or any combination of amide linkages within R⁷-Xaa¹)₁₋₄-N(R⁶)—R⁵—R^{4a}—R^{3a} is optionally independently replaced by linkages selected from the group consisting of, —NHC(S)—, —C(S)NH—, —NHC(O)—,

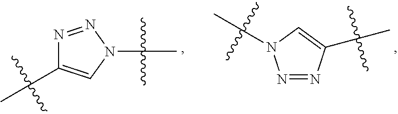 [SMILES: *c1cn(*)nn1 ; *c1cn(*)nn1]

—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

The present disclosure relates to a compound of Formula I-a:

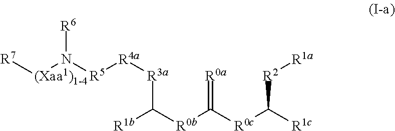

or a salt, a solvate, or a stereoisomer thereof, wherein:
R^{0a} is O or S;
R^{0b} is —O—, —S—, —NH—, or

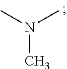

R^{0c} is —O—, —S—, —NH—, or

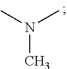

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

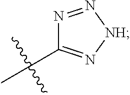 [SMILES: *c1nn[nH]n1]

R^{1b} is —CO₂H, —SO₂H, —SO₃H, —PO₂H, —PO₃H₂, —B(OH)₂, or

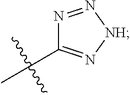 [SMILES: *c1nn[nH]n1]

R^{1c} is —CO₂H, —SO₂H, —SO₃H, —PO₂H, —PO₃H₂, —B(OH)₂, or

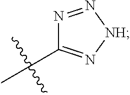 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl;
R^{4a} is —O—, —S—, —Se—, —S(O)—, —S(O)₂—,

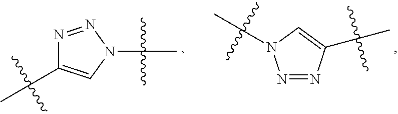 [SMILES: *c1cn(*)nn1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
   R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

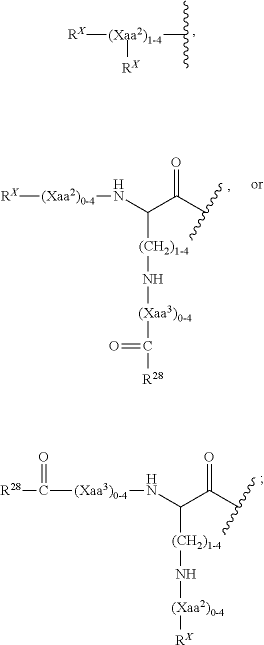

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

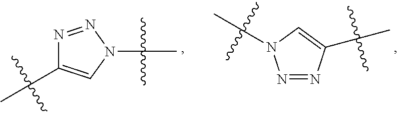 [SMILES: *c1cn(*)nn1 ; *c1cn(*)nn1]

C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

In some embodiments of the compounds of Formula A′, A, and/or I-a, R^{0b} is —O— or —NH—; R^{0c} is —O— or —NH—; and one of R^{0b} and R^{0c} is not —NH—.

In some embodiments of the compounds of Formula A′, A, and/or I-a, R² is —CH₂CHF—, —CHFCH₂—, —(CH₂)₃—, —CH₂OCH₂—, or —CH₂SCH₂—.

In some embodiments of the compounds of Formula A′, A, and/or I-a:
R^{3a} is —CH₂—; —(CH₂)₂—; —(CH₂)₃; —(CH₂)₄—; —(CH₂)₅—; —CH₂—O—CH₂—; —CH₂—S—CH₂—; —CH₂—O—(CH₂)₂—; —(CH₂)₃—O—; —CH₂—S—CH₂—CH(CO₂H)—; —(CH₂)₃—CH(CO₂H)—; —CH₂—O—CH₂—CH(CO₂H)—; —CH₂—Se—CH₂—CH(CO₂H)—; —(CH₂)₁₋₂—R^{3h}—(CH₂)₀₋₂—; —(CH₂)₀₋₂—R^{3h}—(CH₂)₁₋₂—; or —(CH₂)₁₋₃—NH—C(O)—C(R^{3b})₂—;
R^{3h} is:

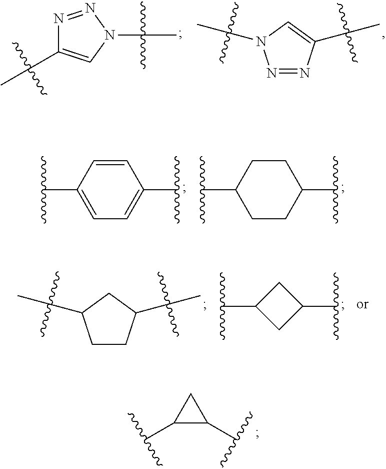

and

each R^{3b} is independently hydrogen, methyl, or ethyl, or together —C(R^{3b})₂-forms cyclopropylenyl.

In some embodiments of the compounds of Formula A′, A, and/or I-a: R^{3a} is —CH₂—NH—C(O)—CH₂—, —CH₂—O—(CH₂)₂—, —(CH₂)₃—O—, —CH₂—S—CH₂—CH(CO₂H)—, —(CH₂)₁₋₂— R^{3h}—(CH₂)₀₋₂— or —(CH₂)₀₋₂-R^{3h}—(CH₂)₁₋₂—; and R^{3h} is

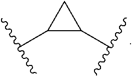 [SMILES: *C1CC1*]

In some embodiments of the compounds of Formula A′, A, and/or I-a, R^{4a} is —C(O)NH—.

In some embodiments of the compounds of Formula A′, A, and/or I-a, R^{4b} is benzyl optionally substituted with one or a combination of OH, NH₂, NO₂, halogen, C₁-C₆ alkyl, and/or C₁-C₆ alkoxyl groups.

In some embodiments of the compounds of Formula A′, A, and/or I-a, R^{4b} is benzyl optionally para-substituted with a halogen.

In some embodiments of the compounds of Formula A′, A, and/or I-a, R⁵ is —CH(R¹⁰)—and wherein R¹⁰ is

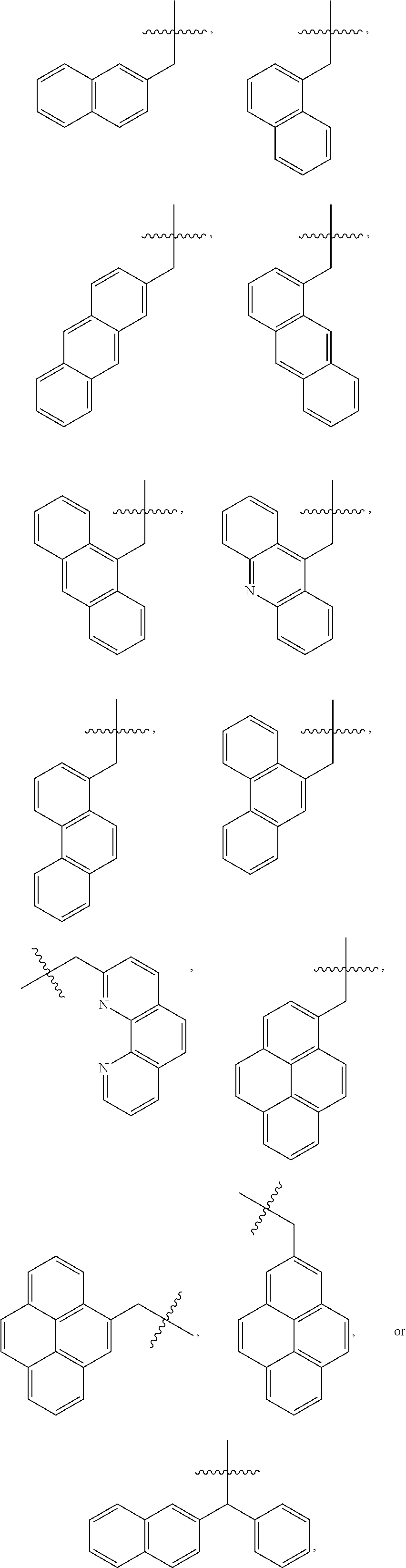

each R¹⁰ is optionally substituted with one or more substituent selected from halogen, —OMe, —SMe, —NH₂, —NO₂, —CN, or —OH; and up to 5 carbon ring atoms of an endocyclic ring of R¹⁰ is optionally replaced with a nitrogen atom such that R¹⁰ can contain up to a maximum of 5 ring nitrogens.

In some embodiments of the compounds of Formula A′, A, and/or I-a, R¹⁰ is,

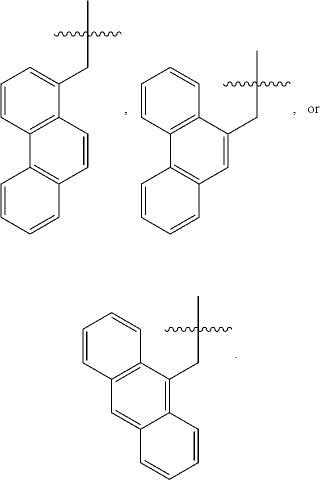 [SMILES: *Cc1cccc2c1ccc1ccccc12 ; *Cc1cc2ccccc2c2ccccc12 ; *Cc1c2ccccc2cc2ccccc12]

In some embodiments of the compounds of Formula A′, A, and/or I-a, -(Xaa¹)₁₋₄-N(R⁶)—R⁵—R^{4a}— is

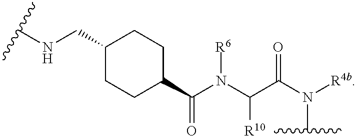

In some embodiments of the compounds of Formula A′, A, and/or I-a,

R⁷ is R^{X}-(Xaa²)₀₋₄ wherein (Xaa²)₀₋₄ is absent;

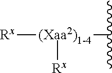

wherein (Xaa²)₁₋₄ is a tripeptide; or

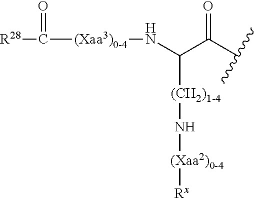

wherein (Xaa²)₀₋₄ is absent;
R²⁸ is

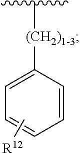

R¹² is I, Br, F, Cl, H, —OH, —OCH₃, —NH₂, or —CH₃; and
R^{X} is a radiometal chelator optionally bound to a radiometal, or a prosthetic group containing a trifluoroborate.

In some embodiments of the compounds of Formula A′, A, and/or I-a,
R⁷ is R^{X}-(Xaa²)₀₋₄ or

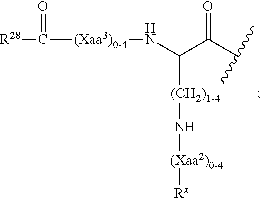

R²⁸ is

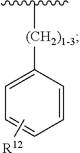

Xaa² is absent;
Xaa³ is absent or is a single amino acid residue; and
R¹² is —OCH₃ or Cl.

In some embodiments of the compounds of Formula A′, A, and/or I-a, R⁷ is R^{X}-(Xaa²)₀₋₄- and R^{X} is DOTA, optionally chelated with a radiometal.

In some embodiments of the compounds of Formula A′, A, and/or I-a,
R⁷ is

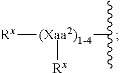

each R^{X} is independently —C(O)—(CH₂)₀₋₅R¹⁸—(CH₂)₁₋₅R¹⁷BF₃;
R¹⁸ is absent,

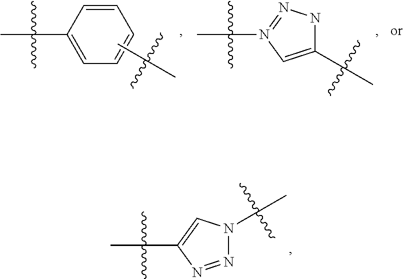

R¹⁷BF₃ is

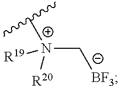

and
R¹⁹ and R²⁰ are independently C₁-C₅ linear or branched alkyl groups.

In some embodiments of the compounds of Formula A′, A, and/or I-a, R^{0a} is O, R^{1a} is —CO₂H; R^{1b} is —CO₂H; and R^{1c} is —CO₂H.

In some embodiments of the compounds of Formula A′, A, and/or I-a,
R^{0a} is O;
R^{1a} is —CO₂H;
R^{1b} is —CO₂H;
R^{1c} is —CO₂H;
R² is —CH₂—, —CH₂CHF—, —CHFCH₂—, —(CH₂)₂—, —(CH₂)₃—, —CH₂OCH₂—, or —CH₂SCH₂—;
-(Xaa¹)₁₋₄-N(R⁶)—R⁵—R^{4a}-is

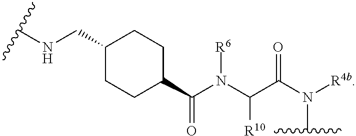

R^{4b} is hydrogen, methyl or ethyl;
R⁶ is hydrogen, methyl or ethyl;
R¹⁰ is

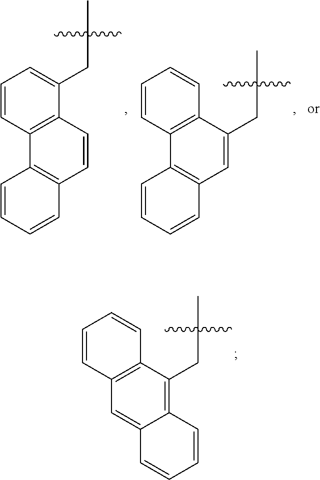 [SMILES: *Cc1cccc2c1ccc1ccccc12 ; *Cc1cc2ccccc2c2ccccc12 ; *Cc1c2ccccc2cc2ccccc12]

R⁷ is R^{X}-(Xaa²)₀₋₄ or

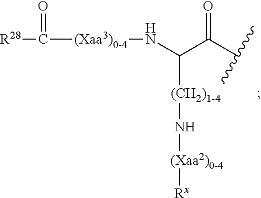

R²⁸ is

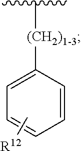

Xaa³ is absent or is a single amino acid residue;
Xaa² is absent;
R¹² is —OCH₃ or Cl; and
R^{X} is a radiometal chelator optionally bound to a radiometal.

In some embodiments of the compounds of Formula A′, A, and/or I-a, the radiometal chelator is selected from Table 2; and wherein the radiometal chelator is optionally bound to a radiometal.

In some embodiments of the compounds of Formula A′, A, and/or I-a, the radiolabeling group is a prosthetic group containing a trifluoroborate.

In some embodiments of the compounds of Formula A′ or A, the compound is selected from AR-2-050-1, AR-2-050-2, AR-2-113-1 or AR-2-113-2, or a salt or a solvate thereof, wherein each compound is optionally bound to a radiometal.

The present disclosure relates to a compound of Formula B′:

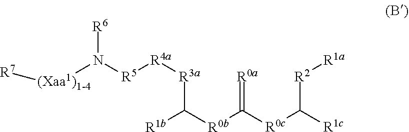

or a salt, a solvate, or a stereoisomer thereof, wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

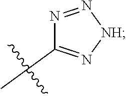 [SMILES: *c1nn[nH]n1]

R^{1b} is —CO₂H, —SO₂H, —SO₃H, —PO₂H, —PO₃H₂, —B(OH)₂, or

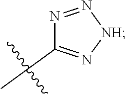 [SMILES: *c1nn[nH]n1]

R^{1c} is —CO₂H, —SO₂H, —SO₃H, —PO₂H, —PO₃H₂, —B(OH)₂, or

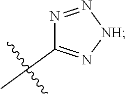 [SMILES: *c1nn[nH]n1]

R² is —CH₂—, —(CH₂)₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein each R^{3a} is optionally substituted;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, N₃, CN, SMe, CF₃, CHF₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
each Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R^{X}-(Xaa²)₀₋₄-,

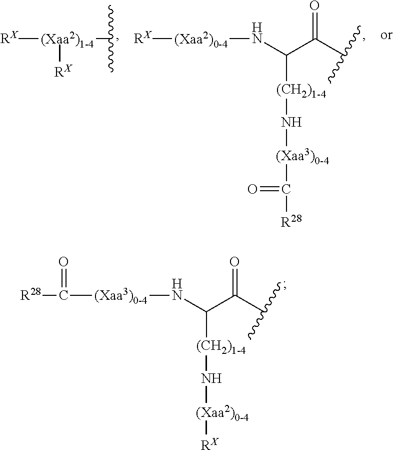

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl, or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —NHC(S)—, —C(S)NH—, —C(O)NH—,

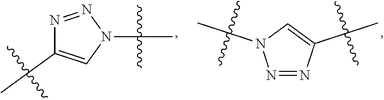 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

In some embodiments, R^{3a} is optionally substituted with —CO₂H. In some embodiments, R^{3a} is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂—O—CH(CO₂H)—CH₂—, —CH₂—Se—CH(CO₂H)—CH₂—, —CH₂—CH(CO₂H)—(CH₂)₂—, or —(CH₂)₂—CH(CO₂H)—, —CH₂—CH(CO₂H)—CH₂—.

In some embodiments, R^{3a} is optionally substituted with oxo. In some embodiments, R^{3a} is a heteroalkylenyl, which is optionally substituted. In some embodiments, heteroalkylenyl optionally substituted with at least one oxo forms an amide group within the heteroalkyleneyl. In some embodiments, heteroalkylenyl substituted with at least one oxo is —(CH₂)₁₋₃—NH—C(O)—C(R^{3b})₂-, wherein each R^{3b} is, independently, hydrogen, methyl, or ethyl, or together —C(R^{3b})₂— forms cyclopropylenyl.

The present disclosure relates to a compound of Formula B:

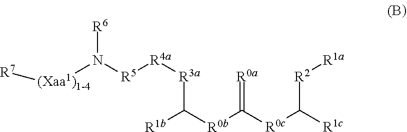

or a salt, a solvate, or a stereoisomer thereof, wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

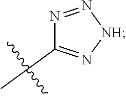 [SMILES: *c1nn[nH]n1]

R^{1b} is —CO₂H, —SO₂H, —SO₃H, —PO₂H, —PO₃H₂—B(OH)₂, or

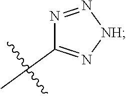 [SMILES: *c1nn[nH]n1]

R^{1c} is —CO₂H, —SO₂H, —SO₃H, —PO₂H, —PO₃H₂, —B(OH)₂, or

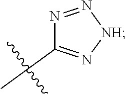 [SMILES: *c1nn[nH]n1]

R² is —CH₂—, —(CH₂)₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂—O—CH(CO₂H)—CH₂—, —CH₂—Se—CH(CO₂H)—CH₂—, —CH₂—CH(CO₂H)—(CH₂)₂—, —(CH₂)₂—CH(CO₂H)—, —CH₂—CH(CO₂H)—CH₂—, —(CH₂)₁₋₂—R^{3h}—(CH₂)₀₋₂—, —(CH₂)₀₋₂—R^{3h}—(CH₂)₁₋₂— or —(CH₂)₁₋₃—NH—C(O)—C(R^{3b})₂—;
R^{3h} is

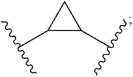 [SMILES: *C1CC1*]

each R^{3b} is, independently, hydrogen, methyl, or ethyl, or together —C(R^{3b})₂— forms cyclopropylenyl;
R⁴° is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, N₃, CN, SMe, CF₃, CHF₂, halogen, C₁-C₆ alkyl, or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl; R^{X}-(Xaa²)₀₋₄-,

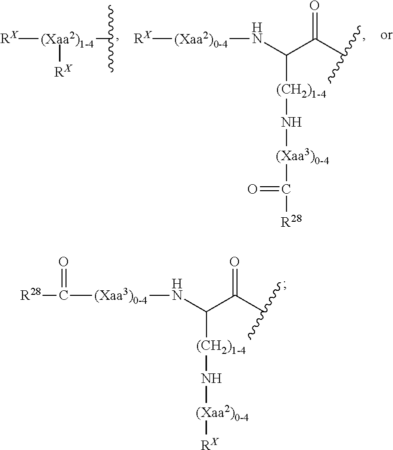

R²⁸ is an albumin binder;
Xaa² and Xaa³, when present, are each independently —N(R¹³)R¹⁴C(O)—, wherein each R¹³ is independently hydrogen or methyl, and wherein each R¹⁴ is, independently, a linear or branched, cyclic or acyclic, and/or aromatic or non-aromatic C₁-C₂₀ alkylenyl, alkenylenyl or alkynylenyl, or a linear or branched, cyclic or acyclic, and/or aromatic or non-aromatic C₂-C₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof; and
wherein any one or any combination of amide linkages within R⁷-Xaa¹)₁₋₄-N(R⁶)—R⁵—R^{4a}—R^{3a} is optionally independently replaced by linkages selected from the group consisting of, —NHC(S)—, —C(S)NH—, —NHC(O)—,

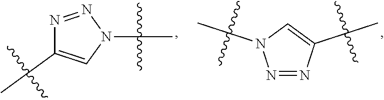 [SMILES: *c1cn(*)nn1 ; *c1cn(*)nn1]

—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

The present disclosure also relates to a compound of Formula I-b:

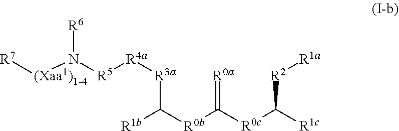

or a salt, a solvate, or a stereoisomer thereof, wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

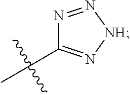 [SMILES: *c1nn[nH]n1]

R^{1b} is —CO₂H, —SO₂H, —SO₃H, —PO₂H, —PO₃H₂, —B(OH)₂, or

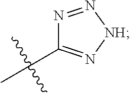 [SMILES: *c1nn[nH]n1]

R^{1c} is —CO₂H, —SO₂H, —SO₃H, —PO₂H, —PO₃H₂, —B(OH)₂, or

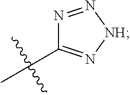 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

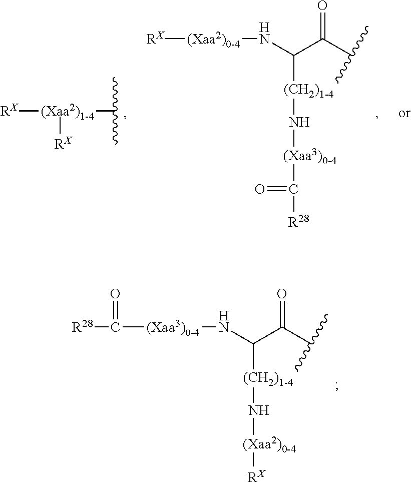

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

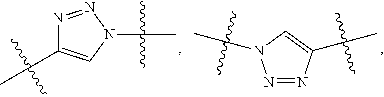 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

In some embodiments of the compounds of Formula B′, B, and/or I-b, R^{3a} is —CH₂—NH—C(O)—CH₂—, —CH₂—O—(CH₂)₂—, —(CH₂)₃—O—, —CH₂—S—CH₂—CH(CO₂H)—, —(CH₂)₁₋₂— R^{3h}—(CH₂)₀₋₂— or —(CH₂)₀₋₂—R^{3h}—(CH₂)₁₋₂—; and wherein R^{3h} is

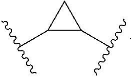 [SMILES: *C1CC1*]

In some embodiments of the compounds of Formula B′, B, and/or I-b, R² is —CH₂—, —(CH₂)₂—, —CH₂CHF—, —CHFCH₂—, —(CH₂)₃—, —CH₂OCH₂—, or —CH₂SCH₂—.

In some embodiments of the compounds of Formula B′, B, and/or I-b, R^{4a} is —C(O)NH—.

In some embodiments of the compounds of Formula B′, B, and/or I-b, R^{4b} is benzyl optionally substituted with one or a combination of OH, NH₂, NO₂, N₃, CN, SMe, CF₃, CHF₂, halogen, C₁-C₆ alkyl, and/or C₁-C₆ alkoxyl groups. In some embodiments, R^{4b} is benzyl optionally para-substituted with a halogen.

In some embodiments of the compounds of Formula B′, B, and/or I-b, R⁵ is —CH(R¹⁰)—; and wherein R¹⁰ is

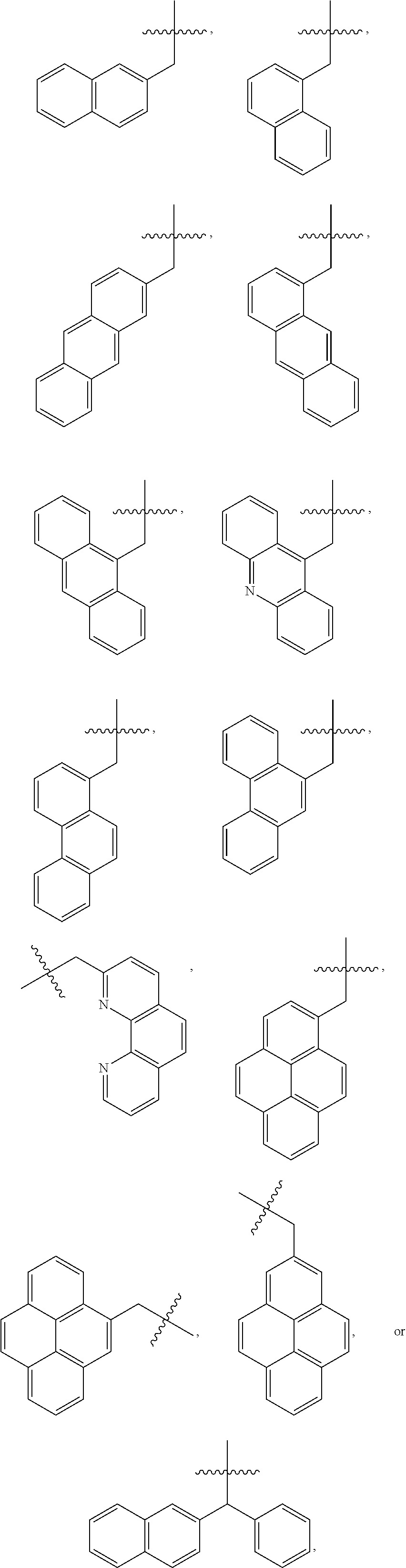

each R¹⁰ is optionally substituted with one or more substituent selected from halogen, —OMe, —SMe, —NH₂, —NO₂, —CN, or —OH; and up to 5 carbon ring atoms of an endocyclic ring of R¹⁰ is optionally replaced with a nitrogen atom such that R¹⁰ can contain up to a maximum of 5 ring nitrogens.

In some embodiments of the compounds of Formula B′, B, and/or I-b, R¹⁰ is

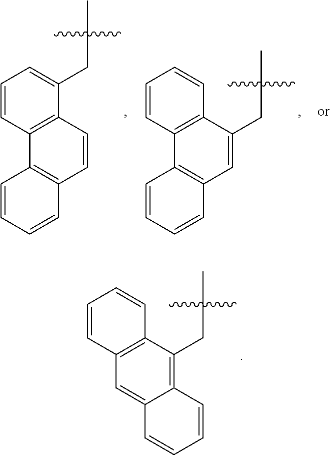 [SMILES: *Cc1cccc2c1ccc1ccccc12 ; *Cc1cc2ccccc2c2ccccc12 ; *Cc1c2ccccc2cc2ccccc12]

In some embodiments of the compounds of Formula B′, B, and/or I-b, -(Xaa¹)₁₋₄-N(R⁶)—R⁵—R^{4a}-is

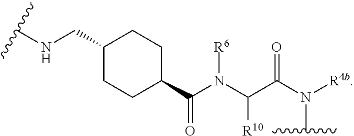

In some embodiments of the compounds of Formula B′, B, and/or I-b,
R⁷ is: R^{X}-(Xaa²)₀₋₄ wherein (Xaa²)₀₋₄ is absent;

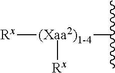

wherein (Xaa²)₁₋₄ is a tripeptide; or

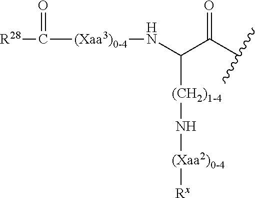

wherein (Xaa²)₀₋₄ is absent;
R²⁸ is

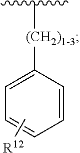

R¹² is I, Br, F, Cl, H, —OH, —OCH₃, —NH₂, or —CH₃; and
R^{X} is a radiometal chelator optionally bound to a radiometal, or a prosthetic group containing a trifluoroborate.

In some embodiments of the compounds of Formula B′, B, and/or I-b,
R⁷ is R^{X}-(Xaa²)₀₋₄ or

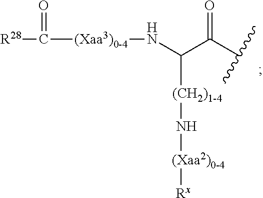

R²⁸ is

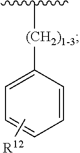

Xaa² is absent
Xaa³ is absent or is a single amino acid residue; and
R¹² is —OCH₃ or Cl.

In some embodiments of the compounds of Formula B′, B, and/or I-b, R⁷ is R^{X}-(Xaa²)₀₋₄- and R^{X} is DOTA, optionally chelated with a radiometal.

In some embodiments of the compounds of Formula B′, B, and/or I-b,
R⁷ is

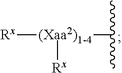

each R^{X} is independently —C(O)—(CH₂)₀₋₅R¹⁸—(CH₂)₁₋₅R¹⁷BF₃;
R¹⁸ is absent,

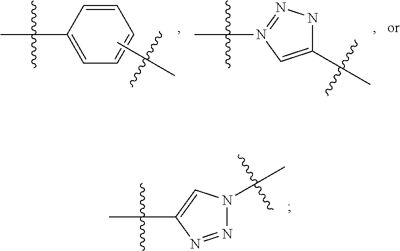

R¹⁷BF₃ is

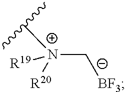

and
R¹⁹ and R²⁰ are each independently C₁-C₅ linear or branched alkyl groups.

In some embodiments of the compounds of Formula B′, B, and/or I-b, R^{0a} is O; R^{1a} is —CO₂H; R^{1b} is —CO₂H; and R^{1c} is —CO₂H.

In some embodiments of the compounds of Formula B′, B, and/or I-b,
R^{0a is O;}
R^{1a} is —CO₂H;
R^{1b} is —CO₂H;
R^{1c} is —CO₂H;
R² is —CH₂—, —CH₂CHF—, —CHFCH₂—, —(CH₂)₂—, —(CH₂)₃—, —CH₂OCH₂—, or
-(Xaa¹)₁₋₄-N(R⁶)—R⁵—R^{4a}-is

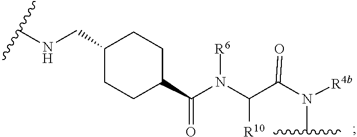

R^{4b} is hydrogen, methyl or ethyl;
R⁶ is hydrogen, methyl or ethyl;
R¹⁰ is

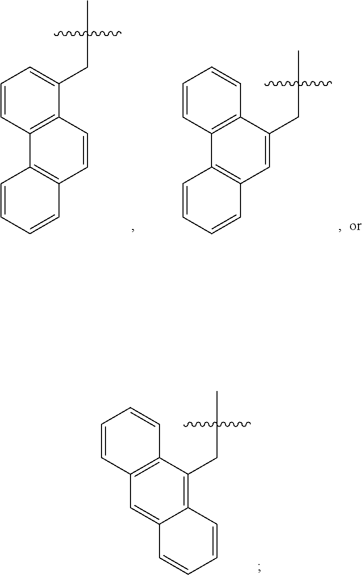 [SMILES: *Cc1cccc2c1ccc1ccccc12 ; *Cc1cc2ccccc2c2ccccc12 ; *Cc1c2ccccc2cc2ccccc12]

R⁷ is R^{X}-(Xaa²)₀₋₄ or

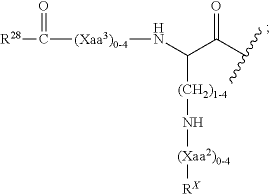

R²⁸ is

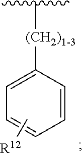

Xaa³ is absent or is a single amino acid residue; and
Xaa² is absent;
R¹² is —OCH₃ or Cl; and
R^{X} is a radiometal chelator optionally bound to a radiometal.

In some embodiments of the compounds of Formula B′, B, and/or I-b, the radiometal chelator is selected from Table 2; and wherein the radiometal chelator is optionally bound to a radiometal.

In some embodiments of the compounds of Formula B′, B, and/or I-b, the radiolabeling group is a prosthetic group containing a trifluoroborate.

In some embodiments of the compounds of Formula B′ or B, the compound is selected from CCZ02010, CCZ02011, CCZ02018, CCZ01194, CCZ01198, CCZ02032, CCZ02033, ADZ-4-101, PD-6-49, PD-5-131 or PD-5-159, or a salt or a solvate thereof, wherein each compound is optionally bound to a radiometal. In some embodiments, the compound is a mixture of PD-5-131 and PD-5-159.

In some embodiments, the compounds of the invention comprise a prostate specific membrane antigen (PSMA)-targeting moiety of Formula II:

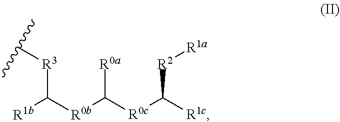

or a salt, a solvate, or a stereoisomer thereof, wherein:
R^{0a} is O or S;
R^{0b} is —O—, —S—, —NH—, or

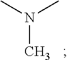

R^{0c} is —O—, —S—, —NH—, or

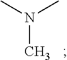

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

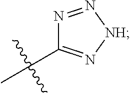 [SMILES: *c1nn[nH]n1]

R^{1b} is —CO₂H, —SO₂H, —SO₃H, —PO₂H, —PO₃H₂, —B(OH)₂, or

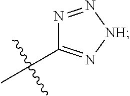 [SMILES: *c1nn[nH]n1]

R^{1c} is —CO₂H, —SO₂H, —SO₃H, —PO₂H, —PO₃H₂, —B(OH)₂, or

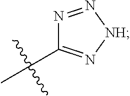 [SMILES: *c1nn[nH]n1]

R² is —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring; and
R³ is a linker.

In some embodiments, R³ in Formula II is R^{3a} as defined for A′, A, B′, B, I-a, I-b, III-a, III-b, IV-a, or IV-b.

The present disclosure also relates to a compound of Formula III-a:

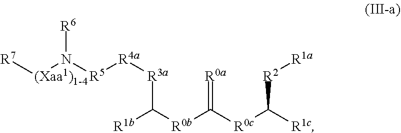

or a salt, a solvate, or a stereoisomer thereof, wherein:
R^{0a} is S or O;
R^{0b} is —O—, —S—, —NH—, or

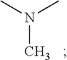

R^{0c} is —O—, —S—, —NH—, or

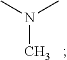

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

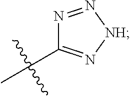 [SMILES: *c1nn[nH]n1]

R^{1b} is —CO₂H, —SO₂H, —SO₃H, —PO₂H, —PO₃H₂, —B(OH)₂, or

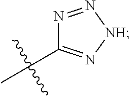 [SMILES: *c1nn[nH]n1]

R^{1c} is —CO₂H, —SO₂H, —SO₃H, —PO₂H, —PO₃H₂, —B(OH)₂, or

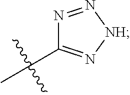 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —O—, —S—, —Se—, —S(O)—, —S(O)₂—,

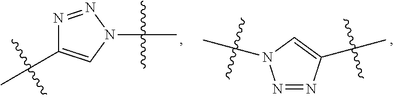 [SMILES: *c1cn(*)nn1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—,

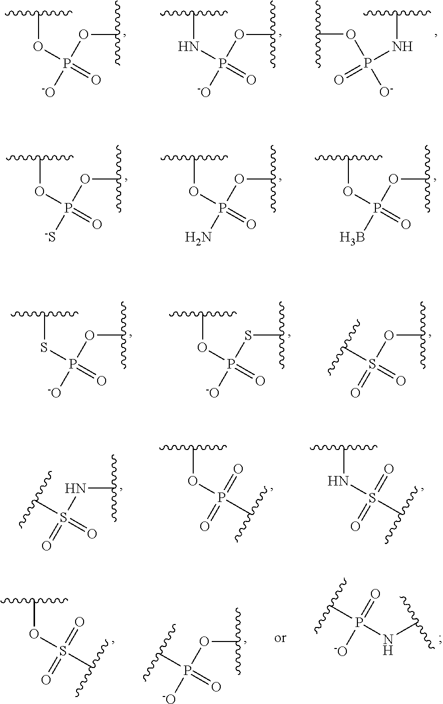

R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂—R^{23d}—R^{23a} wherein R^{23d} is absent, CH₂, O, NH, or S, and wherein R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R^{X}-(Xaa²)₀₋₄,

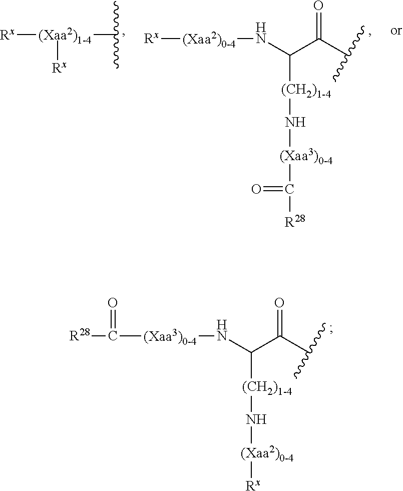

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

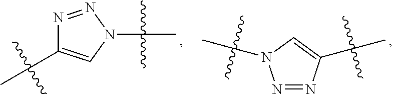 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

The present disclosure also relates to a compound of Formula III-b:

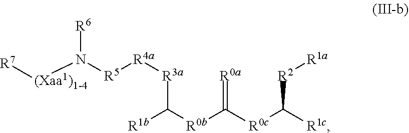

or a salt, a solvate, or a stereoisomer thereof, wherein:
R^{0a} is S or O;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

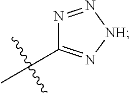 [SMILES: *c1nn[nH]n1]

R^{1b} is —CO₂H, —SO₂H, —SO₃H, —PO₂H, —PO₃H₂, —B(OH)₂, or

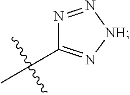 [SMILES: *c1nn[nH]n1]

R^{1c} is —CO₂H, —SO₂H, —SO₃H, —PO₂H, —PO₃H₂, —B(OH)₂, or

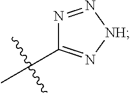 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R²³°, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

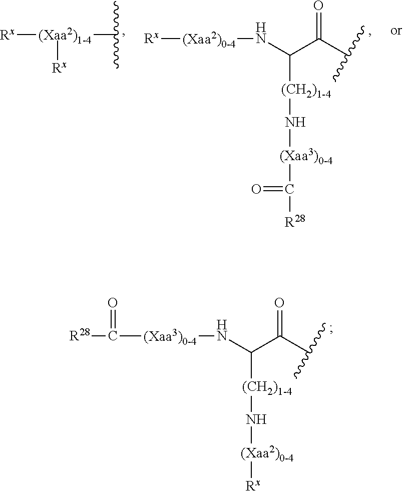

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

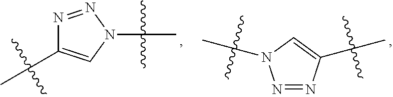 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

The present disclosure also relates to a compound of Formula IV-a:

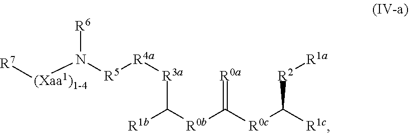

or a salt, a solvate, or a stereoisomer thereof, wherein:
R^{0a} is S or O;
R^{0b} is —O—, —S—, —NH—, or

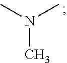

R^{0c} is —O—, —S—, —NH—, or

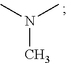

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

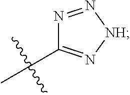 [SMILES: *c1nn[nH]n1]

R^{1b} is —CO₂H, —SO₂H, —SO₃H, —PO₂H, —PO₃H₂, —B(OH)₂, or

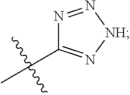 [SMILES: *c1nn[nH]n1]

R^{1c} is —CO₂H, —SO₂H, —SO₃H, —PO₂H, —PO₃H₂, —B(OH)₂, or

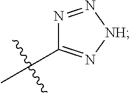

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —O—, —S—, —Se—, —S(O)—, —S(O)₂—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—,

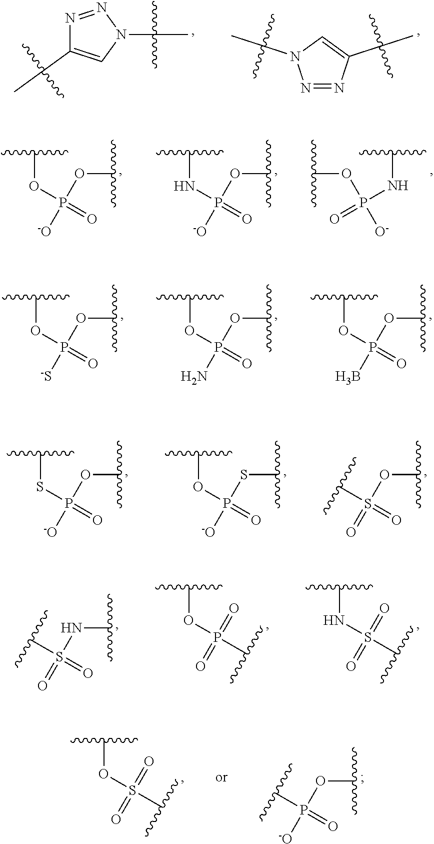

R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R²³°, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is:
   hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups; or
   a carbonyl, a phosphoryl or a sulfonyl group that is linked to the alpha-nitrogen in Xaa¹ to respectively give an amide, phosphoramidate/phosphonamidate, or sulfonamide linkage; or —NHC(O)—, —(NH)₂—C(O)—, —C(O)—(NH)₂—C(O)—, —OC(O)—, —OC(S)—,
   —NHC(S)—, —NHC(O)C(O)—, or —NH—NH—C(O)—, to enjoin the alpha-nitrogen in Xaa¹;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;

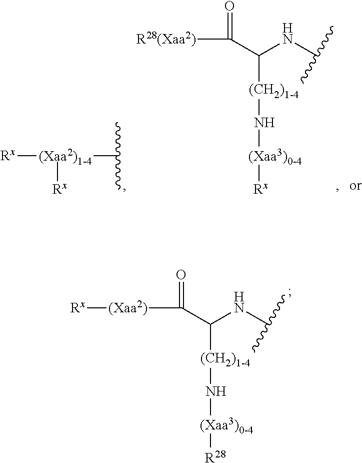

R⁷ is R^{X}-(Xaa²)₀₋₄-,
R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

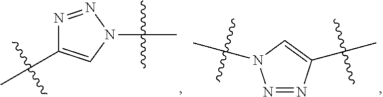

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

The present disclosure also relates to a compound of Formula IV-b:

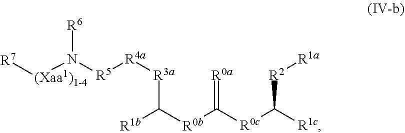

or a salt, a solvate, or a stereoisomer thereof, wherein: R^{0a} is S or O;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

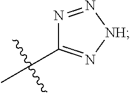

R^{1b} is —CO₂H, —SO₂H, —SO₃H, —PO₂H, —PO₃H₂, —B(OH)₂, or

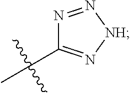

R^{1c} is —CO₂H, —SO₂H, —SO₃H, —PO₂H, —PO₃H₂, —B(OH)₂, or

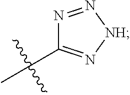

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
   a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
   —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
   —CH(R^{23b})—R²³°, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is:
   hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups; or
   a carbonyl, a phosphoryl or a sulfonyl group that is linked to the alpha-nitrogen in Xaa¹ to respectively give an amide, phosphoramidate/phosphonamidate, or sulfonamide linkage; or
   —NHC(O)—, —(NH)₂—C(O)—, —C(O)—(NH)₂—C(O)—, —OC(O)—, —OC(S)—, —NHC(S)—, —NHC(O)C(O)—, or —NH—NH—C(O)—, to enjoin the alpha-nitrogen in Xaa¹;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

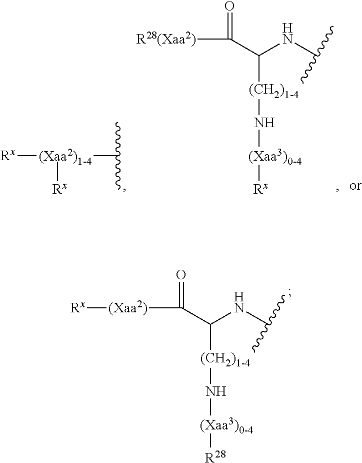

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

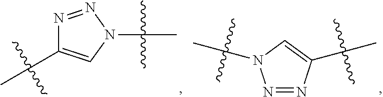

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

In some embodiments, the Formula I-a, I-b, III-a, III-b, IV-a, or IV-b the compound has the opposite stereocenter at the carbon adjacent to R² than what is depicted (e.g., stereoisomer of the compound of Formula I-a, I-b, III-a, III-b, IV-a, or IV-b).

In some embodiments, the Formula A′, A, B′, B, I-a, I-b, III-a, III-b, IV-a, IV-b compounds (and salts, solvates, stereoisomers thereof) have the stereochemical configuration shown below:

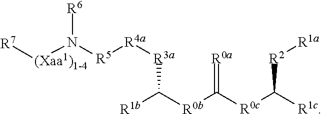

In some embodiments, the compounds comprising a Formula II PSMA-binding moiety (or a salts, solvates, stereoisomers thereof) have the stereochemical configuration shown below:

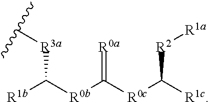

The following definitions apply to Formula A′, A, I-a, III-a, and IV-a compounds (and salts, solvates, stereoisomers thereof), and compounds comprising a PSMA-binding moiety of Formula II (and salts, solvates, stereoisomers thereof).

In some embodiments, R^{0b} is —O—. In some embodiments, R^{0b} is —S—. In some embodiments, In some embodiments, R^{0b} is

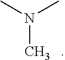

In some embodiments, R^{0b} is —NH—, and R^{0c} is —O—, —S—, or

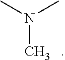

In some embodiments, R^{0c} is —O—. In some embodiments, R^{0c} is —S—. In some embodiments, In some embodiments, R^{0C is}

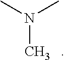

In some embodiments, R^{0c} is —NH—, and R^{0b} is —O—, —S—, or

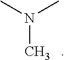

In some embodiments, R^{0b} is —O— and R^{0c} is —NH—. In some embodiments, R^{0b} is —NH— and R^{0c} is —O—. In some embodiments, R^{0b} is —S— and R^{0c} is —NH—. In some embodiments, R^{0b} is —NH— and R^{0c} is —S—.

The following definitions apply to Formula A′, A, B′, B, I-a and I-b compounds (and salts, solvates, stereoisomers thereof).

In some embodiments, R² is —CH₂—. In some embodiments, R² is —CH(OH)—. In some embodiments, R² is —CHF—. In some embodiments, R² is —CF₂—. In some embodiments, R² is —CH(CH₃)—. In some embodiments, R² is —C(CH₃)₂—.

In some embodiments, R² is —CH₂CH(OH)—. In some embodiments, R² is —CH₂CHF—. In some embodiments, R² is —CHFCH₂—. In some embodiments, R² is —CF₂CH₂—. In some embodiments, R² is —CH₂CF₂—. In some embodiments, R² is —CH(OH)CH₂—. In some embodiments, R² is —CH(CH₃)CH₂—. In some embodiments, R² is —CH₂CH(CH₃)—. In some embodiments, R² is —C(CH₃)₂CH₂—. In some embodiments, R² is —CH₂C(CH₃)₂—.

In some embodiments, R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

In some embodiments, R² is —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂CH(COOH)CH₂—, or —CH₂CH₂CH(COOH)—. In some embodiments, R² is —CH₂OCH₂— or —CH₂SCH₂—.

In some embodiments, R² is —CH₂—, —CH(OH)—, —CHF—, —CF₂—, —CH(CH₃)—, —C(CH₃)₂—, —CHFCH₂—, —CF₂CH₂—, —CH(OH)CH₂—, —CH(CH₃)CH₂—, —C(CH₃)₂CH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—.

In some embodiments, R² is —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂—O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

In some embodiments, R² is —CH₂CH(OH)—, —CH₂CHF—, —CH₂CH(CH₃)—, —CH₂CH(COOH)—, —CH₂CH(OH)CH₂—, —CH₂CH(F)CH₂—, or —CH₂CH(CH₃)CH₂—, wherein the second carbon in R² has R-configuration. In some embodiments, R² is —CH₂CH(OH)—, —CH₂CHF—, or —CH₂CH(CH₃)—, wherein the second carbon in R² has R-configuration. In some embodiments, R² is —CH₂CHF—, wherein the second carbon in R² has R-configuration.

In some embodiments, R² is —CH₂CH(OH)CH₂—. In some embodiments, R² is —CH₂CHFCH₂—. In some embodiments, R² is —(CH₂)₂CH(OH)—. In some embodiments, R² is —(CH₂)₂CHF—. In some embodiments, R² is —(CH₂)₃—. In some embodiments, R² is —CH₂OCH₂—. In some embodiments, R² is —CH₂SCH₂—. In some embodiments, R² is —CHFCH₂CH₂—. In some embodiments, R² is —CH(OH)CH₂CH₂—. In some embodiments, R² is —CH(CH₃)CH₂CH₂—. In some embodiments, R² is —CH₂CH(CH₃)CH₂—. In some embodiments, R² is —CH₂CH₂CH(CH₃)—. In some embodiments, R² is —C(CH₃)₂CH₂CH₂—. In some embodiments, R² is —CH₂C(CH₃)₂CH₂—. In some embodiments, R² is —CH₂CH₂C(CH₃)₂—. In some embodiments, R² is —CH(CH₃)—O—CH₂—. In some embodiments, R² is —C(CH₃)₂O—CH₂—. In some embodiments, R² is —CH₂—O—CH(CH₃)—. In some embodiments, R² is —CH₂—O—C(CH₃)₂—. In some embodiments, R² is —CH₂—S(O)—CH₂—. In some embodiments, R² is —CH₂—S(O)₂—CH₂—. In some embodiments, R² is —CH(CH₃)—S—CH₂—. In some embodiments, R² is —C(CH₃)₂—S—CH₂—. In some embodiments, R² is —CH₂—S—CH(CH₃)—. In some embodiments, R² is —CH₂—S—C(CH₃)₂—. In some embodiments, R² is —CH(CH₃)—S(O)—CH₂—. In some embodiments, R² is —C(CH₃)₂—S(O)—CH₂—. In some embodiments, R² is —CH₂—S(O)—CH(CH₃)—. In some embodiments, R² is —CH₂—S(O)—C(CH₃)₂—. In some embodiments, R² is —CH(CH₃)—S(O)₂—CH₂—. In some embodiments, R² is —C(CH₃)₂—S(O)₂—CH₂—. In some embodiments, R² is —CH₂—S(O)₂—CH(CH₃)—. In some embodiments, R² is —CH₂—S(O)₂—C(CH₃)₂—. In some embodiments, R² is —CH₂—NH—C(O)—. In some embodiments, R² is —C(O)—NH—CH₂—. In some embodiments, R² is —C(O)—NH—CH(CH₃)—. In some embodiments, R² is —C(O)—NH—C(CH₃)₂—.

In some embodiments, R² is —CH₂—, —(CH₂)₂—, —CH₂CHF—, —CHFCH₂—, —(CH₂)₃—, —CH₂OCH₂—, or —CH₂SCH₂—. In some embodiments, R² is —(CH₂)₃—. In some embodiments, R² is —(CH₂)₂—, —(CH₂)₃—, or —CH₂SCH₂—. In some embodiments, R² is —(CH₂)₃- or —CH₂SCH₂—.

In some embodiments, R² is —HC[CH₂]CH— or —HC[CH₂]CHCH₂—, wherein HC[CH₂]CH represents a cyclopropyl ring. In some embodiments, R² is —HC[CH₂]CH—.

The following definitions apply to compounds comprising a PSMA-binding moiety of Formula II (and salts, solvates, stereoisomers thereof).

In some embodiments, R² is —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

In some embodiments, R² is —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, or —CH₂—S(O)₂—C(CH₃)₂

In some embodiments, R² is —CH(CH₃)CH₂CH₂—. In some embodiments, R² is —CH₂CH(CH₃)CH₂—. In some embodiments, R² is —CH₂CH₂CH(CH₃)—. In some embodiments, R² is —C(CH₃)₂CH₂CH₂—. In some embodiments, R² is —CH₂C(CH₃)₂CH₂—. In some embodiments, R² is —CH₂CH₂C(CH₃)₂—. In some embodiments, R² is —CH(CH₃)—O—CH₂—. In some embodiments, R² is —C(CH₃)₂O—CH₂—. In some embodiments, R² is —CH₂—O—CH(CH₃)—. In some embodiments, R² is —CH₂—O—C(CH₃)₂—. In some embodiments, R² is —CH₂—S(O)—CH₂—. In some embodiments, R² is —CH₂—S(O)₂—CH₂—. In some embodiments, R² is —CH(CH₃)—S—CH₂—. In some embodiments, R² is —C(CH₃)₂—S—CH₂—. In some embodiments, R² is —CH₂—S—CH(CH₃)—. In some embodiments, R² is —CH₂—S—C(CH₃)₂—. In some embodiments, R² is —CH(CH₃)—S(O)—CH₂—. In some embodiments, R² is —C(CH₃)₂—S(O)—CH₂—. In some embodiments, R² is —CH₂—S(O)—CH(CH₃)—. In some embodiments, R² is —CH₂—S(O)—C(CH₃)₂—. In some embodiments, R² is —CH(CH₃)—S(O)₂—CH₂—. In some embodiments, R² is —C(CH₃)₂—S(O)₂—CH₂—. In some embodiments, R² is —CH₂—S(O)₂—CH(CH₃)—. In some embodiments, R² is —CH₂—S(O)₂—C(CH₃)₂—. In some embodiments, R² is —C(O)—NH—CH₂—. In some embodiments, R² is —C(O)—NH—CH(CH₃)—. In some embodiments, R² is —C(O)—NH—C(CH₃)₂—.

In some embodiments, R² is —CH₂CH(CH₃)CH₂—, wherein the second carbon in R² has R-configuration.

In some embodiments, R² is —(CH₂)₃—. In some embodiments, R² is —(CH₂)₂—, —(CH₂)₃—, or —CH₂SCH₂—. In some embodiments, R² is —(CH₂)₃- or —CH₂SCH₂—.

The linker (R³) may be any linker. In some embodiments, R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl. In some embodiments, R³ is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl. In some embodiments, R³ is a linear or branched peptide linker.

In some embodiments, R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein R³ is optionally substituted.

In some embodiments, R³ is —CH₂—; —(CH₂)₂—; —(CH₂)₃; —(CH₂)₄—; —(CH₂)₅—; —CH₂—O—CH₂—; —CH₂—S—CH₂—; —CH₂—O—(CH₂)₂—; —(CH₂)₃—O—; —CH₂—S—CH₂—CH(CO₂H)—; —(CH₂)₃—CH(CO₂H)—; —CH₂—O—CH₂—CH(CO₂H)—; —CH₂—Se—CH₂—CH(CO₂H)—; —(CH₂)₁₋₂— R^{3h}—(CH₂)₂—; —(CH₂)₀₋₂-R^{3h}—(CH₂)₁₋₂—; or —(CH₂)₁₋₃-NH—C(O)—C(R^{3b})₂—; R^{3h} is:

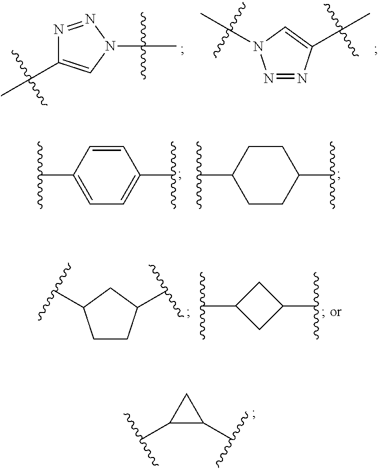

and each R^{3b} is independently hydrogen, methyl, or ethyl, or together —C(R^{3b})₂-forms cyclopropylenyl.

In some embodiments, R³ is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂—O—CH(CO₂H)—CH₂—, —CH₂—Se—CH(CO₂H)—CH₂—, —CH₂—CH(CO₂H)—(CH₂)₂—, —(CH₂)₂—CH(CO₂H)—, —CH₂—CH(CO₂H)—CH₂—, —(CH₂)₁₋₂—R^{3h}—(CH₂)₀₋₂—, —(CH₂)_{0- 2}—R^{3h}—(CH₂)₁₋₂- or (CH₂)₁₋₃—NH—C(O)—C(R R^{3h} is

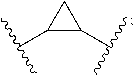 [SMILES: *C1CC1*]

and each R^{3b} is, independently, hydrogen, methyl, or ethyl, or together —C(R^{3b})₂— forms cyclopropylenyl.

In some embodiments, R³ is —CH₂—NH—C(O)—CH₂—, —CH₂—O—(CH₂)₂—, —(CH₂)₃—O—, —CH₂—S—CH₂—CH(CO₂H)—, —(CH₂)₁₋₂— R^{3h}—(CH₂)₀₋₂— or —(CH₂)₀₋₂—R^{3h}—(CH₂)₁₋₂—; and wherein R^{3h} is

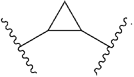 [SMILES: *C1CC1*]

In some embodiments, the compound further comprises one or more radiolabeling groups connected to the linker, independently selected from: a radiometal chelator optionally bound by a radiometal; an aryl or heteroaryl substituted with a radiohalogen; a prosthetic group containing a trifluoroborate; or a prosthetic group containing a silicon-fluorine-acceptor moiety, a fluorophosphate, a fluorosulfate, or a sulfonylfluoride. In some embodiments, the compound comprises a radiometal chelator. In some embodiments, the radiometal chelator is bound by a radiometal. In some embodiments, the compound comprises an aryl substituted with a radiohalogen. In some embodiments, the compound comprises a prosthetic group containing a trifluoroborate. In some embodiments, the compound comprises a prosthetic group containing a silicon-fluorine-acceptor moiety. In some embodiments, the compound comprises a prosthetic group containing a fluorophosphate. In some embodiments, the compound comprises a prosthetic group containing a fluorosulfate. In some embodiments, the compound comprises a prosthetic group containing a sulfonylfluoride. In some embodiments, a fluorine in the aforementioned groups is ¹⁸F.

In some embodiments, the one or more radiolabeling groups comprise: a radiometal chelator optionally bound by a radiometal; and a prosthetic group containing a trifluoroborate, optionally wherein 1, 2 or 3 fluorines in the trifluoroborate are ¹⁸F.

In some embodiments, the compound comprising a PSMA-targeting moiety of Formula II is a compound of Formula I or is a salt or solvate of Formula I, wherein R² is —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

The following definitions apply to Formula A′, A, B′, B, III-a and III-b compounds (and salts, solvates, stereoisomers thereof).

In some embodiments, R² is —CH₂—. In some embodiments, R² is —CH(OH)—. In some embodiments, R² is —CHF—. In some embodiments, R² is —CF₂—. In some embodiments, R² is —CH(CH₃)—. In some embodiments, R² is —C(CH₃)₂—. In some embodiments, R² is —CH₂CH(OH)—. In some embodiments, R² is —CH₂CHF—. In some embodiments, R² is —CHFCH₂—. In some embodiments, R² is —CF₂CH₂—. In some embodiments, R² is —CH₂CF₂—. In some embodiments, R² is —CH(OH)CH₂—. In some embodiments, R² is —CH(CH₃)CH₂—. In some embodiments, R² is —CH₂CH(CH₃)—. In some embodiments, R² is —C(CH₃)₂CH₂—. In some embodiments, R² is —CH₂C(CH₃)₂—. In some embodiments, R² is —CH₂CH(OH)CH₂—. In some embodiments, R² is —CH₂CHFCH₂—. In some embodiments, R² is —(CH₂)₂CH(OH)—. In some embodiments, R² is —(CH₂)₂CHF—. In some embodiments, R² is —(CH₂)₃—. In some embodiments, R² is —CH₂OCH₂—. In some embodiments, R² is —CH₂SCH₂—. In some embodiments, R² is —CHFCH₂CH₂—. In some embodiments, R² is —CH(OH)CH₂CH₂—. In some embodiments, R² is —CH(CH₃)CH₂CH₂—. In some embodiments, R² is —CH₂CH(CH₃)CH₂—. In some embodiments, R² is —CH₂CH₂CH(CH₃)—. In some embodiments, R² is —C(CH₃)₂CH₂CH₂—. In some embodiments, R² is —CH₂C(CH₃)₂CH₂—. In some embodiments, R² is —CH₂CH₂C(CH₃)₂—. In some embodiments, R² is —CH(CH₃)—O—CH₂—. In some embodiments, R² is —C(CH₃)₂O—CH₂—. In some embodiments, R² is —CH₂—O—CH(CH₃)—. In some embodiments, R² is —CH₂—O—C(CH₃)₂—. In some embodiments, R² is —CH₂—S(O)—CH₂—. In some embodiments, R² is —CH₂—S(O)₂—CH₂—. In some embodiments, R² is —CH(CH₃)—S—CH₂—. In some embodiments, R² is —C(CH₃)₂—S—CH₂—. In some embodiments, R² is —CH₂—S—CH(CH₃)—. In some embodiments, R² is —CH₂—S—C(CH₃)₂—. In some embodiments, R² is —CH(CH₃)—S(O)—. In some embodiments, R² is CH₂—. In some embodiments, R² is —C(CH₃)₂—S(O)—CH₂—. In some embodiments, R² is —CH₂—S(O)—CH(CH₃)—. In some embodiments, R² is —CH₂—S(O)—C(CH₃)₂—. In some embodiments, R² is —CH(CH₃)—S(O)₂—CH₂—. In some embodiments, R² is —C(CH₃)₂—S(O)₂—CH₂—. In some embodiments, R² is —CH₂—S(O)₂—CH(CH₃)—. In some embodiments, R² is —CH₂—S(O)₂—C(CH₃)₂—. In some embodiments, R² is —CH₂—NH—C(O)—. In some embodiments, R² is —C(O)—NH—CH₂—. In some embodiments, R² is —C(O)—NH—CH(CH₃)—. In some embodiments, R² is —C(O)—NH—C(CH₃)₂—. In some embodiments, R² is —CH₂SeCH₂—. In some embodiments, R² is —CH(COOH)—. In some embodiments, R² is —CH₂CH(COOH)—. In some embodiments, R² is —CH₂CH(COOH)CH₂—. In some embodiments, R² is —CH₂CH₂CH(COOH)—. In some embodiments, R² is —CH═CH—. In some embodiments, R² is —CH═CHCH₂—. In some embodiments, R² is —C≡CCH₂—. In some embodiments, R² is —HC[CH₂]CH—. In some embodiments, R² is —HC[CH₂]CHCH₂—.

In some embodiments, R² is —CH₂—, —(CH₂)₂—, —CH₂CHF—, —CHFCH₂—, —(CH₂)₃—, —CH₂OCH₂—, or —CH₂SCH₂—. In some embodiments, R² is —(CH₂)₃—. In some embodiments, R² is —(CH₂)₂—, —(CH₂)₃—, or —CH₂SCH₂—. In some embodiments, R² is —(CH₂)₃- or —CH₂SCH₂—.

The following definitions apply to Formula A′, A, and I-a compounds (and salts, solvates, stereoisomers thereof).

In some embodiments, R^{4a} is —O—, —S—, —Se—, —S(O)—, or. —S(O)₂—. In some embodiments, R^{4a} is

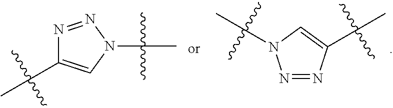 [SMILES: *c1cn(*)nn1 ; *c1cn(*)nn1]

In some embodiments, R^{4a} is —S—S— or —S—CH₂—S—.

In some embodiments, R^{4a} is —N(R^{4b})—C(O)—. In some embodiments, R^{4a} is —C(O)—N(R^{4b})—. In some embodiments, R^{4a} is —C(O)—N(R^{4b})—NH—C(O)—. In some embodiments, R^{4a} is —C(O)—NH—N(R^{4b})—C(O)—. In some embodiments, R^{4a} is —O—C(O)—N(R^{4b})—. In some embodiments, R^{4a} is —N(R^{4b})—C(O)—O—. In some embodiments, R^{4a} is —N(R^{4b})—C(O)—NH—. In some embodiments, R^{4a} is —NH—C(O)—N(R^{4b})—. In some embodiments, R^{4a} is —O—C(S)—N(R^{4b})—. In some embodiments, R^{4a} is —N(R^{4b})—C(S)—O—. In some embodiments, R^{4a} is —N(R^{4b})—C(S)—NH—. In some embodiments, R^{4a} is —NH—C(S)—N(R^{4b})—. In some embodiments, R^{4a} is —N(R^{4b})—C(O)—C(O)—NH—. In some embodiments, R^{4a} is —NH—C(O)—C(O)—N(R^{4b})—. In some embodiments, R^{4a} is —N(R^{4b})—NH—C(O)—. In some embodiments, R^{4a} is —NH—N(R^{4b})—C(O)—. In some embodiments, R^{4a} is —C(O)—N(R^{4b})—NH—. In some embodiments, R^{4a} is —C(O)—NH—N(R^{4b})—. In some embodiments, R^{4a} is or —C(O)—N(R^{4b})—O—.

In some embodiments, R^{4b} is hydrogen.

In some embodiments, R^{4a} is —NHC(O)—. In some embodiments, R^{4a} is —C(O)NH—.

In some embodiments, R^{4b} is methyl. In some embodiments, R^{4b} is ethyl.

In some embodiments, R^{4b} is non-substituted phenyl. In some embodiments, R^{4b} is phenyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, the one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is non-substituted benzyl. In some embodiments, R^{4b} is benzyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is benzyl optionally substituted with one or a combination of OH, NH₂, NO₂, halogen, C₁-C₆ alkyl, and/or C₁-C₆ alkoxyl groups. In some embodiments, R^{4b} is benzyl optionally para-substituted with a halogen.

In some embodiments, R^{4a} is —N(R^{4b})—C(O)— or —C(O)—N(R^{4b})—, wherein R^{4b} is —(CH₂)₀₋₁-(phenyl), wherein 1-5 (i.e., 1, 2, 3, 4, or 5) of the phenyl ring hydrogens are optionally substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

The following definitions apply to Formula B′, B, I-b, Ill-b, and IV-b compounds (and salts, solvates, stereoisomers thereof).

In some embodiments, R^{4a} is —N(R^{4b})—C(O)—. In some embodiments, R^{4a} is —C(O)—N(R^{4b})—. In some embodiments, R^{4a} is —C(O)—N(R^{4b})—NH—C(O)—. In some embodiments, R^{4a} is —C(O)—NH—N(R^{4b})—C(O)—. In some embodiments, R^{4a} is —O—C(O)—N(R^{4b})—. In some embodiments, R^{4a} is —N(R^{4b})—C(O)—O—. In some embodiments, R^{4a} is —N(R^{4b})—C(O)—NH—. In some embodiments, R^{4a} is —NH—C(O)—N(R^{4b})—. In some embodiments, R^{4a} is —O—C(S)—N(R^{4b})—. In some embodiments, R^{4a} is —N(R^{4b})—C(S)—O—. In some embodiments, R^{4a} is —N(R^{4b})—C(S)—NH—. In some embodiments, R^{4a} is —NH—C(S)—N(R^{4b})—. In some embodiments, R^{4a} is —N(R^{4b})—C(O)—C(O)—NH—. In some embodiments, R^{4a} is —NH—C(O)—C(O)—N(R^{4b})—. In some embodiments, R^{4a} is —N(R^{4b})—NH—C(O)—. In some embodiments, R^{4a} is —NH—N(R^{4b})—C(O)—. In some embodiments, R^{4a} is —C(O)—N(R^{4b})—NH—. In some embodiments, R^{4a} is —C(O)—NH—N(R^{4b})—. In some embodiments, R^{4a} is or —C(O)—N(R^{4b})—O—.

In some embodiments, R^{4a} is —NHC(O)—. In some embodiments, R^{4a} is —C(O)NH—.

In some embodiments, R^{4b} is methyl. In some embodiments, R^{4b} is ethyl.

In some embodiments, R^{4b} is non-substituted phenyl. In some embodiments, R^{4b} is phenyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is non-substituted benzyl. In some embodiments, R^{4b} is benzyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is benzyl optionally substituted with one or a combination of OH, NH₂, NO₂, N₃, CN, SMe, CF₃, CHF₂, halogen, C₁-C₆ alkyl, and/or C₁-C₆ alkoxyl groups. In some embodiments, R^{4b} is benzyl optionally para-substituted with a halogen.

In some embodiments, R^{4a} is —N(R^{4b})—C(O)— or —C(O)—N(R^{4b})—, wherein R^{4b} is —(CH₂)₀₋₁-(phenyl), wherein 1-5 (i.e., 1, 2, 3, 4, or 5) of the phenyl ring hydrogens are optionally substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, C, or I.

The following definitions apply to Formula A′, A, III-a and IV-a compounds (and salts, solvates, stereoisomers thereof).

In some embodiments, R^{4a} is —O—. In some embodiments, R^{4a} is —S—. In some embodiments, R^{4a} is —Se—. In some embodiments, R^{4a} is —S(O)— In some embodiments, R⁴° is-S(O)₂—.

In some embodiments, R^{4a} is

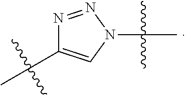 [SMILES: *c1cn(*)nn1]

In some embodiments, R^{4a} is

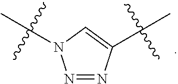 [SMILES: *c1cn(*)nn1]

In some embodiments, R^{4a} is —S—S—. In some embodiments, R^{4a} is —S—CH₂—S—.

In some embodiments, R^{4a} is

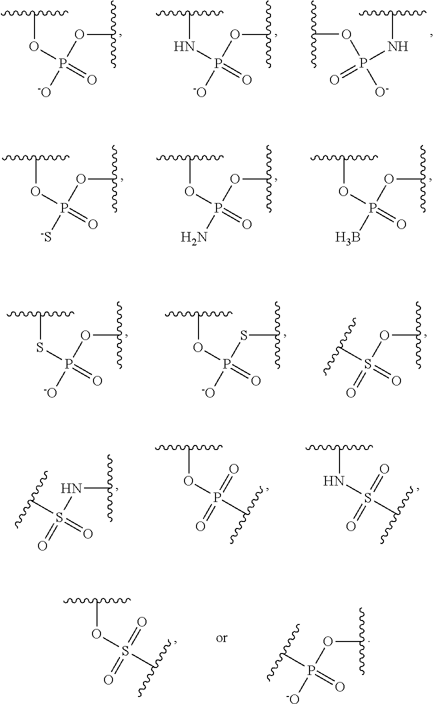

In some embodiments, R^{4a} is —N(R^{4b})—C(O)—. In some embodiments, R^{4a} is —C(O)—N(R^{4b})—. In some embodiments, R^{4a} is —C(O)—N(R^{4b})—NH—C(O)—. In some embodiments, R^{4a} is —C(O)—NH—N(R^{4b})—C(O)—. In some embodiments, R^{4a} is —O—C(O)—N(R^{4b})—. In some embodiments, R^{4a} is —N(R^{4b})—C(O)—O—. In some embodiments, R^{4a} is —N(R^{4b})—C(O)—NH—. In some embodiments, R^{4a} is —NH—C(O)—N(R^{4b})—. In some embodiments, R^{4a} is —O—C(S)—N(R^{4b})—. In some embodiments, R^{4a} is —N(R^{4b})—C(S)—O—. In some embodiments, R^{4a} is —N(R^{4b})—C(S)—NH—. In some embodiments, R^{4a} is —NH—C(S)—N(R^{4b})—. In some embodiments, R^{4a} is —N(R^{4b})—C(O)—C(O)—NH—. In some embodiments, R^{4a} is —NH—C(O)—C(O)—N(R^{4b})—. In some embodiments, R^{4a} is —N(R^{4b})—NH—C(O)—. In some embodiments, R^{4a} is —NH—N(R^{4b})—C(O)—. In some embodiments, R^{4a} is —C(O)—N(R^{4b})—NH—. In some embodiments, R^{4a} is —C(O)—NH—N(R^{4b})—. In some embodiments, R^{4a} is or —C(O)—N(R^{4b})—O—.

In some embodiments, R^{4b} is hydrogen.

In some embodiments, R^{4a} is —NHC(O)—. In some embodiments, R^{4a} is —C(O)NH—.

In some embodiments, R^{4b} is methyl. In some embodiments, R^{4b} is ethyl.

In some embodiments, R^{4b} is non-substituted phenyl. In some embodiments, R^{4b} is phenyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is non-substituted benzyl. In some embodiments, R^{4b} is benzyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R^{4b} is benzyl optionally substituted with one or a combination of OH, NH₂, NO₂, halogen, C₁-C₆ alkyl, and/or C₁-C₆ alkoxyl groups. In some embodiments, R^{4b} is benzyl optionally para-substituted with a halogen.

In some embodiments, R^{4a} is —N(R^{4b})—C(O)— or —C(O)—N(R^{4b})—, wherein R^{4b} is —(CH₂)₀₋₁-(phenyl), wherein 1-5 (i.e., 1, 2, 3, 4, or 5) of the phenyl ring hydrogens are optionally substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

The following definitions apply to Formula A′, A, B′, B, IV-a and IV-b compounds (and salts, solvates, stereoisomers thereof).

In some embodiments, R² is —CH₂— In some embodiments, R² is —CH(OH)—. In some embodiments, R² is —CHF—. In some embodiments, R² is —CF₂—. In some embodiments, R² is —CH(CH₃)—. In some embodiments, R² is —C(CH₃)₂—. In some embodiments, R² is —CH₂CH(OH)—. In some embodiments, R² is —CH₂CHF—. In some embodiments, R² is —CHFCH₂—. In some embodiments, R² is —CF₂CH₂—. In some embodiments, R² is —CH₂CF₂—. In some embodiments, R² is —CH(OH)CH₂—. In some embodiments, R² is —CH(CH₃)CH₂—. In some embodiments, R² is —CH₂CH(CH₃)—. In some embodiments, R² is —C(CH₃)₂CH₂—. In some embodiments, R² is —CH₂C(CH₃)₂—. In some embodiments, R² is —CH₂CH(OH)CH₂—. In some embodiments, R² is —CH₂CHFCH₂—. In some embodiments, R² is —(CH₂)₂CH(OH)—. In some embodiments, R² is —(CH₂)₂CHF—. In some embodiments, R² is —(CH₂)₃—. In some embodiments, R² is —CH₂OCH₂—. In some embodiments, R² is —CH₂SCH₂—. In some embodiments, R² is —CHFCH₂CH₂—. In some embodiments, R² is —CH(OH)CH₂CH₂—. In some embodiments, R² is —CH(CH₃)CH₂CH₂—. In some embodiments, R² is —CH₂CH(CH₃)CH₂—. In some embodiments, R² is —CH₂CH₂CH(CH₃)—. In some embodiments, R² is —C(CH₃)₂CH₂CH₂—. In some embodiments, R² is —CH₂C(CH₃)₂CH₂—. In some embodiments, R² is —CH₂CH₂C(CH₃)₂—. In some embodiments, R² is —CH(CH₃)—O—CH₂—. In some embodiments, R² is —C(CH₃)₂O—CH₂—. In some embodiments, R² is —CH₂—O—CH(CH₃)—. In some embodiments, R² is —CH₂—O—C(CH₃)₂—. In some embodiments, R² is —CH₂—S(O)—CH₂—. In some embodiments, R² is —CH₂—S(O)₂—CH₂—. In some embodiments, R² is —CH(CH₃)—S—CH₂—. In some embodiments, R² is —C(CH₃)₂—S—CH₂—. In some embodiments, R² is —CH₂—S—CH(CH₃)—. In some embodiments, R² is —CH₂—S—C(CH₃)₂—. In some embodiments, R² is —CH(CH₃)—S(O)—. In some embodiments, R² is CH₂—. In some embodiments, R² is —C(CH₃)₂—S(O)—CH₂—. In some embodiments, R² is —CH₂—S(O)—CH(CH₃)—. In some embodiments, R² is —CH₂—S(O)—C(CH₃)₂—. In some embodiments, R² is —CH(CH₃)—S(O)₂—CH₂—. In some embodiments, R² is —C(CH₃)₂—S(O)₂—CH₂—. In some embodiments, R² is —CH₂—S(O)₂—CH(CH₃)—. In some embodiments, R² is —CH₂—S(O)₂—C(CH₃)₂—. In some embodiments, R² is —CH₂—NH—C(O)—. In some embodiments, R² is —C(O)—NH—CH₂—. In some embodiments, R² is —C(O)—NH—CH(CH₃)—. In some embodiments, R² is —C(O)—NH—C(CH₃)₂—. In some embodiments, R² is —CH₂SeCH₂—. In some embodiments, R² is —CH(COOH)—. In some embodiments, R² is —CH₂CH(COOH)—. In some embodiments, R² is —CH₂CH(COOH)CH₂—. In some embodiments, R² is —CH₂CH₂CH(COOH)—. In some embodiments, R² is —CH═CH—, —CH═CHCH₂—. In some embodiments, R² is —C≡CCH₂—. In some embodiments, R² is —HC[CH₂]CH—. In some embodiments, R² is —HC[CH₂]CHCH₂—.

In some embodiments, R² is —CH₂—, —(CH₂)₂—, —CH₂CHF—, —CHFCH₂—, —(CH₂)₃—, —CH₂OCH₂—, or —CH₂SCH₂—. In some embodiments, R² is —(CH₂)₃—. In some embodiments, R² is —(CH₂)₂—, —(CH₂)₃—, or —CH₂SCH₂—. In some embodiments, R² is —(CH₂)₃- or —CH₂SCH₂—.

In some embodiments, R⁶ is hydrogen.

In some embodiments, R⁶ is methyl. In some embodiments, R⁶ is ethyl.

In some embodiments, R⁶ is non-substituted phenyl. In some embodiments, R⁶ is phenyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, C, or I.

In some embodiments, R⁶ is non-substituted benzyl. In some embodiments, R⁶ is benzyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, one of the ring hydrogens is para-substituted with halogen. In some embodiments, the halogen is Br. In some embodiments, the halogen is F, Cl, or I.

In some embodiments, R⁶ is a carbonyl, a phosphoryl or a sulfonyl group that is linked to the alpha-nitrogen in Xaa¹ to respectively give an amide, phosphoramidate/phosphonamidate, or sulfonamide linkage; or —NHC(O)—, —(NH)₂—C(O)—, —C(O)—(NH)₂—C(O)—, —OC(O)—, —OC(S)—, —NHC(S)—, —NHC(O)C(O)—, or —NH—NH—C(O)—, to enjoin the alpha-nitrogen in Xaa¹.

The following definitions apply to Formula A′, A, B′, B, I-a, I-b, Ill-a, and Ill-b.

In some embodiments, R⁶ is hydrogen.

In some embodiments, R⁶ is methyl. In some embodiments, R⁶ is ethyl.

In some embodiments, R⁶ is non-substituted phenyl. In some embodiments, R⁶ is phenyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, the one of the ring hydrogens is para-substituted with halogen. In some embodiments, the halogen is Br. In some embodiments, the halogen is F, Cl, or I.

In some embodiments, R⁶ is non-substituted benzyl. In some embodiments, R⁶ is benzyl wherein 1-5 (i.e., 1, 2, 3, 4, or 5) of the phenyl ring hydrogens are substituted with one or a combination of OH, NH₂, NO₂, halogen, C₁-C₆ alkyl, and/or C₁-C₆ alkoxyl groups. In some embodiments, one of the ring hydrogens is substituted (e.g. para-substituted, ortho-substituted, or meta-substituted). In some embodiments, one of the ring hydrogens is substituted with halogen. In some embodiments, the one of the ring hydrogens is para-substituted with halogen. In some embodiments, the halogen is Br. In some embodiments, the halogen is F, Cl, or I.

Unless otherwise specified, the following definitions apply to all Formula A′, A, B′, B, I-a, I-b, Ill-a, Ill-b, IV-a, and IV-b compounds (or salts, solvates, stereoisomers thereof) as well as compounds comprising a PSMA-targeting moiety of Formula II (or a salts, solvates, stereoisomers thereof). The following definitions therefore apply to compounds comprising Formula II PSMA-targeting moieties, including but not necessarily limited to when such compounds are Formula I-a compounds.

In some embodiments, R⁰ is O. In other embodiments, R⁰ is S.

In some embodiments, R^{1a} is —CO₂H. In some embodiments, R^{1a} is —SO₂H. In some embodiments, R^{1a} is —SO₃H, —PO₂H. In some embodiments, R^{1a} is —PO₃H₂. In some embodiments, R^{1a} is —OPO₃H₂. In some embodiments, R^{1a} is —OSO₃H. In some embodiments, R^{1a} is —B(OH)₂. In some embodiments, R^{1a} is

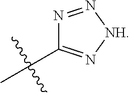 [SMILES: *c1nn[nH]n1]

In some embodiments, R^{1a} is an anionic or metallated salt of any of the foregoing.

In some embodiments, R^{1b} is —CO₂H. In some embodiments, R^{1a} is —SO₂H. In some embodiments, R^{1a} is —SO₃H. In some embodiments, R^{1a} is —PO₂H. In some embodiments, R^{1a} is —PO₃H₂. In some embodiments, R^{1b} is —B(OH)₂. In some embodiments, R^{1b} is

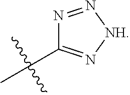 [SMILES: *c1nn[nH]n1]

In some embodiments, R^{1a} is an anionic or metallated salt of any of the foregoing.

In some embodiments, R^{1c} is —CO₂H. In some embodiments, R^{1a} is —SO₂H. In some embodiments, R^{1a} is —SO₃H. In some embodiments, R^{1a} is —PO₂H. In some embodiments, R^{1a} is —PO₃H₂. In some embodiments, R^{1c} is —B(OH)₂. In some embodiments, R^{1c} is

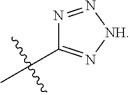 [SMILES: *c1nn[nH]n1]

In some embodiments, R^{1a} is an anionic or metallated salt of any of the foregoing.

In some embodiments, R^{1a} is —CO₂H. In some embodiments, R^{1b} is —CO₂H. In some embodiments, R^{1c} is —CO₂H. In some embodiments, R^{1a} and R^{1b} are each —CO₂H. In some embodiments, R^{1a} and R^{1c} are each —CO₂H. In some embodiments, R^{1b} and R^{1c} are each —CO₂H. In some embodiments, Ria, R^{1b}, and R^{1c} are anionic or metallated salts of any of the foregoing.

In some embodiments, R^{1a}, R^{1b} and R^{1c} are each —CO₂H (or an anionic or metallated salt thereof).

In some embodiments, R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl.

In some embodiments, R^{3a} is a linear acyclic C₃-C₁₅ alkylenyl. In some embodiments, R^{3a} is a linear acyclic C₃-C₁₅ alkylenyl in which 1-5 carbons are (independently) replaced with N, S and/or O heteroatoms. In some embodiments, R^{3a} is a linear acyclic saturated C₃-C₁₀ alkylenyl, optionally independently substituted with 1-5 amine, amide, oxo, hydroxyl, thiol, methyl and/or ethyl groups. In some embodiments, R^{3a} is —(CH₂)₃-1₅—. In some embodiments, R^{3a} is —CH₂—. In some embodiments, R^{3a} is —(CH₂)₂—. In some embodiments, R³ is —(CH₂)₃—. In some embodiments, R^{3a} is —(CH₂)₄—. In some embodiments, R^{3a} is —(CH₂)₅—. In some embodiments, R^{3a} is —CH—O—CH₂—. In some embodiments, R^{3a} is —CH₂—S—CH₂—. In some embodiments, R^{3a} is —CH═CH—. In some embodiments, R^{3a} is —CH₂—C≡C—. In some embodiments, R^{3a} is a linear C₃—C alkenylenyl and/or alkynylenyl.

In some embodiments, R^{3a} is: a linear C₃-C₅ alkylenyl, optionally wherein one methylene is replaced with —S—, —O—, —S—CH(CH₃)—, —O—CH(CH₃)—, —CH(CH₃)—S—, —CH(CH₃)—O—, wherein the S and O heteroatoms are spaced apart from other heteroatoms in the compound by at least 2 carbons, and optionally wherein one ethylene is replaced with —CH═CH—, —CC—, a 3-6 membered cycloalkylenyl or arylenyl,

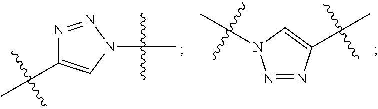 [SMILES: *c1cn(*)nn1 ; *c1cn(*)nn1]

In some embodiments, R^{3a} is optionally substituted with oxo. In some embodiments, R^{3a} is a heteroalkylenyl, which is optionally substituted. In some embodiments, heteroalkylenyl optionally substituted with at least one oxo forms an amide group within the heteroalkyleneyl. In some embodiments, heteroalkylenyl substituted with at least one oxo is —(CH₂)₁₋₃—NH—C(O)—C(R^{3b})₂-, wherein each R^{3b} is, independently, hydrogen, methyl, or ethyl, or together —C(R^{3b})₂— forms cyclopropylenyl.

In some embodiments, R^{3a} is —(CH₂)₁₋₃—NH—C(O)—C(R^{3b})₂-, wherein each R^{3b} is independently hydrogen, methyl, or ethyl, or together —C(R^{3b})₂— forms cyclopropyl-enyl (i.e. —CH[CH₂]CH—), and which is oriented in the compound as shown below:

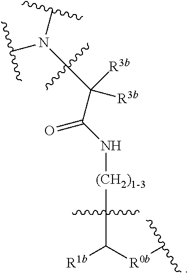

In some embodiments, R^{3a} is —(CH₂)₃—. In some embodiments, R^{3a} is —(CH₂)₄—. In some embodiments, R^{3a} is —(CH₂)₅—. In some embodiments, R^{3a} is —CH₂—CH═CH—CH₂—. In some embodiments, R^{3a} is —CH₂—CH₂—CH═CH—, wherein the terminal alkenyl carbon is bonded to a carbon in the compound. In some embodiments, R^{3a} is —CH₂—C≡C—CH₂—. In some embodiments, R^{3a} is —C(R^{3b})₂—C(O)—NH—(CH₂)₁₋₂— wherein the leftmost carbon is bonded to a nitrogen of R^{4a} and each R^{3b} is independently hydrogen, methyl, or ethyl, or together —C(R^{3b})₂— forms cyclopropyl-enyl (i.e. —CH[CH₂]CH—). In some embodiments, R^{3a} is —CH₂—CH₂—S—CH(R^{3c})—, wherein R^{3c} is hydrogen or methyl. In some embodiments, R^{3a} is —CH₂—CH₂O—CH(R^{3c})—, wherein R^{3c} is hydrogen or methyl.

In some embodiments, R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic C₂-C₂₀ heteroalkylenyl, heteroalkenylenyl, or heteroalkynylenyl, wherein R^{3a} is optionally substituted.

In some embodiments, R^{3a} is —CH₂—; —(CH₂)₂—; —(CH₂)₃; —(CH₂)₄—; —(CH₂)₅—; —CH₂—O—CH₂—; —CH₂—S—CH₂—; —CH₂—O—(CH₂)₂—; —(CH₂)₃—O—; —CH₂—S—CH₂—CH(CO₂H)—; —(CH₂)₃—CH(CO₂H)—; —CH₂—O—CH₂—CH(CO₂H)—; —CH₂—Se—CH₂—CH(CO₂H)—; —(CH₂)₁₋₂—R^{3h}—(CH₂)₀₋₂—; —(CH₂)₀₋₂—R^{3h}—(CH₂)₁₋₂—; or —(CH₂)₁₋₃—NH—C(O)—C(R^{3b})₂—; R^{3h} is:

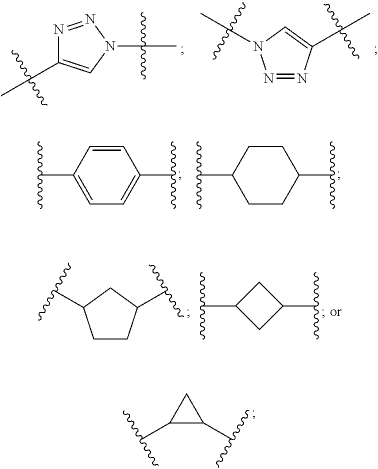

and each R^{3b} is independently hydrogen, methyl, or ethyl, or together —C(R^{3b})₂-forms cyclopropylenyl.

In some embodiments, R^{3a} is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂—O—CH(CO₂H)—CH₂—, —CH₂—Se—CH(CO₂H)—CH₂—, —CH₂—CH(CO₂H)—(CH₂)₂—, —(CH₂)₂—CH(CO₂H)—, —CH₂—CH(CO₂H)—CH₂—, —(CH₂)₁₋₂— R^{3h}—(CH₂)₀₋₂-, —(CH₂)_{0- 2}—R^{3h}—(CH₂)₁₋₂- or —(CH₂)₁₋₃—NH—C(O)—C(R^{3b})₂—; R^{3h} is

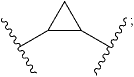 [SMILES: *C1CC1*]

and each R^{3b} is, independently, hydrogen, methyl, or ethyl, or together —C(R^{3b})₂— forms cyclopropylenyl.

In some embodiments, R^{3a} is —CH₂—NH—C(O)—CH₂—, —CH₂—O—(CH₂)₂—, —(CH₂)₃—O—, —CH₂—S—CH₂—CH(CO₂H)—, —(CH₂)₁₋₂—R^{3h}—(CH₂)₀₋₂— or —(CH₂)₀₋₂—R^{3h}—(CH₂)_{1- 2}—; and wherein R^{3h} is

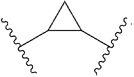 [SMILES: *C1CC1*]

In some embodiments, R^{3a} is —(CH₂)₄—, —(CH₂)₅—, —CH₂—O—(CH₂)₂—, —(CH₂)₃—O—, —CH₂—NH—C(O)—CH₂—, —CH₂—S—CH₂—CH(CO₂H)—, or —CH₂CH[CH₂]CHCH₂—. In some embodiments, R^{3a} is —(CH₂)₅—, —CH₂—O—(CH₂)₂—, —(CH₂)₃—O—, —CH₂—NH—C(O)—CH₂—, —CH₂—S—CH₂—CH(CO₂H)—, or —CH₂CH[CH₂]CHCH₂—.

In some embodiments, R^{3a} is —(CH₂)₁₋₂—R^{3h}—(CH₂)₀₋₂— or —(CH₂)₀₋₂-R^{3h}—(CH₂)₁₋₂—, wherein R^{3h} is:

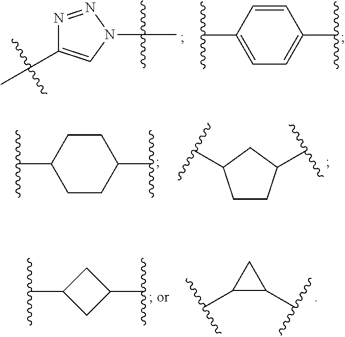

In some embodiments, R^{3a} is —(CH₂)₁₋₂— R^{3h}—(CH₂)₀₋₂— or —(CH₂)₀₋₂-R^{3h}—(CH₂)₁₋₂—, wherein R^{3h} i:

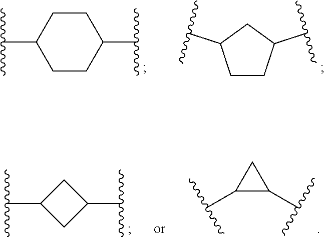 [SMILES: *C1CCC(*)CC1 ; *C1CCC(*)C1 ; *C1CC(*)C1 ; *C1CC1*]

In some embodiments, R^{3a} is

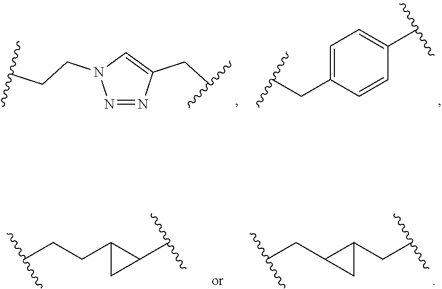 [SMILES: *CCn1cc(C*)nn1 ; *Cc1ccc(*)cc1 ; *CCC1CC1* ; *CC1CC1C*]

In some embodiments, R^{3a} is

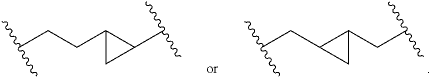 [SMILES: *CCC1CC1* ; *CC1CC1C*]

In some embodiments, —R^{4a}—R^{3a}— is —C(O)—N(R^{4b})—(CH₂)₁₋₃—R^{3d}—R^{3e}—, wherein R^{3d} is

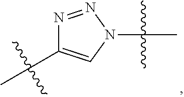 [SMILES: *c1cn(*)nn1]

and wherein R^{3e} is —CH₂—, —(CH₂)₂—, —(CH₂)₂—O—CH₂—, —(CH₂)₂—S—CH₂—, —(CH₂)₂O—CH(CH₃)—, or —(CH₂)₂—S—CH(CH₃)—. In some such embodiments, R^{3e} is —CH₂—. In some such embodiments, R^{3e} is —(CH₂)₂—. In some such embodiments, R^{3e} is —(CH₂)₂—O—CH₂—. In some such embodiments, R^{3e} is —(CH₂)₂—S—CH₂—. In some such embodiments, R^{3e} is —(CH₂)₂O—CH(CH₃)—. In some such embodiments, R^{3e} is —(CH₂)₂—S—CH(CH₃)—.

In some embodiments, —R^{4a}—R^{3a}— is —C(O)—N(R^{4b})—(CH₂)₂-3-R^{3f}—R^{3g}—, wherein R^{3f} is

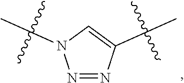 [SMILES: *c1cn(*)nn1]

and wherein R^{3g} is absent, —CH₂—, —(CH₂)₂—, —(CH₂)₀₋₂-O—CH₂—, —(CH₂)₀₋₂-S—CH₂—, —(CH₂)₀₋₂-O—CH(CH₃)—, or —(CH₂)₀₋₂-S—CH(CH₃)—. In some such embodiments, R^{3g} is absent. In some such embodiments, R^{3g} is —CH₂—. In some such embodiments, R^{3g} is —(CH₂)₂—. In some such embodiments, R^{3g} is —(CH₂)₀₋₂-O—CH₂—. In some such embodiments, R^{3g} is —(CH₂)₀₋₂-S—CH₂—. In some such embodiments, R^{3g} is —(CH₂)₀₋₂-O—CH(CH₃)—. In some such embodiments, R^{3g} is —(CH₂)₀₋₂-S—CH(CH₃)—.

R⁵ is —(CH₂)₀₋₃CH(R¹⁰)(CH₂)₀₋₃—. In some embodiments, R⁵ is —CH(R¹⁰)—. In some embodiments, R⁵ is —CH₂CH(R¹⁰)—. In some embodiments, R⁵ is —CH(R¹⁰)CH₂—. In some embodiments, R⁵ is —CH₂CH(R¹⁰)CH₂—.

In some embodiments, R¹⁰ is a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms (e.g. selected from N, O, and/or S).

In some embodiments, R¹⁰ is —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups. In some embodiments, R^{23a} is an optionally substituted C₆-C₁₆ aromatic ring or aromatic fused ring, wherein 0-3 carbons in the aromatic ring or aromatic fused ring are independently replaced with N, S and/or O heteroatoms. In some embodiments, R^{23a} is an optionally substituted C₁₀-C₁₆ aromatic ring or aromatic fused ring, wherein 0-3 carbons in the aromatic ring or aromatic fused ring are independently replaced with N.

In some embodiments, R¹⁰ is

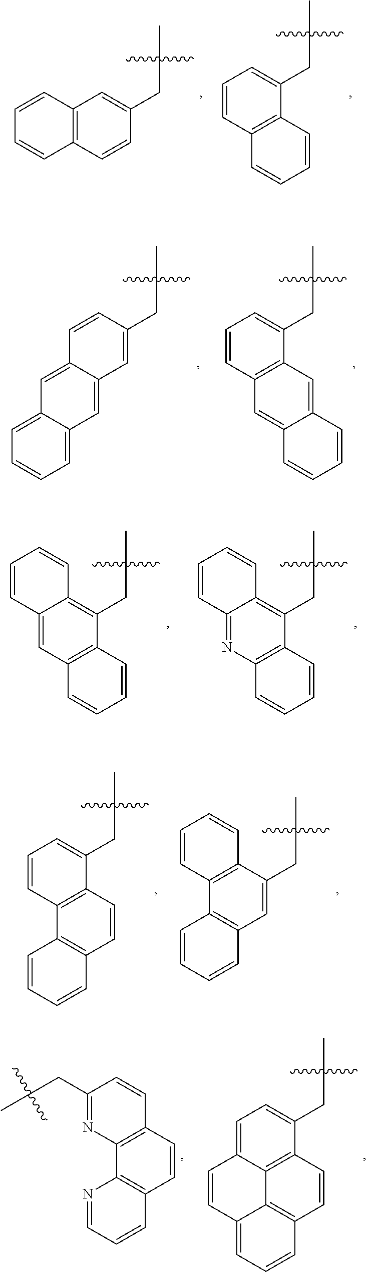

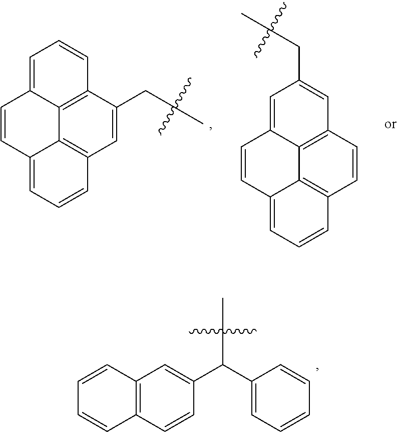 [SMILES: *Cc1cc2cccc3ccc4cccc1c4c32 ; *Cc1cc2ccc3cccc4ccc(c1)c2c34 ; *C(c1ccccc1)c1ccc2ccccc2c1]

optionally modified with one, more than one, or a combination of: halogen, OMe, SMe, NH₂, NO₂, CN, OH, or one or more additional endocyclic ring nitrogen atoms up to a maximum of 5 ring nitrogens.

In some embodiments, R¹⁰ is an alkenyl containing either a C₆-C₁₆ aryl or X₆—X₁₆ heteroaryl having 1-3 heteroatoms independently selected from N, S and/or O. In some embodiments, the C₆-C₁₆ aryl is benzyl. In some embodiments, the X₆-X₁₆ heteroaryl is benzyloxyl or benzylthio.

In some embodiments, R¹⁰ is:

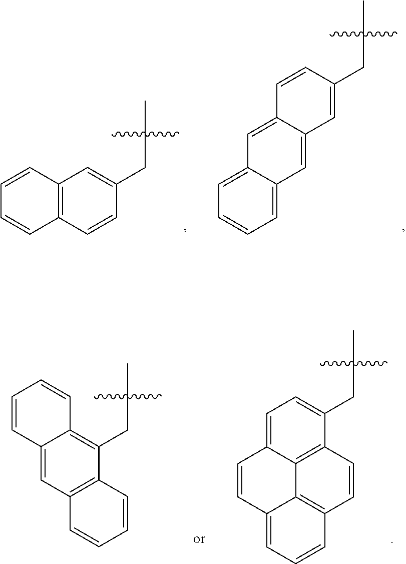 [SMILES: *Cc1ccc2ccccc2c1 ; *Cc1ccc2cc3ccccc3cc2c1 ; *Cc1c2ccccc2cc2ccccc12 ; *Cc1ccc2ccc3cccc4ccc1c2c34]

In some embodiments, R¹⁰ is

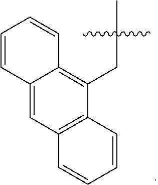 [SMILES: *Cc1c2ccccc2cc2ccccc12]

In some embodiments, R¹⁰ is

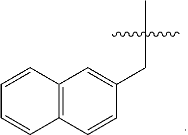 [SMILES: *Cc1ccc2ccccc2c1]

In some embodiments, R¹⁰ is

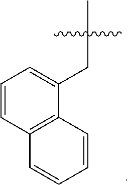 [SMILES: *Cc1cccc2ccccc12]

In some embodiments, R¹⁰ is

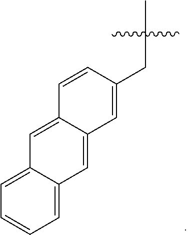 [SMILES: *Cc1ccc2cc3ccccc3cc2c1]

In some embodiments, R¹⁰ is

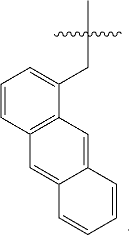 [SMILES: *Cc1cccc2cc3ccccc3cc12]

In some embodiments, R¹⁰ is

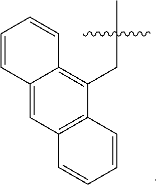 [SMILES: *Cc1c2ccccc2cc2ccccc12]

In some embodiments, R¹⁰ is

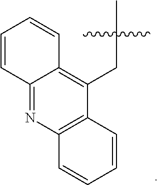 [SMILES: *Cc1c2ccccc2nc2ccccc12]

In some embodiments, R¹⁰ is

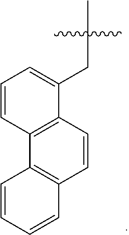 [SMILES: *Cc1cccc2c1ccc1ccccc12]

In some embodiments, R¹⁰ is

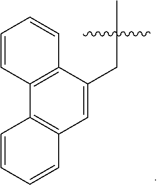 [SMILES: *Cc1cc2ccccc2c2ccccc12]

In some embodiments, R¹⁰ is

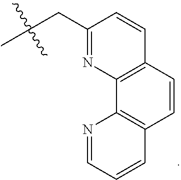 [SMILES: *Cc1ccc2ccc3cccnc3c2n1]

In some embodiments, R¹⁰ is

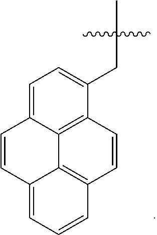 [SMILES: *Cc1ccc2ccc3cccc4ccc1c2c34]

In some embodiments, R¹⁰ is

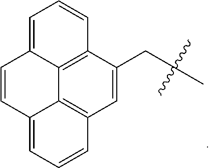 [SMILES: *Cc1cc2cccc3ccc4cccc1c4c32]

In some embodiments, R¹⁰ is

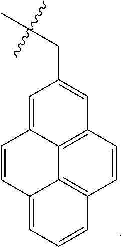 [SMILES: *Cc1cc2ccc3cccc4ccc(c1)c2c34]

In some embodiments, R¹⁰

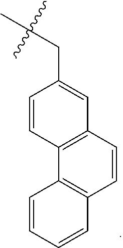 [SMILES: *Cc1ccc2c(ccc3ccccc32)c1]

In some embodiments, R¹⁰ is

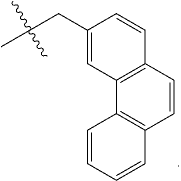 [SMILES: *Cc1ccc2ccc3ccccc3c2c1]

In some embodiments, R¹⁰ is

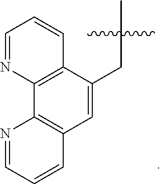 [SMILES: *Cc1cc2cccnc2c2ncccc12]

In some embodiments, R¹⁰ is

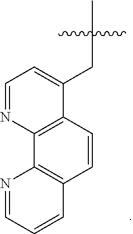 [SMILES: *Cc1ccnc2c1ccc1cccnc12]

In some embodiments, R¹⁰ is

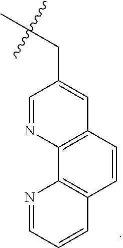 [SMILES: *Cc1cnc2c(ccc3cccnc32)c1]

In some embodiments, R¹⁰ is

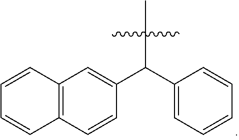 [SMILES: *C(c1ccccc1)c1ccc2ccccc2c1]

In some embodiments, R¹⁰ is:

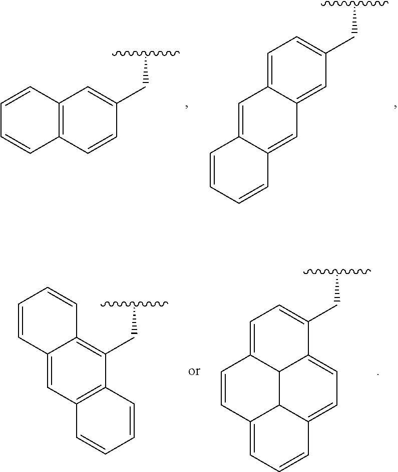

In some embodiments, R¹⁰ is

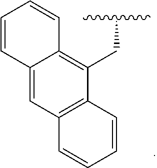

In some
embodiments, R¹⁰ is:

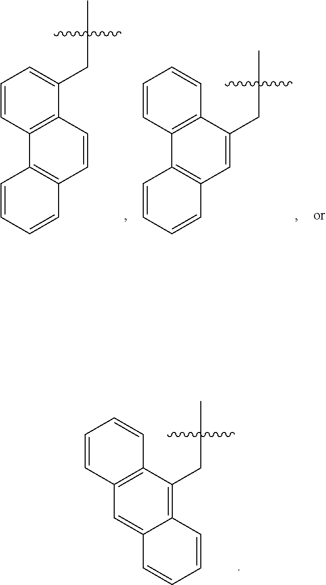 [SMILES: *Cc1cccc2c1ccc1ccccc12 ; *Cc1cc2ccccc2c2ccccc12 ; *Cc1c2ccccc2cc2ccccc12]

In some embodiments, R⁵ is —CH(R¹⁰)— wherein R¹⁰ is as defined in any embodiment above.

In some embodiments, R⁵ is —(CH₂)₀₋₃CH(R¹⁰)(CH₂)₀₋₃— and R¹⁰ is —(CH₂)₅CH₃. In some embodiments, R⁵ is —CH(R¹⁰)— and R¹⁰ is —(CH₂)₅CH₃. In some embodiments, R⁵ is —(CH₂)₀₋₃CH(R¹⁰)(CH₂)₀₋₃—.

In some embodiments, R¹⁰ is —CH²—R^{23a}. In some embodiments, R^{23a} is phenyl substituted with 1 or 2 iodo groups and optionally further substituted with 1 oxy group. In some embodiments, R⁵ is —(CH₂)₀₋₃CH(R¹⁰)(CH₂)₀₋₃— wherein R¹⁰ is —CH₂R^{23a} and R^{23a} is phenyl substituted with 1 or 2 iodo groups and optionally further substituted with 1 oxy group. In some embodiments, R^{23c} is

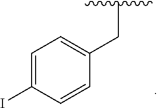 [SMILES: *Cc1ccc(I)cc1]

In some embodiments, R^{23c} is

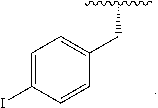

In some embodiments, R^{23a} is

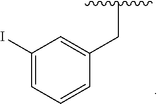 [SMILES: *Cc1cccc(I)c1]

In some embodiments, R^{23a} is

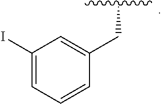

In some embodiments, R^{23a} is

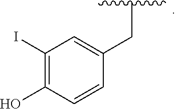 [SMILES: *Cc1ccc(O)c(I)c1]

In some embodiments, R^{23a} is

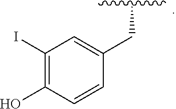

In some embodiments, R^{23a} is

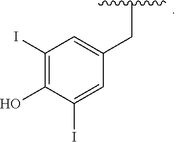 [SMILES: *Cc1cc(I)c(O)c(I)c1]

In some embodiments, R^{23a} is

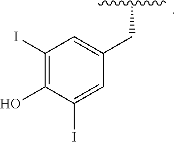

In some embodiments, R^{23a} is a radical of anthracene, phenanthene, naphthalene, acridine, or quinoline, wherein each of the foregoing is optionally substituted with one, more than one, or a combination of: halogen, OMe, SMe, NH₂, NO₂, CN, and/or OH. In some embodiments, R^{23a} is a radical of anthracene, phenanthene, naphthalene, acridine, or quinoline. In some embodiments, R^{23a} is a radical of naphthalene or quinoline, wherein each of the foregoing is optionally substituted with one, more than one, or a combination of: halogen, OMe, SMe, NH₂, NO₂, CN, and/or OH. In some embodiments, R^{23a} is a radical of naphthalene or quinoline.

In some embodiments, R¹⁰ is —CH(R^{23b})—R²³°. In some embodiments, R^{23b} is phenyl. In some embodiments, R^{23b} is naphthyl. In some embodiments, R^{23c} is phenyl. In some embodiments, R^{23c} is naphthyl. In some embodiments, wherein 0-5 (i.e. 0, 1, 2, 3, 4, or 5) carbons in each naphthyl ring and 0-3 (i.e. 0, 1, 2, or 3) carbons in each phenyl ring are independently replaced with N, S and/or O heteroatoms. In some embodiments, each naphthyl and each phenyl are independently substituted with —OH, —NH₂, —NO₂, halogen, —SMe, —CN, C₁-C₆ alkyl, and/or C₁-C₆ alkoxyl groups. In some embodiments, each naphthyl and each phenyl are non-substituted. In some embodiments, R^{23b} is phenyl and R^{23c} is naphthyl. In some embodiments, R^{23b} is naphthyl and R^{23c} is phenyl. In some embodiments, R^{23b} is phenyl and R^{23c} is phenyl. In some embodiments, R^{23b} is naphthyl and R^{23c} is naphthyl.

In some embodiments, R¹⁰ is

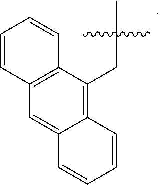 [SMILES: *Cc1c2ccccc2cc2ccccc12]

In some embodiments of the Formula III-a compounds (or salts/solvates thereof), —N(R⁶)—R⁵—R^{4a}— is

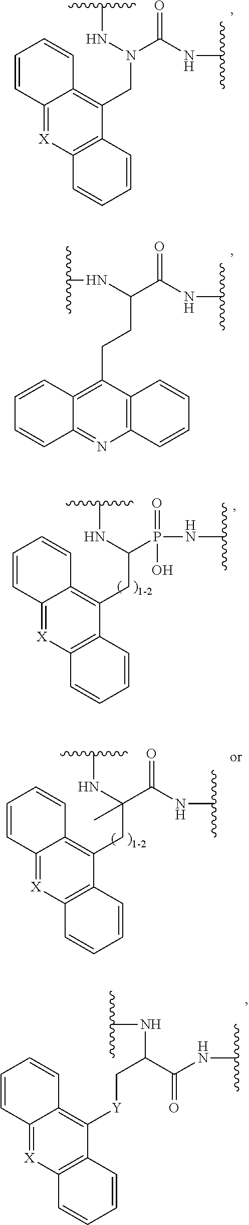

wherein X═CH or N, and Y═NH, S or O, and wherein any of these triaryl/heteroaryl groups is modified optionally with one, more than one, or a combination of halogen, OMe, SMe, NH₂, NO₂, CN, OH, or one or more additional endocyclic ring nitrogen atoms up to a maximum of 5 ring nitrogens.

In some embodiments, (Xaa¹)₁₋₄ consists of a single amino acid residue. In some embodiments, (Xaa¹)₁₋₄ is a dipeptide, wherein each Xaa¹ may be the same or different. In some embodiments, (Xaa¹)₁₋₄ is a tripeptide, wherein each Xaa¹ may be the same, different or a combination thereof. In some embodiments, (Xaa¹)₁₋₄ consists of 4 amino acid residues connected by peptide bonds, wherein each Xaa¹ may be the same, different or a combination thereof. In some embodiments, each Xaa¹ is independently selected from proteinogenic amino acids and the non-proteinogenic amino acids listed in Table 1, wherein each peptide backbone amino group is optionally methylated.

In some embodiments, at least one R⁹

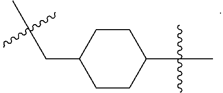 [SMILES: *CC1CCC(*)CC1]

In some embodiments, at least one R⁹

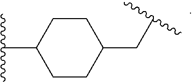 [SMILES: *CC1CCC(*)CC1]

In some embodiments, at least one R⁹.

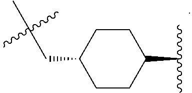

In some embodiments, at least one R⁹ is R²⁴—R²⁵—R²⁶, wherein R²⁴—R²⁵—R²⁶ are independently selected from: —(CH₂)₀₋₃—; C₃-C₈ cycloalkylene in which 0-3 carbons are (independently) replaced with N, S and/or O heteroatoms, and optionally substituted with one or more OH, NH₂, NO₂, halogen, C₁-C₆ alkyl and/or C₁-C₆ alkoxyl groups; and C₄-C₁₆ arylene in which 0-3 carbons are independently replaced with N, S and/or O heteroatoms, and optionally substituted with one or more OH, NH₂, NO₂, halogen, C₁-C₆ alkyl and/or C₁-C₆ alkoxyl groups.

In some embodiments, -(Xaa¹)₁₋₄— is -(Xaa¹)₀—N(R^{27a})—R^{27b}—C(O)—, wherein R²⁷a is hydrogen or methyl, and wherein R^{27b} is

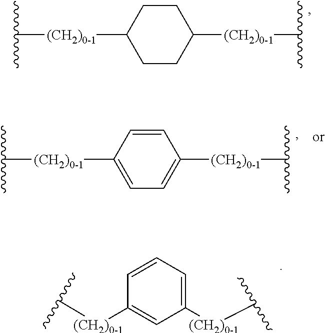 [SMILES: *CC1CCC(C*)CC1 ; *Cc1ccc(C*)cc1 ; *Cc1cccc(C*)c1]

In some embodiments, R²⁷a is hydrogen.

In some embodiments, at least one R⁸ is hydrogen. In some embodiments, all R⁸ are hydrogen.

In some embodiments, at least one Xaa¹ is a tranexamic acid residue. In some embodiments, (Xaa¹)₁₋₄ consists of a single tranexamic acid residue.

In some embodiments, -(Xaa)₁₋₄-N(R⁶)—R⁵—R^{4a}— is

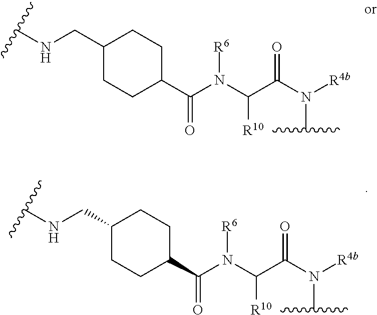

In some such embodiments, R^{4b} is hydrogen. In some such embodiments, R^{3a} is —(CH₂)₄—. In some such embodiments, R¹⁰ is any R¹⁰ defined above. In some such embodiments, R¹⁰ is —CH²—R^{23a} and R^{23a} is phenyl substituted with 1 or 2 iodo groups and optionally further substituted with 1 oxy group.

R⁷ may include a radiolabeling group optionally spaced apart using an amino acid or peptide linker. Accordingly, in some embodiments R⁷ is R^{X}-(Xaa²)₀₋₄-, wherein R^{X} bonds to the N-terminus of the N-terminal Xaa² or an amino acid group of Xaa² capable of forming an amide bond (e.g. a side chain of an alpha amino acid). An example of a Xaa² sidechain capable of forming an amide bond with R^{X} is an amino group. Non-limiting examples of amino acid residues capable of forming an amide with R^{X} include Lys, Orn, Dab, Dap, Arg, homo-Arg, and the like. In some embodiments, R^{X} bonds to the N-terminus of the N-terminal Xaa². In other embodiments, Xaa² is absent.

In some embodiments, R⁷ may include two radiolabeling groups in which the amino acid or peptide linker provides two attachment points for the radiolabeling groups. Accordingly, in some embodiments, R⁷ is

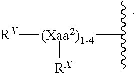

For example, a first R^{X} may bond to the N-terminus of the N-terminal Xaa² and a second R^{X} may bond to a side chain functional group (e.g. an amino group) of a Xaa². Alternatively, both R^{X} groups may bond to different Xaa² side chains or other functional groups.

In some embodiments, R⁷ is

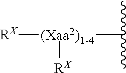

and (Xaa²)₁₋₄ is a tripeptide. In some embodiments, R⁷ is

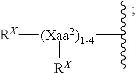

(Xaa²)₁₋₄ is a tripeptide; and R^{X} is a radiometal chelator optionally bound to a radiometal, or a prosthetic group containing a trifluoroborate.

R⁷ may include both a radiolabeling group and an albumin-binding group.

Accordingly, in some embodiments with a single R^{X} group, R⁷ is

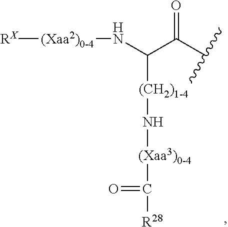

wherein when (Xaa²)₀₋₄ is (Xaa²)₁₋₄ then R^{X} bonds to the N-terminus of the N-terminal Xaa² or an amino group of Xaa² (e.g. a side chain of an alpha amino acid) capable of forming an amide bond, and wherein when (Xaa³)₀₋₄ is (Xaa³)₁₋₄ then (Xaa³)₁₋₄ is oriented to form amide bonds with the adjacent carbonyl and amine groups. In other embodiments with a single R^{X} group, R⁷ is

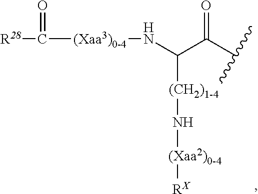

wherein when (Xaa²)₀₋₄ is (Xaa²),_4 then R^{X} bonds to the N-terminus of the N-terminal Xaa² or an amino group of Xaa² (e.g. a side chain of an alpha amino acid) capable of forming an amide bond, and wherein when (Xaa³)₀₋₄ is (Xaa³)₁₋₄ then (Xaa³)₁₋₄ is oriented to form amide bonds with the adjacent carbonyl and amine groups. In some embodiments, (Xaa²)₀₋₄ is absent. In some embodiments, Xaa³ is absent or is a single amino acid residue.

The albumin binding group R²⁸ may be any albumin binding group.

In some embodiments, the albumin binding group R²⁸ is

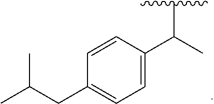 [SMILES: *C(C)c1ccc(CC(C)C)cc1]

In some embodiments, the albumin binding group R²⁸ is

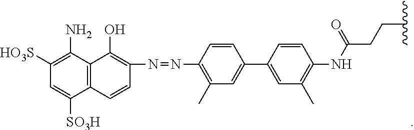 [SMILES: *CCC(=O)Nc1ccc(-c2ccc(N=Nc3ccc4c(S(=O)(=O)O)cc(S(=O)(=O)O)c(N)c4c3O)c(C)c2)cc1C]

In some embodiments, the albumin binding group R²⁸ is

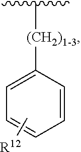

wherein R¹² is I, Br, F, Cl, H, OH, OCH₃, NH₂, NO₂ or CH₃. In some embodiments, R²⁸ is

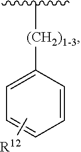

wherein R¹² is I, Br, F, Cl, H, —OH, —OCH₃, —NH₂, or —CH₃. In some embodiments, R²⁸ is

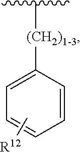

wherein R¹² is Cl or —OCH₃.

In some embodiments, R⁷ is

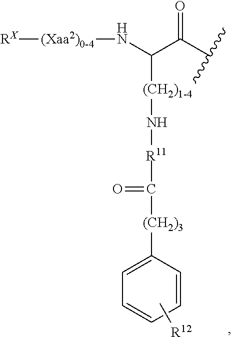

wherein when (Xaa²)₀₋₄ is (Xaa²)₁₋₄ then R^{X} bonds to the N-terminus of the N-terminal Xaa² or an amino group of Xaa² (e.g. a side chain of an alpha amino acid) capable of forming an amide bond.

In other embodiments, R⁷ is

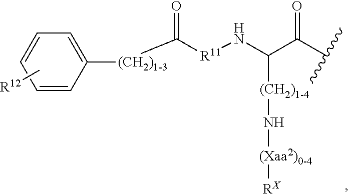

wherein when (Xaa²)₀₋₄ is (Xaa²)₁₋₄ then R^{X} bonds to the N-terminus of the N-terminal Xaa² or an amino group of Xaa² (e.g. a side chain of an alpha amino acid) capable of forming an amide bond.

In other embodiments, R⁷ is

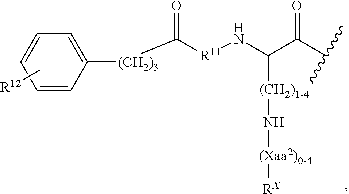

wherein when (Xaa²)₀₋₄ is (Xaa²)₁₋₄ then R^{X} bonds to the N-terminus of the N-terminal Xaa² or an amino group of Xaa² (e.g. a side chain of an alpha amino acid) capable of forming an amide bond.

In some embodiments, R¹¹ is absent. In some embodiments, R¹¹ is

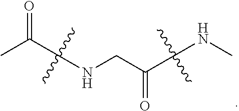

In some embodiments, R¹¹ is

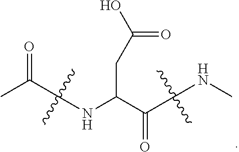

In some embodiments, R¹¹ is

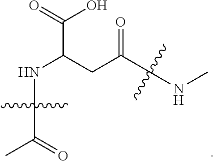

In some embodiments, R¹¹ is

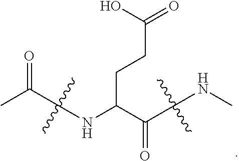

In some embodiments, R¹¹ is

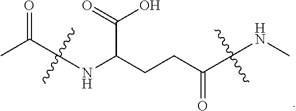

In some embodiments, R¹¹ is

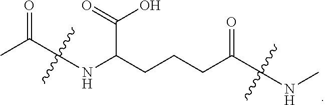

In some embodiments, R¹¹ is

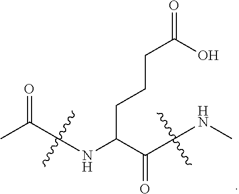

In some embodiments, R¹¹ is

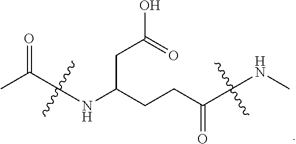

In some embodiments, R¹¹ is

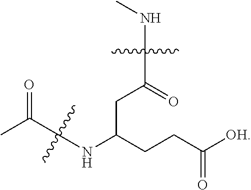

In some embodiments, R¹² is I, Br, F, Cl, H, —OH, —OCH₃, —NH₂, or —CH₃

In some embodiments, R¹² is ortho. In some embodiments, R¹² is para. In some embodiments, R¹² is meta. In some embodiments, R¹² is iodine. In some embodiments, R¹² is fluorine. In some embodiments, R¹² is chlorine. In some embodiments, R¹² is hydrogen. In some embodiments, R¹² is hydroxide. In some embodiments, R¹² is OCH₃. In some embodiments, R¹² is NH₂. In some embodiments, R¹² is NO₂. In some embodiments, R¹² is CH₃. In some embodiments, R¹² is CH₃ in para position. In some embodiments, R¹² is iodine in para position. In some embodiments, R¹² is chlorine in para position. In some embodiments, R¹² is OCH₃ in para position.

In some embodiments, Xaa² is absent. In some embodiments, (Xaa²)₀₋₄ is a single amino acid residue. In some embodiments, (Xaa²)₀₋₄ is a dipeptide, wherein each Xaa² may be the same or different. In some embodiments, (Xaa²)₀₋₄ is a tripeptide, wherein each Xaa² may be the same, different or a combination thereof. In some embodiments, (Xaa²)₀₋₄ consists of 4 amino acid residues connected by peptide bonds, wherein each Xaa² may be the same, different or a combination thereof. In some embodiments, each Xaa² is independently selected from proteinogenic amino acids and the non-proteinogenic amino acids listed in Table 1, wherein each peptide backbone amino group is optionally methylated. In some embodiments, each R¹³ in (Xaa²)₁₋₄ is hydrogen. In some embodiments, at least one R¹³ in (Xaa²)₁₋₄ is methyl. In some embodiments, at least one R¹⁴ in (Xaa²)₁₋₄ is —(CH₂)₂[O(CH₂)₂]₁₋₆— (e.g. when Xaa² is a residue of Amino-dPEG™₄-acid or Amino-dPEG™₆-acid).

In some embodiments, Xaa³ is absent. In some embodiments, (Xaa³)₀₋₄ is a single amino acid residue. In some embodiments, (Xaa³)₀₋₄ is a dipeptide, wherein each Xaa³ may be the same or different. In some embodiments, (Xaa³)₀₋₄ is a tripeptide, wherein each Xaa³ may be the same, different or a combination thereof. In some embodiments, (Xaa³)₀₋₄ consists of 4 amino acid residues connected by peptide bonds, wherein each Xaa³ may be the same, different or a combination thereof. In some embodiments, each Xaa³ is independently selected from proteinogenic amino acids and the non-proteinogenic amino acids listed in Table 1, wherein each peptide backbone amino group is optionally methylated. In some embodiments, each R¹³ in (Xaa³)₁₋₄ is hydrogen. In some embodiments, at least one R¹³ in (Xaa³)₁₋₄ is methyl. In some embodiments, at least one R¹⁴ in (Xaa³)₁₋₄ is —(CH₂)₂[O(CH₂)₂]₁₋₆— (e.g. when Xaa³ is a residue of Amino-dPEG™₄-acid or Amino-dPEG™₆-acid).

Any one or any combination of amide linkages within R⁷-Xaa¹)₁₋₄-N(R⁶)—R⁵—R^{4a}-R^{3a}may be optionally independently replaced by linkages selected from the group consisting of —O—, —S—, —Se—, —S(O)—, —S(O)₂—, —NHC(O)—, —C(O)NH—,

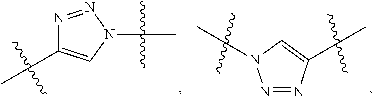 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—. In some embodiments, only one amide linkage within R⁷-(Xaa¹)₁₋₄ is replaced. In other embodiments, no amide linkages within R⁷-Xaa¹)₁₋₄-N(R⁶)—R⁵—R^{4a}—R^{3a} are replaced.

In some embodiments of the compounds of the invention, the compound is CCZ02010, CCZ02011, CCZ02018, CCZ01186, CCZ01188, CCZ01194, CCZ01198, CCZ02032, CCZ02033, ADZ-4-101, PD-6-49, PD-5-131, PD-5-159, AR-2-050-1, AR-2-050-2, AR-2-113-1 or AR-2-113-2.

In some embodiments of the compounds of the invention, one or more R^{X}comprises a radiometal chelator optionally bound by or in complex with a radiometal, or bound by or in complex with a radioisotope-bound metal. The radiometal chelator may be any radiometal chelator suitable for binding to the radiometal and which is functionalized for attachment to an amino group. Many suitable radiometal chelators are known, e.g. as summarized in Price and Orvig, Chem. Soc. Rev., 2014, 43, 260-290, which is incorporated by reference in its entirety. Non-limiting examples of radioisotope chelators include chelators selected from the group consisting of: DOTA and derivatives; DOTAGA; NOTA; NODAGA; NODASA; CB-DO2A; 3p-C-DEPA; TCMC; DO3A; DTPA and DTPA analogues optionally selected from CHX-A″-DTPA and 1B4M-DTPA; TETA; NOPO; Me-3,2-HOPO; CB-TE1A1P; CB-TE2P; MM-TE2A; DM-TE2A; sarcophagine and sarcophagine derivatives optionally selected from SarAr, SarAr-NCS, diamSar, AmBaSar, and BaBaSar; TRAP; AAZTA; DATA and DATA derivatives; H2-macropa or a derivative thereof; CROWN or a derivative thereof; H₂dedpa, H₄octapa, H₄py4pa, H₄Pypa, H₂azapa, H₅decapa, and other picolinic acid derivatives; CP256; PCTA; C-NETA; C-NE3TA; HBED; SHBED; BCPA; CP256; YM103; desferrioxamine (DFO) and DFO derivatives; and H₆phospa. Exemplary non-limiting examples of radioisotope chelators and example radioisotopes chelated by these chelators are shown in Table 2. In alternative embodiments, R^{X} comprises a radioisotope chelator selected from those listed above or in Table 2, or is any other radioisotope chelator. One skilled in the art could replace any of the chelators listed herein with another chelator.

TABLE 2
Exemplary chelators and exemplary isotopes which bind said chelators

| Chelator | Isotopes |
|---|---|
| 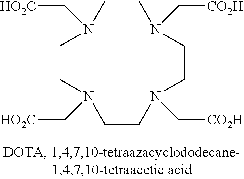 | Cu-64/67 Ga-67/68 In-111 Lu-177 Y-86/90 Bi-203/212/213 Pb-212 Ac-225 Gd-159 Yb-175 Ho-166 |
| | As-211 |
| | Sc-44/47 |
| | Pm-149 |
| | Pr-142 |
| | Sn-117m |
| | Sm-153 |
| | Tb-149/152/155 |
| | /161 |
| | Er-165 |
| | Ra-223/224 |
| | Th-227 |
| 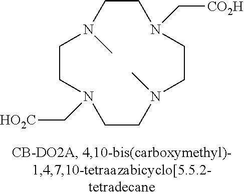 [SMILES: CN1CCN(CC(=O)O)CCN(C)CCN(CC(=O)O)CC1] | Cu-64/67 |
| 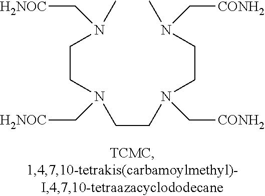 [SMILES: CN(CCN(CCN(CCN(C)CC(N)=O)CC(N)=O)CC(N)=O)CC(N)=O] | Pb-212 |
| 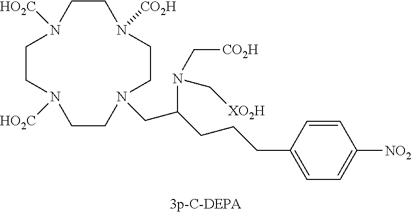 | Bi-212/213 |
| 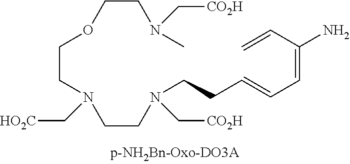 | Cu-64/67 |
| 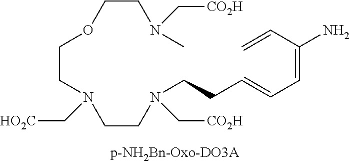 | Cu-64/67 |
| 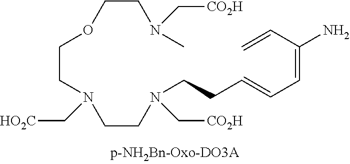 | Cu-64/67 |
| 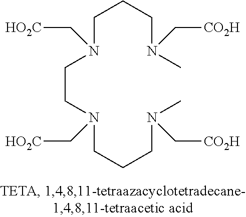 [SMILES: CN(CCCN(CCN(CCCN(C)CC(=O)O)CC(=O)O)CC(=O)O)CC(=O)O] | Cu-64/67 |
| 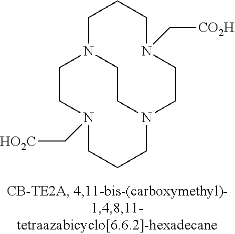 [SMILES: O=C(O)CN1CCCN2CCN(CCCN(CC(=O)O)CC2)CC1] | Cu-64/67 |
| 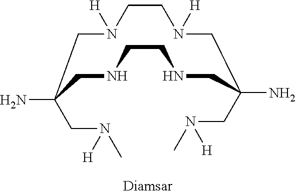 | Cu-64/67 |
| 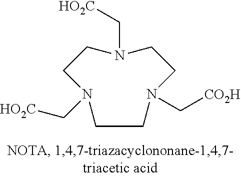 [SMILES: O=C(O)CN1CCN(CC(=O)O)CCN(CC(=O)O)CC1] | Cu-64/67 Ga-68 In-111 Sc-44/47 |
| 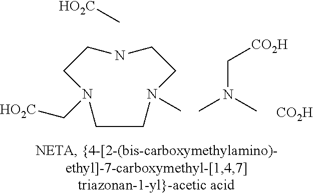 | Cu-64/67 Ga-68 Lu-177 Y-86/90 Bi-213 Pb-212 |
| 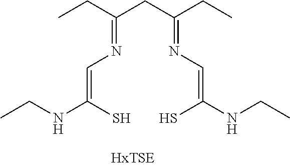 [SMILES: CCN/C(S)=C/N=C(\CC)C/C(CC)=N/C=C(\S)NCC] | Au-198/199 |
| 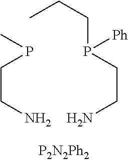 | Rh-105 |
| 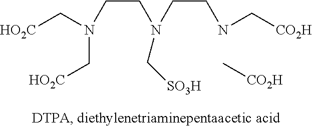 | In-111 Sc-44/47 Lu-177 Y-86/90 Sn-117m Pd-109 |
| 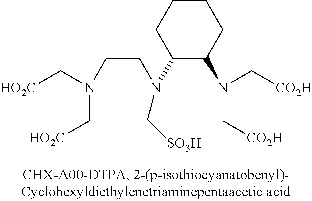 | In-111 Lu-177 Y-86/90 Bi-212/213 |
| 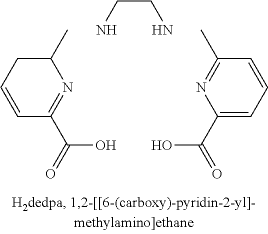 | Cu-64/67 |
| 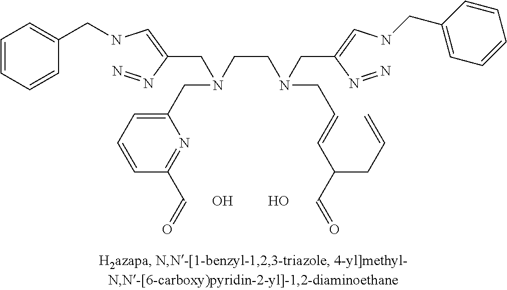 | Cu-64/67 |
| 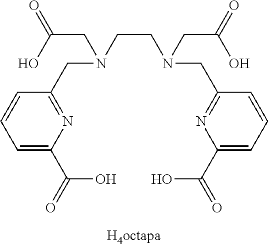 [SMILES: O=C(O)CN(CCN(CC(=O)O)Cc1cccc(C(=O)O)n1)Cc1cccc(C(=O)O)n1] | In-111 Lu-177 Y-86/90 Ac-225 |
| 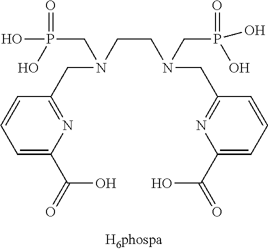 [SMILES: O=C(O)c1cccc(CN(CCN(Cc2cccc(C(=O)O)n2)CP(=O)(O)O)CP(=O)(O)O)n1] | Ac-225 |
| 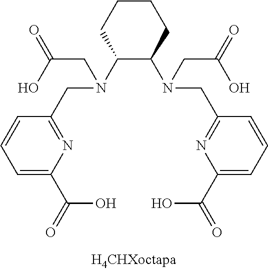 [SMILES: O=C(O)CN(Cc1cccc(C(=O)O)n1)[C@@H]1CCCC[C@H]1N(CC(=O)O)Cc1cccc(C(=O)O)n1] | In-111 Ac-225 |
| 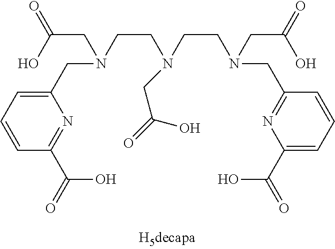 [SMILES: O=C(O)CN(CCN(CC(=O)O)Cc1cccc(C(=O)O)n1)CCN(CC(=O)O)Cc1cccc(C(=O)O)n1] | In-111 Lu-177 Ac-225 |
| 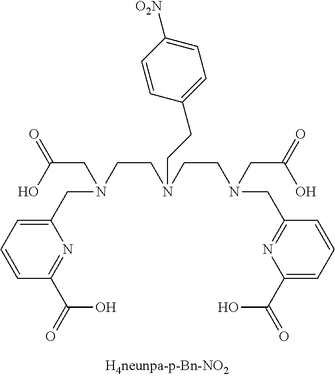 [SMILES: O=C(O)CN(CCN(CCc1ccc([N+](=O)[O-])cc1)CCN(CC(=O)O)Cc1cccc(C(=O)O)n1)Cc1cccc(C(=O)O)n1] | In-111 Lu-177 Ac-225 |
| 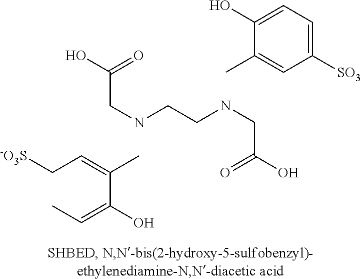 | In-111 Ga-68 |
| 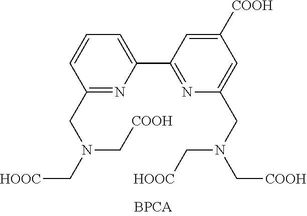 [SMILES: O=C(O)CN(CC(=O)O)Cc1cccc(-c2cc(C(=O)O)cc(CN(CC(=O)O)CC(=O)O)n2)n1] | In-111 |
| 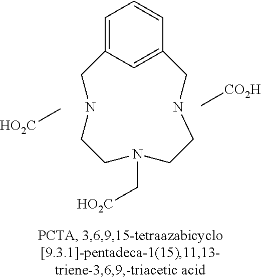 | Cu-64/67 |
| 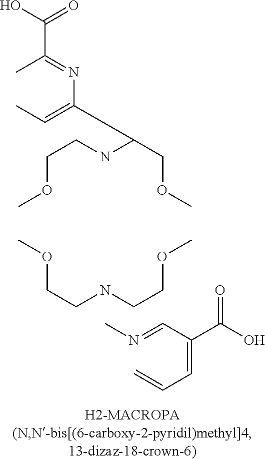 | Ac-225 |
| 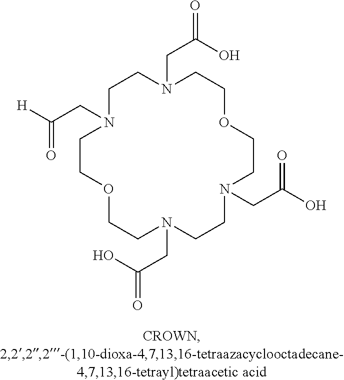 [SMILES: O=CCN1CCOCCN(CC(=O)O)CCN(CC(=O)O)CCOCCN(CC(=O)O)CC1] | Bis-213 Lu-177 Ac-225 |

It would be understood by one skilled in the art how the metal chelators, such as those listed in Table 2, can be connected to the compounds of the invention by replacing one or more atoms or chemical groups of the metal chelators to form the connection. For example, one of the carboxylic acids present in the metal chelator structure can form an amide or an ester bond with the linker or the peptide. In one embodiment, the link formed between the linker and the metal chelator can be covered by the definition of Xaa² (e.g., if an amide bond connects to the metal chelator, even if the carbonyl group could be coming from the metal chelator as drawn in Table 2).

In some embodiments, the radioisotope chelator is conjugated with a radioisotope. The conjugated radioisotope may be, without limitation, ⁶⁸Ga, ⁶¹Cu, ⁶⁴Cu, ⁶⁷Ga, ^{99m}Tc, ¹¹¹1n, ⁴⁴Sc, ⁸⁶Y ⁸⁹Zr, ⁹⁰Nb, ¹⁷⁷Lu, ^{117m}Sn, ¹⁶⁵Er, ⁹⁰Y, ²²⁷Th, ²²⁵Ac, ²¹³Bi, ²¹²Bi, ²¹¹As, ²⁰³Pb, ²¹²Pb, ⁴⁷Sc, ¹⁶⁶Ho, ¹⁸⁸Re, ¹⁸⁶Re, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ^{11m}In, ¹⁵²Tb, ¹⁵⁵Tb, ¹⁶¹Tb, and the like. In some embodiments, the chelator is a chelator from Table 2 and the conjugated radioisotope is a radioisotope indicated in Table 2 as a binder of the chelator.

In some embodiments, the radiometal is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹²Bi, ²²⁷Th, ⁶⁴Cu, or ⁶⁷Cu. In some embodiments, the radiometal is ⁶⁸Ga, ¹⁷⁷Lu, ¹⁵²Tb, ¹⁵⁵Tb, ¹⁶¹Tb, or ²²⁵Ac.

In some embodiments, the radioisotope chelator is not conjugated to a radioisotope.

In some embodiments, the chelator is: DOTA or a derivative thereof, conjugated with ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb ¹⁵⁵Tb, ¹⁶¹Tb, ¹⁶⁵Er, ²¹³Bi, ²²⁴Ra, ²¹²Bi, ²²³Ra, ⁶⁴Cu or ⁶⁷Cu; H2-MACROPA conjugated with ²²⁵Ac; Me-3,2-HOPO conjugated with ²²⁷Th; H₄py4pa conjugated with ²²⁵Ac, ²²⁷Th or ¹⁷⁷L; H₄pypa conjugated with ¹⁷⁷Lu; NODAGA conjugated with ⁶⁸Ga; DTPA conjugated with ¹¹¹In; or DFO conjugated with ⁸⁹Zr.

In some embodiments, the radiometal chelator is DOTA. In some embodiments, DOTA is chelated with ⁶⁸Ga, ¹⁷⁷Lu, ¹⁵²Tb, ¹⁵⁵Tb, ¹⁶¹Tb, or ²²⁵Ac. In some embodiments, DOTA is chelated with ⁶⁸Ga, ¹⁷⁷Lu, ¹⁶¹Tb, or ²²⁵Ac.

In some embodiments, the chelator is TETA (1,4,8,11-tetraazacyclotetradecane-1,4,8,11-tetraacetic acid), SarAr (1-N-(4-Aminobenzyl)-3,6,10,13,16,19-hexaazabicyclo[6.6.6]-eicosane-1,8-diamine), NOTA (1,4,7-triazacyclononane-1,4,7-triacetic acid), TRAP (1,4,7-triazacyclononane-1,4,7-tris[methyl(2-carboxyethyl)phosphinic acid), HBED (N,N′-bis(2-hydroxybenzyl)-ethylenediamine-N,N′-diacetic acid), 2,3-HOPO (3-hydroxypyridin-2-one), PCTA (3,6,9,15-tetraazabicyclo[9.3.1]-pentadeca-1(15), 11,13-triene-3,6,9,-triacetic acid), DFO (desferrioxamine), DTPA (diethylenetriaminepentaacetic acid), OCTAPA (N,N′-bis(6-carboxy-2-pyridylmethyl)-ethylenediamine-N,N′-diacetic acid) or another picolinic acid derivative.

One or more R^{X} may comprise a chelator for radiolabelling with ^{99m}Tc, ^{94m}Tc, ¹⁸⁶Re, or ¹⁸⁸Re, such as mercaptoacetyl, hydrazinonicotinamide, dimercaptosuccinic acid, 1,2-ethylenediylbis-L-cysteine diethyl ester, methylenediphosphonate, hexamethylpropyleneamineoxime and hexakis(methoxy isobutyl isonitrile, and the like. In some embodiments, one or more R^{X} comprises a chelator, wherein the chelator is mercaptoacetyl, hydrazinonicotinamide, dimercaptosuccinic acid, 1,2-ethylenediylbis-L-cysteine diethyl ester, methylenediphosphonate, hexamethylpropyleneamineoxime or hexakis(methoxy isobutyl isonitrile). In some of these embodiments, the chelator is bound by a radioisotope. In some such embodiments, the radioisotope is ^{99m}Tc, ^{94m}Tc, ¹⁸⁶Re, or ¹⁸⁸Re.

One or more R^{X} may comprise a chelator that can bind ¹⁸F-aluminum fluoride ([¹⁸F]AlF), such as 1,4,7-triazacyclononane-1,4-diacetate (NODA) and the like. In some embodiments, the chelator is NODA. In some embodiments, the chelator is bound by [¹⁸F]AlF.

One or more R^{X} may comprise a chelator that can bind ⁷²As or ⁷⁷As, such as a trithiol chelate and the like. In some embodiments, the chelator is a trithiol chelate. In some embodiments, the chelator is conjugated to ⁷²As. In some embodiments, the chelator is conjugated to ⁷⁷As.

One or more R^{X} may comprise an aryl group substituted with a radioisotope. In some embodiments, one or more R^{X} is

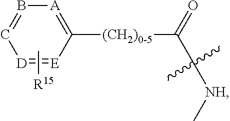

wherein A, B, C, D and E are independently C or N, and R¹⁵ is a radiohalogen. In some embodiments, one or more R^{X} is

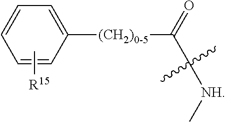

In some embodiments, one or more R^{X} is

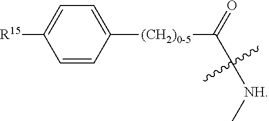

In some embodiments, one or more R^{X} is

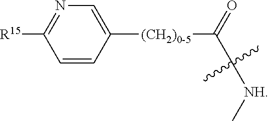

In some embodiments, one or more R^{X} is

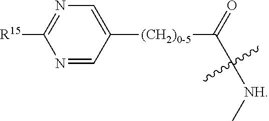

In some embodiments, one or more R

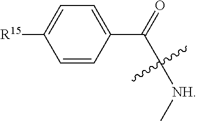

In some embodiments, one or more R^{X} is

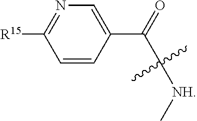

In some embodiments, one or more R^{X} is

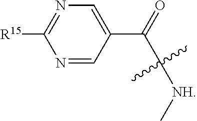

In some embodiments, one or more R^{X} is

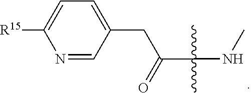

In some of these embodiments, R¹⁵ is independently ²¹¹At, ¹³¹I, ¹²⁴I, ¹²³I, ⁷⁷Br or ¹⁸F. In some of these embodiments, R¹⁵ is ¹⁸F.

In some embodiments, one or more R^{X} may comprise a prosthetic group containing a trifluoroborate (BF₃), capable of ¹⁸F/¹⁹F exchange radiolabeling. In such embodiments, one or more R^{X} may be R¹⁶R¹⁷BF₃, wherein each R¹⁶ is independently

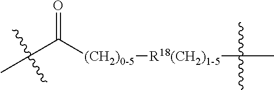

and R¹⁸ is absent,

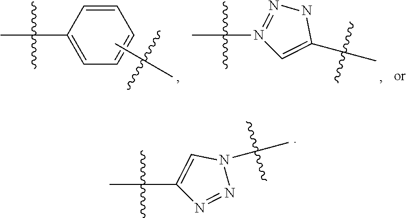

Each —R¹⁷BF₃ may independently be selected from one or a combination of those listed in Table 3 (below), Table 4 (below), or

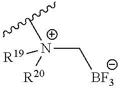

wherein R¹⁹ and R²⁰ are independently C₁-C₅ linear or branched alkyl groups. For Tables 3 and 4, the R in the pyridine substituted with —OR, —SR, —NR—, —NHR or —NR₂ groups is C₁-C₅ branched or linear alkyl. In some embodiments, one or more —R¹⁷BF₃ is independently selected from one or a combination of those listed in Table 3. In some embodiments, one or more —R¹⁷BF₃ is independently selected from one or a combination of those listed in Table 4. In some embodiments, one fluorine is ¹⁸F. In some embodiments, all three fluorines are ¹⁹F.

TABLE 3
Exemplary R¹⁷BF₃ groups.

| | | | |
|---|---|---|---|
| 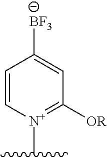 | 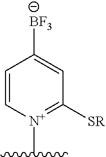 | 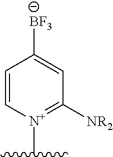 | 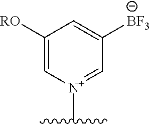 |
| 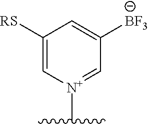 | 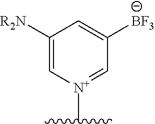 | 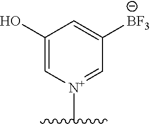 | 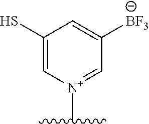 |
| 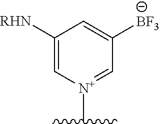 | 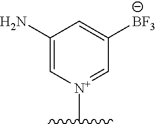 | 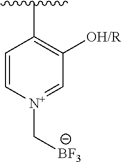 | 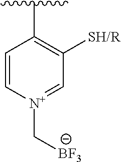 |
| 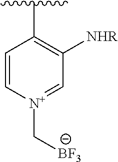 | 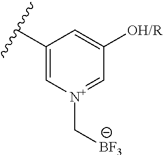 | 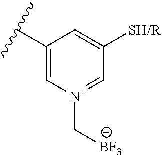 | 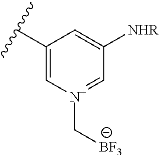 |
| 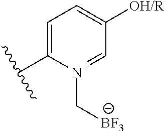 | 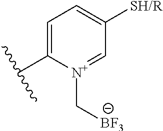 | 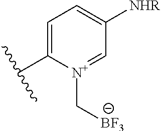 | 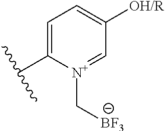 |
| 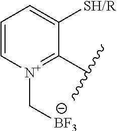 | 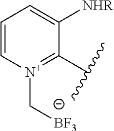 | 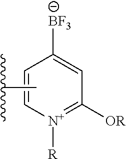 | 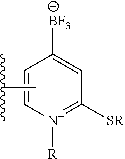 |
| 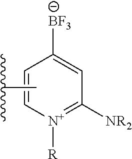 | 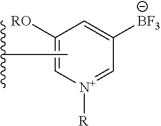 | 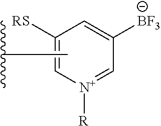 | 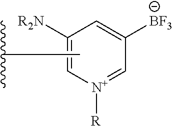 |
| 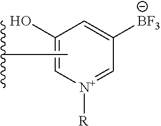 | 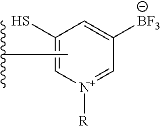 | 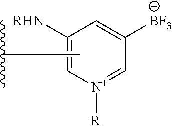 | 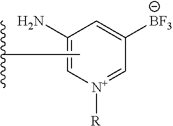 |
| 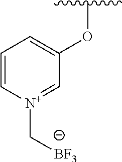 | 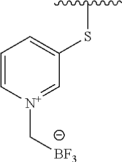 | 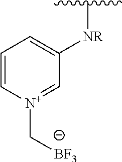 | 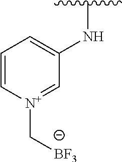 |
| 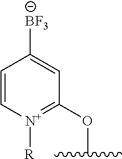 | 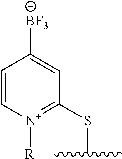 | 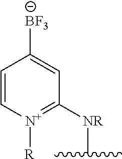 | 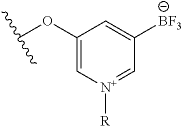 |
| 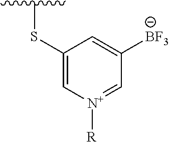 | 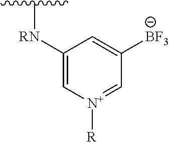 | 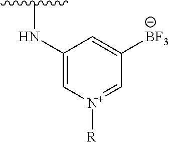 | 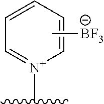 |

TABLE 4
Exemplary R¹⁷BF₃ groups.

| | | | |
|---|---|---|---|
| 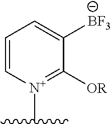 | 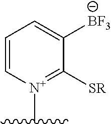 | 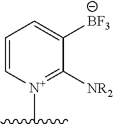 | 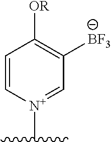 |
| 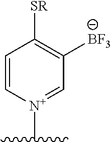 | 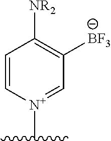 | 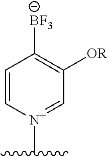 | 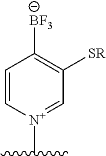 |
| 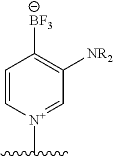 | 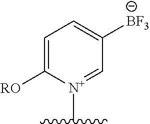 | 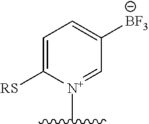 | 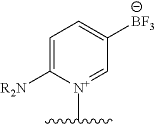 |
| 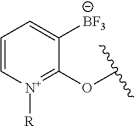 | 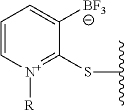 | 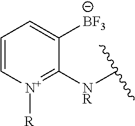 | 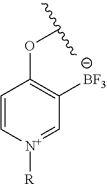 |
| 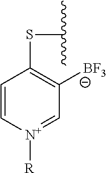 | 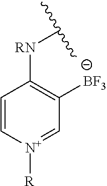 | 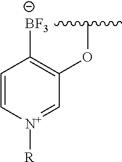 | 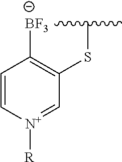 |
| 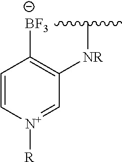 | 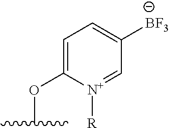 | 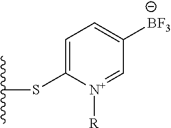 | 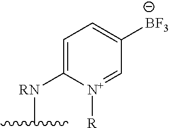 |
| 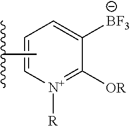 | 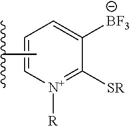 | 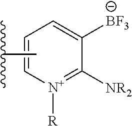 | 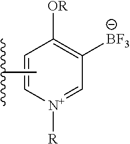 |
| 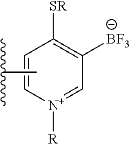 | 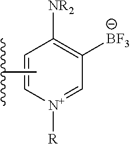 | 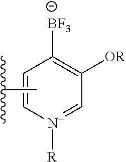 | 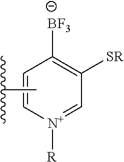 |
| 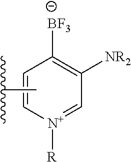 | 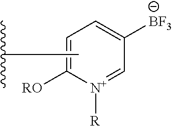 | 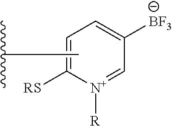 | 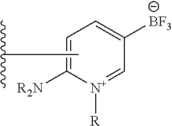 |
| 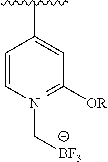 | 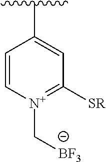 | 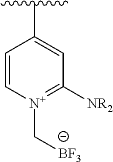 | 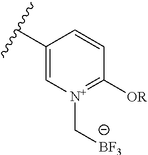 |
| 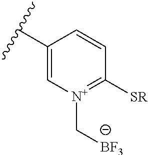 | 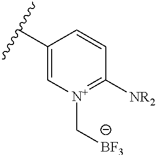 | 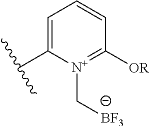 | 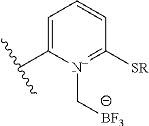 |
| 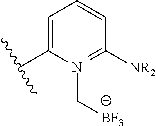 | 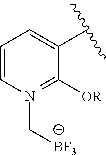 | 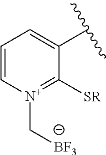 | 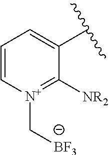 |
| 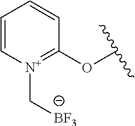 | 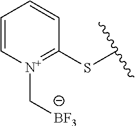 | 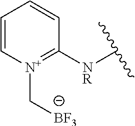 | 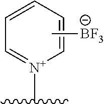 |

In some embodiments, R¹⁷BF₃ may form

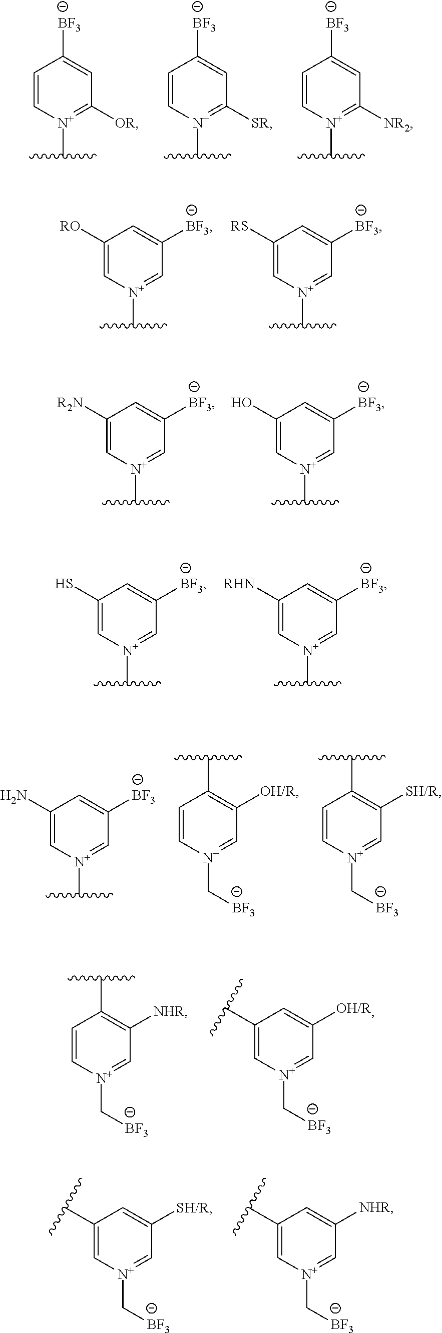

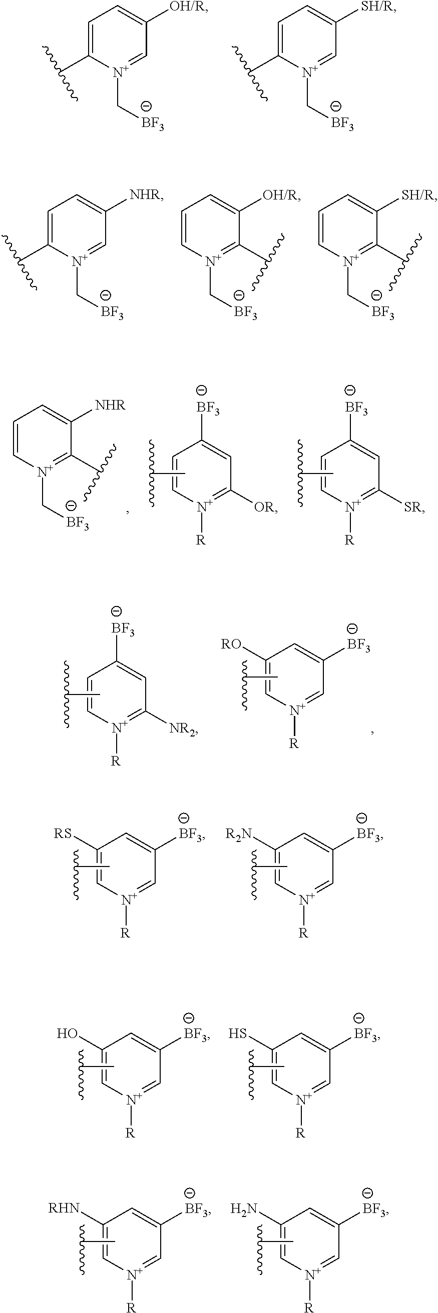

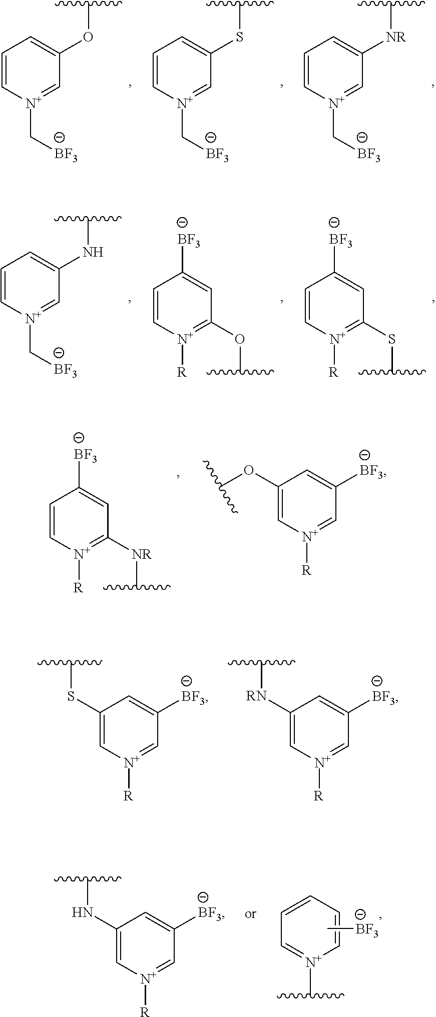

in which the R (when present) in the pyridine substituted —OR, —SR, —NR—, —NHR or —NR₂ is a branched or linear C₁-C₅ alkyl. In some embodiments, R is a branched or linear C₁-C₅ saturated alkyl. In some embodiments, R is methyl. In some embodiments, R is ethyl. In some embodiments, R is propyl. In some embodiments, R is isopropyl. In some embodiments, R is n-butyl. In some embodiments, one fluorine is ¹⁸F. In some embodiments, all three fluorines are ¹⁹F.

In some embodiments, R¹⁷BF₃ may form

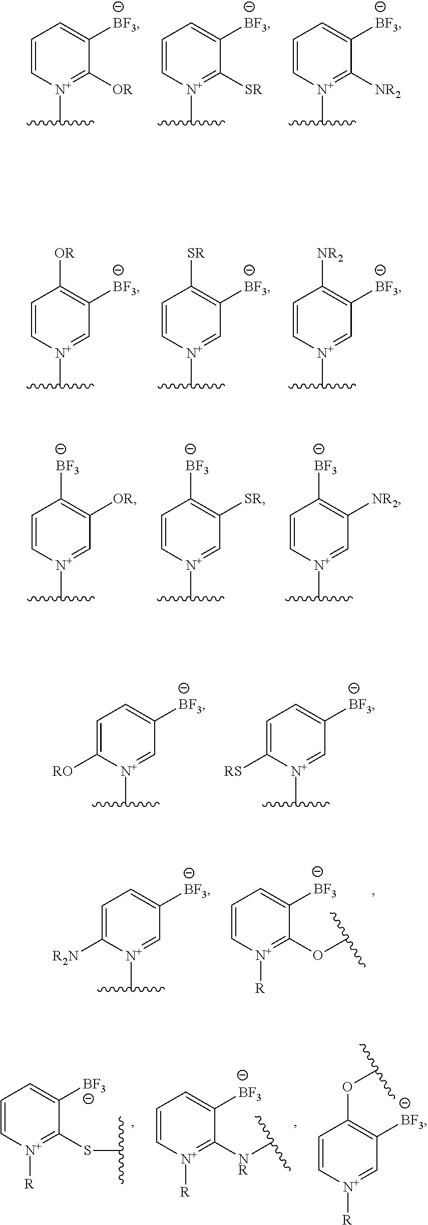

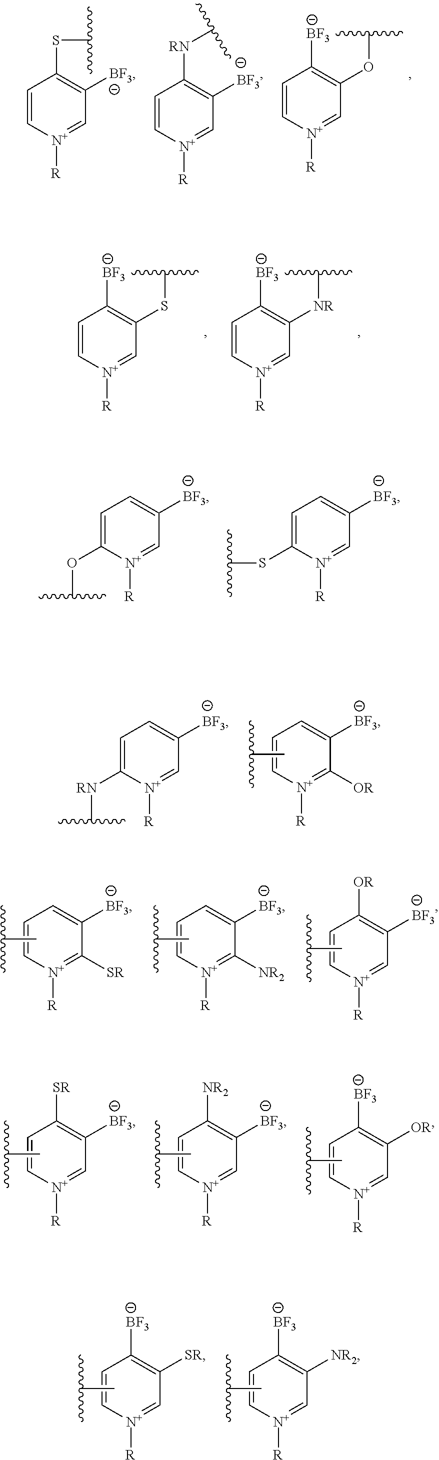

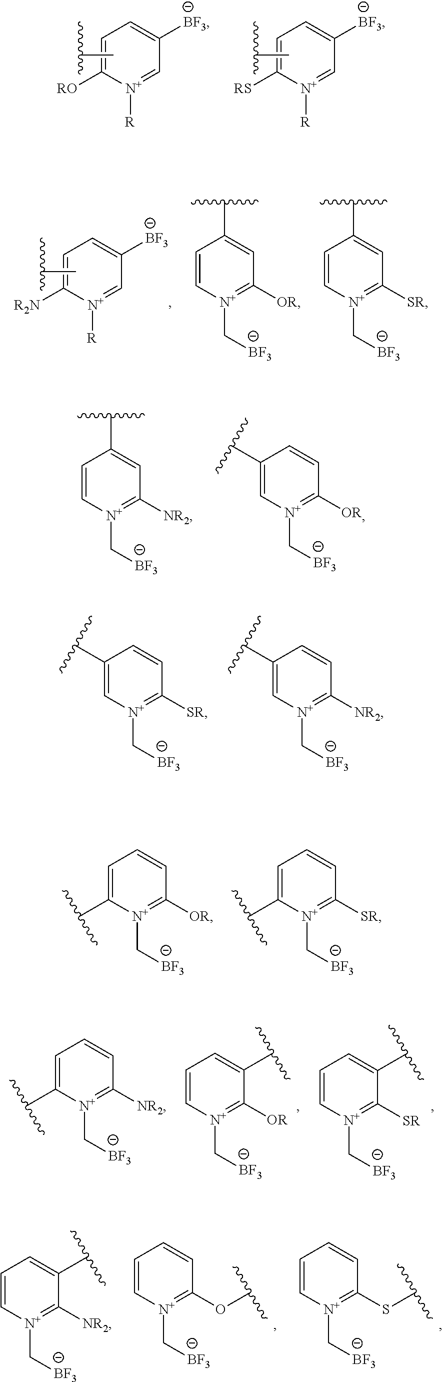

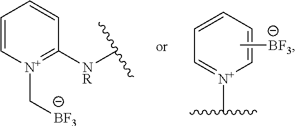

in which the R (when present) in the pyridine substituted —OR, —SR, —NR— or —NR₂ is branched or linear C₁-C₅ alkyl. In some embodiments, R is a branched or linear C₁-C₅ saturated alkyl. In some embodiments, R is methyl. In some embodiments, R is ethyl. In some embodiments, R is propyl. In some embodiments, R is isopropyl. In some embodiments, R is n-butyl. In some embodiments, one or more —R¹⁷BF₃ is

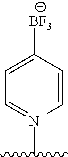

In some embodiments, one fluorine is ¹⁸F. In some embodiments, all three fluorines are ¹⁹F.

In some embodiments, one or more —R¹⁷BF₃ is

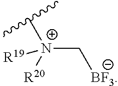

In some embodiments, R¹⁹ is methyl. In some embodiments, R¹⁹ is ethyl. In some embodiments, R¹⁹ is propyl. In some embodiments, R¹⁹ is isopropyl. In some embodiments, R¹⁹ is butyl. In some embodiments, R¹⁹ is n-butyl. In some embodiments, R¹⁹ is pentyl. In some embodiments, R²⁰ is methyl. In some embodiments, R²⁰ is ethyl. In some embodiments, R²⁰ is propyl. In some embodiments, R²⁰ is isopropyl. In some embodiments, R²⁰ is butyl. In some embodiments, R²⁰ is n-butyl. In some embodiments, R²⁰ is pentyl. In some embodiments, R¹⁹ and R²⁰ are both methyl. In some embodiments, one fluorine is ¹⁸F. In some embodiments, all three fluorines are ¹⁹F.

In some embodiments, one or more R^{X} may comprise a prosthetic group containing a silicon-fluorine-acceptor moiety. In some embodiments, the fluorine of the silicon-fluorine acceptor moiety is ¹⁸F. The prosthetic groups containing a silicon-fluorine-acceptor moiety may be independently selected from one or a combination of the following:

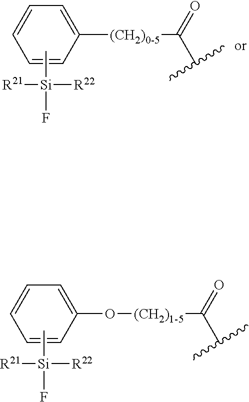

wherein R²¹ and R²² are independently a linear or branched, cyclic or acyclic, and/or aromatic or non-aromatic C₁-C₁o alkyl, alkenyl or alkynyl group. In some embodiments, R²¹ and R²² are independently selected from the group consisting of phenyl, tert-butyl, sec-propyl or methyl. In some embodiments, the prosthetic group is

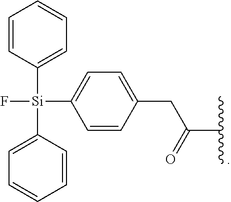 [SMILES: *C(=O)Cc1ccc([Si](F)(c2ccccc2)c2ccccc2)cc1 ; "."]

In some embodiments, the prosthetic group is

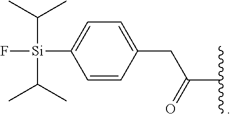 [SMILES: *C(=O)Cc1ccc([Si](F)(C(C)C)C(C)C)cc1 ; "."]

In some embodiments, the prosthetic group is

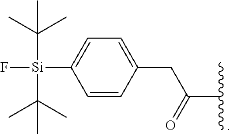 [SMILES: *C(=O)Cc1ccc([Si](F)(C(C)(C)C)C(C)(C)C)cc1 ; "."]

In some embodiments, the prosthetic group is

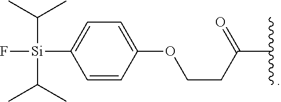 [SMILES: *C(=O)CCOc1ccc([Si](F)(C(C)C)C(C)C)cc1 ; "."]

In some embodiments, one or more R^{X} comprise a prosthetic group containing a fluorophosphate. In some embodiments, one or more R^{X} comprise a prosthetic group containing a fluorosulfate. In some embodiments, one or more R^{X} comprise a prosthetic group containing a sulfonylfluoride. Such prosthetic groups are well known and are commercially available, and are facile to attach (e.g. via an amide linkage). In some embodiments, the fluorine atom in the fluorophosphate, fluorosulfate or sulfonylfuloride is ¹⁸F. In some embodiments, the fluorine atom in the fluorophosphate, fluorosulfate or sulfonylfuloride is ¹⁹F.

Certain dual labeled compounds (i.e. when R⁷ comprises two R^{X} groups), have only a single radioactive atom. For example, but without limitation, one R^{X} group may be ¹⁸F labeled and the other R^{X} group may comprise only ¹⁹F or the other R^{X} group may comprise a chelator that is not chelated with a radiometal or is chelated with a metal that is not a radioisotope. In another non-limiting example, one R^{X} group may comprise an aryl substituted with a radioisotope and the other R^{X} group may comprise only ¹⁹F or the other R^{X} group may comprise a chelator that is not chelated with a radiometal or is chelated with a metal that is not a radioisotope. In yet another non-limiting example, one R^{X} group may comprise a chelator conjugated with a radioisotope and the other R^{X} group may comprise only ¹⁹F.

In some embodiments, R⁷ comprises a first R^{X} group and a second R^{X} group, wherein the first R^{X} group is a radiometal chelator optionally bound by a radiometal and the second R^{X} group is a prosthetic group containing a trifluoroborate. In some embodiments, R⁷ comprises a first R^{X} group and a second R^{X} group, wherein the first R^{X} group is a radiometal chelator optionally bound by a radiometal and the second R^{X} group is a prosthetic group containing a trifluoroborate.

In certain embodiments, the compound is conjugated with a radioisotope for positron emission tomography (PET) or single photon emission computed tomography (SPECT) imaging of PSMA expressing tumors, wherein the compound is conjugated with a radioisotope that is a positron emitter or a gamma emitter. Without limitation, the positron or gamma emitting radioisotope is ⁶⁸Ga, ⁶⁷Ga, ⁶¹Cu, ⁶⁴Cu, ^{99m}Tc, 110mi ¹¹¹In, ⁴⁴Sc, ⁸⁶Y, ⁸⁹Zr, ⁹⁰Nb, ¹⁸F, ¹³¹I, ¹²³I, ¹²⁴I and ⁷²As. In some embodiments, radioisotope useful for imaging is ⁶⁸Ga, ⁶⁷Ga, ⁶¹Cu, ⁶⁴CU, ^{99m}Tc, ^{114m}In ¹¹¹In, ⁴⁴Sc, ⁸⁶Y, ⁸⁹Zr, ⁹⁰Nb, ¹⁸F, ¹³¹I, ¹²³I, ¹²⁴I, or ⁷²As. In one embodiment, the radioisotope useful for imaging is ⁶⁸Ga, ⁶⁷Ga, ⁶¹Cu, ⁶⁴Cu, ^{99m}Tc, ^{114m}In, ¹¹¹In, ⁴⁴Sc, ⁸⁶Y, ⁸⁹Zr, ⁹⁰Nb, ¹³¹I, ¹²³I, ¹²⁴I, or ⁷²As.

In certain embodiments the compound is conjugated with a radioisotope that is used for therapy of PSMA-expressing tumors. This includes radioisotopes such as ¹⁶⁵Er, ²¹²Bi, ²¹¹At, ¹⁶⁶Ho, ¹⁴⁹Pm, ¹⁵⁹Gd, ¹⁰⁵Rh, ¹⁰⁹Pd, ¹⁹⁸Au, ¹⁹⁹Au, ¹⁷⁵Yb, ¹⁴²Pr, ¹⁷⁷Lu, ¹¹¹In, ²¹³Bi, ²⁰³Pb, ²¹²Pb, ⁴⁴Sc, ⁴⁷Sc, ⁹⁰Y ²²⁵Ac, ^{117m}Sn ¹⁵³Sm, ¹⁴⁹Tb, ¹⁵²Tb, ¹⁵⁵Tb, ¹⁶¹Tb, ²²⁴Ra, ²²⁷Th, ²²³Ra, ⁷⁷As, ⁶⁴Cu or ⁶⁷cu.

The compound may be CCZ02010, CCZ02011, CCZ02018, CCZ01186, CCZ01188, CCZ01194, CCZ01198, CCZ02032, CCZ02033, ADZ-4-101, PD-6-49, PD-5-131, PD-5-159, AR-2-050-1, AR-2-050-2, AR-2-113-1 or AR-2-113-2 or a salt or solvate thereof, optionally conjugated with a radiometal. In some embodiments, the radiometal is ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y ⁸⁶Y, ²²⁵Ac, ^{117m}Sn, ¹⁵³Sm, ¹⁴⁹Tb, ¹⁶¹Tb, ¹⁶⁵Er, ²²⁴Ra, ²¹²Bi, ²²⁷Th, ²²³Ra, ⁶⁴Cu or ⁶⁷Cu. In some embodiments, the radiometal is ⁶⁸Ga. In some embodiments, the radiometal is ¹⁷⁷Lu.

In some embodiments, AR-2-113-1 or AR-2-113-2 is complexed with ⁶⁸Ga.

In some embodiments, CCZ02010, CCZ02011, CCZ02018, CCZ01186, CCZ01188, CCZ01194, CCZ01198, CCZ02032, CCZ02033, ADZ-4-101, PD-6-49, PD-5-131, PD-5-159, AR-2-050-1, or AR-2-050-2 is complexed with ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn ¹⁵³Sm, ¹⁴⁹Tb, ¹⁵²Tb, ¹⁵⁵Tb, ¹⁶¹Tb, ¹⁶⁵Er, ²¹³Bi, ²²⁴Ra, ²¹²Bi, ²²³Ra, ⁶⁴Cu or ⁶⁷Cu. In some embodiments, CCZ02010, CCZ02011, CCZ02018, CCZ01186, CCZ01188, CCZ01194, CCZ01198, CCZ02032, CCZ02033, ADZ-4-101, PD-6-49, PD-5-131, PD-5-159, AR-2-050-1, or AR-2-050-2 is complexed with ⁶⁸Ga, ¹⁷⁷Lu, ¹⁶¹Tb, or ²²⁵Ac.

When the radiolabeling group comprises or is conjugated to a diagnostic radioisotope, there is disclosed use of certain embodiments of the compound for preparation of a radiolabelled tracer for imaging PSMA-expressing tissues in a subject. There is also disclosed a method of imaging PSMA-expressing tissues in a subject, in which the method comprises: administering to the subject a composition comprising certain embodiments of the compound and a pharmaceutically acceptable excipient; and imaging tissue of the subject, e.g. using PET or SPECT. When the tissue is a diseased tissue (e.g. a PSMA-expressing cancer), PSMA-targeted treatment may then be selected for treating the subject.

When the radiolabeling group comprises a therapeutic radioisotope, there is disclosed use of certain embodiments of the compound (or a pharmaceutical composition thereof) for the treatment of PSMA-expressing conditions or diseases (e.g. cancer and the like) in a subject. Accordingly, there is provided use of the compound in preparation of a medicament for treating a PSMA-expressing condition or disease in a subject. There is also provided a method of treating PSMA-expressing disease in a subject, in which the method comprises: administering to the subject a composition comprising the compound and a pharmaceutically acceptable excipient. For example, but without limitation, the disease may be a PSMA-expressing cancer.

PSMA expression has been detected in various cancers (e.g. Rowe et al., 2015, Annals of Nuclear Medicine 29:877-882; Sathekge et al., 2015, Eur J Nucl Med Mol Imaging 42:1482-1483; Verburg et al., 2015, Eur J Nucl Med Mol/maging 42:1622-1623; and Pyka et al., J Nucl Med Nov. 19, 2015 jnumed.115.164442). Accordingly, without limitation, the PSMA-expressing cancer may be prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma. In some embodiments, the cancer is prostate cancer.

Compounds Comprising Retro-Inverso Peptide Linkers

It is well known to those skilled in the art that the concept of retro-inverso peptide design can be applied to further vary the linker constructs defined for the various compounds above. Without prejudice for a given stereoisomer and not necessarily being bound by a given stereoisomer, the use of the retro-inverso approach would require that the preferred stereochemical configuration at certain stereogenic atoms be inverted provided that the polarity of the linking group(s) that bracket the stereogenic atom in question, e.g. N-termini and C-termini have been inverted in the design of a retro-inverso peptide fragment. It is also well known that amide linkages in peptidic linkers can be substituted with alternative linkages and in certain cases extended by an additional group of atoms, e.g. a CH₂ or C═O at a given amino acid. As such, any such linker defined above (or elsewhere herein, e.g. in the Examples) may be replaced with a linker in which the polarity of an amino acid is inverted and/or in which an amide linkage is replaced with an alternative linkage wherein the overall position and 3D conformation of the linker is retained. This principle is demonstrated in the following non-limiting examples of embodiments to illustrate how parts of the molecule that have the same or similar functional groups have been replaced with retro-inverso counterparts, as would be readily appreciated by those skilled in the art of peptide chemistry:

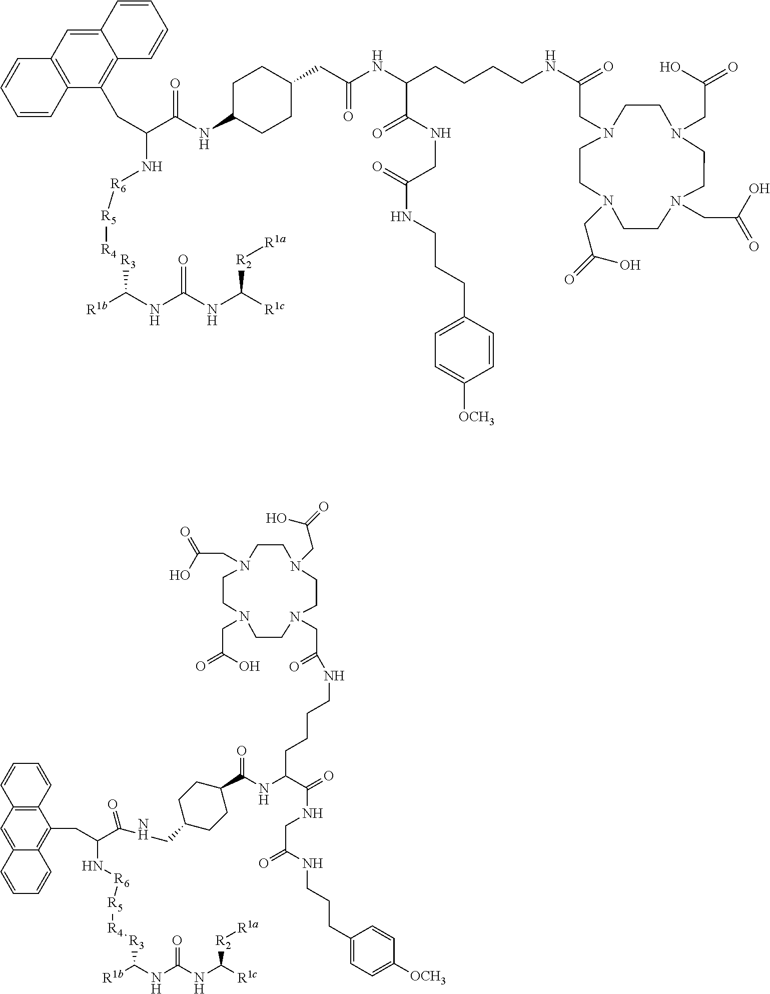

The compounds presented herein incorporate peptides, which may be synthesized by any of a variety of methods established in the art. This includes but is not limited to liquid-phase as well as solid-phase peptide synthesis using methods employing 9-fluorenylmethoxycarbonyl (Fmoc) and/or t-butyloxycarbonyl (Boc) chemistries, and/or other synthetic approaches.

For example, the PSMA-targeting peptidomimetic can be synthesized on solid phase. In a non-limiting example, the PSMA-binding moiety is linker-ureido-(amino acid). Exemplary, but non-limiting, linkers include Fmoc-protected homolysine, Ornithine (Orn), diaminopimelic acid, diaminobutyric Acid, 4-NH₂-Phenyl-alanine, where the side chain amine group is optionally protected by ivDde or Alloc; the linker may also include an Fmoc-protected unnatural amino acid with a side chain alkyne or azide group. Exemplary, but non-limiting, amino acid (AA) groups include 2-aminoadipic acid (Aad), carboxymethylcysteine, carboxymethylserine, and the like. The formation of a ureido linkage between the amino groups of the linker and the AA may be constructed on solid phase by attaching the linker to 2-chlorotrityl resin, for example, Fmoc-Orn(ivDde)-OH) (2 eq.) in presence of N,N-diisopropylethylamine (DIPEA, 8 eq.) in dichloromethane (DCM). The Fmoc-protecting group is then removed by 20% piperidine in N,N-dimethylformamide (DMF). To form the ureido linkage, the freed amino group of the solid-phase-attached amino acid is reacted with the AA which has its carboxylate group protected with a t-butyl group and its amino group activated and converted to an isocyanate group (—N═C═O). The activation and conversion of an amino group to an isocyanate group can be achieved by reacting the amino group with phosgene or triphosgene. After the formation of the ureido linkage, the side chain protecting group of the linker (for example the ivDde on Orn) can be removed. In the case of a side chain alkyne or azide group, copper-catalyzed cycloaddition with an amine containing azide or alkyne can be performed to give 1, 2, 3-triazole. Subsequently, other linkers, albumin-binding motif, and/or radiolabeling groups (e.g. radiometal chelator and the like) can be subsequently coupled to the PSMA-binding moiety using standard activation/coupling strategy, for example, Fmoc-protected amino acid (4 eq.), 2-(7-Aza-1H-benzotriazole-1-yl)-1,1,3,3-tetramethyluronium hexafluorophosphate (HATU, 4 eq.) and DIPEA (7 eq.) in DMF. The peptidomimetic is then deprotected and simultaneously cleaved from the resin by treating with 95/5 trifluoroacetic acid (TFA)/triisopropylsilane (TIS) for 4 h at room temperature. After filtration, the peptidomimetic is precipitated by cold diethyl ether. The crude peptide is purified by high performance liquid chromatography (HPLC) using a preparative or semi-preparative C18 column. The eluates containing the desired product are collected and lyophilized. The identity of the compounds is verified by mass spectrometry, and the purity is determined by HPLC using an analytical C18 column. Each step is described in more detail below, and in the Examples.

Solid-phase peptide synthesis methods and technology are well-established in the art. For example, peptides may be synthesized by sequential incorporation of the amino acid residues of interest one at a time. In such methods, peptide synthesis is typically initiated by attaching the C-terminal amino acid of the peptide of interest to a suitable resin. Prior to this, reactive side chain and alpha amino groups of the amino acids are protected from reaction by suitable protecting groups, allowing only the alpha carboxyl group to react with a functional group such as an amine group, a hydroxyl group, or an alkyl halide group on the solid support. Following coupling of the C-terminal amino acid to the support, the protecting group on the side chain and/or the alpha amino group of the amino acid is selectively removed, allowing the coupling of the next amino acid of interest. This process is repeated until the desired peptide is fully synthesized, at which point the peptide can be cleaved from the support and purified. A non-limiting example of an instrument for solid-phase peptide synthesis is the Aapptec Endeavor 90 peptide synthesizer.

To allow coupling of additional amino acids, Fmoc protecting groups may be removed from the amino acid on the solid support, e.g. under mild basic conditions, such as piperidine (20-50% v/v) in DMF. The amino acid to be added must also have been activated for coupling (e.g. at the alpha carboxylate). Non-limiting examples of activating reagents include without limitation 2-(1H-benzotriazol-1-yl)-1,1,3,3-tetramethyluronium hexafluorophosphate (HBTU), 2-(1H-benzotriazol-1-yl)-1,1,3,3-tetramethyluronium tetrafluoroborate (TBTU), 2-(7-Aza-1H-benzotriazole-1-yl)-1,1,3,3-tetramethyluronium hexafluorophosphate (HATU), benzotriazole-1-yl-oxy-tris(dimethylamino)phosphoniumhexafluorophosphate (BOP), benzotriazole-1-yl-oxy-tris(pyrrolidino)phosphoniumhexafluorophosphate (PyBOP). Racemization is minimized by using triazoles, such as 1-hydroxy-benzotriazole (HOBt) and 1-hydroxy-7-aza-benzotriazole (HOAt). Coupling may be performed in the presence of a suitable base, such as N,N-diisopropylethylamine (DIPEA/DIEA) and the like. For long peptides or if desired, peptide synthesis and ligation may be used.

Apart from forming typical peptide bonds to elongate a peptide, peptides may be elongated in a branched fashion by attaching to side chain functional groups (e.g. carboxylic acid groups or amino groups), either: side chain to side chain; or side chain to backbone amino or carboxylate. Coupling to amino acid side chains may be performed by any known method, and may be performed on-resin or off-resin. Non-limiting examples include: forming an amide between an amino acid side chain containing a carboxyl group (e.g. Asp, D-Asp, Glu, D-Glu, Aad, and the like) and an amino acid side chain containing an amino group (e.g. Lys, D-Lys, Orn, D-Orn, Dab, D-Dab, Dap, D-Dap, and the like) or the peptide N-terminus; forming an amide between an amino acid side chain containing an amino group (e.g. Lys, D-Lys, Orn, D-Orn, Dab, D-Dab, Dap, D-Dap, and the like) and either an amino acid side chain containing a carboxyl group (e.g. Asp, D-Asp, Glu, D-Glu, and the like) or the peptide C-terminus; and forming a 1, 2, 3-triazole via click chemistry between an amino acid side chain containing an azide group (e.g. Lys(N₃), D-Lys(N₃), and the like) and an alkyne group (e.g. Pra, D-Pra, and the like). The protecting groups on the appropriate functional groups must be selectively removed before amide bond formation, whereas the reaction between an alkyne and an azido groups via the click reaction to form an 1,2,3-triazole does not require selective deprotection. Non-limiting examples of selectively removable protecting groups include 2-phenylisopropyl esters (O-2-PhiPr) (e.g. on Asp/Glu) as well as 4-methyltrityl (Mtt), allyloxycarbonyl (alloc), 1-(4,4-dimethyl-2,6-dioxocyclohex-1-ylidene))ethyl (Dde), and 1-(4,4-dimethyl-2,6-dioxocyclohex-1-ylidene)-3-methylbutyl (ivDde) (e.g. on Lys/Orn/Dab/Dap). O-2-PhiPr and Mtt protecting groups can be selectively deprotected under mild acidic conditions, such as 2.5% trifluoroacetic acid (TFA) in DCM. Alloc protecting groups can be selectively deprotected using tetrakis(triphenylphosphine)palladium(0) and phenyl silane in DCM. Dde and ivDde protecting groups can be selectively deprotected using 2-5% of hydrazine in DMF. Deprotected side chains of Asp/Glu (L- or D-forms) and Lys/Orn/Dab/Dap (L- or D-forms) can then be coupled, e.g. by using the coupling reaction conditions described above.

An example of the synthesis of a PSMA-targeting compound with a 1, 2, 3-triazole Linker-ureido-Aad backbone is illustrated in Scheme 1, below. Fmoc-Dap(N₃)—OH (2 eq.) is loaded onto 2-chlorotrityl resin in presence of DIPEA (8 eq.) in DCM, followed by Fmoc deprotection. To generate the isocyanate of the 2-aminoadipyl moiety, a solution of Aad di-t-butyl ester hydrochloride (10 eq.) and DIPEA (33 eq.) in DCM is cooled to −78° C. in a dry ice/acetone bath. Triphosgene (3.3 eq.) is dissolved in DCM and added dropwise. The reaction is then allowed to warm to room temperature and stir for 30 minutes to give a solution of the isocyanate of the 2-aminoadipyl moiety (Scheme 1, compound 1), which is then added to the NH₂-Dap(N₃)-immobilized resin and mix for 16 h to give 2. After washing the resin with DMF, propargylamine (5 eq.), CuSO₄ (5 eq.), and sodium ascorbate (10 eq.), DIPEA (10 eq.) in DMF are added and allowed to mix for 16 h to give 3. Fmoc-Ala(9-Anth)-OH, Fmoc-tranexamic acid, and finally DOTA-tris(t-bu)ester (2-(4,7,10-tris(2-(t-butoxy)-2-oxoehtyl)-1,4,7,10)-tetraazacyclododecan-1-yl)acetic acid) are coupled to the amine group in presence of HATU (4 eq.) and DIPEA (7 eq.), followed by side chain deprotection and cleavage by TFA/TIS, and HPLC purification to afford 4.

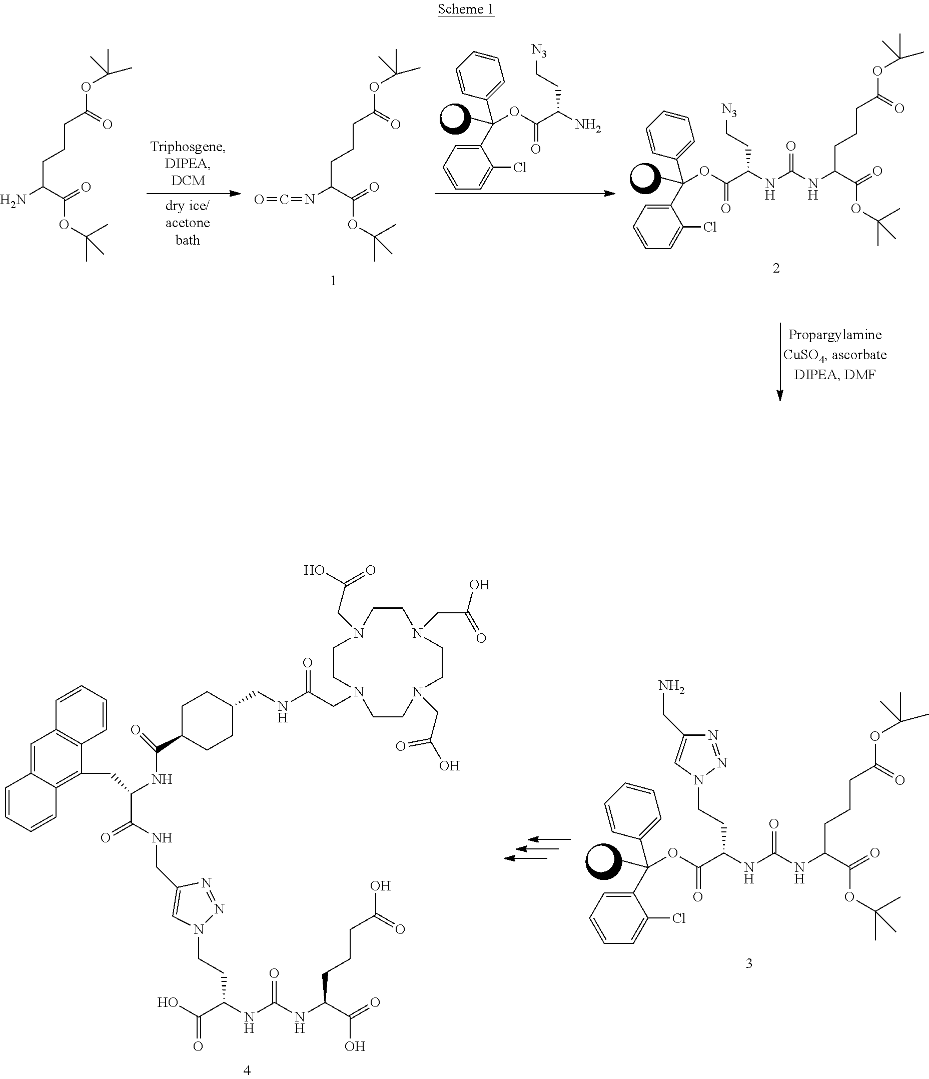

The PSMA-binding moiety (e.g. Lys-ureido-Aad, and the like) may be constructed on solid phase via the formation of a ureido linkage between the amino groups of two amino acids. This can be done by attaching an Fmoc-protecting amino acid (for example Fmoc-Lys(ivDde)-OH) to Wang resin using standard activation/coupling strategy (for example, Fmoc-protected amino acid (4 eq.), HATU (4 eq.) and N,N-diisopropylethylamine (7 eq.) in N,N-dimethylformamide). The Fmoc-protecting group is then removed by 20% piperidine in N,N-dimethylformamide. To form the ureido linkage, the freed amino group of the solid-phase-attached amino acid is reacted with the 2^{nd} amino acid which has its carboxylate group protected with a t-butyl group and its amino group activated and converted to an isocyanate group (—N═C═O). The activation and conversion of an amino group to an isocyanate group can be achieved by reacting the amino group with phosgene or triphosgene. After the formation of the ureido linkage, the side chain functional group of the amino acid (for example ivDde on Lys) can be removed, and then the linker, albumin-binding motif, and/or radiolabeling group (e.g. radiometal chelator and the like) can be subsequently coupled to the PSMA-binding moiety.

PSMA-binding moieties containing thiourea instead of urea may be made by generating the isothiocyanate of the 2-aminoadipyl moiety. Aad di-t-butyl ester hydrochloride is mixed with carbon disulfide in NH₄0H, which is then treated with Pb(NO₃)₂ to convert the amine group to isothiocyanate (—N═C═S). This replaces the first reaction in Scheme 1, the rest would be the same to produce the thiourea version of the compound. Alternatively, an amine can be treated with thiocarbonyldiimidazole or thiophosgene in the presence of Dipea.

In Formulas I-a, II, III-a, and IV-a, the PSMA-binding moiety modifies the ureido group by replacing one or both —NH— groups with —S—, —O—, or —N(Me)—. As shown in Scheme 2, below, the formation of linker-carbamate-AA (e.g. Orn-carbamate-Aad), can be achieved by the conjugation of NH₂—Orn(ivDde)-loaded 2-chlorotrityl-resin to an Aad derivative, di-t-butyl 2-(((4-nitrophenoxy)carbonyl)oxy)hexanedioate (Scheme 2, compound 8). Briefly, diethyl glutarate (1 eq.) and diethyl oxalate (1 eq.) are added to sodium ethoxide (1 eq.) in Et₂O, and stirred at room temperature for 1 d. Following extraction and rotary evaporation, the residue is dissolved with 4 M HCl and refluxed for 4 h. The mixture is filtered to isolate the intermediate, 2-oxohexanedioic acid 5. Intermediate 5 (1 eq.) is reacted with t-butyl (E)-N,N′-diisopropylcarbamimidate (6.7 eq.) to give the intermediate, di-t-butyl 2-oxohexanedioate 6, which is then dissolved in MeCN (2.8 M) and NaBH₄ (10 eq.) is added to the solution. The suspension is then stirred at room temperature for 3 h. HCl (0.6 M) is used to slowly quench the reaction with sat. NaHCO₃ (aq) neutralizing the mixture to pH 8. The solution is then filtered and extracted with EtOAc, and dried by rotary evaporation to give the intermediate, di-t-butyl 2-hydroxyhexanedioate (7). Intermediate 7 is then reacted with p-nitrophenyl chloroformate (p-NPC) in presence of pyridine in DCM and purified to give the intermediate, di-t-butyl 2-(((4-nitrophenoxy)carbonyl)oxy)hexanedioate (8). Intermediate 8 is conjugated to NH₂—Orn(ivDde)-2-chlorotrityl-resin using HATU/DIPEA in DMF give 9. Finally, Fmoc-Ala(9-anth)-OH, Fmoc-tranexamic acid, and DOTA-tris(t-bu)ester are conjugated, followed by side chain deprotection/cleavage, and purification to afford 10. Similarly, PSMA-binding moieties containing —S— may be made by replacing compound 5 in Scheme 2 with 2-mercaptohexanedioic acid (commercially available). Alternatively, the hydroxyacid can be inverted with Tos-Cl and AcSH, then saponified. Alternatively, PSMA-binding moieties containing —S— may be made directly from most amino acids via diazotization and thioacetate addition. PSMA-binding moieties containing —N(Me)- may be made by methylating the ureido amides under Mitsunobu conditions, e.g. as discussed in further detail below.

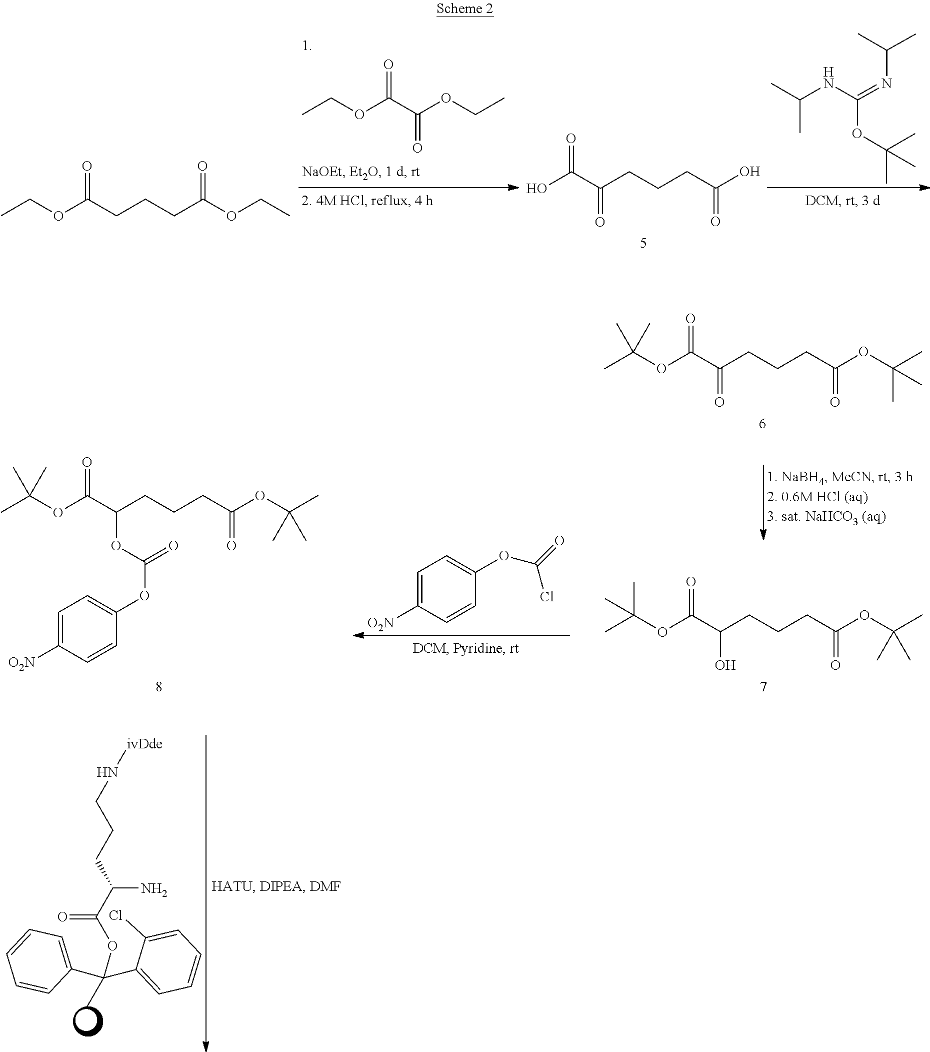

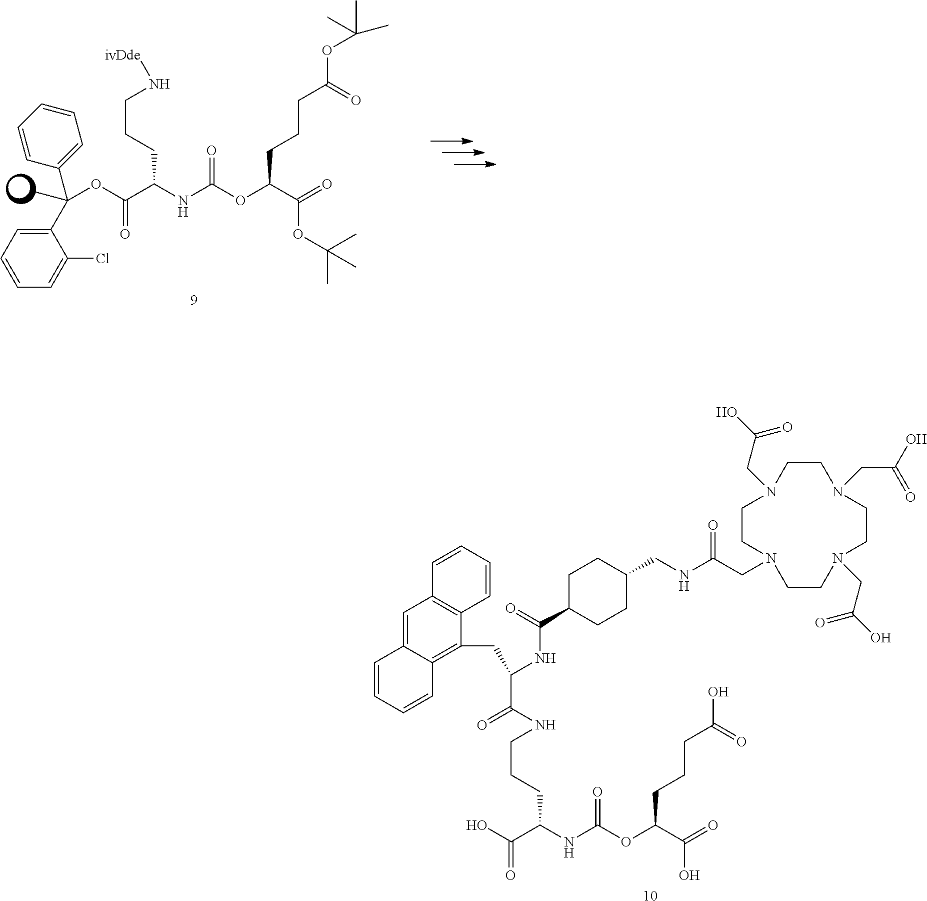

The formation of the thioether (—S—) and ether (—O—) linkages (e.g. for R⁴) can be achieved either on solid phase or in solution phase. For example, the formation of thioether (—S—) linkage can be achieved by coupling between a thiol-containing compound (such as the thiol group on cysteine side chain) and an alkyl halide (such as 3-(Fmoc-amino)propyl bromide and the like) in an appropriate solvent (such as N,N-dimethylformamide and the like) in the presence of base (such as N,N-diisopropylethylamine and the like). The formation of an ether (—O—) linkage can be achieved via the Mitsunobu reaction between an alcohol (such as the hydroxyl group on the side chain of serine or threonine, for example) and a phenol group (such as the side chain of tyrosine, for example) in the presence of triphenylphosphine and diisopropyl azidicarboxylate (DIAD) in an aprotic solvent (such as 1,4-dioxane and the like). If the reactions are carried out in solution phase, the reactants used are preferably in equivalent molar ratio (1 to 1), and the desired products can be purified by flash column chromatography or high performance liquid chromatography (HPLC). If the reactions are carried out on solid phase, meaning one reactant has been attached to a solid phase, then the other reactant is normally used in excess amount (≥3 equivalents of the reactant attached to the solid phase). After the reactions, the excess unreacted reactant and reagents can be removed by sequentially washing the solid phase (resin) using a combination of solvents, such as N,N-dimethylformamide, methanol and dichloromethane, for example.

Amides (e.g. peptide backbone amides, or ureido amides in the PMSA-binding moieties, etc.) may be N-methylated (i.e. alpha amino methylated) or otherwise N-modified. N-methylation may be achieved by directly using Fmoc-N-methylated amino acids during peptide synthesis. Alternatively, N-methylation under Mitsunobu conditions may be performed. First, a free primary amine group is protected using a solution of 4-nitrobenzenesulfonyl chloride (Ns-Cl) and 2,4,6-trimethylpyridine (collidine) in NMP. N-methylation may then be achieved in the presence of triphenylphosphine, diisopropyl azodicarboxylate (DIAD) and methanol. Subsequently, N-deprotection may be performed using mercaptoethanol and 1,8-diazabicyclo[5.4.0]undec-7-ene (DBU) in NMP. For coupling protected amino acids to N-methylated alpha amino groups, HATU, HOAt and DIEA may be used.

In some embodiments, the compounds are N-benzyl substituted. An example of a synthetic route for a PSMA-targeting compound with a N-4-bromobenzyl-substituted Orn-carbamate-Aad backbone is illustrated in Scheme 3, below. The ivDde protecting group in compound 9 can be deprotected by treating with 2% hydrazine in DMF to give compound 11. N-benzyl-substitution can be achieved via Mitsunobu conditions. 2-Nitrobenzenesulfonyl chloride (o-Ns-Cl, 5 eq.) and collidine (10 eq.) in N-Methyl-2-pyrrolidone (NMP) is added to 11 and mix for 15 min to give 12. N-alkylation is performed by adding triphenylphosphine (5 eq.), diisopropyl azodicarboxylate (DIAD, 5 eq.) and 4-bromobenzyl alcohol (10 eq.) in dry THF to give 13. For o-Ns deprotection, mercaptoethanol (10 eq.) and 1,8-diazabicyclo(5.4.0)undec-7-ene (DBU, 5 eq.) in NMP are added and allowed to mix for 5 min, and this step is repeated one more time to give 14. Then, Fmoc-Ala(9-anth)-OH, Fmoc-tranexamic acid, and DOTA-tris(t-bu)ester are conjugated in presence of HATU/DIPEA in DMF, followed by side chain deprotection/cleavage, and purification to afford 15.

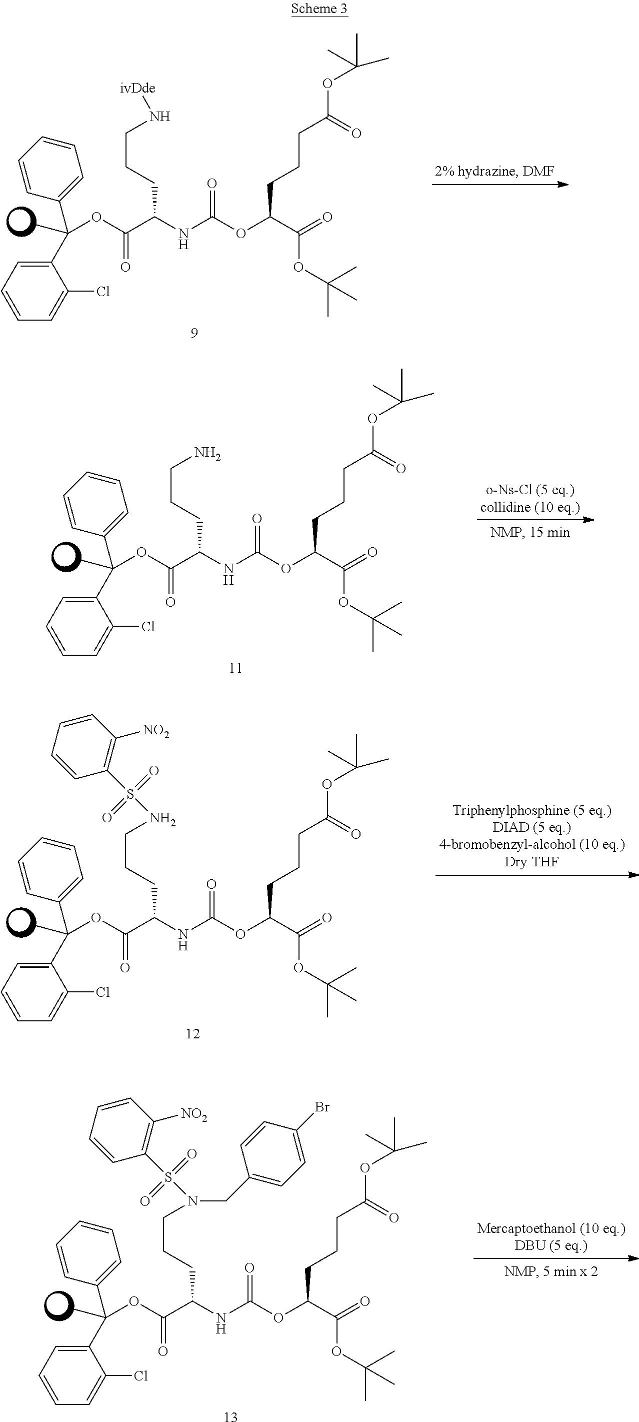

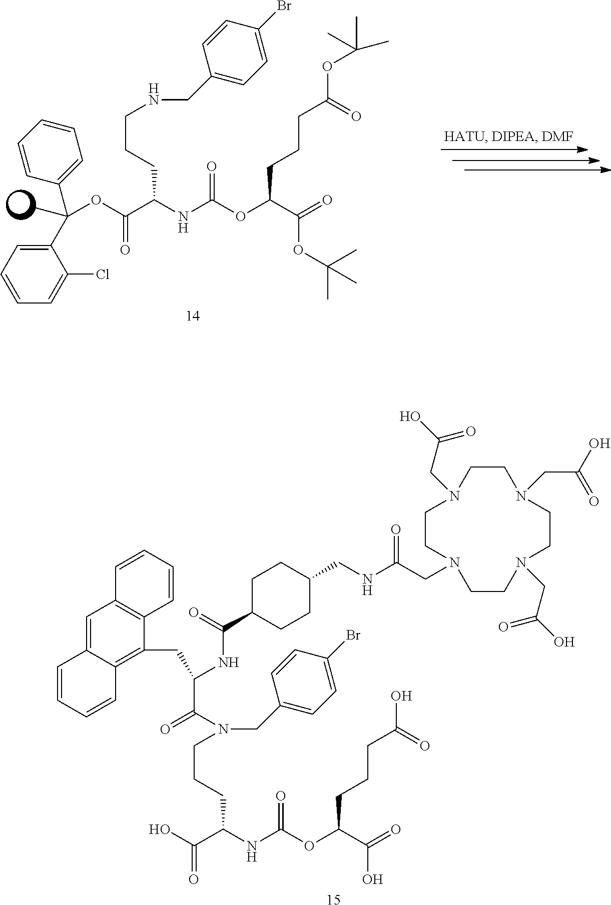

Non-peptide moieties (e.g. radiolabeling groups, albumin-binding groups and/or linkers) may be coupled to the peptide N-terminus while the peptide is attached to the solid support. This is facile when the non-peptide moiety comprises an activated carboxylate (and protected groups if necessary) so that coupling can be performed on resin. For example, but without limitation, a bifunctional chelator, such as 1,4,7,10-tetraazacyclododecane-1,4,7,10-tetraacetic acid (DOTA) tris(tert-butyl ester) may be activated in the presence of N-hydroxysuccinimide (NHS) and N,N′-dicyclohexylcarbodiimide (DCC) for coupling to a peptide. Alternatively, a non-peptide moiety may be incorporated into the compound via a copper-catalyzed click reaction under either liquid or solid phase conditions. Copper-catalyzed click reactions are well established in the art. For example, 2-azidoacetic acid is first activated by NHS and DCC and coupled to a peptide. Then, an alkyne-containing non-peptide moiety may be clicked to the azide-containing peptide in the presence of Cu²⁺ and sodium ascorbate in water and organic solvent, such as acetonitrile (ACN) and DMF and the like.

The synthesis of radiometal chelators is well-known and many chelators are commercially available (e.g. from Sigma-Aldrich™/Milipore Sigma™ and others). Protocols for conjugation of radiometals to the chelators are also well known (e.g. see Example 1, below). The synthesis of the silicon-fluorine-acceptor moieties can be achieved following previously reported procedures (e.g. Bernard-Gauthier et al. Biomed Res Int. 2014 2014:454503; Kostikov et al. Nature Protocols 2012 7:1956-1963; Kostikov et al. Bioconjug Chem. 2012 18:23:106-114; each of which is incorporated by reference in its entirety). The synthesis or acquisition of radioisotope-substituted aryl groups is likewise facile.

The synthesis of the R¹⁶R¹⁷BF₃ component on the PSMA-targeting compounds can be achieved following previously reported procedures (Liu et al. Angew Chem Int Ed 2014 53:11876-11880; Liu et al. J Nucl Med 2015 55:1499-1505; Liu et al. Nat Protoc 2015 10:1423-1432; Kuo et al. J Nucl Med, in press, doi:10.2967/jnumed.118.216598; each of which is incorporated by reference in its entirety). Generally, the BF₃-containing motif can be coupled to the linker via click chemistry by forming a 1,2,3-triazole ring between a BF₃-containing azido (or alkynyl) group and an alkynyl (or azido) group on the linker, or by forming an amide linkage between a BF₃-containing carboxylate and an amino group on the linker. To make the BF₃-containing azide, alkyne or carboxylate, a boronic acid ester-containing azide, alkyne or carboxylate is first prepared following by the conversion of the boronic acid ester to BF₃ in a mixture of HCl, DMF and KHF₂. For alkyl BF₃, the boronic acid ester-containing azide, alkyne or carboxylate can be prepared by coupling boronic acid ester-containing alkyl halide (such as iodomethylboronic acid pinacol ester) with an amine-containing azide, alkyne or carboxylate (such as N,N-dimethylpropargylamine). For aryl BF₃, the boronic acid ester can be prepared via Suzuki coupling using aryl halide (iodine or bromide) and bis(pinacolato)diboron.

¹⁸F-Fluorination of the BF₃-containing PSMA-targeting compounds via ¹⁸F-¹⁹F isotope exchange reaction can be achieved following previously published procedures (Liu et al. Nat Protoc 2015 10:1423-1432, incorporated by reference in its entirety). Generally, ˜100 nmol of the BF₃-containing compound is dissolved in a mixture of 15 μl of pyridazine-HCl buffer (pH=2.0-2.5, 1 M), 15 μl of DMF and 1 μl of a 7.5 mM KHF₂ aqueous solution. ¹⁸F-Fluoride solution (in saline, 60 μl) is added to the reaction mixture, and the resulting solution is heated at 80° C. for 20 min. At the end of the reaction, the desired product can be purified by solid phase extraction or by reversed high performance liquid chromatography (HPLC) using a mixture of water and acetonitrile as the mobile phase.

When the peptide has been fully synthesized on the solid support, the desired peptide may be cleaved from the solid support using suitable reagents, such as TFA, tri-isopropylsilane (TIS) and water. Side chain protecting groups, such as Boc, pentamethyldihydrobenzofuran-5-sulfonyl (Pbf), trityl (Trt) and tert-butyl (tBu) are simultaneously removed (i.e. deprotection). The crude peptide may be precipitated and collected from the solution by adding cold ether followed by centrifugation. Purification and characterization of the peptides may be performed by standard separation techniques, such as high performance liquid chromatography (HPLC) based on the size, charge and polarity of the peptides. The identity of the purified peptides may be confirmed by mass spectrometry or other similar approaches.

The present invention will be further illustrated in the following examples.

### Example 1: CCZ02011

### General Methods

All chemicals and solvents were obtained from commercial sources, and used without further purification. PSMA-targeted peptides were synthesized using a solid phase approach on an AAPPTec (Louisville, KY) Endeavor 90 peptide synthesizer. Purification of peptides was performed on an Agilent 1260 Infinity II Preparative System equipped with a model 1260 Infinity II preparative binary pump, a model 1260 Infinity variable wavelength detector (set at 220 nm), and a 1290 Infinity II preparative open-bed fraction collector. The HPLC column used was a preparative column (Gemini, NX—C18, 5 μ, 50×30 mm) purchased from Phenomenex. The collected HPLC eluates containing the desired peptide were lyophilized using a Labconco (Kansas City, MO) FreeZone 4.5 Plus freeze-drier. Mass analyses were performed using a Waters LC-MS system with an ESI ion source. C18 Sep-Pak cartridges (1 cm³, 50 mg) were obtained from Waters (Milford, MA). ⁶⁸Ga was eluted from an iThemba Labs (Somerset West, South Africa) generator. Radioactivity of ⁶⁸Ga-labeled peptides was measured using a Capintec (Ramsey, NJ) CRC®-25R/W dose calibrator, and the radioactivity of mouse tissues collected from biodistribution studies were counted using a Perkin Elmer (Waltham, MA) Wizard2 2480 automatic gamma counter.

### Synthesis of CCZ02011

The structure of CCZ02011 is shown below:

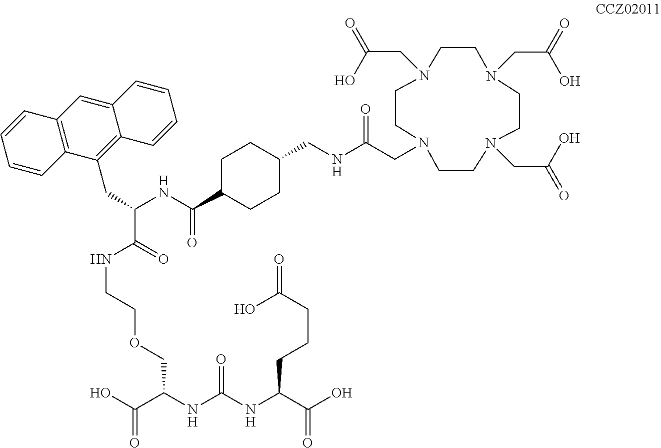

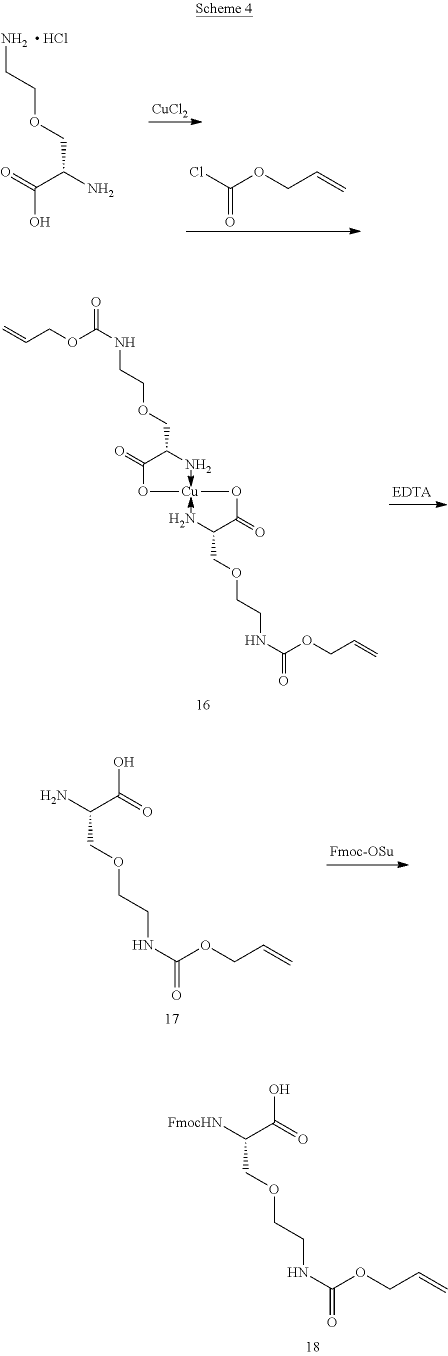

To synthesize CCZ02011, Fmoc-aminoethylserine(Alloc)-OH (compound 18, Scheme 4) was first synthesized. To a solution of NaOH (0.22 g, 10.86 mmol) in 30 mL of deionised water was added L-4-Oxalysine hydrochloride (1.00 g, 5.43 mmol). CuCl₂ was then added and the resulted mixture was refluxed for 1 h. After cooling down to room temperature, NaHCO₃ (0.46 g, 5.43 mmol) was added and the mixture was then cooled in ice bath at 0° C. Allyl chloroformate (0.98 g, 8.14 mmol) was added dropwise and the reaction mixture was stirred for 2 hours and allowed to warm to room temperature and then stirred overnight. The suspension was filtered with frits and the precipitate was collected and washed with deionised water (10 mL×3). The solid of compound 16 was dried under vacuum and then suspended in 30 mL deionised water. EDTA was added and the mixture was stirred at reflux for 2 hours. The product 17 was isolated by filtration and dried under vacuum and used directly for the next step. The white solid 17 (0.84 g, 3.62 mmol) was suspended in 30 mL deionised water and NaHCO₃ (0.30 g, 3.62 mmol) was added. Fmoc-OSu (1.22 g, 3.62 mmol) in 50 mL 1,4-dioxane was added to the resulted solution and the mixture was then stirred overnight at room temperature. The volume was reduced by rotary evaporator and the product of compound 18 was extracted into 50 mL ethyl acetate and then washed with brine (50 mL×2). Solvent was evaporated and the residual was purified by silica gel flash chromatography with hexanes and ethyl acetate to give the final product of compound 18 as a white foam 1.35 g, total yield 55%.

Fmoc-aminoethylserine(Alloc)-OH was loaded onto pre-swelled 2-Chlorotrityl resin in CH₂Cl₂ in present of DIEA overnight. Fmoc was then removed by treating the resin with 20% piperidine in DMF (3×8 min). To generate the isocyanate of the 2-aminoadipyl moiety, a solution of L-2-aminoadipic acid (Aad) di-tertbutyl ester hydrochloride (154.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes to give a solution of the isocyanate of the 2-aminoadipyl moiety. After which another 87.1 μL DIEA (0.5 mmol) was added, and then added to the aminoethylserine-immobilized resin and reacted for 16 h. After washing the resin with DMF, the Alloc-protecting group was removed with Pd(PPh₃)₄ in presence of phenylsilane (2×10 min). Fmoc-Ala(9-Anth)-OH was then coupled to the side chain of aminoethylserine using Fmoc-protected amino acid (4 eq.), HATU (4 eq.), and DIEA (7 eq.). Afterwards, elongation was continued with the addition of Fmoc-tranexamic acid, and finally DOTA-tris(t-bu)ester (2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+H]⁺ for CCZ02011 1108.51; found [M+H]⁺ 1108.72.

In vitro competitive binding assay result for CCZ02011 was Ki=1.23 nM (n=1).

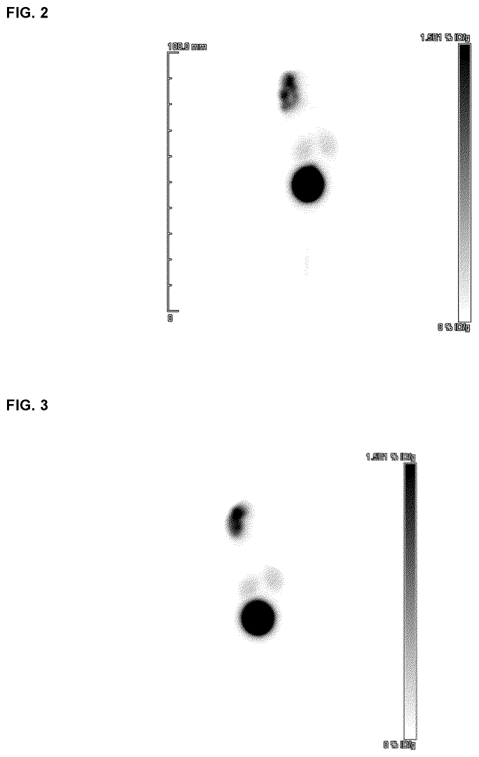

FIG. 2 shows PET image obtained at 1 h following the intravenous injection of ⁶⁸Ga—CCZ02011. Table 5 shows the biodistribution data for ⁶⁸Ga—CCZ02011 at 1 h post-injection in mice bearing LNCaP xenograft.

TABLE 5
Biodistribution data of ⁶⁸Ga-CCZ02011 in mice bearing
LNCaP xenograft at 1 h p.i., unit is in % ID/g.

1 h p.i. (n = 4)

| ⁶⁸Ga-CCZ02011 | Avg | Std |
|---|---|---|
| Blood | 0.58 | 0.13 |
| Urine | 343.93 | 179.61 |
| Fat | 0.12 | 0.03 |
| Seminal | 11.24 | 18.61 |
| Testes | 0.51 | 0.57 |
| Intestine | 0.40 | 0.21 |
| Spleen | 0.23 | 0.10 |
| Pancreas | 0.22 | 0.21 |
| Stomach | 0.07 | 0.04 |
| Liver | 0.27 | 0.03 |
| Adrenal | 0.36 | 0.16 |
| Kidney | 4.10 | 0.89 |
| Heart | 0.18 | 0.04 |
| Lungs | 0.43 | 0.07 |
| LNCaP tumor | 14.08 | 3.45 |
| Bone | 0.15 | 0.07 |
| Muscle | 0.15 | 0.09 |
| Brain | 0.02 | 0.01 |
| Salivary gland | 0.27 | 0.21 |
| Thyroid | 0.16 | 0.03 |
| Lacrimal | 0.33 | 0.15 |

### Example 2: CCZ02018

### Synthesis of CCZ02018

The structure of CCZ02018 is shown below:

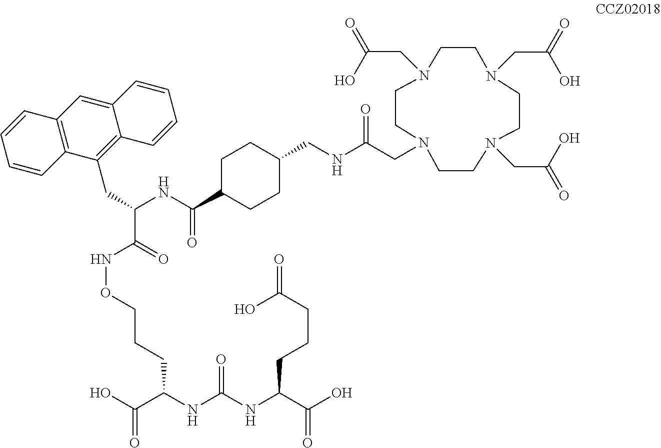

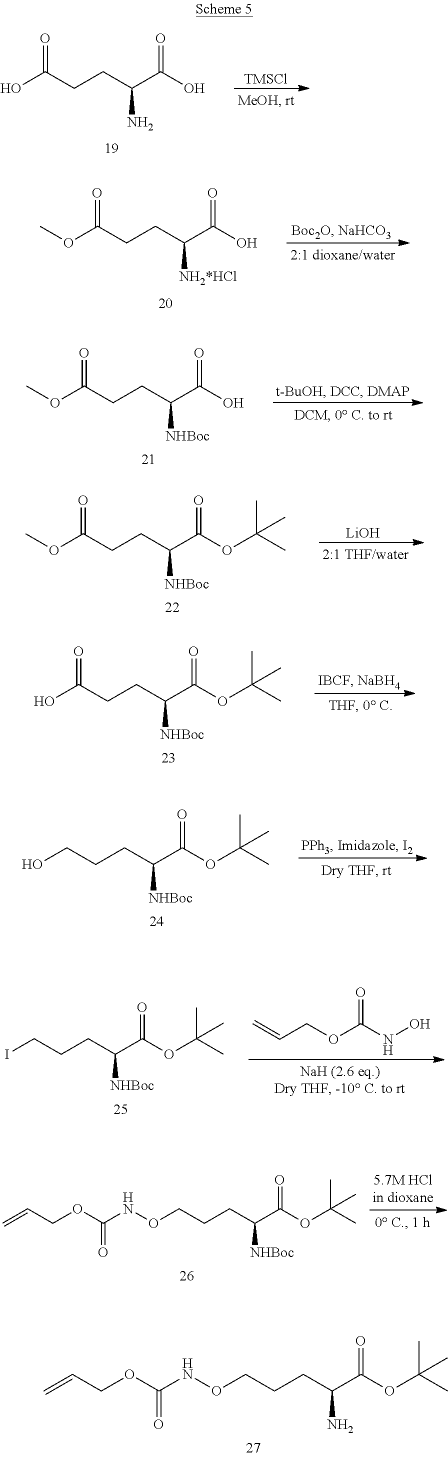

To synthesize CCZ02018, tert-butyl (S)-5-((((allyloxy)carbonyl)amino)oxy)-2-aminopentanoate (compound 27, scheme 5) was first made. To a suspension of L-glutamic acid 19 (20 g, 0.14 mol, 1 eq.) in dry MeOH (0.1 M, 350 mL) under Argon was added TMS-Cl (39 mL, 0.31 mol, 2.2 eq.) over 5 min. The clear solution was stirred at room temperature for 30 minutes. The reaction mixture was concentrated then co-evaporated with 1:1 toluene/DCM to yield (S)-2-amino-5-methoxy-5-oxopentanoic acid 20 (27.7 g, 0.14 mol, quantitative) as an off-white solid. Mass found for non-HCl salt product [M+H]⁺=176.3 m/z (S)-2-amino-5-methoxy-5-oxopentanoic acid 20 (27.7 g, 0.14 mol) was dissolved in 2:1 dioxane/water (465 mL, 0.3M) at 0° C. Boc₂O (37.1 g, 0.17 mol, 1.2 eq.) and NaHCO₃ (29.4 g, 0.35 mol, 2.5 eq.) were then added to the solution and stirred overnight. After overnight stirring, the mixture was concentrated. The aqueous solution was washed with diethyl ether (3×100 mL). Then 1M HCl (160 mL) was used to adjust the pH to 3-4. Extract the aqueous layer with ethyl acetate (4×200 mL). The combined organic layers were washed with water (400 mL) and brine (500 mL) and dried over Na₂SO₄. The combined organic extracts were concentrated to yield (S)-2-((tert-butoxycarbonyl)amino)-5-methoxy-5-oxopentanoic acid 21 (36.6 g, 0.14 mol, quantitative). Mass found for non-HCl salt product [M+Na]⁺=284.2 m/z. To an ice-cold DCM (170 mL) solution of DCC (9.47g, 45.92 mol, 1.2 eq.), DMAP (0.47 g, 3.83 mol, 0.1 eq.) and tBuOH (37 mL, 382.7 mol, 10 eq.) was added (S)-2-((tert-butoxycarbonyl)amino)-5-methoxy-5-oxopentanoic acid 21 (10 g, 38.27 mol, 1 eq.) dissolved in DCM (0.2M, 20 mL) over 30 minutes. The reaction was stirred at 0° C. for 1 h and then stirred overnight at room temperature. After overnight stirring, the suspension was filtered through a celite pad to remove DCU byproduct. The filtrate was washed with 0.1M HCl (200 mL), sat. NaHCO₃ solution (250 mL) and brine (300 mL). The organic phase was dried over Na₂SO₄, then filtered and concentrated. The crude was purified via flash chromatography (EA/Hex) to yield 1-(tert-butyl) 5-methyl (tert-butylcarbonyl)-L-glutamate 22 (7.89 g, 24.8 mmol, 65%). Mass found for product [M+H]+=318.3 m/z. To a solution of 1-(tert-butyl) 5-methyl (tert-butylcarbonyl)-L-glutamate 22 (5.09 g, 16.03 mmol, 1eq.) dissolved in THF (0.2M, 75 mL) was added 1M LiOH (0.77 g, 32 mL, 2 eq.) over 30 minutes. After reaction completion, the solution was cooled to 0° C. and 0.1M HCl was added to adjust the pH to 3-4. The suspension was extracted with ethyl acetate (6×50 mL). The combined organic layers are washed with brine (200 mL), dried over Na₂SO₄, filtered and concentrated. (S)-5-(tert-butoxy)-4-((tert-butoxycarbonyl)amino)-5-oxopentanoic acid 5 (4.975 g, 16.3 mmol) was immediately carried to the next step without purification. Mass of product found at [M+H]⁺ =304.3 m/z. (S)-5-(tert-butoxy)-4-((tert-butoxycarbonyl)amino)-5-oxopentanoic acid 23 (4.975 g, 16.4 mmol, 1 eq.) was dissolved in dry THF (0.5M, 33 mL). The reaction mixture was first cooled to −15° C. and then triethylamine (2.3 mL, 16.4 mmol, 1 eq.) was added. After 5 minutes, isobutyl chloroformate (3.2 mL, 24.6 mmol, 1.5 eq.) was added dropwise under Argon and stirred for 30 minutes. Sodium borohydride (3.102 g, 82 mmol, 5 eq.) was added to the reaction mixture and was stirred for another 30 minutes. After reaction completion, THF was evaporated under pressure and the excess sodium borohydride was quenched with 10% HCl solution. The reaction mixture was extracted with ethyl acetate (6×50 mL). The combined organic layers were washed with 10% HCl solution (3×50 mL), 10% Na₂CO₃ solution (3×50 mL) and brine (3×50 mL). The organic extracts are dried over Na₂SO₄ and purified via flash column chromatography (EA/Hex) to yield tert-butyl (S)-2-((tert-butoxycarbonyl)amino)-5-hydroxypentanoate 24 (2.394 g, 8.27 mmol, 56%) as a clear gel. Mass of product found [M+Na]⁺=312.2 m/z. Tert-butyl (S)-2-((tert-butoxycarbonyl)amino)-5-hydroxypentanoate 24 (2.6394 g, 9.12 mmol, 1 eq.) was dissolved in dry THF (0.3M, 30 mL) under Argon. Triphenylphosphine (3.5934 g, 13.7 mmol, 1.5 eq.), imidazole (0.933 g, 13.7 mmol, 1.5 eq.) and iodine (3.4772 g, 13.7 mmol, 1.5 eq.) were added, respectively. After reaction completion, the reaction mixture was concentrated under vacuum. The crude oil was diluted with ethyl acetate and filtered through a silica plug. The filtrate was then washed with 10% Na₂S₂O₃ solution (3×50 mL) and brine (3×50 mL). The organic layer was dried over Na₂SO₄, filtered and concentrated. The crude product was purified via flash column chromatography (EA/Hex) to yield tert-butyl (S)-2-((tert-butoxycarbonyl)amino)-5-iodopentanoate 25 (2.8485 g, 7.13 mmol, 78%) as a white powder. Mass of product found [M+3ACN+2H]⁺=462.4 m/z. N-allyloxycarbonate hydroxylamine (2.4216 g, 20.7 mmol, 2.6 eq.) was dissolved in dry THF (6 mL) and cooled to −10° C. and 60% NaH in mineral oil (0.742 g, 18.5 mmol, 2.6 eq.) was added in three portions. The reaction mixture was adjusted to 0° C. and then a solution of tert-butyl (S)-2-((tert-butoxycarbonyl)amino)-5-iodopentanoate 25 (2.8485 g, 7.13 mmol, 1 eq.) in dry THF (18 mL) was added to the mixture. After reaction completion, the reaction was quenched with saturated NH₄Cl (50 mL) and extracted with ethyl acetate (3×50 mL). The combined organic layers were washed with brine (100 mL). The organic layer was dried over Na₂SO₄, filtered and concentrated. The crude product is purified via flash column chromatography to afford tert-butyl (S)-5-((((allyloxy)carbonyl)amino)oxy)-2-((tert-butoxycarbonyl)amino)pentanoate 26 (1.4572 g, 3.75 mmol, 53%) as a clear gel. Mass of product found [M+H]⁺=389.2 m/z. tert-butyl (S)-5-((((allyloxy)carbonyl)amino)oxy)-2-((tert-butoxycarbonyl)amino)pentanoate 26 (424.8 mg, 1.09 mmol, 1 eq.) was dissolved in dioxane (0.3M, 3.6 mL) and cooled down to 0° C. Once cooled, 5.7M HCl in dioxane (5 mL) was added and stirred for 30 minutes. After reaction completion, the mixture was diluted with ethyl acetate (10 mL) and quenched with sat. NaHCO₃ (10 mL). The organic layer was washed with sat. NaHCO₃ (2×10 mL) and brine (2×15 mL). The organic layer was dried over Na₂SO₄ then filtered and concentrated. Post concentration yielded tert-butyl (S)-5-((((allyloxy)carbonyl)amino)oxy)-2-aminopentanoate 27 (271.4 mg, 0.94 mmol, 80%) as a yellowish gel. Mass of product found [M+H]⁺=289.3 m/z.

Fmoc-Aad(OtBu)-OH was loaded onto pre-swelled 2-Chlorotrityl resin in CH₂Cl₂ in present of DIEA overnight. Fmoc was then removed by treating the resin with 20% piperidine in DMF (3×8 min). To generate the isocyanate of tert-butyl (S)-5-((((allyloxy)carbonyl)amino)oxy)-2-aminopentanoate, a solution of tert-butyl (S)-5-((((allyloxy)carbonyl)amino)oxy)-2-aminopentanoate (144.7 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes to give a solution of the isocyanate of the tert-butyl (S)-5-((((allyloxy)carbonyl)amino)oxy)-2-aminopentanoate moiety. After which another 87.1 μL DIEA (0.5 mmol) was added, and then added to the Aad(OtBu)-immobilized resin and reacted for 16 h. After washing the resin with DMF, the Alloc-protecting group was removed with Pd(PPh₃)₄ in presence of phenylsilane (2×10 min). Fmoc-Ala(9-Anth)-OH was then coupled to the side chain of aminoethylserine using Fmoc-protected amino acid (4 eq.), HATU (4 eq.), and DIEA (7 eq.). Afterwards, elongation was continued with the addition of Fmoc-tranexamic acid, and finally DOTA-tris(t-bu)ester(2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+H]⁺ for CCZ02018 1108.51; found [M+H]⁺ 1108.61.

In vitro competitive binding assay result for CCZ02018 was Ki=1.61±0.04 nM (n=2).

FIG. 3 shows PET image obtained at 1 h following the intravenous injection of ⁶⁸Ga—CCZ02018. Table 6 shows the biodistribution data for ⁶⁸Ga—CCZ02018 at 1 h post-injection in mice bearing LNCaP xenograft

TABLE 6
Biodistribution data of ⁶⁸Ga-CCZ02018 in mice bearing
LNCaP xenograft at 1 h p.i., unit is in % ID/g.

1 h p.i. (n = 4)

| ⁶⁸Ga-CCZ02018 | Avg | Std |
|---|---|---|
| Blood | 0.67 | 0.28 |
| Urine | 613.72 | 197.86 |
| Fat | 0.17 | 0.10 |
| Seminal | 0.27 | 0.30 |
| Testes | 0.22 | 0.05 |
| Intestine | 0.25 | 0.07 |
| Spleen | 0.24 | 0.07 |
| Pancreas | 0.13 | 0.04 |
| Stomach | 0.07 | 0.04 |
| Liver | 0.21 | 0.05 |
| Adrenal | 0.53 | 0.35 |
| Kidney | 6.91 | 4.06 |
| Heart | 0.18 | 0.07 |
| Lungs | 0.58 | 0.16 |
| LNCaP tumor | 18.88 | 1.66 |
| Bone | 0.13 | 0.04 |
| Muscle | 0.10 | 0.03 |
| Brain | 0.03 | 0.01 |
| Salivary gland | 0.22 | 0.08 |
| Thyroid | 0.20 | 0.07 |

### Example 3: CCZ01194 and CCZ01198

### Synthesis of CCZ01194 and CCZ01198

The structures of CCZ01194 and CCZ01198 are shown below:

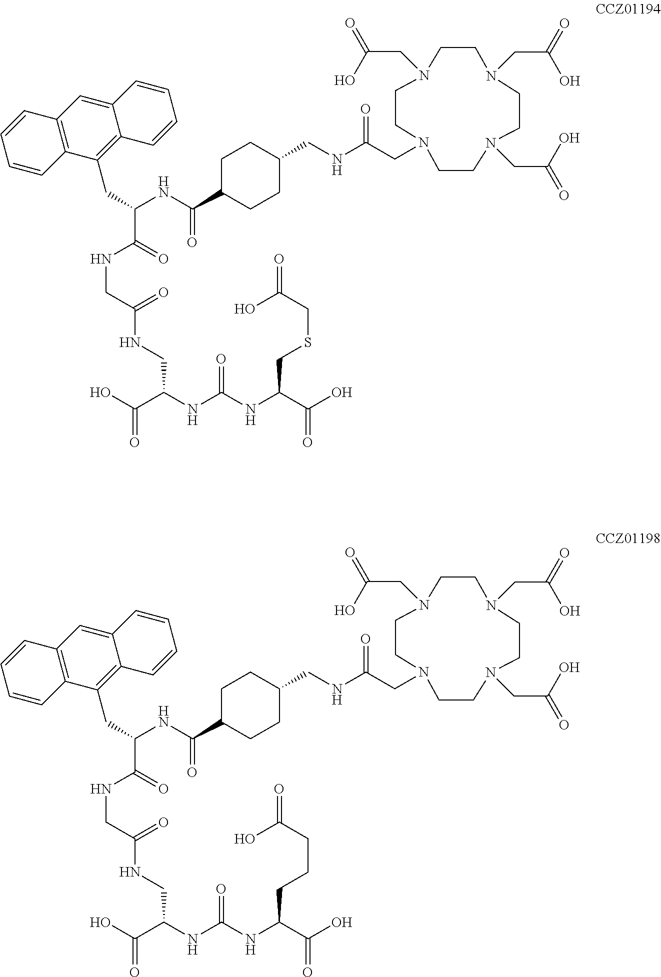

Fmoc-Dap(ivDde)-OH was loaded onto pre-swelled 2-Chlorotrityl resin in CH₂Cl₂ in present of DIEA overnight. Fmoc was then removed by treating the resin with 20% piperidine in DMF (3×8 min). A solution of S-carboxymethycysteine di-tertbutyl ester hydrochloride (for CCZ01194, 163.9 mg, 0.5 mmol, 10 eq relative to resin) or L-2-aminoadipic acid (Aad) di-tertbutyl ester hydrochloride (for CCZ01198, 154.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the Dap(ivDde)-immobilized resin and reacted for 16 h. After washing the resin with DMF, the ivDde-protecting group was removed with 2% hydrazine (5×5 min). Fmoc-Gly-OH, Fmoc-Ala(9-Anth)-OH and Fmoc-tranexamic acid were then coupled to the side chain of Dap using Fmoc-protected amino acid (4 eq.), HATU (4 eq.), and DIEA (7 eq.). Finally DOTA-tris(t-bu)ester (2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+H]⁺ for CCZ01194 1139.46; found [M+H]⁺ 1139.90 calculated [M+H]⁺ for CCZ01198 1120.51; found [M+H]+1120.82.

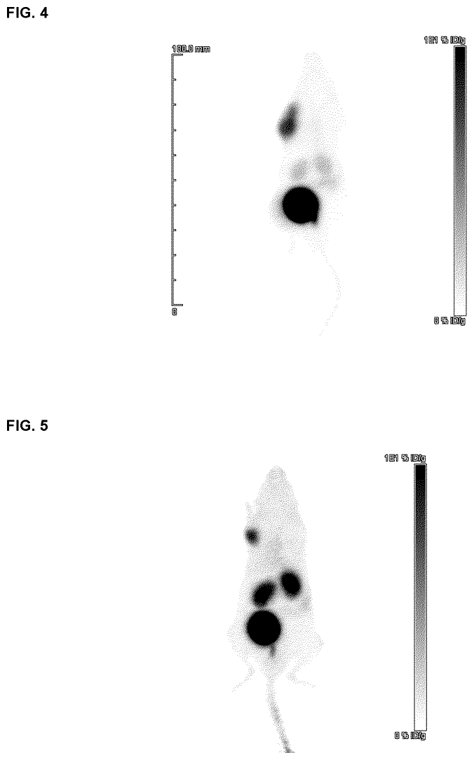

FIG. 4 shows PET image obtained at 1 h following the intravenous injection of ⁶⁸Ga—CCZ01194. Table 7 shows the biodistribution data for ⁶⁸Ga—CCZ01194 and ⁶⁸Ga—CCZ01198, respectively, at 1 hr post-injection in mice bearing LNCaP xenograft.

TABLE 7
Biodistribution data for ⁶⁸Ga-CCZ01194 and
⁶⁸Ga-CCZ01198 at 1 h post-injection in mice
bearing LNCaP xenograft, unit is in % ID/g.

| | |
|---|---|
| 68Ga-CCZ01194 | 68Ga-CCZ01198 |
| (n = 4) | (n = 4) |

| | Avg | Std | Avg | Std |
|---|---|---|---|---|
| Blood | 0.98 | 0.12 | 0.43 | 0.01 |
| Urine | 311.63 | 101.01 | 384.68 | 227.74 |
| Fat | 0.27 | 0.04 | 0.07 | 0.01 |
| Seminal | 0.13 | 0.04 | 0.47 | 0.84 |
| Testes | 0.23 | 0.04 | 0.14 | 0.03 |
| Intestine | 0.79 | 0.16 | 0.16 | 0.01 |
| Spleen | 0.24 | 0.04 | 0.12 | 0.01 |
| Pancreas | 0.15 | 0.02 | 0.08 | 0.00 |
| Stomach | 0.09 | 0.02 | 0.03 | 0.01 |
| Liver | 0.35 | 0.06 | 0.15 | 0.02 |
| Adrenal | 0.65 | 0.39 | 0.19 | 0.03 |
| Kidney | 7.33 | 3.99 | 2.18 | 0.52 |
| Heart | 0.27 | 0.01 | 0.11 | 0.01 |
| Lungs | 0.70 | 0.04 | 0.32 | 0.00 |
| LNCaP tumor | 9.12 | 1.34 | 4.84 | 1.00 |
| Bone | 0.14 | 0.02 | 0.07 | 0.03 |
| Muscle | 0.14 | 0.01 | 0.07 | 0.01 |
| Brain | 0.03 | 0.00 | 0.01 | 0.00 |
| Salivary gland | 0.34 | 0.24 | 0.12 | 0.01 |
| Thyroid | 0.29 | 0.02 | 0.13 | 0.01 |

### Example 4: CCZ02010, CCZ01186 and CCZ01188

### Synthesis of CCZ01186, CCZ01188 and CCZ02010

The structures of CCZ01186, CCZ01188 and CCZ02010 are shown below:

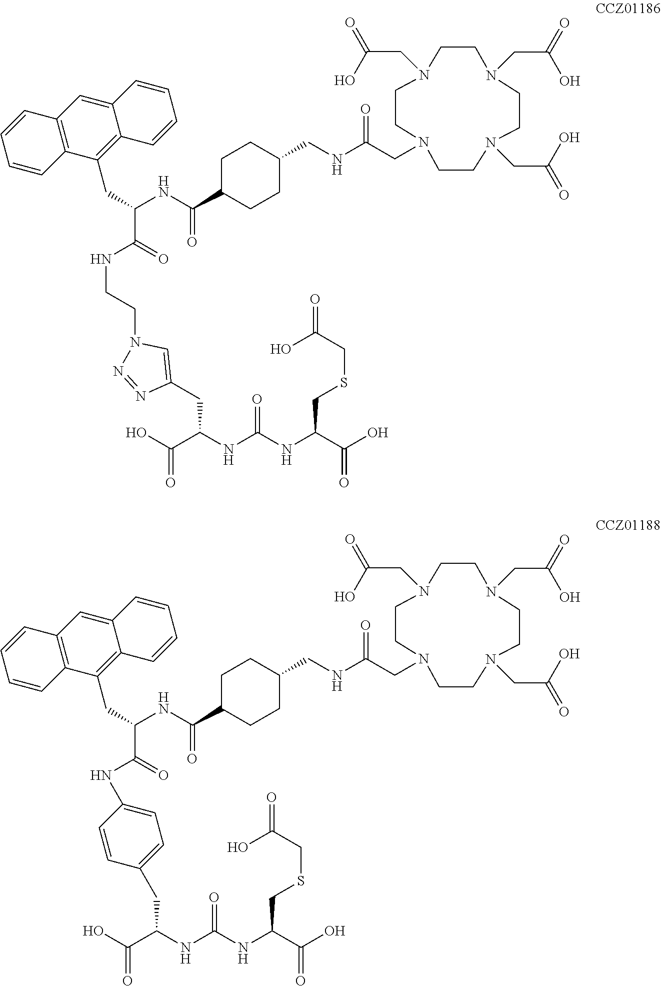

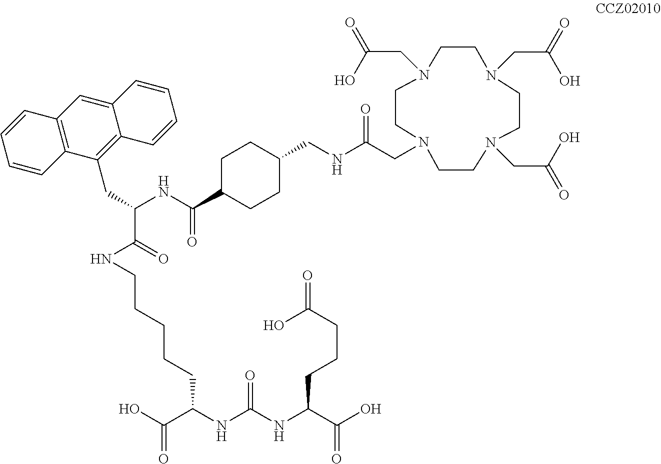

For CCZ01186, Fmoc-propargyl-Gly-OH was loaded onto pre-swelled 2-Chlorotrityl resin in CH₂Cl_{z} in present of DIEA overnight. Fmoc was then removed by treating the resin with 20% piperidine in DMF (3×8 min). A solution of S-carboxymethylcysteine di-tertbutyl ester hydrochloride (154.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the propargyl-Gly-immobilized resin and reacted for 16 h. 2-Azidoethanamine was added in presence of CuSO₄ and sodium ascorbate, and reacted overnight. After washing the resin with DMF, Fmoc-Ala(9-Anth)-OH and Fmoc-tranexamic acid were then coupled to the resin using Fmoc-protected amino acid (4 eq.), HATU (4 eq.), and DIEA (7 eq.). Finally DOTA-tris(t-bu)ester (2-(4,7,10-tris(2-(t-butoxy)-2-oxoehtyl)-1,4,7,10)-tetraazacyclododecan-1-yl)acetic acid).

For CCZ01188, Fmoc-Phe(4-NH-Alloc)-OH was loaded onto pre-swelled 2-Chlorotrityl resin in CH₂Cl₂ in present of DIEA overnight. Fmoc was then removed by treating the resin with 20% piperidine in DMF (3×8 min). A solution of S-carboxymethylcysteine di-tertbutyl ester hydrochloride (154.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the Phe(4-NH-Alloc)-immobilized resin and reacted for 16 h. After washing the resin with DMF, the Alloc-protecting group was removed with Pd(PPh₃)₄ in presence of phenylsilane (2×10 min). Fmoc-Ala(9-Anth)-OH and Fmoc-tranexamic acid were then coupled to the resin using Fmoc-protected amino acid (4 eq.), HATU (4 eq.), and DIEA (7 eq.). Finally DOTA-tris(t-bu)ester (2-(4,7,10-tris(2-(t-butoxy)-2-oxoehtyl)-1,4,7,10)-tetraazacyclododecan-1-yl)acetic acid).

For CCZ02010, Fmoc-homolysine(ivDde)-OH was loaded onto pre-swelled 2-Chlorotrityl resin in CH₂Cl₂ in present of DIEA overnight. Fmoc was then removed by treating the resin with 20% piperidine in DMF (3×8 min). A solution of L-2-aminoadipic acid (Aad) di-tertbutyl ester hydrochloride (154.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the homolysine (ivDde)-immobilized resin and reacted for 16 h. After washing the resin with DMF, the ivDde-protecting group was removed with 2% hydrazine (5×5 min). Fmoc-Ala(9-Anth)-OH and Fmoc-tranexamic acid were then coupled to the resin using Fmoc-protected amino acid (4 eq.), HATU (4 eq.), and DIEA (7 eq.). Finally DOTA-tris(t-bu)ester (2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+H]⁺ for CCZ01186 1177.49; found [M+H]⁺ 1177.69; calculated [M+H]⁺ for CCZ01188 1158.47; found [M+H]⁺ 1158.85; calculated [M+H]⁺ for CCZ02010 1120.55; found [M+H]⁺ 1121.00.

In vitro competitive binding assay result for CCZ02010 was Ki=17 nM (n=1).

Table 8 shows the biodistribution data for ⁶⁸Ga—CCZ01186 and ⁶⁸Ga—CCZ01188, respectively, at 1 h post-injection in mice bearing LNCaP xenograft.

TABLE 8
Biodistribution data for ⁶⁸Ga-CCZ01186 and
⁶⁸Ga-CCZ01188 at 1 h post-injection in mice
bearing LNCaP xenograft, unit is in % ID/g.

| | |
|---|---|
| ⁶⁸Ga-CCZ01186 | ⁶⁸Ga-CCZ01188 |
| (n = 4) | (n = 3) |

| | Avg | Std | Avg | Std |
|---|---|---|---|---|
| Blood | 0.72 | 0.20 | 3.91 | 0.75 |
| Urine | 439.81 | 48.85 | 367.12 | 111.59 |
| Fat | 0.16 | 0.07 | 0.62 | 0.17 |
| Seminal | 6.65 | 13.14 | 0.85 | 0.53 |
| Testes | 0.17 | 0.05 | 0.82 | 0.09 |
| Intestine | 0.08 | 0.04 | 0.56 | 0.08 |
| Spleen | 0.34 | 0.10 | 0.61 | 0.08 |
| Pancreas | 0.16 | 0.02 | 0.38 | 0.04 |
| Stomach | 0.14 | 0.06 | 0.19 | 0.04 |
| Liver | 0.23 | 0.04 | 0.81 | 0.18 |
| Adrenal | 0.29 | 0.15 | 0.98 | 0.22 |
| Kidney | 2.20 | 0.33 | 4.02 | 1.05 |
| Heart | 0.57 | 0.14 | 1.03 | 0.13 |
| Lungs | 0.21 | 0.06 | 2.15 | 0.43 |
| LNCaP tumor | 0.89 | 0.31 | 1.43 | 0.11 |
| Bone | 0.13 | 0.02 | 0.23 | 0.02 |
| Muscle | 0.23 | 0.09 | 0.41 | 0.05 |
| Brain | 0.02 | 0.00 | 0.07 | 0.01 |
| Salivary gland | 0.40 | 0.27 | 1.56 | 0.36 |
| Thyroid | 2.49 | 4.49 | 0.67 | 0.10 |

### Example 5: CCZ02032 and CCZ02033

### Synthesis of CCZ02032 and CCZ02033

The structures of CCZ02032 and CCZ02033 are shown below:

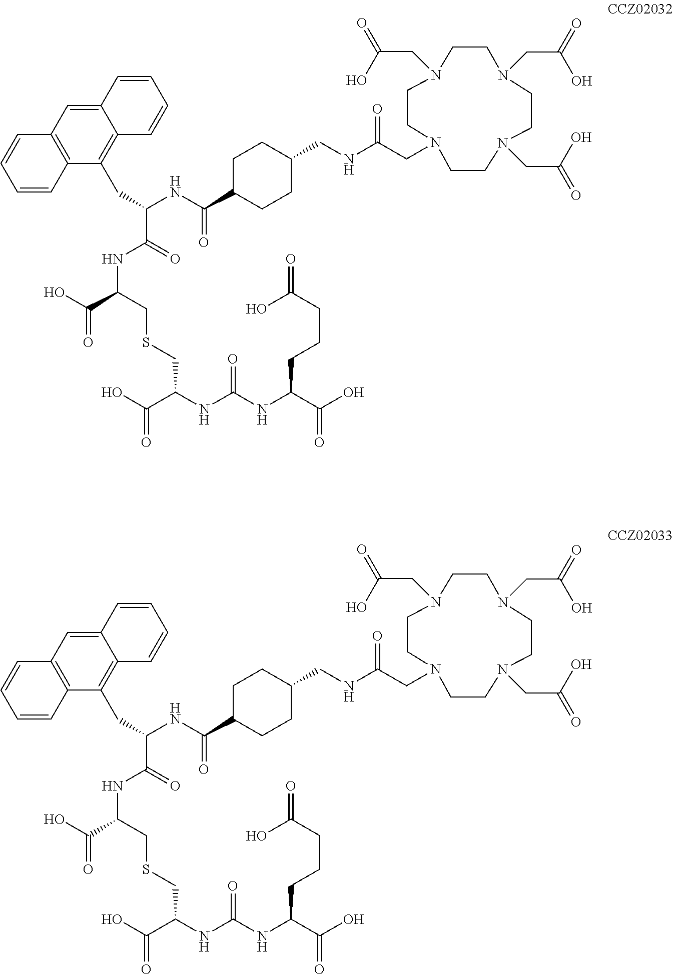

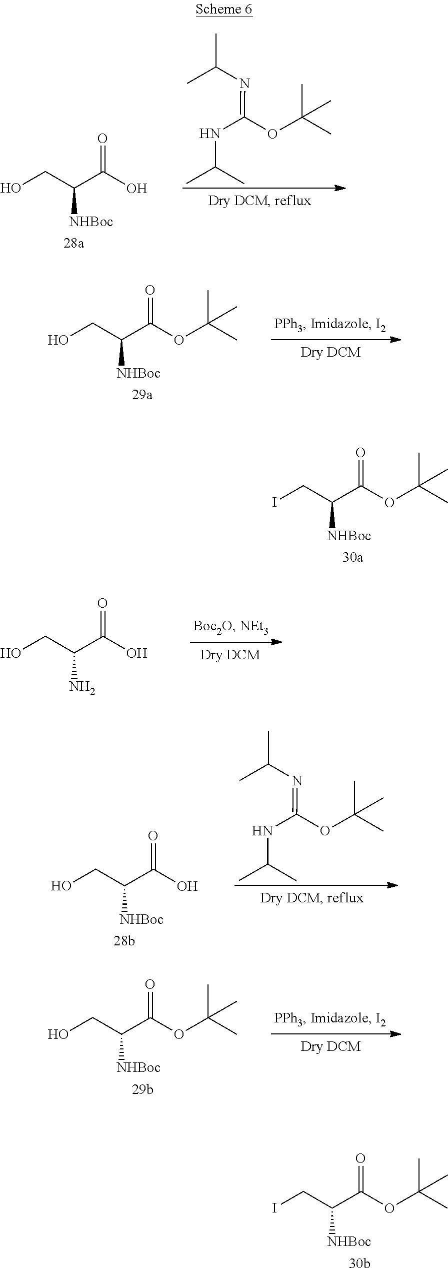

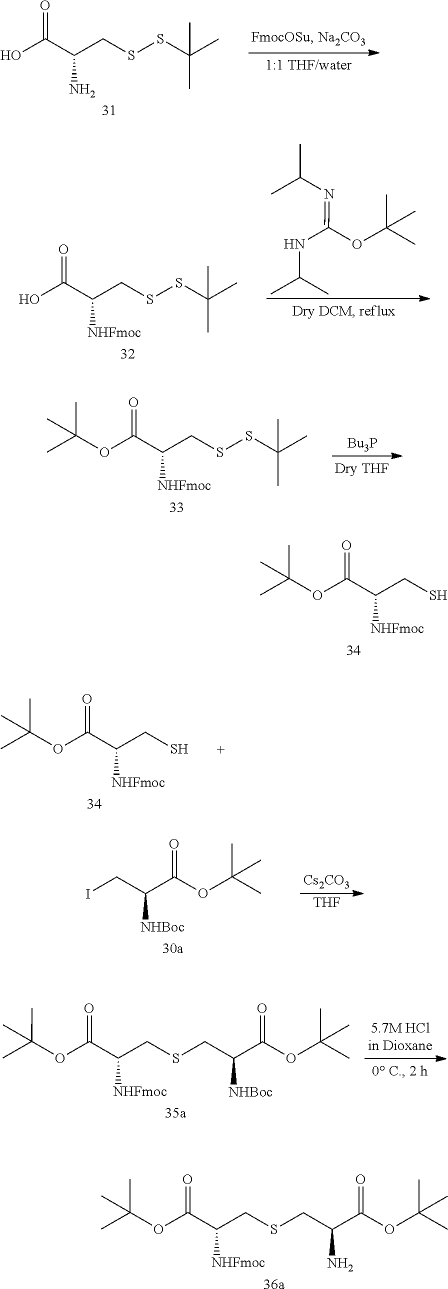

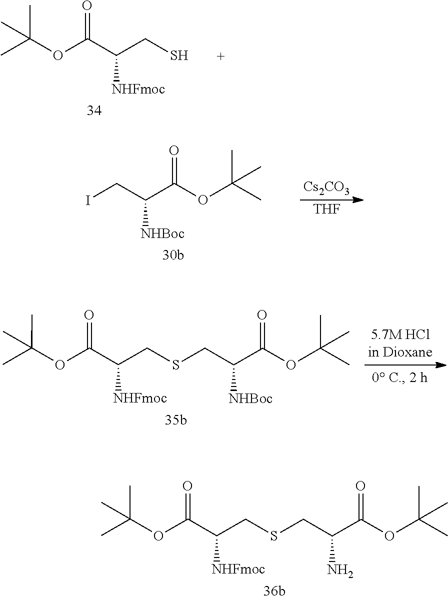

To synthesize CCZ02032 and CCZ02033, tert-butyl N-(((9H-fluoren-9-yl)methoxy)carbonyl)-S—(R-2-amino-3-(tert-butoxy)-3-oxopropyl)-L-cysteina te (36a, scheme 6) and N-(((9H-fluoren-9-yl)methoxy)carbonyl)-S—(S-2-amino-3-(tert-butoxy)-3-oxopropyl)-L-cysteina te (36b, scheme 5) were first synthesized, respectively. Into a solution of (tert-butoxycarbonyl)-L-serine 28a (2000 mg, 9.75 mmol, 1 eq.) in dry DCM (0.52M, 19 mL) was added N,N-diisopropylcarbamimidate (7421.9 mg, 27.05 mmol, 3.8 eq.). The reaction was stirred in an ice bath for 30 minutes before allowing to warm to room temperature to stir overnight. Hexanes (30 mL) was added to the reaction and stirred for 15 minutes. The suspension was filtered through a celite pad and concentrated under vacuum. The crude product was purified via flash column chromatography (EA/Hex) to yield tert-butyl (tert-butoxycarbonyl)-L-serinate 29a (1.801 g, 6.89 mmol, 71%) as a colourless gel. Mass of product found [M+H]⁺=262.4 m/z. Tert-butyl (tert-butoxycarbonyl)-L-serinate 29a (900 mg, 3.44 mmol, 1 eq.) was dissolved in dry THF (0.3M, 12 mL) under Argon. Triphenylphosphine (1353.4 mg, 5.16 mmol, 1.5 eq.), imidazole (351.3 mg, 5.16 mmol, 1.5 eq.) and iodine (1310.0 mg, 5.16 mmol, 1.5 eq.) were added, respectively. After reaction completion, the reaction mixture was concentrated under vacuum. The crude oil was diluted with ethyl acetate and filtered through a silica plug. The filtrate was then washed with 10% Na₂S₂O₃ solution (3×50 mL) and brine (3×50 mL). The organic layer was dried over Na₂SO₄, filtered and concentrated. The crude product was purified via flash column chromatography (EA/Hex) to yield tert-butyl R-2-((tert-butoxycarbonyl)amino)-3-iodopropanoate 30a (1.1338 g, 3.05 mmol, 89%) as a colourless oil. Mass of product found [M+H]⁺=372.1 m/z. D-Serine (5000 mg, 24.4 mmol, 1 eq.) was dissolved in 1M NaOH (25 mL) and cooled to 0° C. A solution of Boc₂O (6394.7 mg, 29.3 mmol, 1.2 eq.) in 1,4-dioxane (1M, 25 mL) was added and then warmed to room temperature. Upon completion, 1,4-dioxane was evaporated and the aqueous layer was washed with hexanes (3×50 mL). The aqueous phase was acidified to pH 1-2 with sat. KHSO₄ solution. This mixture was then extracted with ethyl acetate (4×60 mL). The combined organic layers are dried over MgSO₄, filtered, and concentrated. The crude product was used for the next reaction. The crude isolated yield was quantitative. Into a solution of (tert-butoxycarbonyl)-D-serine 28b (500 mg, 2.44 mmol, 1eq.) in dry DCM (0.52M, 5 mL) was added N,N-diisopropylcarbamimidate (1857.4 mg, 9.27 mmol, 3.8 eq.). The reaction was stirred in an ice bath for 30 minutes before allowing to warm to room temperature to stir overnight. Hexanes (8 mL) was added to the reaction and stirred for 15 minutes. The suspension was filtered through a celite pad and concentrated under vacuum. The crude product was purified via flash column chromatography (EA/Hex) to yield tert-butyl (tert-butoxycarbonyl)-D-serinate 29b (351.5 mg, 1.35 mmol, 55%) as a colourless oil. Mass of product found [M+H]⁺=262.4 m/z. Tert-butyl (tert-butoxycarbonyl)-D-serinate 29b (336.4 mg, 1.29 mmol, 1eq.) was dissolved in dry THF (0.3M, 4.5 mL) under Argon. Triphenylphosphine (508.8 g, 1.94 mmol, 1.5 eq.), imidazole (132.1 mg, 1.94 mmol, 1.5 eq.) and iodine (492.4 mg, 1.94 mmol, 1.5 eq.) were added, respectively. After reaction completion, the reaction mixture was concentrated under vacuum. The crude oil was diluted with ethyl acetate and filtered through a silica plug. The filtrate was then washed with 10% Na₂S₂O₃ solution (3×10 mL) and brine (3×10 mL). The organic layer was dried over Na₂SO₄, filtered and concentrated. The crude product was purified via flash column chromatography (EA/Hex) to yield tert-butyl S-2-((tert-butoxycarbonyl)amino)-3-iodopropanoate 30b (444.7 mg, 2.69 mmol, 93%) as a colourless oil. Mass of product found [M+H]⁺=372.1 m/z. To a solution of L-S—(StBu)-cysteine 31 (500 mg, 2.4 mmol, 1eq.) in 10% Na₂CO₃ solution (0.16M, 15 mL) was added a solution of FmocOSu (809.6 mg, 2.4 mmol, 1 eq.) in 1,4-dioxane (0.16M, 15 mL). The suspension was stirred for 1 h at room temperature. Wash the aqueous mixture with diethyl ether (3×15 mL) and acidify with 1M HCl until white emulsion forms. Extract the aqueous layer with ethyl acetate (3×20 mL). The combined organic layers are dried over MgSO₄, filtered, and concentrated to yield N-(((9H-fluoren-9-yl)methoxy)carbonyl)-S-(tert-butylthio)-L-cysteine 32 (828.6 mg, 1.88 mmol, 80%) as an off-white solid. Mass of product found [M+H]⁺=432.1 m/z. Into a solution of N-(((9H-fluoren-9-yl)methoxy)carbonyl)-S-(tert-butylthio)-L-cysteine 32 (541.5 mg, 1.25 mmol, 1 eq.) in dry DCM (0.52M, 2.5 mL) was added N,N-diisopropylcarbamimidate (951.5 mg, 4.75 mmol, 3.8 eq.). The reaction was stirred in an ice bath for 30 minutes before allowing to warm to room temperature to stir overnight. Hexanes (6 mL) was added to the reaction and stirred for 15 minutes. The suspension was filtered through a celite pad and concentrated. The crude product was purified via flash column chromatography (EA/Hex) to yield tert-butyl N-(((9H-fluoren-9-yl)methoxy)carbonyl)-S-(tert-butylthio)-L-cysteinate 33 (541.3 mg, 1.11 mmol, 89%) as a colourless oil. Mass of product found at [M+H]⁺=488.2 m/z. Tert-butyl N-(((9H-fluoren-9-yl)methoxy)carbonyl)-S-(tert-butylthio)-L-cysteinate 33 (568.6 mg, 1.17 mmol, 1 eq.) was dissolved in THF (0.13M, 9 mL), followed by dropwise addition of tributylphosphine (0.44 mL, 1.76 mmol, 1.5 eq.). The reaction was stirred under Argon for 30 minutes. Then, water (0.62 mL) was added to the solution. The reaction mixture was stirred for 2 hours at room temperature. The reaction was concentrated and dissolved in ethyl acetate. The organic layer was washed with 10% citric acid solution (40 mL) and brine (75 mL). The organic layer was dried over Na₂SO₄, filtered, and concentrated. The crude product was purified via flash column chromatography (EA/Hex) to yield tert-butyl N-(((9H-fluoren-9-yl)methoxy)carbonyl)-L-cysteinate 34 (311.5 mg, 0.78 mmol, 68%) as a clear yellowish oil. Mass of product found [M+H]⁺=400.2 m/z. Tert-butyl N-(((9H-fluoren-9-yl)methoxy)carbonyl)-L-cysteinate 34 (145 mg, 0.36 mmol, 1 eq.) and tert-butyl R-2-((tert-butoxycarbonyl)amino)-3-iodopropanoate 30a (148.5 mg, 0.40 mmol, 1.1 eq.) were dissolved in DMF (0.06M, 6 mL). Once dissolved, Cs₂CO₃ (117.3 mg, 0.36 mmol, 1 eq.) was added in three portions over 30 min. Once added, the reaction was stirred at room temperature until completion. Once complete, dilute reaction mixture with ethyl acetate (30 mL). The organic layer was washed with water (7×20 mL) and brine (30 mL). The organic layer was dried over MgSO₄, filtered, and concentrated. The crude product was purified via flash column chromatography (EA/Hex) to yield tert-butyl N-(((9H-fluoren-9-yl)methoxy)carbonyl)-S—(R-3-(tert-butoxy)-2-((tert-butoxycarbonyl)amino)-3-oxopropyl)-L-cysteinate 35a (107.4 mg, 0.17 mmol, 55%) as a clear yellowish oil. Mass of product found [M+H]⁺=643.4 m/z. Tert-butyl N-(((9H-fluoren-9-yl)methoxy)carbonyl)-L-cysteinate 34 (154.6 mg, 0.39 mmol, 1 eq.) and tert-butyl S-2-((tert-butoxycarbonyl)amino)-3-iodopropanoate 30b (159.6 mg, 0.43 mmol, 1.1 eq.) were dissolved in DMF (0.06M, 6.5 mL). Once dissolved, Cs₂CO₃ (127.1 mg, 0.39 mmol, 1 eq.) was added in three portions over 30 min. Once added, the reaction was stirred at room temperature until completion. Once complete, dilute reaction mixture with ethyl acetate (30 mL). The organic layer was washed with water (7×20 mL) and brine (30 mL). The organic layer was dried over MgSO₄, filtered, and concentrated. The crude product was purified via flash column chromatography (EA/Hex) to yield tert-butyl N-(((9H-fluoren-9-yl)methoxy)carbonyl)-S—(S-3-(tert-butoxy)-2-((tert-butoxycarbonyl)amino)-3-oxopropyl)-L-cysteinate 35b (130 mg, 0.20 mmol, 47%) as a clear yellowish oil. Mass of product found [M+H]⁺=643.4 m/z. Tert-butyl N-(((9H-fluoren-9-yl)methoxy)carbonyl)-S—(R-3-(tert-butoxy)-2-((tert-butoxycarbonyl)amino)-3-oxopropyl)-L-cysteinate 35a (175.8 mg, 0.27 mmol, 1 eq.) was dissolved in 1,4-dioxane (0.3M, 1 mL) then 5.7M HCl in dioxane (1.2 mL) was added at 0° C. then stirred for 3 h until room temperature. Dilute the reaction with ethyl acetate (5 mL) and sat. NaHCO₃ (5 mL). The organic layer was washed with sat NaHCO₃ (2×10 mL) and brine (2×15 mL). The combined organic layers are dried over MgSO₄, filtered, and concentrated to yield tert-butyl N-(((9H-fluoren-9-yl)methoxy)carbonyl)-S—(R-2-amino-3-(tert-butoxy)-3-oxopropyl)-L-cysteina te 36a (104 mg, 0.19 mmol, 71%) as clear yellowish oil. Mass of product found [M+H]*=543.3 m/z. Tert-butyl N-(((9H-fluoren-9-yl)methoxy)carbonyl)-S—(S-3-(tert-butoxy)-2-((tert-butoxycarbonyl)amino)-3-oxopropyl)-L-cysteinate 35b (207.1 mg, 0.32 mmol) was dissolved in 1,4-dioxane (0.3M, 1.1 mL) then 5.7M HCl in dioxane (1.5 mL) was added at 0° C. then stirred for 3 h at room temperature. Dilute the reaction with ethyl acetate (5 mL) and sat. NaHCO₃ (5 mL). The organic layer was washed with sat NaHCO₃ (2×10 mL) and brine (2×15 mL). The combined organic layers are dried over MgSO₄, filtered, and concentrated to yield tert-butyl N-(((9H-fluoren-9-yl)methoxy)carbonyl)-S—(S-2-amino-3-(tert-butoxy)-3-oxopropyl)-L-cysteina te 36b (118 mg, 0.22 mmol, 68%) as a clear yellowish oil. Mass of product found [M+H]⁺=543.3 m/z.

Fmoc-Glu(OtBu)-OH was loaded onto pre-swelled 2-Chlorotrityl resin in CH₂Cl₂ in present of DIEA overnight. Fmoc was then removed by treating the resin with 20% piperidine in DMF (3×8 min). A solution of tert-butyl N-(((9H-fluoren-9-yl)methoxy)carbonyl)-S—(R-2-amino-3-(tert-butoxy)-3-oxopropyl)-L-cysteina te (36a, scheme 6, 271.65 mg, 0.5 mmol, 10 eq relative to resin for CCZ02032) or N-(((9H-fluoren-9-yl)methoxy)carbonyl)-S—(S-2-amino-3-(tert-butoxy)-3-oxopropyl)-L-cysteina te (36b, scheme 6) (154.9 mg, 0.5 mmol, 10 eq relative to resin, 271.65 mg, 0.5 mmol, 10 eq relative to resin for CCZ02033) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the Glu(OtBu)-immobilized resin and reacted for 16 h. 2-Azidoethanamine was added in presence of CuSO₄ and sodium ascorbate, and reacted overnight. After washing the resin with DMF, Fmoc-Ala(9-Anth)-OH and Fmoc-tranexamic acid were then coupled to the side chain of Dap using Fmoc-protected amino acid (4 eq.), HATU (4 eq.), and DIEA (7 eq.). Finally DOTA-tris(t-bu)ester (2-(4,7,10-tris(2-(t-butoxy)-2-oxoehtyl)-1,4,7,10)-tetraazacyclododecan-1-yl)acetic acid).

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+H]⁺ for CCZ02032 1154.46; found [M+H]⁺ 1154.85; calculated [M+H]⁺ for CCZ02033 1154.46; found [M+H]⁺1154.24.

In vitro competitive binding assay result for CCZ02032 and CCZ02033 were Ki=3.2 and 460.8 nM (n=1), respectively.

Table 9 shows the biodistribution data for ⁶⁸Ga—CCZ02032 at 1 h post-injection in mice bearing LNCaP xenograft

TABLE 9
Biodistribution data for ⁶⁸Ga-CCZ02032 at 1 h post-injection
in mice bearing LNCaP xenograft, unit is in % ID/g.

⁶⁸Ga-CCZ02032
(n = 4)

| | Avg | Std |
|---|---|---|
| Blood | 0.82 | 0.13 |
| Urine | 93.04 | 13.91 |
| Fat | 0.19 | 0.02 |
| Seminal | 0.08 | 0.02 |
| Testes | 0.24 | 0.03 |
| Intestine | 0.24 | 0.06 |
| Spleen | 0.41 | 0.09 |
| Pancreas | 0.15 | 0.03 |
| Stomach | 0.06 | 0.02 |
| Liver | 0.31 | 0.05 |
| Adrenal | 0.68 | 0.44 |
| Kidney | 7.66 | 1.13 |
| Heart | 0.21 | 0.01 |
| Lungs | 0.62 | 0.06 |
| LNCaP tumor | 9.06 | 1.12 |
| Bone | 0.18 | 0.02 |
| Muscle | 0.12 | 0.00 |
| Brain | 0.02 | 0.00 |
| Salivary gland | 0.31 | 0.04 |
| Thyroid | 0.26 | 0.01 |
| Lacrimal | 0.18 | 0.07 |

### Example 6: ADZ-4-101, PD-6-49, PD-5-131 and PD-5-159

### Synthesis of ADZ-4-101, PD-6-49, PD-5-131 and PD-5-159

The structures of ADZ-4-101, PD-6-49, PD-5-131 and PD-5-159 are shown below:

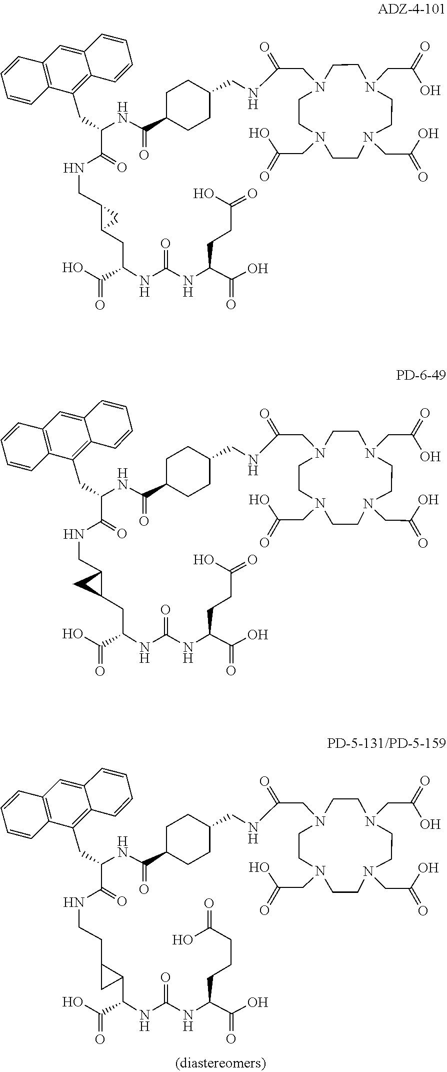 [SMILES: O=C(O)CC[C@H](NC(=O)N[C@@H](C[C@@H]1C[C@H]1CNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CC[C@H](NC(=O)N[C@@H](C[C@H]1C[C@@H]1CNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CCC[C@H](NC(=O)N[C@H](C(=O)O)C1CC1CCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O]

For ADZ-4-101, Fmoc-(S,R,S)-4,5-Cyclopropyl-Lys(alloc)-OH (ADZ-4-89, scheme 7) was first synthesized.

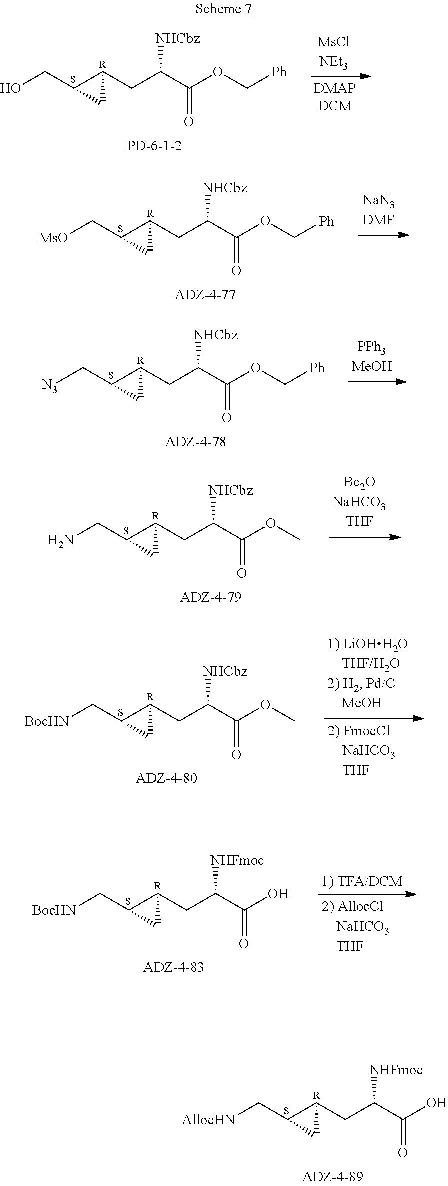

### Synthesis ADZ-4-77

PD-6-1-2 (40 mg, 0.10 mmol, 1 eq, synthesized following literature procedure from Aust. J. Chem. 2013, 66, 1105-1111) was dissolved in DCM (1 mL) and treated with DMAP (4 mg, 0.03 mmol, 1.5 eq), NEt3 (0.02 mL, 0.15 mmol, 1.5 eq), MsCl (0.01 mL, 0.15 mmol, 1.5 eq). The reaction was stirred for 2 h at RT. The reaction was diluted with water (20 mL), extracted with diethylether (3×30 mL). The organic phases were combined, washed with water (20 mL) and brine (10 mL), dried (MgSO4), filtered and evaporated. The product was used without further purification.

### Synthesis ADZ-4-78

The crude oil containing ADZ-4-77 was dissolved in DMF (1 mL) and treated with NaN3 (33 mg, 0.50 mmol, 5 eq). The reaction was stirred for overnight at RT, diluted with EtOAc (50 mL), washed with LiCl (10% w/w, aq, 2×30 mL), dried (MgSO4), filtered and evaporated. The product was used without further purification.

### Synthesis ADZ -4-79

The crude oil containing ADZ-4-78 was dissolved in methanol (2 mL) and treated with PPh3 (39 mg, 0.15 mmol, 1.5 eq). The reaction was refluxed overnight. The volatiles were evaporated and the product was used without further purification.

### Synthesis ADZ -4-80

The crude oil obtaining ADZ-4-79 was dissolved in THF (1 mL) and treated with NaHCO₃ (sat., aq, 1 mL) and Boc2O(33 mg, 0.15 mmol, 1.5 eq). The reaction was stirred overnight at RT, treated with water (20 mL) and extracted with EtOAc (3×20 mL). The organic phases were combined, dried (MgSO4), filtered and evaporated. ADZ-4-80 was isolated by chromatography on silica gel (10% to 20% EtOAc in hexanes) as a pale yellow oil (13 mg, 32%); Rf=0.58 (40% EtOAc in hexanes).

### Synthesis ADZ-4-83

ADZ-4-80 (13 mg, 0.03 mmol, 1 eq) was dissolved in a mixture of THF/H₂O (3:1) (1 mL) and treated with LiOH·H₂O (4 mg, 0.10 mmol, 3 eq). The reaction was stirred 1 h at RT, diluted with water (10 mL), acidified with HCl (4 N) until pH=2, diluted with water (10 mL) and extracted with EtOAc (3×20 mL). The organic phases were combined, dried (MgSO4), filtered and evaporated. The resulting oil was dissolved in methanol (2 mL), treated with Pd/C (5 mg) and stirred for 48 h under H₂ atmosphere. The mixture was filtered over celite and washed with methanol (3×5 mL). The filtrate was evaporated and the resulting oil was dissolved in THF (1 mL), treated with NaHCO₃ (10% w/w, aq, 1 mL) and Fmoc-Cl (10 mg, 0.04 mmol, 1.2 eq). The reaction was stirred overnight at RT, diluted with water (10 mL), acidified with HCl (4 N) until pH=2, diluted with water (10 mL) and extracted with EtOAc (3×20 mL). The organic phases were combined, dried (MgSO4), filtered and evaporated. The crude product was used without further purification.

### Synthesis ADZ-4-89

The crude oil containing ADZ-4-86 was dissolved in DCM (0.5 mL) and treated with TFA (0.5 mL). The reaction was stirred for 1 h. The volatiles were evaporated. The resulting oil was dissolved in THF (0.5 mL) and treated with Na2CO3 (10% w/w, aq, 0.5 mL) and allylchloroformate (4 μL, 0.04 mmol, 1.1 eq). The reaction was stirred overnight at RT, diluted with water (10 mL) and acidified with HCl (4 N) until pH=2. The resulting aqueous phase was extracted with EtOAc (3×20 mL). The organic phases were combined, dried (MgSO4) and filtered. ADZ-4-89 was isolated by chromatography on silica gel (20% EtOAc in hexanes, followed by a gradient of 20% to 40% EtOAc in hexanes with 0.5% FA) as pale yellow oil (7 mg, 47%); Rf=0.21 (50% EtOAc in hexanes with 1% FA).

Fmoc-(S,R,S)-4,5-Cyclopropyl-Lys(alloc)-OH (ADZ-4-89) was loaded onto pre-swelled 2-Chlorotrityl resin in CH₂Cl₂ in present of DIEA overnight. Fmoc was then removed by treating the resin with 20% piperidine in DMF (3×8 min). A solution of Glutamic acid di-tertbutyl ester hydrochloride (147.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the (S,R,S)-4,5-Cyclopropyl-Lys(alloc)-immobilized resin and reacted for 16 h. After washing the resin with DMF, the Alloc-protecting group was removed with Pd(PPh₃)₄ in presence of phenylsilane (2×10 min). Fmoc-Ala(9-Anth)-OH and Fmoc-tranexamic acid were then coupled to the side chain of Dap using Fmoc-protected amino acid (4 eq.), HATU (4 eq.), and DIEA (7 eq.). Finally DOTA-tris(t-bu)ester(2-(4,7,10-tris(2-(t-butoxy)-2-oxoehtyl)-1,4,7,10)-tetraazacyclododecan-1-yl)acetic acid).

For PD-6-49, Fmoc-(S,S,R)-4,5-Cyclopropyl-Lys(alloc)-OH (PD-6-27, scheme 8) was first synthesized.

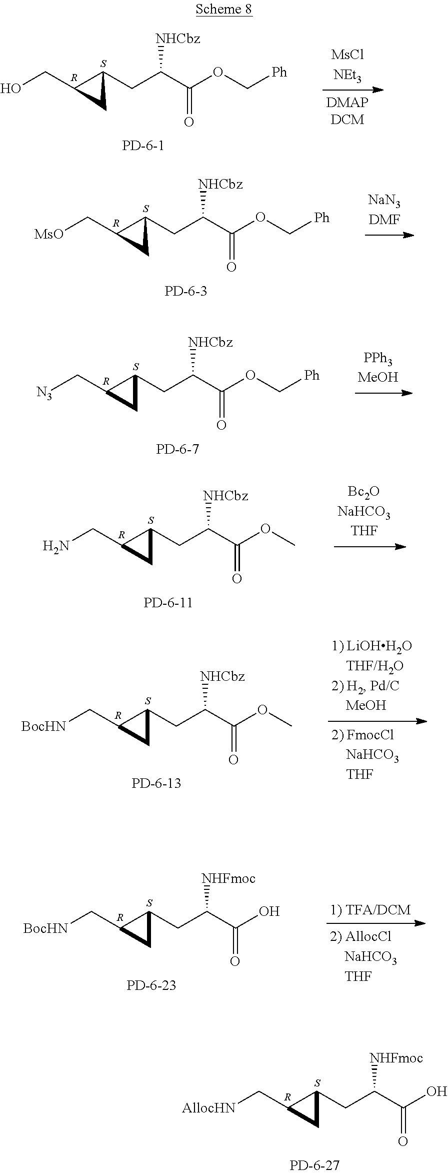

### Synthesis of PD-6-3

PD-6-1-1 (50 mg, 0.13 mmol, 1 eq, synthesized following literature procedure from Aust. J. Chem. 2013, 66, 1105-1111) was dissolved in DCM (1 mL) and treated with DMAP (5 mg, 0.04 mmol, 1.5 eq), NEt3 (0.03 mL, 0.20 mmol, 1.5 eq), MsCl (0.02 mL, 0.20 mmol, 1.5 eq). The reaction was stirred for 2 h at RT. The reaction was diluted with water (20 mL), extracted with diethylether (3×30 mL). The organic phases were combined, washed with water (20 mL) and brine (10 mL), dried (MgSO4), filtered and evaporated. The product was used without further purification.

### Synthesis of PD-6-7

The crude oil containing PD-6-3 was dissolved in DMF (1 mL) and treated with NaN3 (42 mg, 0.65 mmol, 5 eq). The reaction was stirred for 4 h at RT, diluted with EtOAc (50 mL), washed with LiCl (10% w/w, aq, 2×30 mL), dried (MgSO4), filtered and evaporated. The product was used without further purification.

### Synthesis of PD-6-11

The crude oil containing PD-6-7 was dissolved in methanol (2 mL) and treated with PPh3 (51 mg, 0.20 mmol, 1.5 eq). The reaction was refluxed overnight. The volatiles were evaporated and the product was used without further purification.

### Synthesis of PD-6-13

The crude oil obtaining PD-6-11 was dissolved in THF (1 mL) and treated with NaHCO₃ (sat., aq, 1 mL) and Boc2O(43 mg, 0.20 mmol, 1.5 eq). The reaction was stirred overnight at RT, treated with water (20 mL) and extracted with EtOAc (3×20 mL). The organic phases were combined, dried (MgSO4), filtered and evaporated. PD-6-13 was isolated by chromatography on silica gel (10% to 20% EtOAc in hexanes) as a pale yellow oil (15 mg, 28%); Rf=0.75 (40% EtOAc in hexanes).

### Synthesis of PD-6-23

PD-6-13 (15 mg, 0.04 mmol, 1 eq) was dissolved in a mixture of THF/H₂O (3:1) (1 mL) and treated with LiOH·H₂O (5 mg, 0.11 mmol, 3 eq). The reaction was stirred 2 h at RT, diluted with water (10 mL), acidified with HCl (1 N) until pH=2, diluted with water (10 mL) and extracted with EtOAc (3×20 mL). The organic phases were combined, dried (MgSO4), filtered and evaporated. The resulting oil was dissolved in methanol (2 mL), treated with Pd/C (5 mg) and stirred overnight under H₂ atmosphere. The mixture was filtered over celite and washed with methanol (3×5 mL). The filtrate was evaporated and the resulting oil was dissolved in THF (1 mL), treated with NaHCO₃ (10% w/w, aq, 1 mL) and Fmoc-Cl (12 mg, 0.04 mmol, 1.2 eq). The reaction was stirred overnight at RT, diluted with water (10 mL), acidified with HCl (4 N) until pH=2, diluted with water (10 mL) and extracted with EtOAc (3×20 mL). The organic phases were combined, dried (MgSO4), filtered and evaporated. The crude product was used without further purification.

### Synthesis of PD-6-27

The crude oil containing PD-6-23 was dissolved in DCM (0.5 mL) and treated with TFA (0.5 mL). The reaction was stirred for 1 h. The volatiles were evaporated. The resulting oil was dissolved in THF (0.5 mL) and treated with Na2CO3 (10% w/w, aq, 0.5 mL) and allylchloroformate (5 μL, 0.04 mmol, 1.1 eq). The reaction was stirred overnight at RT, diluted with water (10 mL) and acidified with HCl (4 N) until pH=2. The resulting aqueous phase was extracted with EtOAc (3×20 mL). The organic phases were combined, dried (MgSO4) and filtered. PD-6-27 was isolated by chromatography on silica gel (20% EtOAc in hexanes, followed by a gradient of 20% to 40% EtOAc in hexanes with 0.5% FA) as pale yellow oil (6 mg, 35%); Rf=0.33 (50% EtOAc in hexanes with 1% FA).

Fmoc-(S,S,R)-4,5-Cyclopropyl-Lys(alloc)-OH (PD-6-27, scheme 7) was loaded onto pre-swelled 2-Chlorotrityl resin in CH₂Cl₂ in present of DIEA overnight. Fmoc was then removed by treating the resin with 20% piperidine in DMF (3×8 min). A solution of Glutamic acid di-tertbutyl ester hydrochloride (147.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the (S,R,S)-4,5-Cyclopropyl-Lys(alloc)-immobilized resin and reacted for 16 h. After washing the resin with DMF, the Alloc-protecting group was removed with Pd(PPh₃)₄ in presence of phenylsilane (2×10 min). Fmoc-Ala(9-Anth)-OH and Fmoc-tranexamic acid were then coupled to the side chain of Dap using Fmoc-protected amino acid (4 eq.), HATU (4 eq.), and DIEA (7 eq.). Finally DOTA-tris(t-bu)ester(2-(4,7,10-tris(2-(t-butoxy)-2-oxoehtyl)-1,4,7,10)-tetraazacyclododecan-1-yl)acetic acid).

For PD-5-159 and PD-5-131, Fmoc-3,4-Cyclopropyl-Lys(alloc)-OH (diastereomer 1, PD-5-137, scheme 9) and Fmoc-3,4-Cyclopropyl-Lys(alloc)-OH (diastereomer 2, PD-5-107, scheme 8) was first synthesized, respectively.

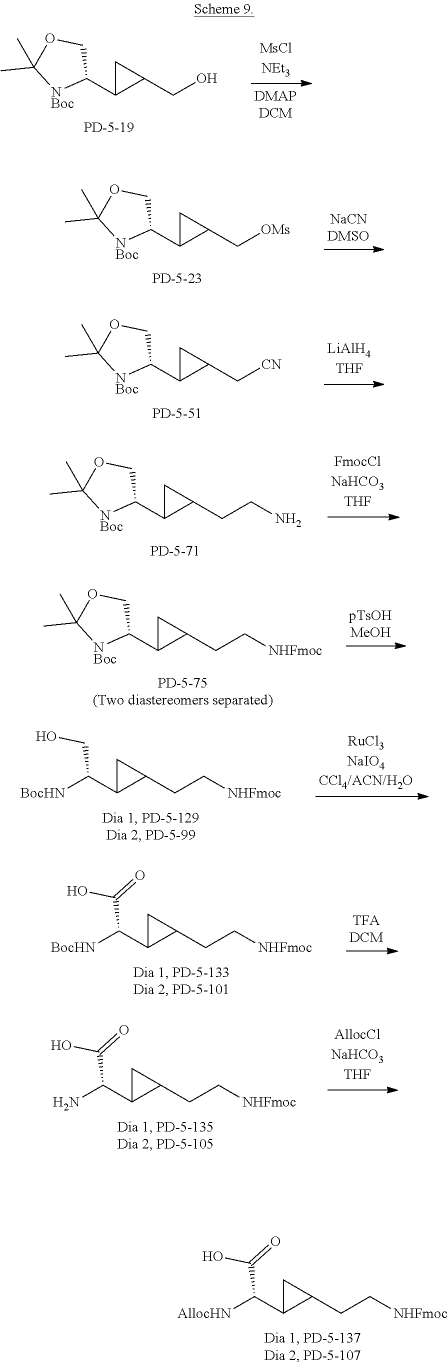

### Synthesis of PD-5-49

PD-5-19 (133 mg, 149 mmol, 1 eq, synthesized following literature procedure from DOI: 10.1039/b105503h) was dissolved in DCM (5 mL) and treated with DMAP (85 mg, 0.74 mmol, 1.5 eq), NEt3 (0.10 mL, 0.74 mmol, 1.5 eq), MsCl (0.06 mL, 0.74 mmol, 1.5 eq). The reaction was stirred for 2 h at RT. The volatiles were evaporated. PD-5-23 was isolated by chromatography on neutral silica gel (gradient 10% to 40% EtOAc in hexanes) as pale yellow oil (141 mg, 82%); Rf=0.33 (30% EtOAc in hexanes).

### Synthesis of PD-5-51

PD-5-49 (245 mg, 0.74 mmol, 1eq) was dissolved in dry DMSO (7 mL) under argon and treated with KCl (241 mg, 3.7 mmol, 5 eq). The reaction was stirred overnight at RT under argon, K2CO3 (10% w/w, aq, 20 mL) was added and extracted with EtOAc (3×20 mL). The organic phases were combined, washed with brine, dried (MgSO4), filtered and evaporated. PD-5-41 was isolated by chromatography on neutral silica gel (gradient 5% to 20% EtOAc in hexanes) as pale yellow oil (126 mg, 61%); Rf=0.83 (50% EtOAc in hexanes).

### Synthesis of PD-5-71

PD-5-51 (126 mg, 0.45 mmol, 1 eq) was dissolved in dry ether (4 mL), cooled to 0° C. under argon, stirred 5 min and treated with LiAlH4. The reaction was stirred for 1 h at 0° C. NH4Cl (sat., aq, 10 mL) was added followed by the addition of water (10 mL). The resulting mixture was extracted with EtOAc (3×20 mL). The organic phases were combined, washed with brine, dried (MgSO4), filtered and evaporated. The product was used without further purification.

### Synthesis of PD-5-75

The crude oil containing PD-5-71 was dissolved in THF (2 mL), treated with NaHCO₃ (10% w/w, aq, 2 mL) and Fmoc-Cl (140 mg, 0.54 mmol, 1.2 eq). The reaction was stirred overnight at RT, treated with water (30 mL) and extracted with EtOAc (3×20 mL). The organic phases were combined, dried (MgSO4), filtered and evaporated. PD-5-75 was isolated by chromatography on neutral silica gel (gradient 5% to 20% EtOAc in hexanes) allowing separation of the two diastereomers. The higher Rf=0.83 (40% EtOAc in hexanes), PD-5-75-1 was obtained as pale yellow oil (15 mg, 7%). The lower Rf=0.77 (40% EtOAc in hexanes), PD-5-75-2 was obtained as a pale yellow oil (39 mg, 17%).

### Synthesis of PD-5-129

PD-5-75-1 (14 mg, 0.03 mmol, 1 eq) was dissolved in methanol (1 mL), treated with pTsOH·H₂O (3 mg, 0.01 mmol, 0.5 eq) and water (0.02 mL). The reaction was stirred overnight at RT, treated with NaHCO₃ (sat., aq, 20 mL) and extracted with EtOAc (3×20 mL). The organic phases were combined, washed with brine, dried (MgSO4), filtered and evaporated. The product was used without further purification.

### Synthesis of PD-5-133

The crude oil containing PD-5-129 was treated with water (0.39 mL), MeCN (0.26 mL) and CCl4 (0.26 mL). The resulting mixture was treated with NaIO4 (24 mg, 0.11 mmol, 4 eq) and RuCl3·×H2O (0.2 mg, 0.001 mmol, 0.03 eq) and stirred 2 h at RT. The reaction was diluted with EtOAc (20 mL), washed with Na2S2O3 (1 N, 2×10 mL) and brine (5 mL), dried (MgSO4), filtered and evaporated. The product was used without further purification.

### Synthesis of PD-5-135

The crude oil containing PD-5-133 was diluted in DCM (0.5 mL) and treated with water (0.02 mL), TIPS (0.02 mL) and TFA (0.5 mL). The reaction was stirred for 1 h at RT. The volatiles were evaporated and the product was used without further purification.

### Synthesis of PD-5-137

The crude oil containing PD-5-135 was dissolved in dioxane (0.5 mL) and treated with Na2CO3 (10% w/w, aq, 0.5 mL) and allylchloroformate (3 μL, 0.03 mmol, 1.1 eq). The reaction was stirred overnight at RT, diluted with water (10 mL) and acidified with HCl (1 N) until pH=2. The resulting aqueous phase was extracted with EtOAc (3×20 mL). The organic phases were combined, dried (MgSO4) and filtered. PD-5-137 was isolated by chromatography on silica gel (20% EtOAc in hexanes, followed by a gradient of 20% to 40% EtOAc in hexanes with 0.5% FA) as pale yellow oil (8 mg, 57%); Rf=0.55 (80% EtOAc in hexanes with 1% FA).

### Synthesis of PD-5-99

PD-5-75-2 (26 mg, 0.05 mmol, 1 eq) was dissolved in methanol (1 mL), treated with pTsOH·H₂O (5 mg, 0.03 mmol, 0.5 eq) and water (0.02 mL). The reaction was stirred overnight at RT, treated with NaHCO₃ (sat., aq, 20 mL) and extracted with EtOAc (3×20 mL). The organic phases were combined, washed with brine, dried (MgSO4), filtered and evaporated. The product was used without further purification.

### Synthesis of PD-5-101

The crude oil containing PD-5-99 was treated with water (0.39 mL), MeCN (0.26 mL) and CCl4 (0.26 mL). The resulting mixture was treated with NaIO4 (43 mg, 0.2 mmol, 4 eq) and RuCl3·×H2O (0.3 mg, 0.002 mmol, 0.03 eq) and stirred 2 h at RT. The reaction was diluted with EtOAc (20 mL), washed with Na2S₂O3 (1 N, 2×10 mL) and brine (5 mL), dried (MgSO4), filtered and evaporated. The product was used without further purification.

### Synthesis of PD-5-105

The crude oil containing PD-5-101 was diluted in DCM (0.5 mL) and treated with water (0.02 mL), TIPS (0.02 mL) and TFA (0.5 mL). The reaction was stirred for 1 h at RT. The volatiles were evaporated and the product was used without further purification.

### Synthesis of PD-5-107

The crude oil containing PD-5-105 was dissolved in dioxane (0.5 mL) and treated with Na2CO3 (10% w/w, aq, 0.5 mL) and allylchloroformate (6 μL, 0.06 mmol, 1.1 eq). The reaction was stirred overnight at RT, diluted with water (10 mL) and acidified with HCl (1 N) until pH=2. The resulting aqueous phase was extracted with EtOAc (3×20 mL). The organic phases were combined, dried (MgSO4) and filtered. PD-5-107 was isolated by chromatography on silica gel (20% EtOAc in hexanes, followed by a gradient of 20% to 40% EtOAc in hexanes with 0.5% FA) as pale yellow oil (5 mg, 22%) The higher Rf=0.83 (40% EtOAc in hexanes), PD-5-75-1 was obtained as pale yellow oil (15 mg, 7%); Rf=0.44 (80% EtOAc in hexanes with 1% FA).

For PD-5-159 and PD-5-131, Fmoc-3,4-Cyclopropyl-Lys(alloc)-OH (diastereomer 1, PD-5-137) and Fmoc-3,4-Cyclopropyl-Lys(alloc)-OH (diastereomer 2, PD-5-107) was loaded onto pre-swelled 2-Chlorotrityl resin in CH₂Cl₂ in present of DIEA overnight. Fmoc was then removed by treating the resin with 20% piperidine in DMF (3×8 min). Fmoc-Ala(9-Anth)-OH and Fmoc-tranexamic acid were then coupled to the side chain of Fmoc-3,4-Cyclopropyl-Lys(alloc)-OH using Fmoc-protected amino acid (4 eq.), HATU (4 eq.), and DIEA (7 eq.) and DOTA-tris(t-bu)ester(2-(4,7,10-tris(2-(t-butoxy)-2-oxoehtyl)-1,4,7,10)-tetraazacyclododecan-1-yl)acetic acid). After washing the resin with DMF, the Alloc-protecting group was removed with Pd(PPh₃)₄ in presence of phenylsilane (2×10 min). A solution of Glutamic acid di-tertbutyl ester hydrochloride (147.9 mg, 0.5 mmol, 10 eq relative to resin) and DIEA (287.4 μL, 1.65 mmol, DIEA) in CH₂Cl₂ (5 mL) was cooled to −78° C. in a dry ice/acetone bath. Triphosgene (49.0 mg, 0.165 mmol) was dissolved in CH₂Cl₂ (5 mL), and the resulting solution was added dropwise to the reaction at −78° C. The reaction was then allowed to warm to room temperature and stirred for 30 minutes. After which another 87.1 μL DIEA (0.5 mmol) was added, and then added to the peptide-immobilized resin and reacted for 16 h.

The peptide was then deprotected and simultaneously cleaved from the resin by treating with 95/5 trifluoroacetic acid (TFA)/triisopropylsilane (TIS) for 4 h at room temperature. After filtration, the peptide was precipitated by the addition of cold diethyl ether to the TFA solution. The crude peptide was purified by HPLC using the preparative column. The eluates containing the desired peptide were collected, pooled, and lyophilized. ESI-MS: calculated [M+Cu]²⁺ for ADZ-4-101, 583.2; found [M+Cu]²+583.3; calculated [M+Cu]²⁺ for PD-6-49 583.2; found [M+Cu]²+583.3; calculated [M+2H]²⁺ for PD-5-159 559.8; found [M+2H]²+559.7; calculated [M+H]⁺ for PD-5-131 1118.5; found [M+H]⁺ 1118.4.

In vitro competitive binding assay results for ADZ-4-101, PD-6-49, PD-5-131 and PD-5-159 were Ki=2.42, 11.91, >1,000, and 25.38 nM (n=1), respectively.

### Example 7: AR-2-050-1, AR-2-050-2, AR-2-113-1 and AR-2-113-2

### Synthesis of AR-2-050-1, AR-2-050-2, AR-2-113-1 and AR-2-113-2

The structures of AR-2-050-1, AR-2-050-2, AR-2-113-1 and AR-2-113-2 are shown below:

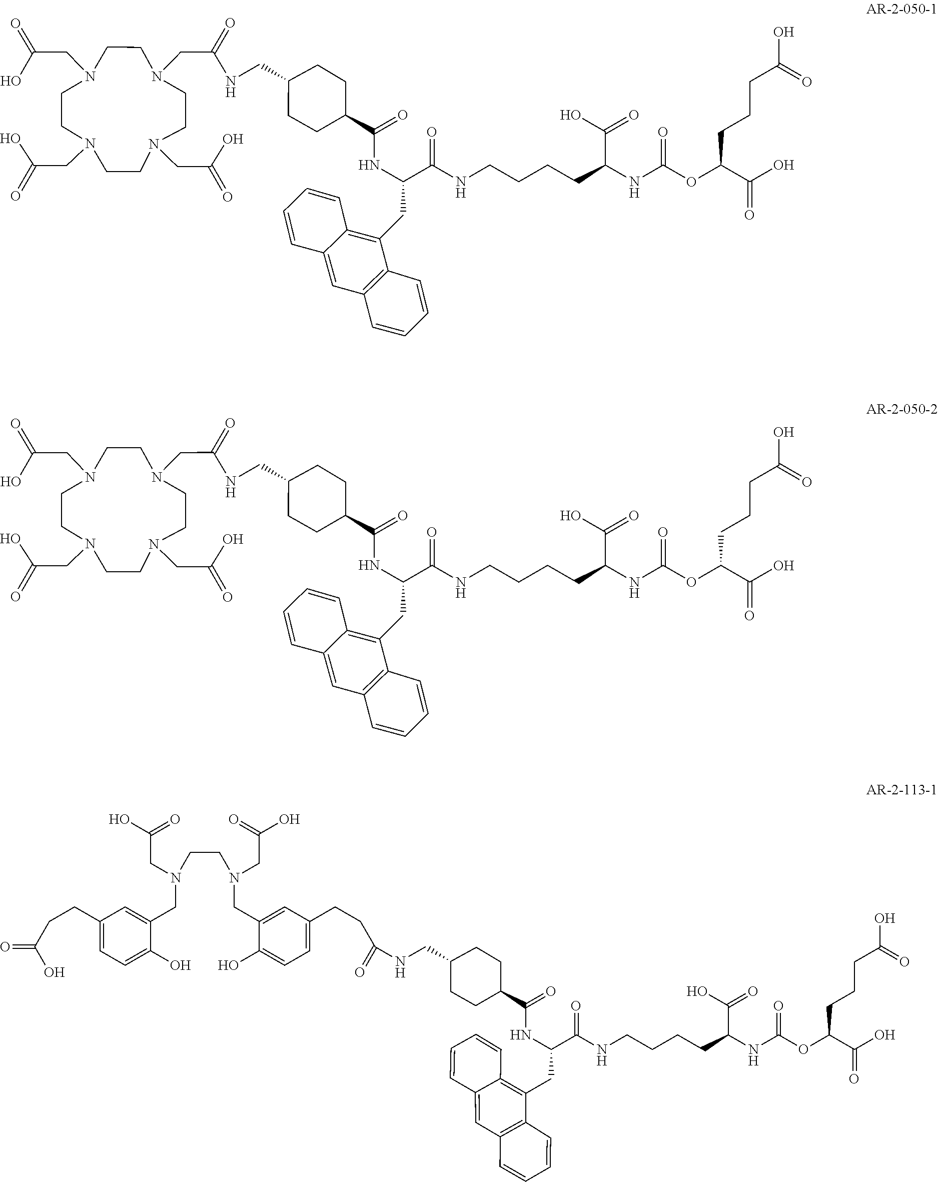 [SMILES: O=C(O)CCC[C@H](OC(=O)N[C@@H](CCCCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CCC[C@@H](OC(=O)N[C@@H](CCCCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CCC[C@H](OC(=O)N[C@@H](CCCCNC(=O)[C@H](Cc1c2ccccc2cc2ccccc12)NC(=O)[C@H]1CC[C@H](CNC(=O)CCc2ccc(O)c(CN(CCN(CC(=O)O)Cc3cc(CCC(=O)O)ccc3O)CC(=O)O)c2)CC1)C(=O)O)C(=O)O]

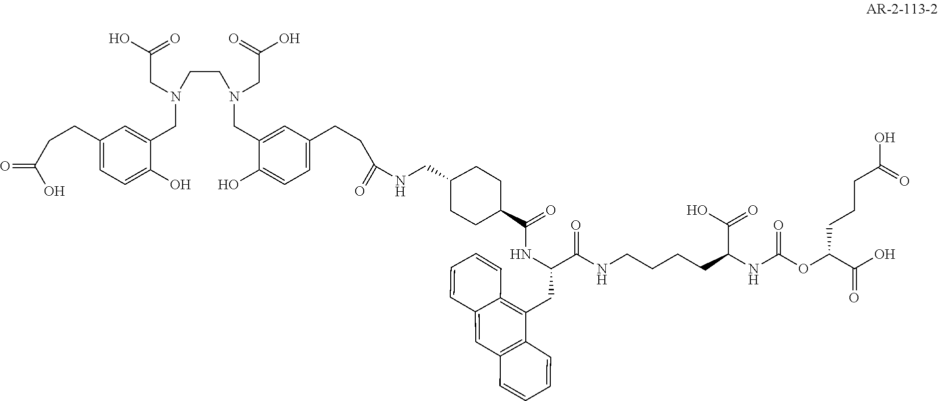 [SMILES: O=C(O)CCC[C@@H](OC(=O)N[C@@H](CCCCNC(=O)[C@H](Cc1c2ccccc2cc2ccccc12)NC(=O)[C@H]1CC[C@H](CNC(=O)CCc2ccc(O)c(CN(CCN(CC(=O)O)Cc3cc(CCC(=O)O)ccc3O)CC(=O)O)c2)CC1)C(=O)O)C(=O)O]

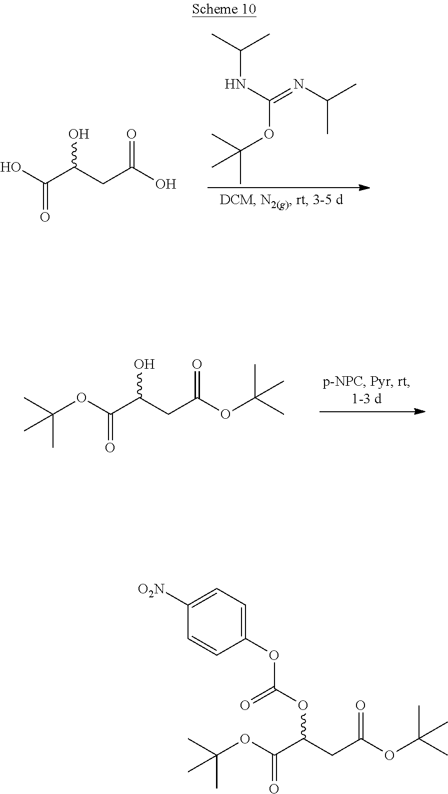 [SMILES: O=C(O)CC(O)C(=O)O ; CC(C)/N=C(/NC(C)C)OC(C)(C)C ; CC(C)(C)OC(=O)CC(O)C(=O)OC(C)(C)C ; CC(C)(C)OC(=O)CC(OC(=O)Oc1ccc([N+](=O)[O-])cc1)C(=O)OC(C)(C)C]

L-(−)-Malic acid or D/L-malic acid (1.08g, 8.055 mmol, 1.0 equiv.) was dissolved 40-80 mL of DCM, sealed with a rubber septum and purged with N2 (g). A 12.13 mL volume of 2-tert-butyl-1,3-diisopropylisourea (53.97 mmol, 6.7 equiv.) was injected over 3-4 min and the suspension was stirred at rt for 70-122 h, as referenced from Allias, et al. Synthesis, 2009, p. 000A-000H. The slurry was concentrated by rotary evaporation, the solids were resuspended with 100 mL of cyclohexane, and the suspension was vacuum filtered through Celite 545. The filtrate was concentrated by rotary evaporation, diluted with (1:1) Hex: EtOAc (v/v), and purified by silica gel chromatography using (1:1) Hex: EtOAc (Rf=0.8, iodine-staining). The pooled fractions were concentrated by rotary evaporation to give 480 mg (1.95 mmol) for the s-isomer and 975 mg (3.96 mmol) for the racemate. These intermediates were then diluted with 4-10 mL of pyridine, mixed with 1.0 equiv. of 4-nitrophenylchloroformate (MW=201.56 g/mol), and stirred in a sealed vessel at rt for 22-70 h. The afforded mixtures were then purified by silica gel chromatography using DCM (Rf=0.65). The pooled fractions were concentrated by rotary evaporation to give 275 mg (668 μmol) for the s-isomer and 1.1 g (2.67 mmol) for the racemate as clear liquids. 1H NMR (400 MHz, CDCl3): 5=1.47 (s, 3×CH3, 9H), 1.49 (s, 3×CH3, 9H), 2.87 (m, CH2, 2H), 5.32 (dd, J=4.7, 2.9 Hz, CH, 1H), 7.41 (td, J=9.2, 2.2 Hz, 2×Ar-H, 2H), 8.27 (td, J=9.2, 3.2 Hz, 2×Ar-H, 2H) ppm.

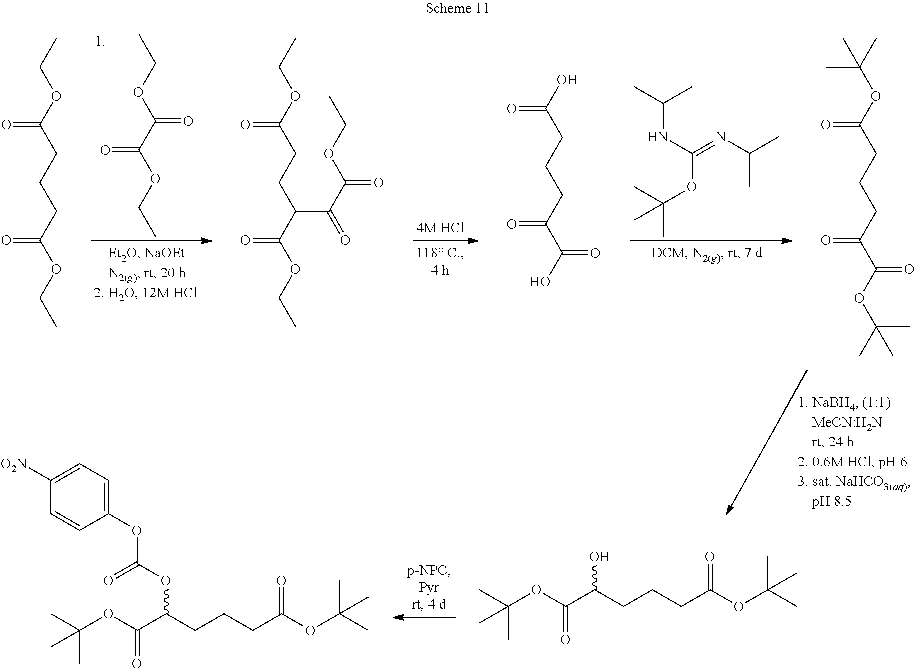

A sample of NaOEt (2.722 g, 40 mmol, 1.0 equiv.) was suspended with 30 mL of Et2O and, while stirring, diethyl oxylate (5.433 mL, 40 mmol, 1.0 equiv.) and diethyl gluterate (7.367 mL, 40 mmol, 1.0 equiv.) were added, as referenced from Nelson, et al. Org. Prep. Proc. 5(2), p. 55-58, 1973. The suspension was purged with N2 (g), sealed and stirred at rt for 20 h. The dark suspension was quenched with 30 mL of H₂O and vacuum filtered. The emulsion was then extracted with 2×30 mL of Et20, and the retained aqueous layer was acidified with 9 mL of 12M HCl and extracted with 2×30 mL of Et20. The collected organic fractions were dried with MgSO4(s), filtered, and concentrated by rotary evaporation to give 8.36 g (29 mmol) of triethyl 1-oxobutane-1,2,4-tricarboxylate. The sample was then resuspended with 35 mL of 4M HCl(aq) and the emulsion was refluxed at 118° C. for 4h. The solution was concentrated by rotary evaporation and dried in vacuo. The solids were vacuum-filtered with 4×25 mL of DCM washing to give 3.85 g (24.1 mmol) of 2-oxohexanedioic acid. A 1.39 g (8.69 mmol, 1.0 equiv.) sample of this intermediate was then di-tert-butyl protected with 11.7 mL of 2-tert-butyl-1,3-diisopropylisourea (52.12 mmol, 6.0 equiv.) in N2(g) purged 25 mL of DCM at rt in 164 h, as previously described. The suspension was concentrated by rotary evaporation, suspended with 100 mL of cyclohexane, and vacuum filtered through Celite 545. The filtrate was concentrated by rotary evaporation, the resulting crude liquid was diluted with 3 mL of DCM and then was purified by silica gel chromatography using (1:1) Hex: EtOAc (v/v) (Rf=0.9, iodine staining). The pooled fractions were concentrated by rotary evaporation to give 1.05 g of di-tert-butyl 2-oxohexanedioate that showed -50% purity by 1H NMR. A 420 mg sample of this crude intermediate was dissolved in 4 mL of MeCN, was diluted with 4 mL of H2O, and then mixed with 583.4 mg (15.42 mmol) of NaBH4(s). The vessel was sealed with a septum and the suspension was stirred at rt for 24h. The reaction was slowly quenched with 20 mL of 0.6M HCl (to pH 6) and then basified with sat. NaHCO₃(aq) to pH 8.5. The suspension was extracted with 3×40 mL of EtOAc, which was then washed with 2×50 mL of H2O and 50 mL of brine. The organic layers were dried with MgSO4, filtered and the filtrate was concentrated by rotary evaporation. This gave 264 mg of clear yellow liquid that showed -40% purity for di-tert-butyl 2-hydroxyhexanedioate as determined by 1H NMR. A 250 mg sample of this crude intermediate was then dissolved with 2 mL of pyridine and 184 mg (911 μmol) of 4-nitrophenylchloroformate. The vessel was sealed and the reaction was stirred at rt for 94h. The afforded suspension was then filtered, concentrated by rotary evaporation, diluted with 2 mL of DCM and purified by silica gel chromatography using DCM (Rf=0.4). The pooled fractions were concentrated by rotary evaporation to give 163 mg (max. 334 μmol) for the racemate as a clear liquid which showed ˜90% purity by 1H NMR. 1H NMR (400 MHz, CDCl3): 5=1.45 (s, 3×CH3, 9H), 1.50 (s, 3×CH3, 9H), 1.74-1.81 (m, CH2, 2H), 1.92-1.98 (m, CH2, 2H), 2.29 (t, J=7.3 Hz, CH2, 2H), 4.90 (t, J=5.7 Hz, CH, 1H), 7.41 (td, J=9.2, 2.2 Hz, 2×Ar-H, 2H), 8.28 (td, J=9.2, 2.1 Hz, 2×Ar—H, 2H) ppm.

The synthesis of each peptide conjugate was performed using Fmoc-Lys(ivDde) Wang resin (100-200 mesh) with 0.58 mmol/g loading on the 35-200 μmol scales using standard Fmoc-synthesis protocols. Coupling and deprotection steps were monitored with Kaiser tests. Post-Fmoc removal, di-tert-butyl-Aad-pNPC was conjugated to N-terminal lysine in either (47.5:47.5:5) DCM: DMF: DIPEA (v/v/v) or (95:5) NMP: DIPEA (v/v) at rt for 1-9 d (2 rounds, as needed). Deprotection of ivDde and Fmoc were achieved using (1:49) hydrazine: DMF (v/v) and (1:4) piperidine: DMF (v/v), respectively. Coupling of remaining residues were accomplished with 3.0 equiv. of Fmoc-Ala(9-anth)-OH, Fmoc-tranexamic acid, and either DOTA(OtBu)₃-OH (DOTA-PSMA-Aad(s/r)-carbamate) or HBED-CC (HBED-CC-PSMA-Aad(s/r)-carbamate), sequentially, with HATU as the coupling reagent and HOAt or HOBt-hydrate, as additives. Two rounds of coupling were performed, as needed. Capping was done using a cocktail of Ac(O)₂ (378 μL 1 M, DCM) and DIPEA (697 μL) in either DMF (6.925 mL) or NMP (5 mL) at rt for 2-2.5h. Resin cleavage and global deprotection of OtBu-groups was accomplished with either (95:5) TFA: TIPS (v/v) or (50:47.5:2.5) TFA: DCM: TIPS (v/v/v) at rt for 5-6h. Post-cleavage, crude solutions were gently concentrated to ˜0.2-0.5 mL with air, aliquoted (˜0.1-0.2 mL) to microcentrifuge tubes, precipitated with Et₂O and pelleted by centrifugation at 10k rpm for 4 min. Supernatents were discarded, pellets were resuspended with 30-50 μL of DMF, and the described Et2O precipitation and centrifugation methods were repeated 2-3 times.

The s- and r-isomers of DOTA-PSMA-Aad-carbamate of the racemic mixture were purified first by prep HPLC (25 mL/min.; λ=254 nm; A) H₂O (0.1% TFA), B) MeCN (0.1% TFA): 0% B for 1 min.; 0-80% B over 8 min.; 80-0% B over 1 min.; tR=5.8 min.) and then with semi-prep HPLC (4.5 mL/min.; λ=254 nm; A) H₂O (0.1% TFA), B) MeCN (0.1% TFA): t=30 min.; 27% B isocratic method; tR s-isomer=18.6 min., tR r-isomer=20.5 min.) from a 29.3 mg sample of crude to obtain 1.7 mg (1.54 μmol) of the s-isomer precursor and 0.85 mg (940 nmol) of the r-isomer precursor post-lyophilization. ESI-MS (+): s-isomer, AR-2-050-1 (calc. 1, 107.2 g/mol): [M+2H]2+=554.8 m/z; r-isomer, AR-2-050-2 (calc. 1, 107.2 g/mol): [M+2H]2+=554.7 m/z. The natGa-standards of the s- and r-isomers were synthesized using 20 equiv. GaCl3 (1 M) in NaHCO₃(aq) at 94-102° C. for 35 min. Each sample was then used as an HPLC standard without further purification. ESI-MS (+): natGa-standard s-isomer (calc. 1, 172.4 g/mol): [M+2H]2+=588.2 m/z, [M+H]⁺=1, 173.8 m/z; natGa-standard r-isomer (calc. 1, 172.4 g/mol): [M+2H]2+=588.3 m/z, [M+H]⁺=1, 173.7 m/z.

The s- and r-isomers of HBED-CC-PSMA-Aad-carbamate were purified by semi-prep HPLC (4.5 mL/min.; λ=254 nm; A) H₂O (0.1% TFA), B) MeCN (0.1% TFA): t=90 min.; 29% B isocratic method; tR s-isomer=67.1 min., tR r-isomer=71.2 min.) from a -35 μmol sample of crude to obtain 0.34 mg (280 nmol) of the s-isomer precursor and 0.5 mg (410 nmol) of the r-isomer precursor post-lyophilization. ESI-MS (+): s-isomer, AR-2-113-1 (calc. 1, 234.5 g/mol): [M+2H]2+=618.6 m/z, [M+H]+=1, 235.6 m/z; r-isomer, AR-2-113-2 (calc. 1, 234.5 g/mol): [M+2H]2+=618.6 m/z, [M+H]+=1, 235.8 m/z. The natGa-standard of the s-isomer was synthesized using 20 equiv. GaCl3 (0.25 M) in NaOAc(aq) pH 4.5 at rt for 20h. This sample was then used as an HPLC standard without further purification. ESI-MS (+): natGa-standard s-isomer (calc. 1, 301.2 g/mol): [M+2H]2+=651.5 m/z.

In vitro competitive binding assay results for AR-2-050-1, AR-2-050-2, AR-2-113-1, and AR-2-113-2 were Ki=8.99, 239.7, 3.44, and 56.3 nM (n=1), respectively.

FIG. 5 shows PET image obtained at 1 h following the intravenous injection of ⁶⁸Ga-AR-113-1. Table 10 shows the biodistribution data for ⁶⁸Ga-AR-113-1 at 1 h post-injection in mice bearing LNCaP xenograft.

TABLE 10
Biodistribution data for ⁶⁸Ga-AR-113-1 at 1 h post-injection
in mice bearing LNCaP xenograft, unit is in % ID/g.

⁶⁸Ga-AR-113-1
(n = 3)

| | Avg | Std |
|---|---|---|
| Blood | 2.42 | 0.45 |
| Urine | 237.55 | 61.10 |
| Fat | 0.56 | 0.09 |
| Testes | 0.79 | 0.09 |
| Intestine | 1.36 | 0.18 |
| Spleen | 0.61 | 0.25 |
| Pancreas | 0.34 | 0.03 |
| Stomach | 0.22 | 0.07 |
| Liver | 1.31 | 0.23 |
| Adrenal | 1.22 | 0.40 |
| Kidney | 27.16 | 5.71 |
| Heart | 0.85 | 0.12 |
| Lungs | 1.93 | 0.39 |
| LNCaP tumor | 11.86 | 4.34 |
| Bone | 0.34 | 0.08 |
| Muscle | 0.49 | 0.07 |
| Brain | 0.05 | 0.01 |
| Salivary gland | 1.05 | 0.62 |
| Lacrimal | 0.13 | 0.04 |

### NUMBERED EMBODIMENTS

1. A compound, wherein the compound has Formula I-a or is a salt or a solvate of Formula I-a:

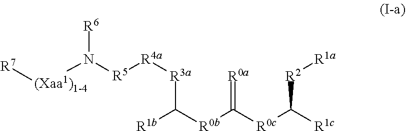

R^{0a} is O or S;
R^{0b} is —O—, —S—, —NH—, or

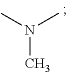

R^{0c} is —O—, —S—, —NH—, or

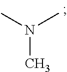

at least one of R^{0b} and R^{0c} is not —NH—;

R^{1a} is —CO₂H, —SO₂H, —SO₃H, —PO₂H, —PO₃H₂, —OPO₃H₂, —OSO₃H, —B(OH)₂, or

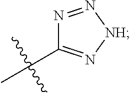 [SMILES: *c1nn[nH]n1]

R^{1b} is —CO₂H, —SO₂H, —SO₃H, —PO₂H, —PO₃H₂, —B(OH)₂, or

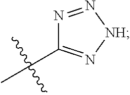 [SMILES: *c1nn[nH]n1]

R^{1c} is —CO₂H, —SO₂H, —SO₃H, —PO₂H, —PO₃H₂—B(OH)₂, or

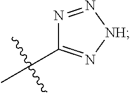 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl; N—N,
R^{4a} is —O—, —S—, —Se—, —S(O)—, —S(O)₂—,

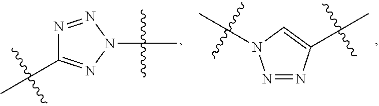 [SMILES: *c1nnn(*)n1 ; *c1cn(*)nn1]

—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or —CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R^{X}-(Xaa²)₀₋₄

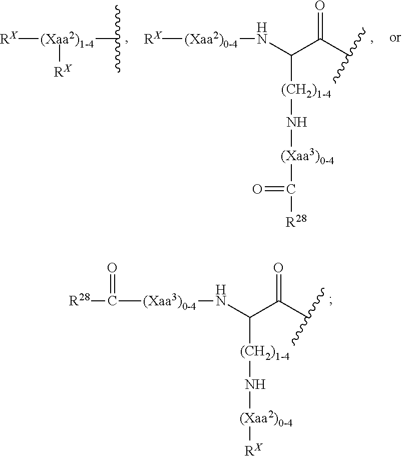

R²⁸ is an albumin binder;
Xaa² and Xaa³, when present, are independently —N(R¹³)R¹⁴C(O)—, wherein each
R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

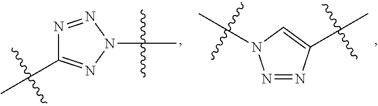 [SMILES: *c1nnn(*)n1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

2. The compound of Embodiment 1, wherein R^{4b} is hydrogen.

3. The compound of Embodiment 1, wherein R^{4a} is —O—, —S—, —S(O)—, —S(O)₂—, —NHC(O)—, —C(O)NH—,

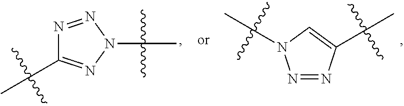 [SMILES: *c1nnn(*)n1 ; *c1cn(*)nn1]

4. The compound of Embodiment 1, wherein R^{4a} is —C(O)—(NH)₂—C(O)—, —OC(O)NH, —NHC(O)C—, —NHC(O)NH—, —OC(S)NH, —NHC(S)O—, —NHC(S)NH—, —NHC(O)C(O)NH—, —S—S—, —S—CH₂—S—, —NH—NH—C(O)—, or —C(O)—NH—NH—.

5. The compound of Embodiment 1, wherein R^{4a} is —O—, —S—, —NHC(O)—, —C(O)NH—,

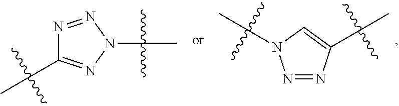 [SMILES: *c1nnn(*)n1 ; *c1cn(*)nn1]

6. The compound of Embodiment 1, wherein R^{4a} is —OC(O)NH—, —NHC(O)O—, —NHC(O)NH—, —OC(S)NH, —NHC(S)O—, or —NHC(S)NH—.

7. The compound of Embodiment 1, wherein R^{4a} is —C(O)NH—.

8. The compound of Embodiment 1, wherein R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups.

9. A compound, wherein the compound has Formula I-b or is a salt or a solvate of Formula I-b:

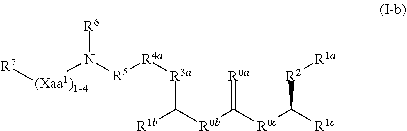

wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

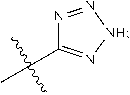 [SMILES: *c1nn[nH]n1]

R^{1b} is —CO₂H, —SO₂H, —SO₃H, —PO₂H, —PO₃H₂—B(OH)₂, or

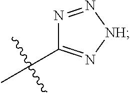 [SMILES: *c1nn[nH]n1]

R^{1c} is —CO₂H, —SO₂H, —SO₃H, —PO₂H, —PO₃H₂, —B(OH)₂, or

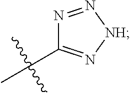 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl;
R^{4a} is —N(R⁴)C(O)—, —C(O)—N(R⁴)—, —C(O)—N(R^{4b})—NH—C(O)—, —C(O)—NH—N(R⁴)C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:

a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or

—CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or

—CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;

R⁷ is R^{X}-(Xaa²)₀₋₄-,

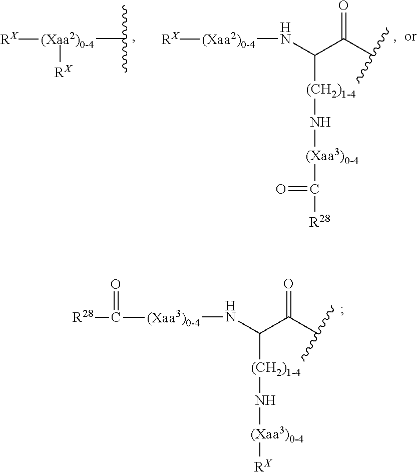

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

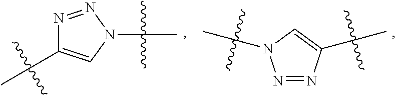 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

10. The compound of Embodiment 8 or 9, wherein R^{4b} is benzyl optionally substituted with one or a combination of OH, NH₂, NO₂, halogen, C₁-C₆ alkyl, and/or C₁-C₆ alkoxyl groups.

11. The compound of Embodiment 8 or 9, wherein R^{4b} is benzyl optionally para-substituted with a halogen.

12. The compound of any one of Embodiments 1 to 11, wherein R^{0a} is O.

13. The compound of any one of Embodiments 1 to 11, wherein R^{0a} is S.

14. The compound of any one of Embodiments 1 to 13, wherein: R^{1a} is —CO₂H, —SO₂H, —SO₃H, —PO₂H, —PO₃H₂, OPO₃H₂, OSO₃H; R^{1b} is —CO₂H, —SO₂H, —SO₃H, —PO₂H, —PO₃H₂; and R^{1c} is —CO₂H, —SO₂H, —SO₃H, —PO₂H, —PO₃H₂.

15. The compound of any one of Embodiments 1 to 13, wherein each of R^{1a}R^{1b} and R^{1c} is —CO₂H.

16. The compound of any one of Embodiments 1 to 13, wherein R² is —CH₂—, —CHOH—, —CHF—, —CH₂CHOH—, —CH₂CHF—, —CH₂CHOHCH₂—, —CH₂CHFCH₂—, —(CH₂)₂CHOH—, —(CH₂)₂CHF—, —(CH₂)₃—, —CH₂OCH₂— or —CH₂SCH₂—.

17. The compound of any one of Embodiments 1 to 13, wherein R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

18. The compound of any one of Embodiments 1 to 13, wherein R²—(CH₂)₃—.

19. The compound of any one of Embodiments 1 to 13, wherein R² is —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—.

20. The compound of Embodiment 19, wherein R² is —CH₂—O—CH₂— or —CH₂—S—CH₂—.

21. The compound of any one of Embodiments 1 to 13, wherein R² is —CH₂—, —CH(OH)—, —CHF—, —CF₂—, —CH(CH₃)—, —C(CH₃)₂—, —CHFCH₂—, —CF₂CH₂—, —CH(OH)CH₂—, —CH(CH₃)CH₂—, —C(CH₃)₂CH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, or —C(O)—NH—C(CH₃)₂—.

22. The compound of any one of Embodiments 1 to 13, wherein R² is —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

23. The compound of any one of Embodiments 1 to 13, wherein R² is —CH₂CH(OH)—, —CH₂CHF—, —CH₂CH(CH₃)—, —CH₂CH(OH)CH₂—, —CH₂CH(F)CH₂—, or —CH₂CH(CH₃)CH₂—, wherein the second carbon in R² has R-configuration.

24. The compound of any one of Embodiments 1 to 13, wherein R² is —HC[CH₂]CH— or —HC[CH₂]CHCH₂—, wherein HC[CH₂]CH represents a cyclopropyl ring.

25. The compound of any one of Embodiments 1 to 24, wherein R^{3a} is: —CH₂—; —(CH₂)₂—; a linear acyclic C₃-C₁₅ alkylenyl; a linear acyclic C₃-C₁₅ alkylenyl in which 1-5 carbons are independently replaced with N, S and/or O heteroatoms; or a linear acyclic saturated C₃-C₁₀ alkylenyl, optionally independently substituted with 1-5 amine, amide, oxo, hydroxyl, thiol, methyl and/or ethyl groups.

26. The compound of any one of Embodiments 1 to 24, wherein R^{3a} is: —CH₂—; —(CH₂)₂—; —(CH₂)₃; —(CH₂)₄—; —(CH₂)₅—; —CH₂—O—CH₂—; or —CH₂—S—CH₂—.

27. The compound of any one of Embodiments 1 to 24, wherein R^{3a} is:—CH═CH—, —CH₂—C≡C—, or a linear C₃-C₅ alkenylenyl or alkynylenyl.

28. The compound of any one of Embodiments 1 to 24, wherein R^{3a} is —(CH₂)₄—.

29. The compound of any one of Embodiments 1 to 24, wherein R^{3a} is:
a linear C₃-C₈ alkylenyl, optionally wherein one methylene is replaced with —S—, —O—, —S—CH(CH₃)—, —O—CH(CH₃)—, —CH(CH₃)—S—, —CH(CH₃)—O—, wherein the S and O heteroatoms are spaced apart from other heteroatoms in the compound by at least 2 carbons, and optionally wherein one ethylene is replaced with —CH═CH—, —C≡C—, a 3-6 membered cycloalkylenyl or arylenyl,

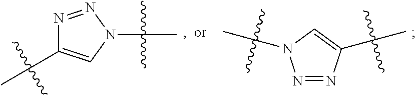 [SMILES: *c1cn(*)nn1 ; *c1cn(*)nn1]

or

—(CH₂)₁₋₃—NH—C(O)—C(R^{3b})₂-, wherein each R^{3b} is independently hydrogen, methyl, or ethyl, or together —C(R^{3b})₂— forms cyclopropylenyl, and which is oriented in the compound as shown:

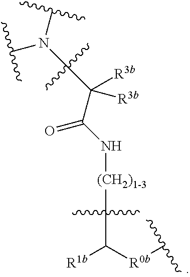

30. The compound of any one of Embodiments 1 to 24, wherein R^{3a} is: —(CH₂)₃—; —(CH₂)₄—; —(CH₂)₅ₐ—; —CH₂—CH═CH—CH₂—; —CH₂—CH₂—CH═CH— wherein the terminal alkenyl carbon is bonded to a carbon in the compound; —CH₂—C≡C—CH₂—; —C(R^{3b})₂—C(O)—NH—(CH₂)₁₋₂— wherein the leftmost carbon is bonded to a nitrogen of R^{4a} and each R^{3b} is independently hydrogen, methyl, or ethyl, or together —C(R^{3b})₂— forms cyclopropyl-enyl; or —CH₂—CH₂—S—CH(R^{3c})_or —CH₂—CH₂O—CH(R^{3e})—, wherein R^{3a} is hydrogen or methyl.

31. The compound of any one of Embodiments 1 to 24, wherein —R^{4a}—R^{3a}— is: C(O)—N(R^{4b})—(CH₂)₁₋₃—R^{3d}—R^{3e}—, wherein R^{3d} is

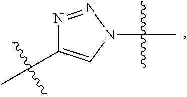 [SMILES: *c1cn(*)nn1]

and wherein R^{3e} is —CH₂—, —(CH₂)₂—, —(CH₂)₂—O—CH₂—, —(CH₂)₂—S—CH₂—, —(CH₂)₂O—CH(CH₃)—, or —(CH₂)₂—S—CH(CH₃)—; or —C(O)—N(R^{4b})—(CH₂)₂-3-R^{3f}—R^{3g}—, wherein R^{3f} is

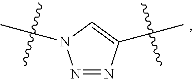 [SMILES: *c1cn(*)nn1]

and wherein R^{3g} is absent, —CH₂—, —(CH₂)₂—, —(CH₂)₀₋₂-O—CH₂—, —(CH₂)₀₋₂-S—CH₂—, —(CH₂)₀₋₂-O—CH(CH₃)—, or —(CH₂)₀₋₂-S—CH(CH₃)—.

32. The compound of any one of Embodiments 1 to 24, wherein R^{3a} is —(CH₂)₁₋₂— R^{3h}—(CH₂)₀₋₂— or —(CH₂)₀₋₂—R^{3h}—(CH₂)₁₋₂—, wherein R^{3h} is:

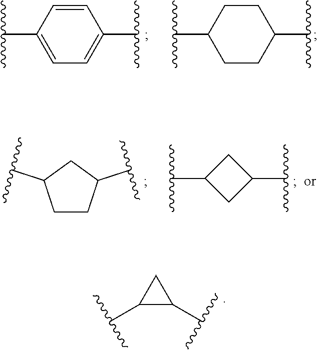

33. The compound of any one of Embodiments 1 to 32, wherein R⁵ is —CH(R¹⁰)—.

34. The compound of any one of Embodiments 1 to 33, wherein R¹⁰ is —CH₂—R^{23a}

35. The compound of Embodiment 34, wherein R^{23a} is phenyl substituted with 1 or 2 iodo groups and optionally further substituted with 1 oxy group.

36. The compound of Embodiment 34, wherein R^{23a} is a radical of anthracene, phenanthene, naphthalene, acridine, or quinoline, wherein each of the foregoing is optionally substituted with one, more than one, or a combination of: halogen, OMe, SMe, NH₂, NO₂, CN, and/or OH.

37. The compound of Embodiment 34, wherein R^{23a} is a radical of naphthalene or quinoline, wherein each of the foregoing is optionally substituted with one, more than one, or a combination of: halogen, OMe, SMe, NH₂, NO₂, CN, and/or OH.

38. The compound of any one of Embodiments 1 to 33, wherein R¹⁰ is a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms.

39. The compound of any one of Embodiments 1 to 33, wherein R¹⁰ is

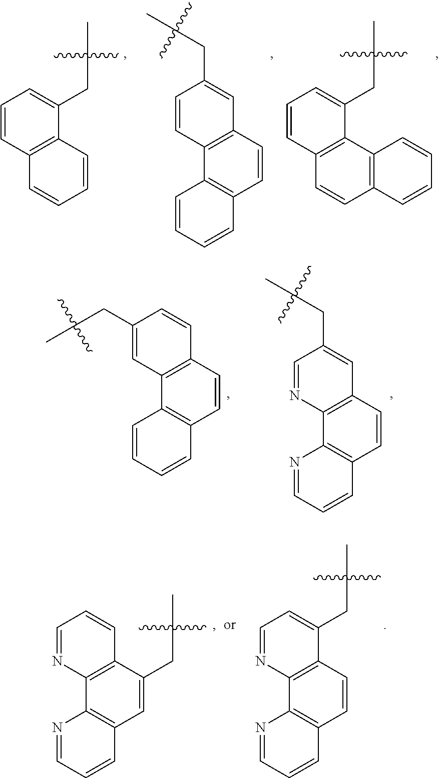

40. The compound of any one of Embodiments 1 to 33, wherein R¹⁰ is

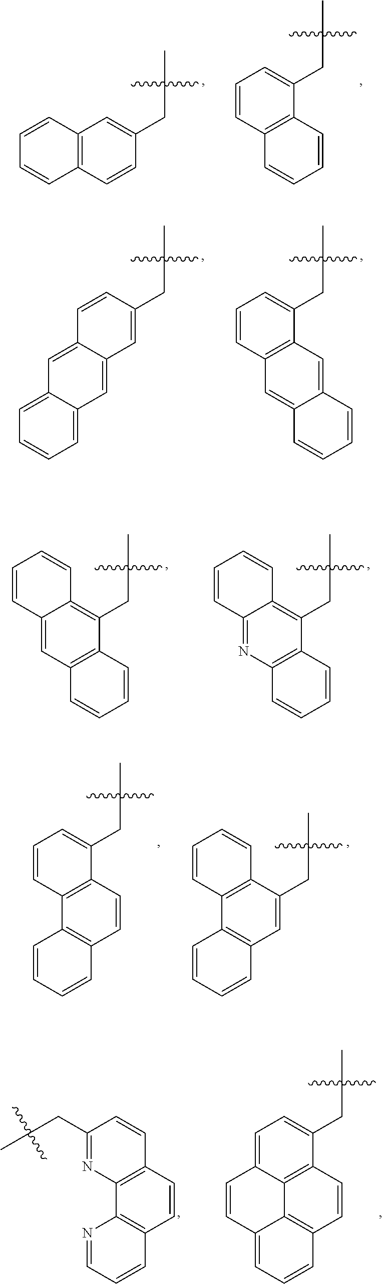

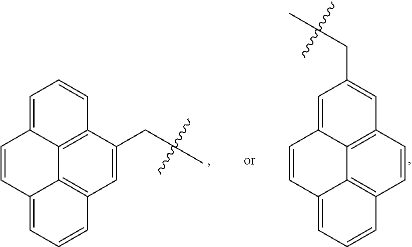 [SMILES: *Cc1cc2cccc3ccc4cccc1c4c32 ; *Cc1cc2ccc3cccc4ccc(c1)c2c34]

optionally modified with one, more than one, or a combination of: halogen, OMe, SMe, NH₂, NO₂, CN, OH, or additional endocyclic ring nitrogen atoms up to a maximum of 5 ring nitrogens.

41. The compound of any one of Embodiments 1 to 33, wherein R¹⁰ is

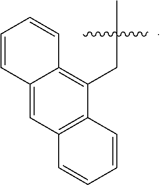 [SMILES: *Cc1c2ccccc2cc2ccccc12]

42. The compound of any one of Embodiments 1 to 33, wherein R¹⁰ is

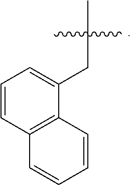 [SMILES: *Cc1cccc2ccccc12]

43. The compound of any one of Embodiments 1 to 33, wherein R¹⁰ is —CH(R²³b)—R^{23c}

44. The compound of Embodiment 43, wherein R^{23b} is phenyl or naphthyl, and wherein R^{23c} is phenyl or naphthyl.

45. The compound of any one of Embodiments 1 to 44, wherein at least one R⁹ is

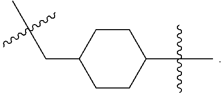 [SMILES: *CC1CCC(*)CC1]

46. The compound of any one of Embodiments 1 to 44, wherein at least one R⁹ is

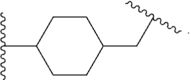 [SMILES: *CC1CCC(*)CC1]

47. The compound of any one of Embodiments 1 to 44, wherein at least one R⁹ is R²⁴—R²⁵—R²⁶, wherein R²⁴—R²⁵—R²⁶ are independently selected from: —(CH₂)₀₋₃—; C₃-C₈ cycloalkylene in which 0-3 carbons are replaced with N, S or O heteroatoms, and optionally substituted with one or more OH, NH₂, NO₂, halogen, C₁-C₆ alkyl and/or C₁-C₆ alkoxyl groups; and C₄-C₁₆ arylene in which 0-3 carbons are replaced with N, S or O heteroatoms, and optionally substituted with one or more OH, NH₂, NO₂, halogen, C₁-C₆ alkyl and/or C₁-C₆ alkoxyl groups.

48. The compound of any one of Embodiments 1 to 44, wherein -(Xaa¹)₁₋₄- is -(Xaa¹)₀₋₃—N(R^{27a})—R^{27b}—C(O)—, wherein R^{27a} is hydrogen or methyl, and wherein R^{27b} is

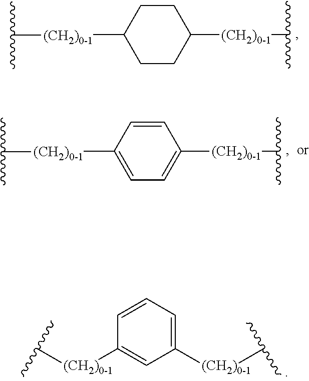 [SMILES: *CC1CCC(C*)CC1 ; *Cc1ccc(C*)cc1 ; *Cc1cccc(C*)c1]

49. The compound of Embodiment 48, wherein R^{27a} is hydrogen.

50. The compound of any one of Embodiments 1 to 48, wherein -(Xaa¹)₁₋₄-N(R⁶)—R⁵—R^{4a}— is

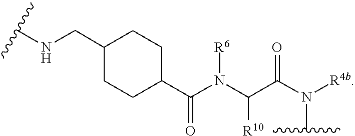

51. The compound of Embodiment 50, wherein R^{4b} is hydrogen.

52. The compound of any one of Embodiments 1 to 51, wherein R⁶ is methyl.

53. The compound of any one of Embodiments 1 to 52, wherein R⁷ is R^{X}-(Xaa²)₀₋₄—.

54. The compound of any one of Embodiments 1 to 52, wherein R²⁸ is

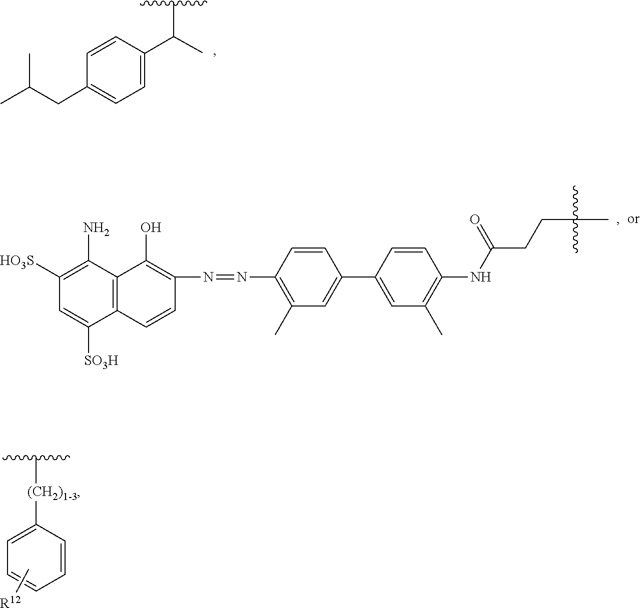

and R¹² is I, Br, F, Cl, H, OH, OCH₃, NH₂, NO₂ or CH₃.

55. The compound of any one of Embodiments 1 to 52, wherein R⁷ is R^{X}-(Xaa²)₀₋₄-,

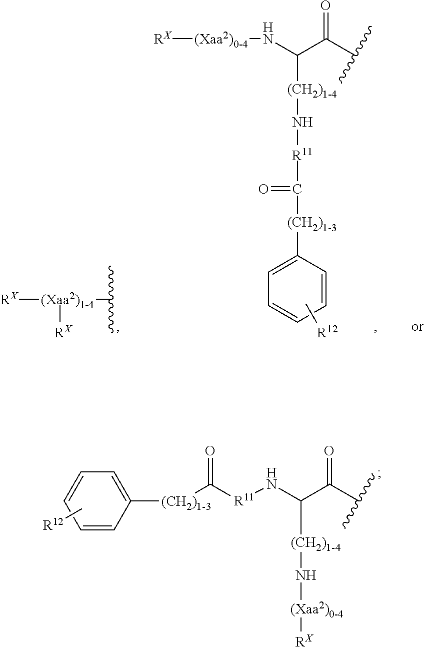

and R¹¹ is absent,

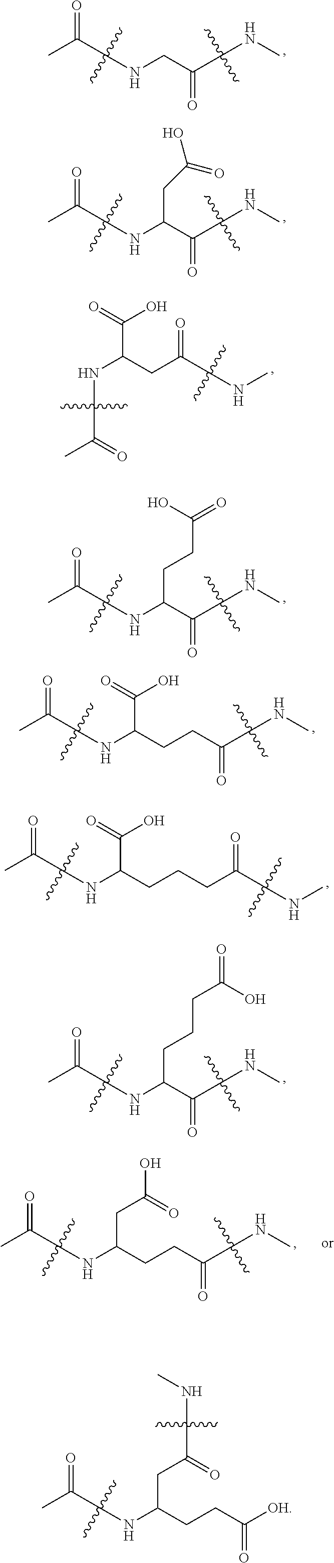

56. The compound of any one of Embodiments 1 to 55, wherein no amide linkages within R⁷-Xaa¹)₁₋₄-N(R⁶)—R⁵—R^{4a}—R^{3a} are replaced.

57. The compound of any one of Embodiments 1 to 55, wherein only one amide linkage within R⁷-(Xaa¹)₁₋₄ is replaced.

58. The compound of any one of Embodiments 1 to 57, wherein R⁷ comprises a first R^{X} group and a second R^{X} group, and wherein the first R^{X} group is a radiometal chelator optionally bound by a radiometal and the second R^{X} group is a prosthetic group containing a trifluoroborate.

59. A compound comprising a prostate specific membrane antigen (PSMA)-targeting moiety of Formula II or of a salt or a solvate of Formula II:

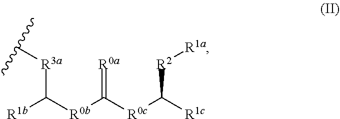

wherein:
R^{0a} is O or S;
R^{0b} is —O—, —S—, —NH—, or

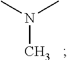

R^{0c} is —O—, —S—, —NH—, or

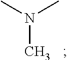

at least one of R^{0b} and R^{0c} is not —NH—;

R^{1a} is —CO₂H, —SO₂H, —SO₃H, —PO₂H, —PO₃H₂, —OPO₃H₂, —OSO₃H, —B(OH)₂, or

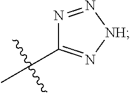 [SMILES: *c1nn[nH]n1]

R^{1b} is —CO₂H, —SO₂H, —SO₃H, —PO₂H, —PO₃H₂—B(OH)₂, or

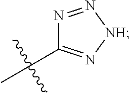 [SMILES: *c1nn[nH]n1]

R^{1c} is —CO₂H, —SO₂H, —SO₃H, —PO₂H, —PO₃H₂, —B(OH)₂, or

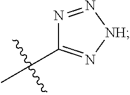 [SMILES: *c1nn[nH]n1]

R² is —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring; and
R³ is a linker.

60. The compound of Embodiment 59, wherein the compound further comprises one or more radiolabeling groups connected to the linker, independently selected from: a radiometal chelator optionally bound by a radiometal; an aryl or heteroaryl substituted with a radiohalogen; a prosthetic group containing a trifluoroborate; or a prosthetic group containing a silicon-fluorine-acceptor moiety, a fluorophosphate, a fluorosulfate, or a sulfonylfluoride.

61. The compound of Embodiment 60, wherein the one or more radiolabeling groups comprise: a radiometal chelator optionally bound by a radiometal; and a prosthetic group containing a trifluoroborate.

62. The compound of any one of Embodiments 59 to 61, wherein R^{0a} is O.

63. The compound of any one of Embodiments 59 to 61, wherein R^{0a} is S.

64. The compound of any one of Embodiments 59 to 63, wherein: R^{1a} is —CO₂H, —SO₂H, —SO₃H, —PO₂H, —PO₃H₂, OPO₃H₂, OSO₃H; R^{2a} is —CO₂H, —SO₂H, —SO₃H, —PO₂H, —PO₃H₂; and R^{3a} is —CO₂H, —SO₂H, —SO₃H, —PO₂H, —PO₃H₂.

65. The compound of any one of Embodiments 59 to 63, wherein each of R^{1a}R^{1b} and R^{1c} is CO₂H.

66. The compound of any one of Embodiments 59 to 65, wherein R² is —CH(CH₃)CH₂CH₂—, —CH₂CH₂CH(CH₃)—, —C(CH₃)₂CH₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —C(O)—NH—CH₂—, —C(O)—NH—CH(CH₃)—, or —C(O)—NH—C(CH₃)₂—.

67. The compound of any one of Embodiments 59 to 65, wherein R² is —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, or —CH₂—S(O)₂—C(CH₃)₂

68. The compound of any one of Embodiments 59 to 65, wherein R² is —CH₂CH(CH₃)CH₂—, and wherein the second carbon in R² has R-configuration.

69. The compound of any one of Embodiments 59 to 68, wherein R³ is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl.

70. The compound of any one of Embodiments 1 to 69 for use in imaging prostate specific membrane antigen (PSMA)-expressing tissues in a subject, wherein the compound comprises a positron or gamma emitting radioisotope.

71. The compound of any one of Embodiments 1 to 69 for use in treatment of a prostate specific membrane antigen (PSMA)-expressing condition or disease in a subject, wherein the compound comprises a therapeutic radioisotope.

72. A compound, wherein the compound has Formula III-a or is a salt or a solvate of Formula III-a:

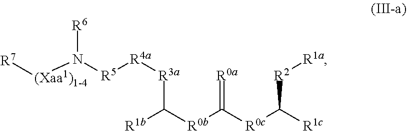

wherein:
R^{0a} is S or O,
R^{0b} is —O—, —S—, —NH—, or

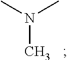

R^{0c} is —O—, —S—, —NH—, or

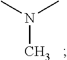

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

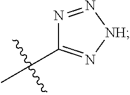 [SMILES: *c1nn[nH]n1]

R^{1b} is —CO₂H, —SO₂H, —SO₃H, —PO₂H, —PO₃H₂, —B(OH)₂, or

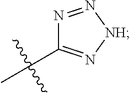 [SMILES: *c1nn[nH]n1]

R^{1c} is —CO₂H, —SO₂H, —SO₃H, —PO₂H, —PO₃H₂, —B(OH)₂, or

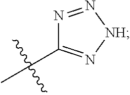 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —O—, —S—, —Se—, —S(O)—, —S(O)₂—,

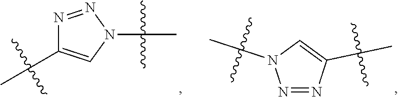 [SMILES: *c1cn(*)nn1 ; *c1cn(*)nn1]

—SS—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—,

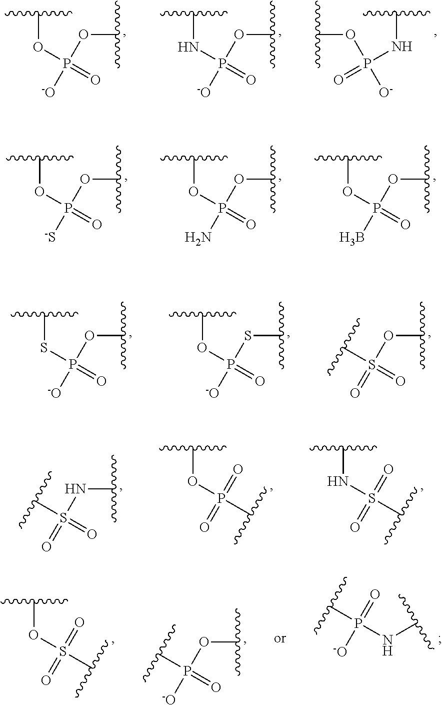

R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
CH₂—R^{23d}—R^{23a} wherein R²³d is absent, CH₂, O, NH or S, and R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;

R⁷ is R^{X}-(Xaa²)₀₋₄-,

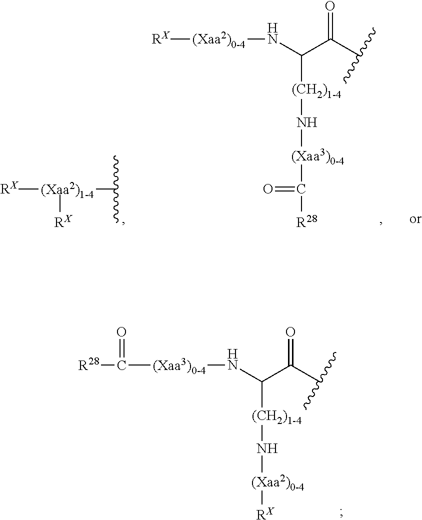

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

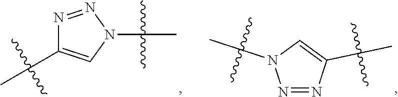 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

73. The compound of Embodiment 72, wherein —N(R⁶)—R⁵—R^{4a}— is

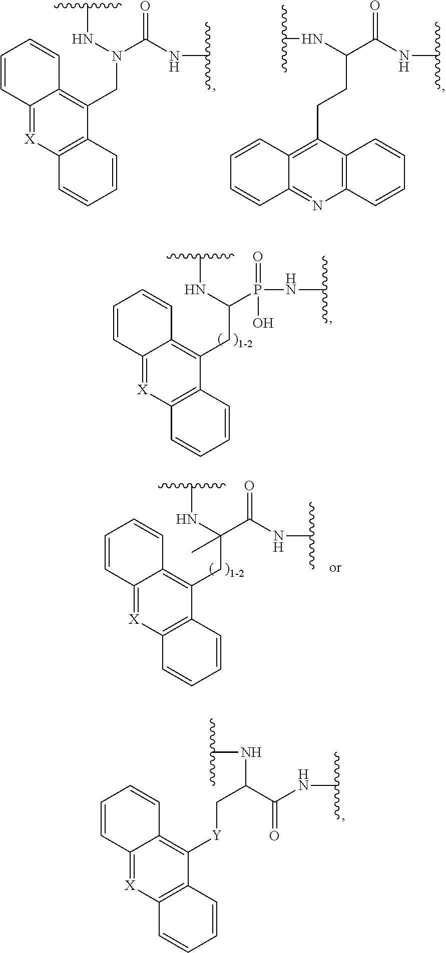

wherein X═CH or N, and Y═NH, S or O, and wherein any of these triaryl/heteroaryl groups is modified optionally with one, more than one, or a combination of halogen, OMe, SMe, NH₂, NO₂, CN, OH, or one or more additional endocyclic ring nitrogen atoms up to a maximum of 5 ring nitrogens.

74. A compound, wherein the compound has Formula III-b or is a salt or a solvate of Formula III-b:

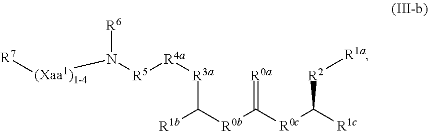

wherein:
R^{0a} is S or 0;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

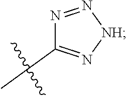 [SMILES: *c1nn[nH]n1]

R^{1b} is —CO₂H, —SO₂H, —SO₃H, —PO₂H, —PO₃H₂, —B(OH)₂, or

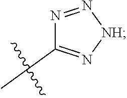 [SMILES: *c1nn[nH]n1]

R^{1c} is —CO₂H, —SO₂H, —SO₃H, —PO₂H, —PO₃H₂, —B(OH)₂, or

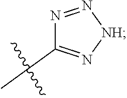 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
—CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})—R^{23c}, in which R²³b is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

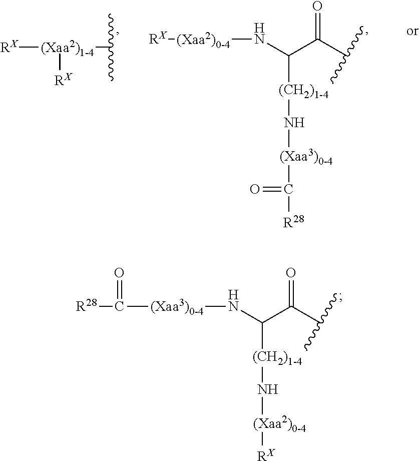

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

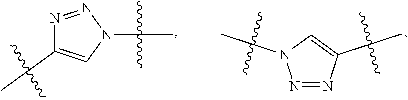 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

75. A compound, wherein the compound has Formula IV-a or is a salt or a solvate of Formula IV-a:

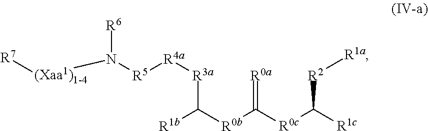

wherein:
R^{0a} is S or O;
R^{0b} is —O—, —S—, —NH—, or CH₃;

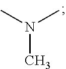

R^{0c} is —O—, —S—, —NH—, or

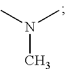

at least one of R^{0b} and R^{0c} is not —NH—;
R^{1a} is —CO₂H, —SO₂H, —SO₃H, —PO₂H, —PO₃H₂, —OPO₃H₂, —OSO₃H, —B(OH)₂, or

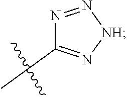 [SMILES: *c1nn[nH]n1]

R^{1b} is —CO₂H, —SO₂H, —SO₃H, —PO₂H, —PO₃H₂, —B(OH)₂, or

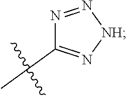 [SMILES: *c1nn[nH]n1]

R^{1c} is —CO₂H, —SO₂H, —SO₃H, —PO₂H, —PO₃H₂, —B(OH)₂, or

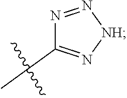 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —O—, —S—, —Se—, —S(O)—, —S(O)₂—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—,

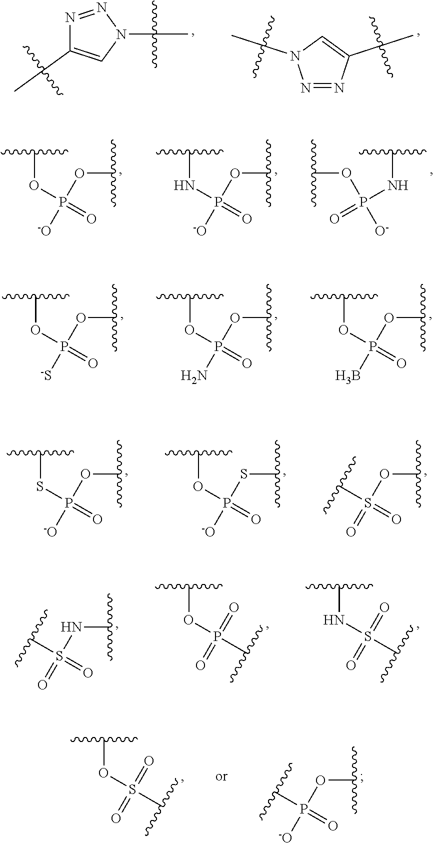

R^{4b} is hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
—CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is:
hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups; or
a carbonyl, a phosphoryl or a sulfonyl group that is linked to the alpha-nitrogen in Xaa¹ to respectively give an amide, phosphoramidate/phosphonamidate, or sulfonamide linkage; or
NHC(O)—, —(NH)₂—C(O)—, —C(O)—(NH)₂—C(O)—, —OC(O)—, —OC(S)—, —NHC(S)—, —NHC(O)C(O)—, or —NH—NH—C(O)—, to enjoin the alpha-nitrogen in Xaa¹;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

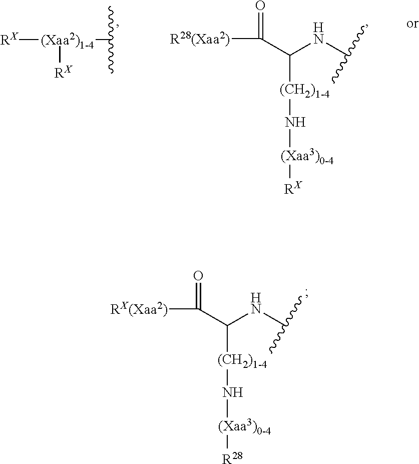

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

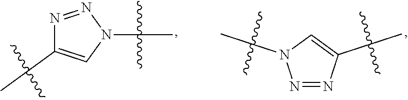 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

76. A compound, wherein the compound has Formula IV-b or is a salt or a solvate of Formula IV-b:

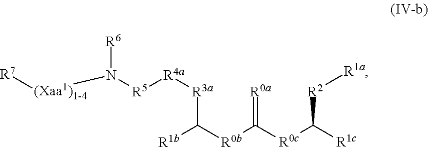

wherein:
R^{0a} is S or O;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or

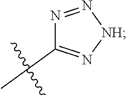 [SMILES: *c1nn[nH]n1]

R^{1b} is —CO₂H, —SO₂H, —SO₃H, —PO₂H, —PO₃H₂, —B(OH)₂, or

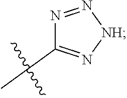 [SMILES: *c1nn[nH]n1]

R^{1c} is —CO₂H, —SO₂H, —SO₃H, —PO₂H, —PO₃H₂, —B(OH)₂, or

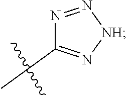 [SMILES: *c1nn[nH]n1]

R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂O—CH₂—, —CH₂—O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—, CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —CH₂SeCH₂—, —CH(COOH)—, —CH₂CH(COOH)—, —CH₂CH(COOH)CH₂—, —CH₂CH₂CH(COOH)—, —CH═CH—, —CH═CHCH₂—, —C≡CCH₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl or alkenylenyl, or a linear or branched, cyclic or acyclic, and/or aromatic or non-aromatic X₂-X₂₀ heteroalkylenyl or heteroalkenylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, —C(O)—N(R^{4b})—O—;
R^{4b} is methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; a linear or branched, cyclic or acyclic, and/or aromatic or non-aromatic X₂-X₁₉ heteroalkyl, heteroalkenyl or heteroalkynyl having only 1-3 heteroatoms; or
—CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})—R^{23c}, in which R^{23b} is phenyl or naphthyl and R^{23c} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is:
hydrogen, methyl, ethyl, or —(CH₂)₀₋₁-(phenyl), wherein 1-5 of the phenyl ring hydrogens are optionally substituted with one or a combination of OH, NH₂, NO₂, halogen, C₁-C₆ alkyl, and/or C₁-C₆ alkoxyl groups; or
a carbonyl, a phosphoryl or a sulfonyl group that is linked to the alpha-nitrogen in Xaa¹ to respectively give an amide, phosphoramidate/phosphonamidate, or sulfonamide linkage; or
NHC(O)—, —(NH)₂—C(O)—, —C(O)—(NH)₂—C(O)—, —OC(O)—, —OC(S)—, —NHC(S)—, —NHC(O)C(O)—, or —NH—NH—C(O)—, to enjoin the alpha-nitrogen in Xaa¹;
Xaa¹ is an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl;
R⁷ is R^{X}-(Xaa²)₀₋₄-,

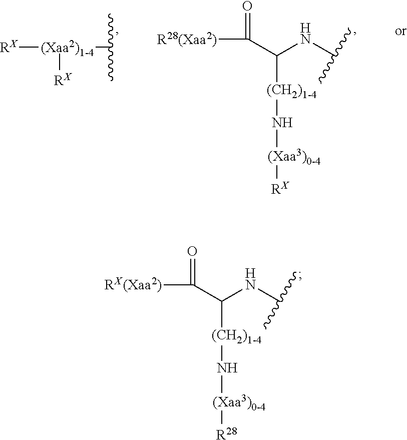

R²⁸ is an albumin binder;
Xaa² and Xaa³, when present, are independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂₀ heteroalkylenyl, heteroalkenylenyl or heteroalkynylenyl; and
each R^{X} is a radiolabeling group independently selected from: a radiometal chelator optionally bound by a metal; an aryl or heteroaryl substituted with a radioisotope; a prosthetic group containing a trifluoroborate; or a prosthetic group containing a silicon-fluorine-acceptor moiety, a fluorophosphate, a fluorosulfate, or a sulfonylfluoride;
and wherein any one or any combination of amide linkages within R⁷-Xaa¹)₁₋₄-N(R⁶)—R⁵—R^{4a}—R^{3a} is optionally independently replaced by linkages selected from the group consisting of —O—, —S—, —Se—, —S(O)—, —S(O)₂—, —NHC(O)—, —C(O)NH—,

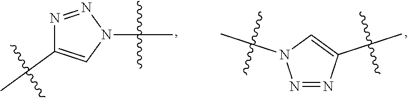 [SMILES: *c1cn(*)nn1 ; *c1cn(*)nn1]

—C(O)—(NH)₂—C(O)—, —OC(O)NH—, —NHC(O)O—, —NHC(O)NH—, —OC(S)NH, —NHC(S)O—, —NHC(S)NH—, —NHC(O)C(O)NH—, —S—S—, —S—CH₂—S—, —NH—NH—C(O)—, and —C(O)—NH—NH—.

77. The compound of any one of Embodiments 72 to 76, wherein R²⁸ is

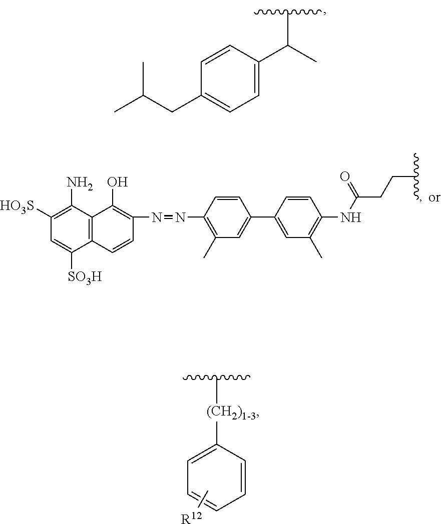

and wherein R¹² is I, Br, F, Cl, H, OH, OCH₃, NH₂, NO₂ or CH₃.

78. The compound of any one of Embodiments 72 to 77, wherein R^{3a} is:
a linear C₃-C₈ alkylenyl, optionally wherein one methylene is replaced with —S—, —O—, —S—CH(CH₃)—, —O—CH(CH₃)—, —CH(CH₃)—S—, —CH(CH₃)—O—, wherein the S and O heteroatoms are spaced apart from other heteroatoms in the compound by at least 2 carbons, and optionally wherein one ethylene is replaced with —CH═CH—, —C≡C—, a 3-6 membered cycloalkylenyl or arylenyl,

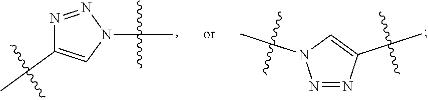 [SMILES: *c1cn(*)nn1 ; *c1cn(*)nn1]

or
—(CH₂)₁₋₃—NH—C(O)—C(R^{3b})₂-, wherein each R^{3b} is independently hydrogen, methyl, or ethyl, or together —C(R^{3b})₂— forms cyclopropylenyl, and which is oriented in the compound as shown:

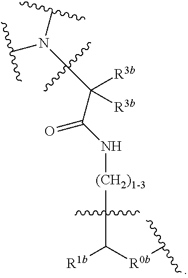

79. The compound of any one of Embodiments 72 to 77, wherein R^{3a} is: —(CH₂)₃—; —(CH₂)₄—; —(CH₂)₅ₐ—; —CH₂—CH═CH—CH₂—; —CH₂—CH₂—CH═CH— wherein the terminal alkenyl carbon is bonded to a carbon in the compound;
—CH₂—C≡C—CH₂—; —C(R^{3b})₂—C(O)—NH—(CH₂)₁₋₂— wherein the leftmost carbon is bonded to a nitrogen of R^{4a} and each R^{3b} is independently hydrogen, methyl, or ethyl, or together —C(R^{3b})₂— forms cyclopropyl-enyl; or —CH₂—CH₂—S—CH(R^{3c})_or —CH₂—CH₂O—CH(R^{3c})—, wherein R^{3c} is hydrogen or methyl.

80. The compound of any one of Embodiments 72 to 77, wherein —R^{4a}—R^{3a}— is:
—C(O)—N(R^{4b})—(CH₂)₁₋₃—R^{3d}—R^{3e}—, wherein R^{3d} is,

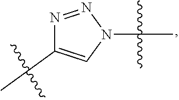 [SMILES: *c1cn(*)nn1]

and wherein R^{3e} is —CH₂—, —(CH₂)₂—, —(CH₂)₂O—CH₂—, —(CH₂)₂—S—CH₂—, —(CH₂)₂O—CH(CH₃)—, or —(CH₂)₂—S—CH(CH₃)—; or
—C(O)—N(R^{4b})—(CH₂)₂-3-R^{3f}—R^{3g}—, wherein R^{3f} is

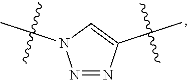 [SMILES: *c1cn(*)nn1]

and wherein R^{3g} is absent, —CH₂—, —(CH₂)₂—, —(CH₂)₀₋₂-O—CH₂—, —(CH₂)₀₋₂-S—CH₂—, —(CH₂)₀₋₂-O—CH(CH₃)—, or —(CH₂)₀₋₂-S—CH(CH₃)—.

81. The compound of any one of Embodiments 72 to 77, wherein R^{3a} is —(CH₂—R^{3h}—(CH—- or —(CH₂)₀₋₂-R^{3h}—(CH₂)₁₋₂-, wherein R^{3h} is:

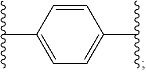 [SMILES: *c1ccc(*)cc1]

82. The compound of any one of Embodiments 72 to 81, wherein no amide linkages within R⁷-Xaa¹),-4-N(R⁶)—R⁵—R^{4a}—R^{3a} are replaced.

83. The compound of any one of Embodiments 72 to 81, wherein only one amide linkage within R⁷-(Xaa¹)₁₋₄ is replaced.

1. A compound of Formula B: 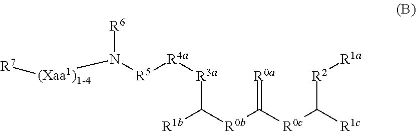
or a salt, a solvate, or a stereoisomer thereof, wherein:
R^{0a} is O or S;
R^{0b} is —NH—;
R^{0c} is —NH—;
R^{1a} is —CO₂H, —SO₂H, —SO₃H, —PO₂H, —PO₃H₂, —OPO₃H₂, —OSO₃H, —B(OH)₂, or 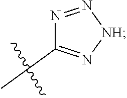 [SMILES: *c1nn[nH]n1]
R^{1b} is —CO₂H, —SO₂H, —SO₃H, —PO₂H, —PO₃H₂, —B(OH)₂, or 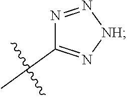 [SMILES: *c1nn[nH]n1]
R^{1c} is —CO₂H, —SO₂H, —SO₃H, —PO₂H, —PO₃H₂, —B(OH)₂, or 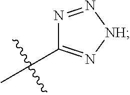 [SMILES: *c1nn[nH]n1]
R² is —CH₂—, —(CH₂)₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, -CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂—O—CH₂—, —CH₂O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH2-, wherein HC[CH₂]CH represents a cyclopropyl ring;
R³a is —(CH₂)₅—, —CH₂—O—(CH₂)₂—, —(CH₂)₃—O—, —CH₂—S—CH₂—CH(CO₂H)—, —(CH₂)₃—CH(CO₂H)—, —CH₂—O—CH₂—CH(CO₂H)—, —CH₂—Se—CH₂—CH(CO₂H)—, —CH₂—S—CH(CO₂H)—CH₂—, —(CH₂)₂—CH(CO₂H)—CH₂—, —CH₂O—CH(CO₂H)—CH₂—, —CH₂—Se—CH(CO₂H)—CH₂—, —CH₂—CH(CO₂H)—(CH₂)₂—, —(CH₂)₂—CH(CO₂H)—, —CH₂—CH(CO₂H)—CH₂—, —(CH₂)₁₋₂— R^{3h}—(CH₂)₀₋₂—, —(CH₂)₀-2-R^{3h}—(CH₂)₁₋₂— or —(CH₂)i-3-NH—C(O)—C(R^{3b})₂—;
R^{3h} is 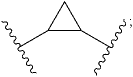 [SMILES: *C1CC1*]
each R^{3b} is, independently, hydrogen, methyl, or ethyl, or together —C(R^{3b})₂— forms cyclopropylenyl;
R^{4a} is —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, N3, CN, SMe, CF₃, CHF₂, halogen, C₁-C₆ alkyl, or C₁-C₆ alkoxyl groups;
R⁵ is —(CH₂)₀₋₃CH(R¹⁰)(CH₂)₀₋₃—, wherein R¹⁰ is:
a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms; or
—CH₂R²³a, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})-R²³c, in which R^{23b} is phenyl or naphthyl and R^{23′} is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R⁸)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
R^{X}-(Xaa²)₀₋₄-, 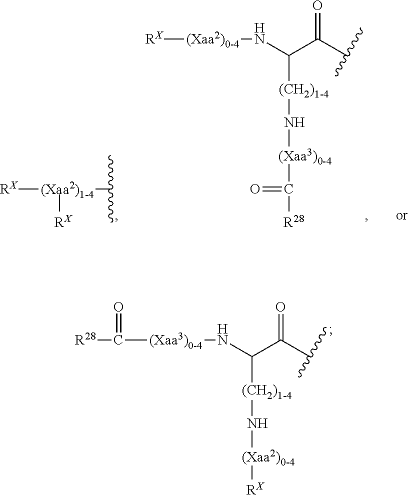
R²⁸ is an albumin binder;
Xaa² and Xaa³, when present, are each independently —N(R¹³)R¹⁴C(O)—, wherein each R¹³ is independently hydrogen or methyl, and wherein each R¹⁴ is, independently, a linear or branched, cyclic or acyclic, and/or aromatic or non-aromatic C₁-C₂₀ alkylenyl, alkenylenyl or alkynylenyl, or a linear or branched, cyclic or acyclic, and/or aromatic or non-aromatic C₂-C₂₀ heteroalkylenyl, heteroalkenylenyl or heteroalkynylenyl; and
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof, and
wherein any one or any combination of amide linkages within R⁷-(Xaa¹)₁₋₄-N(R⁶)—R⁵—R^{4a}—R³a is optionally independently replaced by linkages selected from the group consisting of, —NHC(S)—, —C(S)NH—, —NHC(O)—, 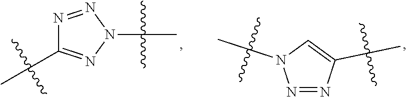 [SMILES: *c1nnn(*)n1 ; *c1cn(*)nn1]
—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

2. The compound of claim 1, wherein R³a is —CH₂—NH—C(O)—CH₂—, —CH₂—O—(CH₂)₂—, —(CH₂)₃—O—, —CH₂—S—CH₂—CH(CO₂H)—, —(CH₂)-2-R^{3h}- or (CH₂)₀₋₂—R^{3h}—(CH₂)₁₋₂—; and wherein R^{3h} is

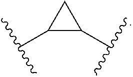 [SMILES: *C1CC1*]

3. The compound of claim 1, wherein R² is —CH₂—, —(CH₂)₂—, —CH₂CHF—, —CHFCH₂—, —(CH₂)₃—, —CH₂OCH₂—, or —CH₂SCH₂—.

4. The compound of claim 1, wherein R^{4a} is —C(O)NH—.

5. The compound of claim 1, wherein R^{4b} is benzyl optionally substituted with one or a combination of OH, NH₂, NO₂, N₃, CN, SMe, CF₃, CHF₂, halogen, C₁-C₆ alkyl, and/or C₁-C₆ alkoxyl groups.

6. The compound of claim 1, wherein R^{4b} is benzyl optionally para-substituted with a halogen.

7. The compound of claim 1, wherein R⁵ is —CH(R¹⁰)—; and wherein R¹⁰ is 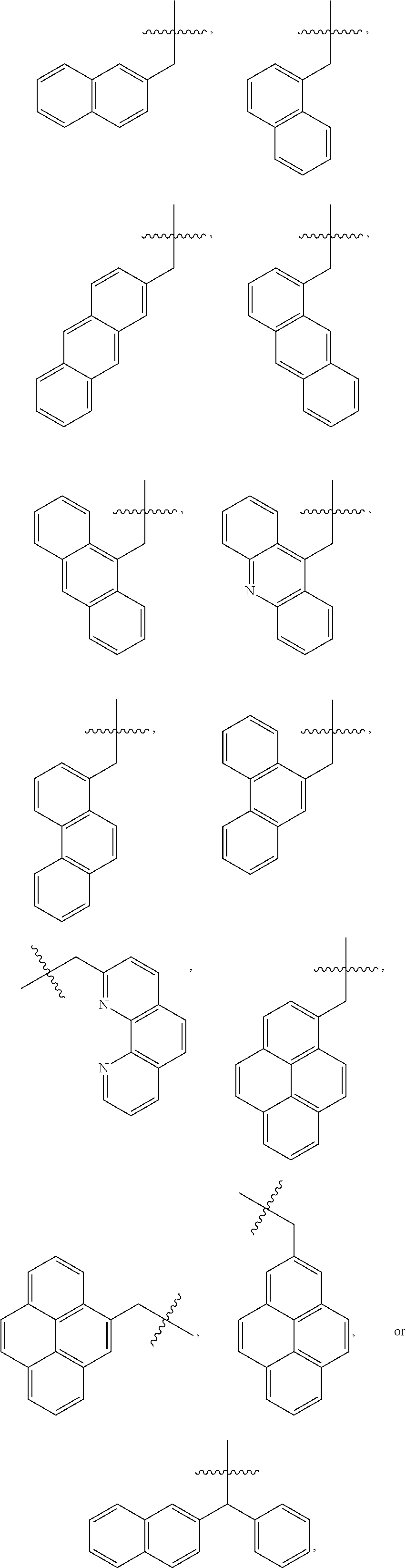
each R¹⁰ is optionally substituted with one or more substituent selected from halogen, —OMe, —SMe, —NH₂, —NO₂, —CN, or —OH; and up to 5 carbon ring atoms of an endocyclic ring of R¹⁰ is optionally replaced with a nitrogen atom such that R¹⁰ can contain up to a maximum of 5 ring nitrogens.

8. The compound of claim 1, wherein R¹⁰ is

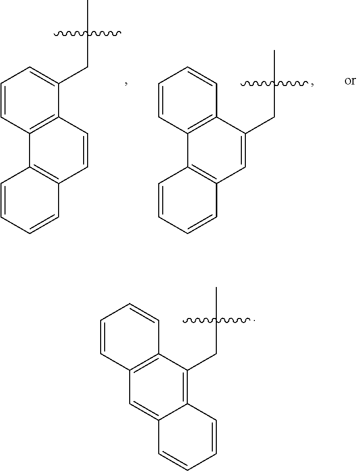 [SMILES: *Cc1cccc2c1ccc1ccccc12 ; *Cc1cc2ccccc2c2ccccc12 ; *Cc1c2ccccc2cc2ccccc12]

9. The compound of any one of claim 1, wherein -(Xaa¹)₁₋₄-N(R⁶)—R⁵—R^{4a}— is

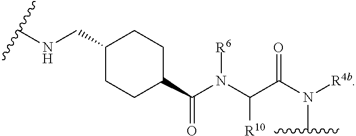

10. The compound of claim 1, wherein: R⁷ is:
R^{X}-(Xaa²)₀₋₄ is absent; 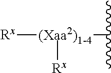
wherein (Xaa²)₁₋₄ is a tripeptide; or 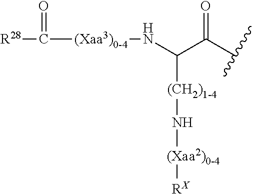
wherein (Xaa²)₀₋₄ is absent;
R²⁸ is 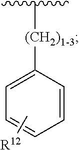
R¹² is I, Br, F, Cl, H, —OH, —OCH₃, —NH₂, or —CH₃; and
R^{X} is a radiometal chelator optionally bound to a radiometal, or a prosthetic group containing a trifluoroborate.

11. The compound of claim 1, wherein:
R⁷ is R^{X}-(Xaa²)₀₋₄ or 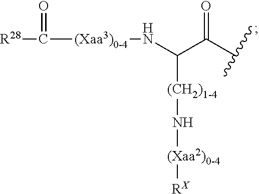
R²⁸ is 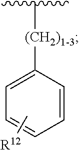
Xaa² is absent;
Xaa³ is absent or is a single amino acid residue; and
R¹² is —OCH₃ or C₁.

12. The compound of claim 1, wherein R⁷ is R^{X}-(Xaa²)₀₋₄- and R^{X} is DOTA, optionally chelated with a radiometal.

13. The compound of claim 1, wherein:
R⁷ is 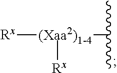
each R^{X} is independently —C(O)—(CH₂)₀₋₅R¹⁸—(CH₂)₁₋₅R¹⁷BF₃;
R¹⁸ is absent, 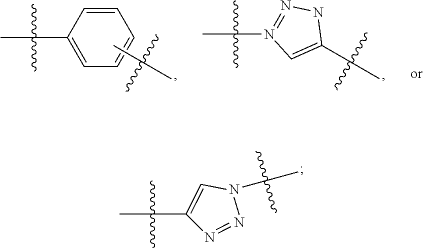
R¹⁷BF₃ is 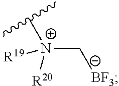
and
R¹⁹ and R²⁰ are each independently C₁-C₅ linear or branched alkyl groups.

14. The compound of claim 1, wherein R^{0a} is O; R^{1a} is —CO₂H; R^{1b} is —CO₂H; and R^{1c} is —CO₂H.

15. The compound of claim 2, wherein:
R^{0a} is O;
R^{1a} is —CO₂H;
R^{1b} is —CO₂H;
R^{1c} is —CO₂H;
R² is —CH₂—, —CH₂CHF—, —CHFCH₂—, —(CH₂)₂—, —(CH₂)₃—, —CH₂OCH₂—, or —CH₂SCH₂—;
(Xaa¹)₁₋₄-N(R⁶)—R⁵—R^{4a}— is 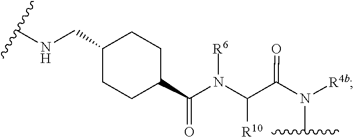
R^{4b} is hydrogen, methyl or ethyl;
R⁶ is hydrogen, methyl or ethyl;
R¹⁰ is 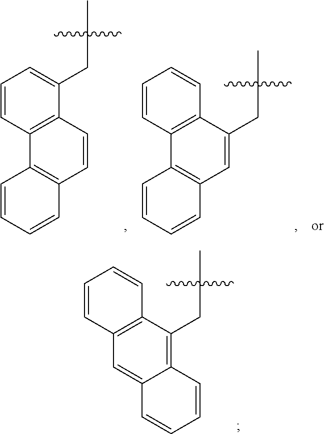 [SMILES: *Cc1cccc2c1ccc1ccccc12 ; *Cc1cc2ccccc2c2ccccc12 ; *Cc1c2ccccc2cc2ccccc12]
R⁷ is R^{X}-(Xaa²)₀₋₄ or 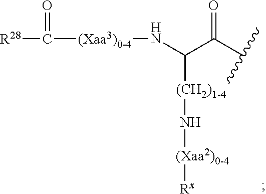
R²⁸ is 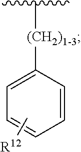
Xaa³ is absent or is a single amino acid residue; and
Xaa² is absent;
R¹² is —OCH₃ or C₁; and
R^{X} is a radiometal chelator optionally bound to a radiometal.

16. The compound of a claim 1, wherein the radiometal chelator is selected from Table 2; and wherein the radiometal chelator is optionally bound to a radiometal.

17. The compound of a claim 1, wherein the radiolabeling group is a prosthetic group containing a trifluoroborate.

18. The compound of claim 1 selected from: 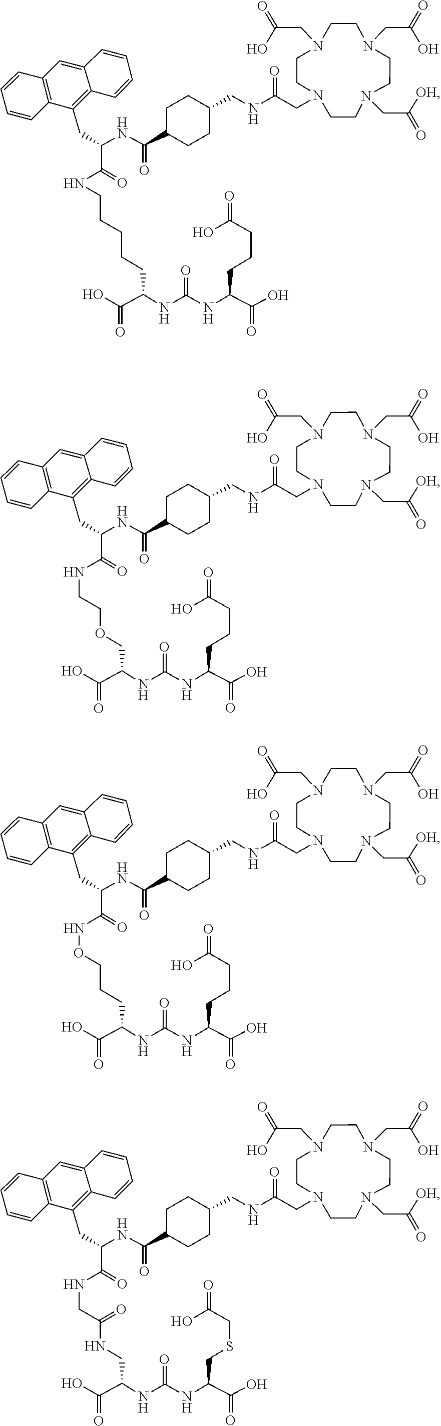 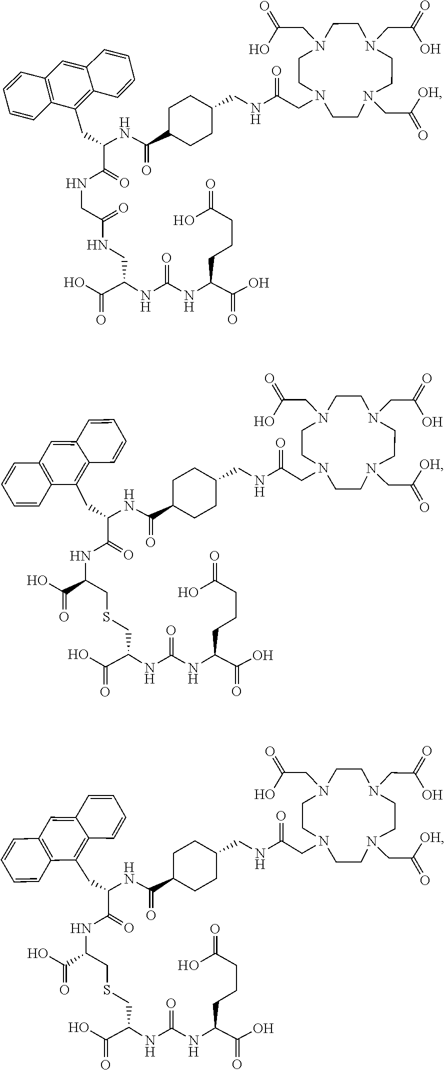 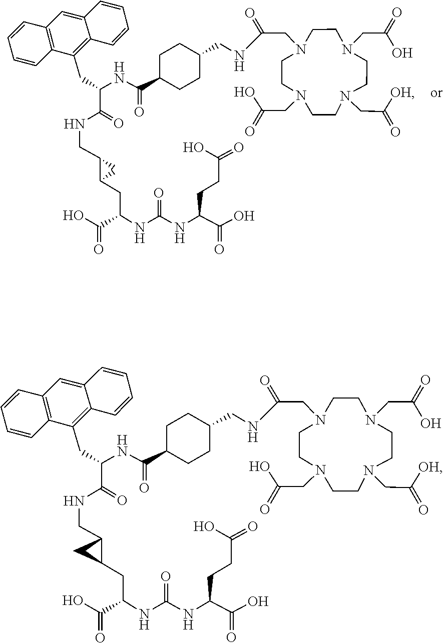 [SMILES: O=C(O)CC[C@H](NC(=O)N[C@@H](C[C@@H]1C[C@H]1CNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CC[C@H](NC(=O)N[C@@H](C[C@H]1C[C@@H]1CNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O]
or a salt or a solvate thereof, wherein each compound is optionally bound to a radiometal.

19. The compound of claim 1, wherein the compound is: 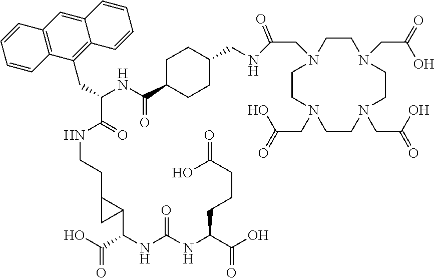 [SMILES: O=C(O)CCC[C@H](NC(=O)N[C@H](C(=O)O)C1CC1CCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O]
or a salt, a solvate, or a stereoisomer thereof, wherein the compound is optionally bound to a radiometal.

20. The compound of claim 19, wherein the compound is: 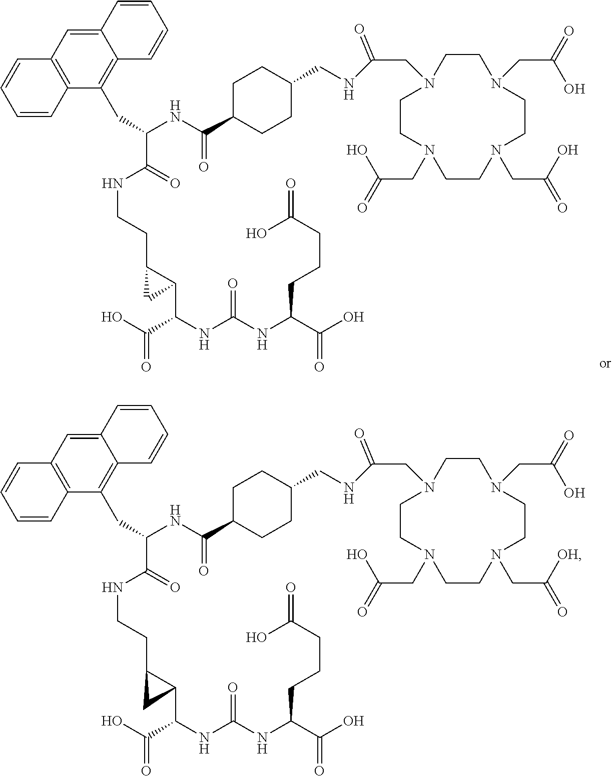
or a salt or a solvate thereof, wherein each compound is optionally bound to a radiometal.

21. A compound of Formula A: 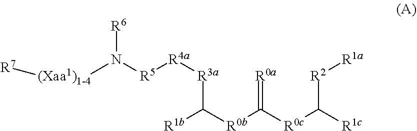
or a salt, a solvate, or a stereoisomer thereof, wherein:
R^{0a} is O or S; 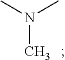
R^{0b} is —O—, —S—, —NH—, or 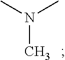
R^{0C} is —O—, —S—, —NH—, or
wherein at least one of R^{0b} and R^{0C} is not —NH—;
R^{1a} is —CO₂H, —SO₂H, —SO₃H, —PO₂H, —PO₃H₂, —OPO₃H₂, —OSO₃H, —B(OH)₂, or 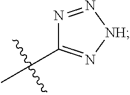 [SMILES: *c1nn[nH]n1]
R^{1b} is —CO₂H, —SO₂H, —SO₃H, —PO₂H, —PO₃H₂, —B(OH)₂, or 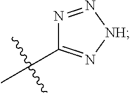 [SMILES: *c1nn[nH]n1]
R^{1c} is —CO₂H—SO₂H—SO₃H, —PO₂H, —PO₃H₂, —B(OH)₂, or 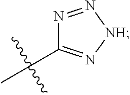 [SMILES: *c1nn[nH]n1]
R² is —CH₂—, —CH(OH)—, —CHF—, —CF₂—, —CH(CH₃)—, —C(CH₃)₂—, —CH₂CH(OH)—, —CH₂CHF—, —CHFCH₂—, —CF₂CH₂—, —CH₂CF₂—, —CH(OH)CH₂—, —CH(CH₃)CH₂—, —CH₂CH(CH₃)—, —C(CH₃)₂CH₂—, —CH₂C(CH₃)₂—, —CH₂CH(OH)CH₂—, —CH₂CHFCH₂—, —(CH₂)₂CH(OH)—, —(CH₂)₂CHF—, —(CH₂)₃—, —CH₂OCH₂—, —CH₂SCH₂—, —CHFCH₂CH₂—, —CH(OH)CH₂CH₂—, —CH(CH₃)CH₂CH₂—, —CH₂CH(CH₃)CH₂—, —CH₂CH₂CH(CH₃)—, —C(CH₃)₂CH₂CH₂—, —CH₂C(CH₃)₂CH₂—, —CH₂CH₂C(CH₃)₂—, —CH(CH₃)—O—CH₂—, —C(CH₃)₂—O—CH₂—, —CH₂O—CH(CH₃)—, —CH₂—O—C(CH₃)₂—, —CH₂—S(O)—CH₂—, —CH₂—S(O)₂—CH₂—, —CH(CH₃)—S—CH₂—, —C(CH₃)₂—S—CH₂—, —CH₂—S—CH(CH₃)—, —CH₂—S—C(CH₃)₂—, —CH(CH₃)—S(O)—CH₂—, —C(CH₃)₂—S(O)—CH₂—, —CH₂—S(O)—CH(CH₃)—, —CH₂—S(O)—C(CH₃)₂—, —CH(CH₃)—S(O)₂—CH₂—, —C(CH₃)₂—S(O)₂—CH₂—, —CH₂—S(O)₂—CH(CH₃)—, —CH₂—S(O)₂—C(CH₃)₂—, —CH₂—NH—C(O)—, —C(O)—NH—CH₂—, —C(O)—NH—CH(CH₃)—, —C(O)—NH—C(CH₃)₂—, —HC[CH₂]CH—, or —HC[CH₂]CHCH₂—, wherein HC[CH₂]CH represents a cyclopropyl ring;
R^{3a} is a linear or branched, cyclic or acyclic, and/or aromatic or non-aromatic C₁-C₂₀ alkylenyl, alkenylenyl, or alkynylenyl, or a linear or branched, cyclic or acyclic, and/or aromatic or non-aromatic X₂-X₂₀ heteroalkylenyl, heteroalkenylenyl, or heteroalkynylenyl, wherein each R^{3a} is optionally substituted;
R^{4a} is —O—, —S—, —Se—, —S(O)—, —S(O)₂—, 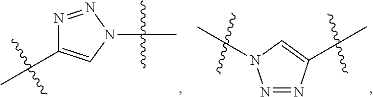 [SMILES: *c1cn(*)nn1 ; *c1cn(*)nn1]
—S—S—, —S—CH₂—S—, —N(R^{4b})—C(O)—, —C(O)—N(R^{4b})—, —C(O)—N(R^{4b})—NH—C(O)—, —C(O)—NH—N(R^{4b})—C(O)—, —O—C(O)—N(R^{4b})—, —N(R^{4b})—C(O)—O—, —N(R^{4b})—C(O)—NH—, —NH—C(O)—N(R^{4b})—, —O—C(S)—N(R^{4b})—, —N(R^{4b})—C(S)—O—, —N(R^{4b})—C(S)—NH—, —NH—C(S)—N(R^{4b})—, —N(R^{4b})—C(O)—C(O)—NH—, —NH—C(O)—C(O)—N(R^{4b})—, —N(R^{4b})—NH—C(O)—, —NH—N(R^{4b})—C(O)—, —C(O)—N(R^{4b})—NH—, —C(O)—NH—N(R^{4b})—, or —C(O)—N(R^{4b})—O—;
R^{4b} is hydrogen, methyl, ethyl, or —(CH₂)₀₋₁-(phenyl), wherein 1-5 of the phenyl ring hydrogens are optionally substituted with —OH, —NH₂, —NO₂, halogen, C₁-C₆ alkyl, or C₁-C₆ alkoxyl groups;
R⁵ is —(CH₂)₀₋₃CH(R¹o)(CH₂)₀₋₃—, wherein R¹⁰ is:
a linear or branched, cyclic or acyclic, and/or aromatic or non-aromatic C₂-C₁₉ alkyl, alkenyl or alkynyl; or a linear or branched, cyclic or acyclic, and/or aromatic or non-aromatic C₂-C₁₉ heteroalkyl, heteroalkenyl or heteroalkynyl having 1-3 heteroatoms;
—CH₂R^{23a}, in which R^{23a} is an optionally substituted C₄-C₁₆ aromatic ring or partially or fully aromatic fused ring system, wherein 0-5 carbons in the aromatic ring or the partially or fully aromatic fused ring system are independently replaced with N, S and/or O heteroatoms, and wherein the optional substitutions are selected from —OH, —NH₂, —NO₂, halogen, —SMe, —CN, C₁-C₆ alkyl, and/or C₁-C₆ alkoxyl groups; or
—CH(R^{23b})-R²³c, in which R^{23b} is phenyl or naphthyl and R²³c is phenyl or naphthyl, wherein 0-5 carbons in each naphthyl ring and 0-3 carbons in each phenyl ring are independently replaced with N, S and/or O heteroatoms, and wherein each naphthyl and each phenyl are independently optionally substituted with —OH, —NH₂, —NO₂, halogen, —SMe, —CN, C₁-C₆ alkyl, and/or C₁-C₆ alkoxyl groups;
R⁶ is hydrogen, methyl, or ethyl;
each Xaa¹ is, independently, an amino acid of formula —N(R′)R⁹C(O)—, wherein each R⁸ is independently hydrogen or methyl, and wherein each R⁹ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic C₂-C₂₀ heteroalkylenyl, heteroalkenylenyl or heteroalkynylenyl;
R⁷ is R^{X}-(Xaa²)₀₋₄-, 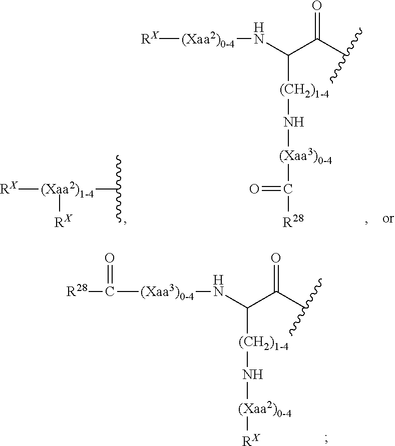
R²⁸ is an albumin binder;
Xaa² and Xaa³, when present, are each independently —N(R¹³)R¹⁴C(O)—, wherein each R¹³ is independently hydrogen or methyl, and wherein each R¹⁴ is independently: a linear or branched, cyclic or acyclic, and/or aromatic or non-aromatic C₁-C₂₀ alkylenyl, alkenylenyl or alkynylenyl; or a linear or branched, cyclic or acyclic, and/or aromatic or non-aromatic X₂-X₂0 heteroalkylenyl, heteroalkenylenyl or heteroalkynylenyl;
each R^{X} is a radiolabeling group independently selected from: a radiometal chelator optionally bound by a radiometal; an aryl or heteroaryl substituted with a radiohalogen; a prosthetic group containing a trifluoroborate; a prosthetic group containing a silicon-fluorine-acceptor moiety; or a prosthetic containing a fluorophosphate, fluorosulfate, sulfonylfluoride, or a combination thereof, and
wherein any one or any combination of amide linkages within R⁷-(Xaa¹)₁₋₄-N(R⁶)—R⁵—R^{4a}—R³a is optionally independently replaced by linkages selected from the group consisting of, —NHC(S)—, —C(S)NH—, —NHC(O)—, 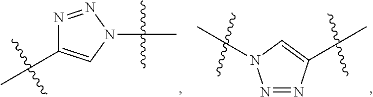 [SMILES: *c1cn(*)nn1 ; *c1cn(*)nn1]
—OC(O)NH—, —NHC(O)O—, —NHC(O)NH—, —OC(S)NH, —NHC(S)O—, —NHC(S)NH—, —NHC(O)C(O)NH—, —NH—NH—C(O)—, and —C(O)—NH—NH—.

22-39. (canceled)

40. The compound of claim 21 selected from: 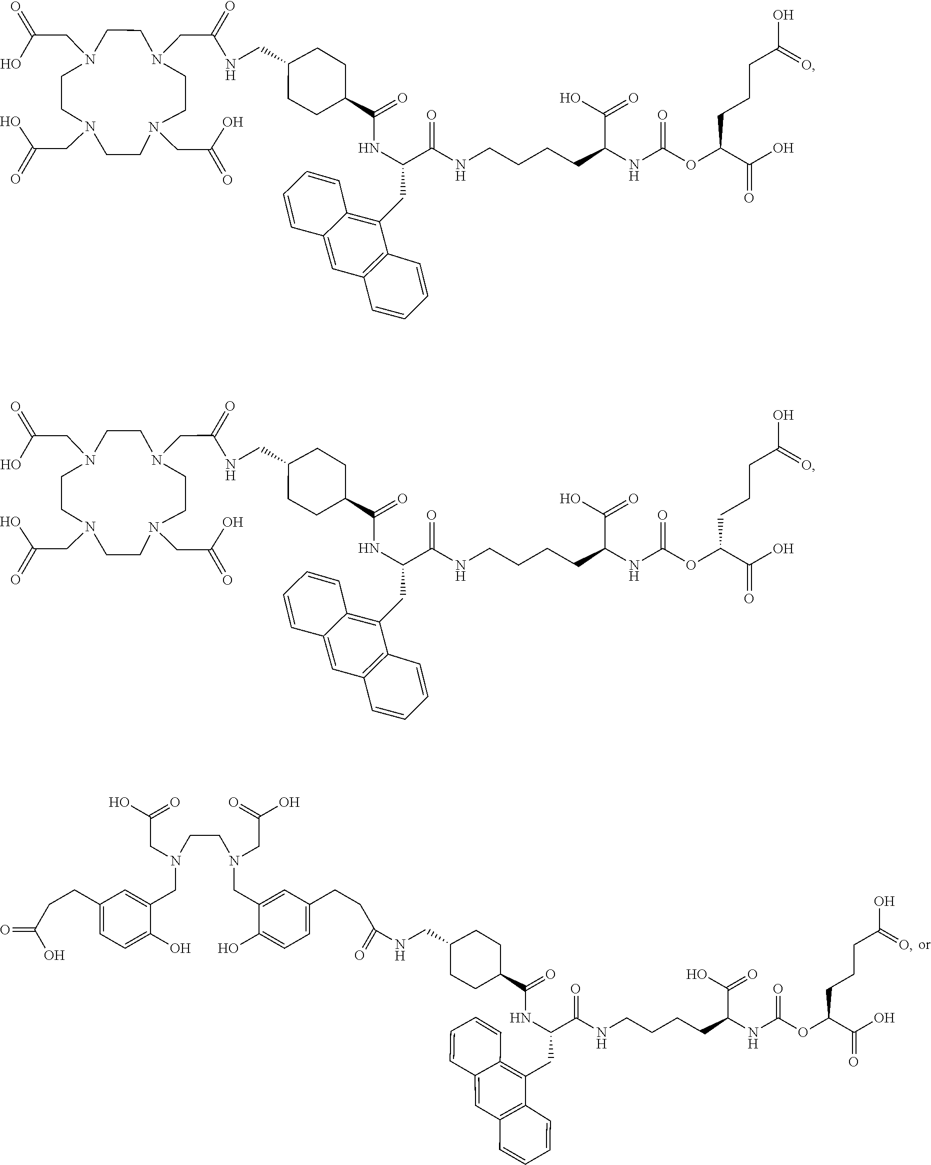 [SMILES: O=C(O)CCC[C@H](OC(=O)N[C@@H](CCCCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CCC[C@@H](OC(=O)N[C@@H](CCCCNC(=O)[C@H](Cc1c2ccccc2cc2ccccc12)NC(=O)[C@H]1CC[C@H](CNC(=O)CN2CCN(CC(=O)O)CCN(CC(=O)O)CCN(CC(=O)O)CC2)CC1)C(=O)O)C(=O)O ; O=C(O)CCC[C@H](OC(=O)N[C@@H](CCCCNC(=O)[C@H](Cc1c2ccccc2cc2ccccc12)NC(=O)[C@H]1CC[C@H](CNC(=O)CCc2ccc(O)c(CN(CCN(CC(=O)O)Cc3cc(CCC(=O)O)ccc3O)CC(=O)O)c2)CC1)C(=O)O)C(=O)O] 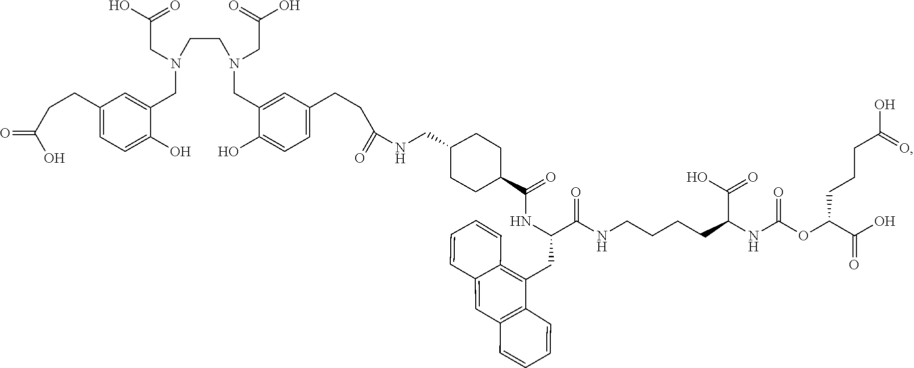 [SMILES: O=C(O)CCC[C@@H](OC(=O)N[C@@H](CCCCNC(=O)[C@H](Cc1c2ccccc2cc2ccccc12)NC(=O)[C@H]1CC[C@H](CNC(=O)CCc2ccc(O)c(CN(CCN(CC(=O)O)Cc3cc(CCC(=O)O)ccc3O)CC(=O)O)c2)CC1)C(=O)O)C(=O)O]
or a salt or a solvate thereof, wherein each compound is optionally bound to a radiometal.

41. The compound of claim 1, wherein the radiometal is selected from the group consisting of ¹⁷⁷Lu, ¹¹¹In, ²¹³Bi, ⁶⁸Ga, ⁶⁷Ga ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, ¹⁴⁹Tb, ¹⁵²Tb, ¹⁵⁵Tb, ¹⁶¹Tb, ¹⁶⁵Er, ²¹²Bi, ²²⁷Th, ⁶⁴Cu, and ⁶⁷Cu.

42. The compound of claim 1, wherein the radiometal chelator is chelated with ⁶⁸Ga, ¹⁷⁷Lu, ¹⁶¹Tb, or ²²⁵Ac.

43. A method of treating a PSMA-expressing condition or disease, comprising administering to a patient in need thereof a therapeutically effective amount of a compound of claim 1.

44. The method of claim 43, wherein the PSMA-expressing condition or disease is a cancer selected from the group consisting of prostate cancer, renal cancer, breast cancer, thyroid cancer, gastric cancer, colorectal cancer, bladder cancer, pancreatic cancer, lung cancer, liver cancer, brain tumor, melanoma, neuroendocrine tumor, ovarian cancer or sarcoma.

45. (canceled)

46. A method of imaging PSMA-expressing tissues comprising administering an effective amount of a compound of claim 1 to a patient in need of such imaging; and imaging the tissues of the subject.

47. The method of claim 46, wherein said imaging is performing PET or SPECT imaging.

48. The compound of claim 21, wherein the radiometal is selected from the group consisting of ¹⁷⁷Lu, ¹¹¹In, ²¹³Bi, ⁶⁸Ga, ⁶⁷Ga, ²⁰³Pb, ²¹²Pb, ⁴⁴Sc, ⁴⁷Sc, ⁹⁰Y, ⁸⁶Y, ²²⁵Ac, ^{117m}Sn, ¹⁵³Sm, 149Tb ¹⁵²Tb, ¹⁵⁵Tb ¹⁶¹Tb, ¹⁶⁵Er, ²¹²Bi, ²²⁷Th, ⁶⁴Cu, and ⁶⁷Cu.

49. The compound of claim 21, wherein the radiometal chelator is chelated with ⁶⁸Ga, ¹⁷⁷Lu, ¹⁶¹Tb, or ²²⁵Ac.
